(19) United States
(12) Patent Application Publication
(10) Pub. No.: US 2021/0032649 A1
(43) Pub. Date: Feb. 4, 2021

(54) METHODS AND COMPOSITIONS FOR SHORT STATURE PLANTS THROUGH MANIPULATION OF GIBBERELLIN METABOLISM TO INCREASE HARVESTABLE YIELD

(71) Applicant: MONSANTO TECHNOLOGY LLC, St. Louis, MO (US)

(72) Inventors: Sivalinganna MANJUNATH, Chesterfield, MO (US); Linda A. RYMARQUIS, High Ridge, MO (US); Thomas L. SLEWINSKI, Chesterfield, MO (US)

(73) Assignee: MONSANTO TECHNOLOGY LLC, St. Louis, MO (US)

(21) Appl. No.: 16/967,038

(22) PCT Filed: Feb. 15, 2019
(86) PCT No.: PCT/US19/18128

§ 371 Date: Aug. 3, 2020

Related U.S. Application Data
(60) Provisional application No. 62/631,412, filed on Feb. 15, 2018.

(51) Int. Cl. C12N 15/82

(52) U.S. Cl. CPC C12N 15/8261

(57) ABSTRACT

The present disclosure provides compositions and methods for altering gibberellin (GA) content in corn or other cereal plants. Methods and compositions are also provided for altering the expression of genes related to gibberellin biosynthesis through suppression, mutagenesis and/or editing of specific subtypes of GA20 or GA3 oxidase genes. Modified plant cells and plants having a suppression element or mutation reducing the expression or activity of a GA oxidase gene are further provided comprising reduced gibberellin levels and improved characteristics, such as reduced plant height and increased lodging resistance, but without off-types.

### CROSS REFERENCE TO RELATED APPLICATIONS

This application claims the benefit of U.S. Provisional Application No. 62/631,412, filed Feb. 15, 2018, which is incorporated herein by reference in its entirety.

### INCORPORATION OF SEQUENCE LISTING

The sequence listing file named “P34603WO00_SEQ.txt” which is 382 kilobytes (measured in MS-WINDOWS) and was created on Feb. 14, 2019, is submitted herewith and incorporated herein by reference in its entirety.

### BACKGROUND

### Field

The present disclosure relates to compositions and methods for improving traits, such as lodging resistance and increased yield, in monocot or cereal plants including corn.

### Related Art

Gibberellins (gibberellic acids or GAs) are plant hormones that regulate a number of major plant growth and developmental processes. Manipulation of GA levels in semi-dwarf wheat, rice and sorghum plant varieties led to increased yield and reduced lodging in these cereal crops during the 20^{th} century, which was largely responsible for the Green Revolution. However, successful yield gains in other cereal crops, such as corn, have not been realized through manipulation of the GA pathway. Indeed, some mutations in the GA pathway genes have been associated with various off-types in corn that are incompatible with yield, which has led researchers away from finding semi-dwarf, high-yielding corn varieties via manipulation of the GA pathway.

There continues to be a need in the art for the development of monocot or cereal crop plants, such as corn, having increased yield and/or resistance to lodging.

### SUMMARY

In an aspect, the present disclosure provides a recombinant DNA construct comprising a transcribable DNA sequence encoding a non-coding RNA molecule, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein in a monocot or cereal plant or plant cell, the endogenous GA oxidase protein being at least 80%, at least 85%, at least 90%, at least 95%, at least 96%, at least 97%, at least 98%, at least 99%, at least 99.5%, or 100% identical to SEQ ID NO: 9, 12, 15, 30 or 33, and wherein the transcribable DNA sequence is operably linked to a plant-expressible promoter.

In another aspect, the present disclosure provides a recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in a monocot or cereal plant or plant cell, the endogenous GA20 oxidase protein being at least 80%, at least 85%, at least 90%, at least 95%, at least 96%, at least 97%, at least 98%, at least 99%, at least 99.5%, or 100% identical to SEQ ID NO: 9, and wherein the transcribable DNA sequence is operably linked to a plant-expressible promoter.

In an aspect, the present disclosure provides a recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in a monocot or cereal plant or plant cell, the endogenous GA20 oxidase protein being at least 80%, at least 85%, at least 90%, at least 95%, at least 96%, at least 97%, at least 98%, at least 99%, at least 99.5%, or 100% identical to SEQ ID NO: 15, and wherein the transcribable DNA sequence is operably linked to a plant-expressible promoter.

In another aspect, the present disclosure provides a recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in a monocot or cereal plant or plant cell, the endogenous GA3 oxidase protein being at least 80%, at least 85%, at least 90%, at least 95%, at least 96%, at least 97%, at least 98%, at least 99%, at least 99.5%, or 100% identical to SEQ ID NO: 30 or 33, and wherein the transcribable DNA sequence is operably linked to a plant-expressible promoter.

In an aspect, the present disclosure provides a recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in a monocot or cereal plant or plant cell, the endogenous GA20 oxidase protein being at least 80%, at least 85%, at least 90%, at least 95%, at least 96%, at least 97%, at least 98%, at least 99%, at least 99.5%, or 100% identical to SEQ ID NO: 12, and wherein the transcribable DNA sequence is operably linked to a plant-expressible promoter.

In another aspect, the present disclosure provides a recombinant DNA construct comprising a transcribable DNA sequence encoding a non-coding RNA molecule, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous protein in a monocot or cereal plant or plant cell, the endogenous protein being at least 80%, at least 85%, at least 90%, at least 95%, at least 96%, at least 97%, at least 98%, at least 99%, at least 99.5%, or 100% identical to SEQ ID NO: 86, 90, 94, 97, 101, 104, 108, 112, 116, 118, 121, 125, 129, 133, or 136, and wherein the transcribable DNA sequence is operably linked to a plant-expressible promoter. In a further aspect, the present disclosure also provides a transformation vector comprising a recombinant DNA construct disclosed herein. In a further aspect, the present disclosure also provides a transgenic monocot or cereal plant, plant part or plant cell comprising a recombinant DNA construct disclosed here. In one aspect, a transgenic corn plant, plant part or plant cell is provided. In another aspect, a method is provided for producing a transgenic cereal plant, comprising: (a) transforming at least one cell of an explant with a recombinant DNA construct disclosed herein, and (b) regenerating or developing the transgenic cereal plant from the transformed explant. In another aspect, a cereal plant is transformed via Agrobacterium mediated transformation or particle bombardment.

In an aspect, the present disclosure provides a method for lowering the level of at least one active GA molecule in the stem or stalk of a corn or cereal plant comprising: suppressing one or more GA3 oxidase or GA20 oxidase genes with a recombinant DNA construct in one or more tissues of the transgenic cereal or corn plant.

In another aspect, the present disclosure provides a transgenic corn or cereal plant comprising a recombinant DNA construct, wherein the recombinant DNA construct comprises a transcribable DNA sequence encoding a non-coding RNA molecule that targets at least one endogenous GA20 or GA3 oxidase gene for suppression, the transcribable DNA sequence being operably linked to a plant-expressible promoter, and wherein the transgenic monocot or cereal plant has a shorter plant height relative to a wild-type control plant.

In an aspect, the present disclosure provides a cereal plant comprising a mutation at or near an endogenous GA oxidase gene introduced by a mutagenesis technique, wherein the expression level of the endogenous GA oxidase gene is reduced or eliminated in the cereal plant, and wherein the cereal plant has a shorter plant height relative to a wild-type control plant.

In another aspect, the present disclosure provides a corn or cereal plant comprising a genomic edit introduced via a targeted genome editing technique at or near the locus of an endogenous GA oxidase gene, wherein the expression level of the endogenous GA oxidase gene is reduced or eliminated relative to a control plant, and wherein the edited cereal plant has a shorter plant height relative to the control plant.

In an aspect, the present disclosure provides a composition comprising a guide RNA, wherein the guide RNA comprises a guide sequence that is at least 95%, at least 96%, at least 97%, at least 99%, or 100% identical or complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of a target DNA sequence at or near the genomic locus of an endogenous GA oxidase gene of a cereal plant. In one aspect, a composition further comprises an RNA-guided endonuclease.

In another aspect, the present disclosure provides a recombinant DNA construct comprising a transcribable DNA sequence encoding a non-coding guide RNA molecule, wherein the guide RNA molecule comprises a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of a target DNA sequence at or near the genomic locus of an endogenous GA oxidase gene of a corn or cereal plant.

In an aspect, the present disclosure provides a recombinant DNA donor template comprising at least one homology sequence, wherein the at least one homology sequence is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence, wherein the target DNA sequence is a genomic sequence at or near the genomic locus of an endogenous GA oxidase gene of a corn or cereal plant.

In another aspect, the present disclosure provides a recombinant DNA donor template comprising two homology arms including a first homology arm and a second homology arm, wherein the first homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a first flanking DNA sequence, wherein the second homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a second flanking DNA sequence, and wherein the first flanking DNA sequence and the second flanking DNA sequence are genomic sequences at or near the genomic locus of an endogenous GA oxidase gene of a corn or cereal plant. In one aspect, further provided is a DNA molecule or vector comprising a recombinant DNA donor template disclosed here. In another aspect, further provided is a bacterial or host cell comprising a recombinant DNA donor template disclosed here. In another aspect, further provided is corn or cereal plant, plant part or plant cell comprising the recombinant DNA construct disclosed here.

In an aspect, the present disclosure provides an engineered site-specific nuclease that binds to a target site at or near the genomic locus of an endogenous GA oxidase gene of a corn or cereal plant and causes a double-strand break or nick at the target site.

In another aspect, the present disclosure provides a recombinant DNA construct comprising a transgene encoding a site-specific nuclease, wherein the site-specific nuclease binds to a target site at or near the genomic locus of an endogenous GA oxidase gene of a monocot or cereal plant and causes a double-strand break or nick at the target site.

In an aspect, the present disclosure provides a method for producing a transgenic corn or cereal plant, comprising: (a) transforming at least one cell of an explant with a recombinant DNA donor template disclosed here, and (b) regenerating or developing the transgenic corn or cereal plant from the transformed explant, wherein the transgenic corn or cereal plant comprises the insertion sequence of the recombinant DNA donor template.

In another aspect, the present disclosure provides a method for producing a corn or cereal plant having a genomic edit at or near an endogenous GA oxidase gene, comprising: (a) introducing into at least one cell of an explant of the corn or cereal plant a site-specific nuclease or a recombinant DNA molecule comprising a transgene encoding the site-specific nuclease, wherein the site-specific nuclease binds to a target site at or near the genomic locus of the endogenous GA oxidase gene and causes a double-strand break or nick at the target site, and (b) regenerating or developing an edited corn or cereal plant from the at least one explant cell comprising the genomic edit at or near the endogenous GA oxidase gene of the edited monocot or cereal plant.

In an aspect, the present disclosure provides a modified corn plant having a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and one or more of (i) an average stem or stalk diameter of greater than 18 mm, greater than 18.5 mm, greater than 19 mm, greater than 19.5 mm, greater than 20 mm, greater than 20.5 mm, greater than 21 mm, greater than 21.5 mm, or greater than 22 mm, (ii) improved lodging resistance relative to a wild type control plant, or (iii) improved drought tolerance relative to a wild type control plant.

In another aspect, the present disclosure provides a modified cereal plant having a reduced plant height relative to a wild type control plant, and (i) an increased stem or stalk diameter relative to a wild type control plant, (ii) improved lodging resistance relative to a wild type control plant, or (iii) improved drought tolerance relative to a wild type control plant.

In an aspect, the present disclosure provides a modified corn plant, or plant part thereof, comprising a mutant allele at GA20 oxidase 3 locus and a mutant allele at GA20 oxidase 5 locus, wherein at least one of the GA20 oxidase_3 and GA20 oxidase_5 loci comprises homozygous mutant alleles.

In another aspect, the present disclosure provides a modified corn plant, or plant part thereof, comprising a first homozygous mutation in one of GA20 oxidase_3 and GA20 oxidase_5 genes and further comprising a second heterozygous or homozygous mutation in the other one of the GA20 oxidase_3 and GA20 oxidase_5 genes.

In an aspect, the present disclosure provides a modified corn plant, or plant part thereof, comprising a homozygous mutant GA20 oxidase_3 gene and a homozygous mutant GA20 oxidase_5 gene.

In another aspect, the present disclosure provides a modified corn plant, or plant part thereof, comprising homozygous mutant alleles at an endogenous GA20 oxidase_3 locus and homozygous mutant alleles at an endogenous GA20 oxidase_5 locus.

In an aspect, the present disclosure provides a modified corn plant, or plant part thereof, comprising homozygous mutant alleles at the GA20 oxidase_3 locus, and exhibiting an at least 2.5%, at least 5%, at least 7.5%, at least 10%, at least 15%, or at least 20% reduction in plant height at maturity relative to an unmodified control plant.

In another aspect, the present disclosure provides a modified corn plant, or plant part thereof, comprising homozygous mutant alleles at the GA20 oxidase_5 locus, and exhibiting an at least 2.5%, at least 5%, at least 7.5%, at least 10%, at least 15%, or at least 20% reduction in plant height at maturity relative to an unmodified control plant.

### BRIEF DESCRIPTION OF THE DRAWINGS

FIG. 1 shows reduced plant heights of corn inbred plants expressing a GA20 oxidase suppression construct across eight transformation events in comparison to inbred control plants;

FIG. 2A shows a reduced plant height on average of hybrid corn plants expressing a GA20 oxidase suppression construct in comparison to hybrid control plants;

FIG. 2B shows an image of a wild type hybrid control plant (left) next to a hybrid corn plant expressing a GA20 oxidase suppression construct (right) having a reduced plant height;

FIG. 3A shows an increased stem diameter on average of hybrid corn plants expressing a GA20 oxidase suppression construct in comparison to hybrid control plants;

FIG. 3B shows an image of a cross-section of the stalk of a wild type hybrid control plant (left) next to a cross-section of the stalk of a hybrid corn plant expressing a GA20 oxidase suppression construct (right) having an increased stem diameter;

FIG. 4 shows an increased fresh ear weight on average of hybrid corn plants expressing a GA20 oxidase suppression construct in comparison to hybrid control plants;

FIG. 5 shows the increased fresh ear weight on average of hybrid corn plants expressing a GA20 oxidase suppression construct in two field trials in comparison to wild type hybrid control plants in response to a wind event that caused greater lodging in the hybrid control plants;

FIG. 6 shows an increased harvest index of hybrid corn plants expressing a GA20 oxidase suppression construct in comparison to hybrid control plants;

FIG. 7 shows an increase in the average grain yield estimate of hybrid corn plants expressing a GA20 oxidase suppression construct in comparison to hybrid control plants;

FIG. 8 shows an increased prolificacy score on average of hybrid corn plants expressing a GA20 oxidase suppression construct in comparison to hybrid control plants;

FIG. 9 shows the change in plant height over time during developmental stages V11 to beyond R1 between transgenic corn plants and control;

FIG. 10 shows a graph comparing measurements of stable oxygen isotope ratios (δ¹⁸O) as an indication of stomatal conductance and water levels in leaf tissue at R5 stage between transgenic corn plants and control;

FIG. 11 shows a graph comparing root front velocity during developmental stages V10 to beyond R2 between transgenic and control plants at both SAP and HD conditions using sensors at different soil depths that detect changes in water levels indicating the presence of roots at that depth;

FIG. 12A shows differences in stomatal conductance during the morning and afternoon between transgenic corn plants and control under normal and drought conditions in the greenhouse;

FIG. 12B shows differences in photosynthesis during the morning and afternoon between transgenic corn plants and control under normal and drought conditions in the greenhouse;

FIG. 13A shows differences in miRNA expression levels in bulk stem tissue, or separated vascular and non-vascular stem tissues, of transgenic corn plants versus control;

FIG. 13B shows differences in GA20 oxidase_3 and GA20 oxidase_5 mRNA transcript expression levels in bulk stem tissue, or separated vascular and non-vascular stem tissues, of transgenic corn plants versus control; and

FIG. 14 shows plant heights of inbred mutant plants having edited mutant GA20 oxidase_3 and/or GA20 oxidase_5 genes in comparison to inbred wild-type control plants and plants expressing a GA20 oxidase suppression construct.

### DETAILED DESCRIPTION

### Definitions

To facilitate understanding of the disclosure, several terms and abbreviations as used herein are defined below as follows:

The term “and/or” when used in a list of two or more items, means that any one of the listed items can be employed by itself or in combination with any one or more of the listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

The term “about” as used herein, is intended to qualify the numerical values that it modifies, denoting such a value as variable within a margin of error. When no particular margin of error, such as a standard deviation to a mean value, is recited, the term “about” should be understood to mean that range which would encompass the recited value and the range which would be included by rounding up or down to that figure, taking into account significant figures.

As used herein, “locus” is a chromosomal locus or region where a polymorphic nucleic acid, trait determinant, gene, or marker is located. A “locus” can be shared by two homologous chromosomes to refer to their corresponding locus or region. As used herein, “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant allele for a gene may have a reduced or eliminated activity or expression level for the gene relative to the wild-type allele. For diploid organisms such as corn, a first allele can occur on one chromosome, and a second allele can occur at the same locus on a second homologous chromosome. If one allele at a locus on one chromosome of a plant is a mutant allele and the other corresponding allele on the homologous chromosome of the plant is wild-type, then the plant is described as being heterozygous for the mutant allele. However, if both alleles at a locus are mutant alleles, then the plant is described as being homozygous for the mutant alleles. A plant homozygous for mutant alleles at a locus may comprise the same mutant allele or different mutant alleles if heteroallelic or biallelic.

The term “cereal plant” as used herein refers a monocotyledonous (monocot) crop plant that is in the Poaceae or Gramineae family of grasses and is typically harvested for its seed, including, for example, wheat, corn, rice, millet, barley, sorghum, oat and rye.

As used herein, a “wild-type gene” or “wild-type allele” refers to a gene or allele having a sequence or genotype that is most common in a particular plant species, or another sequence or genotype with natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. For purposes of calculating “percent identity” between DNA and RNA sequences, a uracil (U) of a RNA sequence is considered identical to a thymine (T) of a DNA sequence. If the window of comparison is defined as a region of alignment between two or more sequences (i.e., excluding nucleotides at the 5′ and 3′ ends of aligned polynucleotide sequences, or amino acids at the N-terminus and C-terminus of aligned protein sequences, that are not identical between the compared sequences), then the “percent identity” may also be referred to as a “percent alignment identity”. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

It is recognized that residue positions of proteins that are not identical often differ by conservative amino acid substitutions, where amino acid residues are substituted for other amino acid residues with similar size and chemical properties (e.g., charge, hydrophobicity, polarity, etc.), and therefore may not change the functional properties of the molecule. When sequences differ in conservative substitutions, the percent sequence similarity may be adjusted upwards to correct for the conservative nature of the non-identical substitution(s). Sequences that differ by such conservative substitutions are said to have “sequence similarity” or “similarity.” Thus, “percent similarity” or “percent similar” as used herein in reference to two or more protein sequences is calculated by (i) comparing two optimally aligned protein sequences over a window of comparison, (ii) determining the number of positions at which the same or similar amino acid residue occurs in both sequences to yield the number of matched positions, (iii) dividing the number of matched positions by the total number of positions in the window of comparison (or the total length of the reference or query protein if a window of comparison is not specified), and then (iv) multiplying this quotient by 100% to yield the percent similarity. Conservative amino acid substitutions for proteins are known in the art.

For optimal alignment of sequences to calculate their percent identity or similarity, various pair-wise or multiple sequence alignment algorithms and programs are known in the art, such as ClustalW, or Basic Local Alignment Search Tool® (BLAST®), etc., that may be used to compare the sequence identity or similarity between two or more nucleotide or protein sequences. Although other alignment and comparison methods are known in the art, the alignment between two sequences (including the percent identity ranges described above) may be as determined by the ClustalW or BLAST® algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); and Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and a RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

The term “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature—e.g., the promoter or regulatory sequence has a different origin relative to the associated polynucleotide sequence and/or the promoter or regulatory sequence is not naturally occurring in a plant species to be transformed with the promoter or regulatory sequence.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., may comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., may also refer to a polynucleotide or protein molecule or sequence that has been genetically engineered and/or constructed outside of a cell. For example, a recombinant DNA molecule may comprise any engineered or man-made plasmid, vector, etc., and may include a linear or circular DNA molecule. Such plasmids, vectors, etc., may contain various maintenance elements including a prokaryotic origin of replication and selectable marker, as well as one or more transgenes or expression cassettes perhaps in addition to a plant selectable marker gene, etc.

As used herein, the term “isolated” refers to at least partially separating a molecule from other molecules typically associated with it in its natural state. In one embodiment, the term “isolated” refers to a DNA molecule that is separated from the nucleic acids that normally flank the DNA molecule in its natural state. For example, a DNA molecule encoding a protein that is naturally present in a bacterium would be an isolated DNA molecule if it was not within the DNA of the bacterium from which the DNA molecule encoding the protein is naturally found. Thus, a DNA molecule fused to or operably linked to one or more other DNA molecule(s) with which it would not be associated in nature, for example as the result of recombinant DNA or plant transformation techniques, is considered isolated herein. Such molecules are considered isolated even when integrated into the chromosome of a host cell or present in a nucleic acid solution with other DNA molecules.

As used herein, an “encoding region” or “coding region” refers to a portion of a polynucleotide that encodes a functional unit or molecule (e.g., without being limiting, a mRNA, protein, or non-coding RNA sequence or molecule).

As used herein, “modified” in the context of a plant, plant seed, plant part, plant cell, and/or plant genome, refers to a plant, plant seed, plant part, plant cell, and/or plant genome comprising an engineered change in the expression level and/or coding sequence of one or more GA oxidase gene(s) relative to a wild-type or control plant, plant seed, plant part, plant cell, and/or plant genome, such as via (A) a transgenic event comprising a suppression construct or transcribable DNA sequence encoding a non-coding RNA that targets one or more GA3 and/or GA20 oxidase genes for suppression, or (B) a genome editing event or mutation affecting (e.g., reducing or eliminating) the expression level or activity of one or more endogenous GA3 and/or GA20 oxidase genes. Indeed, the term “modified” may further refer to a plant, plant seed, plant part, plant cell, and/or plant genome having one or more mutations affecting expression of one or more endogenous GA oxidase genes, such as one or more endogenous GA3 and/or GA20 oxidase genes, introduced through chemical mutagenesis, transposon insertion or excision, or any other known mutagenesis technique, or introduced through genome editing. For clarity, therefore, a modified plant, plant seed, plant part, plant cell, and/or plant genome includes a mutated, edited and/or transgenic plant, plant seed, plant part, plant cell, and/or plant genome having a modified expression level, expression pattern, and/or coding sequence of one or more GA oxidase gene(s) relative to a wild-type or control plant, plant seed, plant part, plant cell, and/or plant genome. Modified plants can be homozygous or heterozygous for any given mutation or edit, and/or may be bi-allelic or heteroallelic at a GA oxidase gene locus. A modified plant is bi-allelic or heteroallelic for a GA oxidase gene if each copy of the GA oxidase gene is a different allele (i.e., comprises different mutation(s) and/or edit(s)), wherein each allele lowers the expression level and/or activity of the GA oxidase gene. Modified plants or seeds may contain various molecular changes that affect expression of GA oxidase gene(s), such as GA3 and/or GA20 oxidase gene(s), including genetic and/or epigenetic modifications. Modified plants, plant parts, seeds, etc., may have been subjected to mutagenesis, genome editing or site-directed integration (e.g., without being limiting, via methods using site-specific nucleases), genetic transformation (e.g., without being limiting, via methods of Agrobacterium transformation or microprojectile bombardment), or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the one or more GA oxidase genes. A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

As used herein, the term “homozygous” refers to a genotype comprising two identical alleles at a given locus in a diploid genome, or a genotype comprising two non-identical mutant alleles at a given locus in a diploid genome. The latter genotype comprising two non-identical mutant alleles is also referred to as being heteroallelic or transheterozygous, or as a heteroallelic combination. As used herein, “heterozygous” describes a genotype comprising a mutant allele and a wild-type allele at a given locus in a diploid genome.

As used herein, the term “control plant” (or likewise a “control” plant seed, plant part, plant cell and/or plant genome) refers to a plant (or plant seed, plant part, plant cell and/or plant genome) that is used for comparison to a modified plant (or modified plant seed, plant part, plant cell and/or plant genome) and has the same or similar genetic background (e.g., same parental lines, hybrid cross, inbred line, testers, etc.) as the modified plant (or plant seed, plant part, plant cell and/or plant genome), except for a transgenic and/or genome editing event(s) affecting one or more GA oxidase genes. For example, a control plant may be an inbred line that is the same as the inbred line used to make the modified plant, or a control plant may be the product of the same hybrid cross of inbred parental lines as the modified plant, except for the absence in the control plant of any transgenic or genome editing event(s) affecting one or more GA oxidase genes. Similarly, an unmodified control plant refers to a plant that shares a substantially similar or essentially identical genetic background as a modified plant, but without the one or more engineered changes to the genome (e.g., transgene, mutation or edit) of the modified plant. For purposes of comparison to a modified plant, plant seed, plant part, plant cell and/or plant genome, a “wild-type plant” (or likewise a “wild-type” plant seed, plant part, plant cell and/or plant genome) refers to a non-transgenic and non-genome edited control plant, plant seed, plant part, plant cell and/or plant genome. As used herein, a “control” plant, plant seed, plant part, plant cell and/or plant genome may also be a plant, plant seed, plant part, plant cell and/or plant genome having a similar (but not the same or identical) genetic background to a modified plant, plant seed, plant part, plant cell and/or plant genome, if deemed sufficiently similar for comparison of the characteristics or traits to be analyzed.

As used herein, a “target site” for genome editing refers to the location of a polynucleotide sequence within a plant genome that is bound and cleaved by a site-specific nuclease introducing a double stranded break (or single-stranded nick) into the nucleic acid backbone of the polynucleotide sequence and/or its complementary DNA strand. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for a RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further below). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by another site-specific nuclease that may not be guided by a non-coding RNA molecule, such as a meganuclease, zinc finger nuclease (ZFN), or a transcription activator-like effector nuclease (TALEN), to introduce a double stranded break (or single-stranded nick) into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region. Apart from genome editing, the term “target site” may also be used in the context of gene suppression to refer to a portion of a mRNA molecule (e.g., a “recognition site”) that is complementary to at least a portion of a non-coding RNA molecule (e.g., a miRNA, siRNA, etc.) encoded by a suppression construct.

As used herein, a “donor molecule”, “donor template”, or “donor template molecule” (collectively a “donor template”), which may be a recombinant DNA donor template, is defined as a nucleic acid molecule having a nucleic acid template or insertion sequence for site-directed, targeted insertion or recombination into the genome of a plant cell via repair of a nick or double-stranded DNA break in the genome of a plant cell. For example, a “donor template” may be used for site-directed integration of a transgene or suppression construct, or as a template to introduce a mutation, such as an insertion, deletion, etc., into a target site within the genome of a plant. A targeted genome editing technique provided herein may comprise the use of one or more, two or more, three or more, four or more, or five or more donor molecules or templates. A “donor template” may be a single-stranded or double-stranded DNA or RNA molecule or plasmid. An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template.

An insertion sequence of a donor template may comprise one or more genes or sequences that each encode a transcribed non-coding RNA or mRNA sequence and/or a translated protein sequence. A transcribed sequence or gene of a donor template may encode a protein or a non-coding RNA molecule. An insertion sequence of a donor template may comprise a polynucleotide sequence that does not comprise a functional gene or an entire gene sequence (e.g., the donor template may simply comprise regulatory sequences, such as a promoter sequence, or only a portion of a gene or coding sequence), or may not contain any identifiable gene expression elements or any actively transcribed gene sequence. Further, the donor template may be linear or circular, and may be single-stranded or double-stranded. A donor template may be delivered to the cell as a naked nucleic acid (e.g., via particle bombardment), as a complex with one or more delivery agents (e.g., liposomes, proteins, poloxamers, T-strand encapsulated with proteins, etc.), or contained in a bacterial or viral delivery vehicle, such as, for example, Agrobacterium tumefaciens or a geminivirus, respectively. An insertion sequence of a donor template provided herein may comprise a transcribable DNA sequence that may be transcribed into an RNA molecule, which may be non-coding and may or may not be operably linked to a promoter and/or other regulatory sequence.

According to some embodiments, a donor template may not comprise an insertion sequence, and instead comprise one or more homology sequences that include(s) one or more mutations, such as an insertion, deletion, substitution, etc., relative to the genomic sequence at a target site within the genome of a plant, such as at or near a GA3 oxidase or GA20 oxidase gene within the genome of a plant. Alternatively, a donor template may comprise an insertion sequence that does not comprise a coding or transcribable DNA sequence, wherein the insertion sequence is used to introduce one or more mutations into a target site within the genome of a plant, such as at or near a GA3 oxidase or GA20 oxidase gene within the genome of a plant.

A donor template provided herein may comprise at least one, at least two, at least three, at least four, at least five, at least six, at least seven, at least eight, at least nine, or at least ten genes or transcribable DNA sequences. Alternatively, a donor template may comprise no genes. Without being limiting, a gene or transcribable DNA sequence of a donor template may include, for example, an insecticidal resistance gene, an herbicide tolerance gene, a nitrogen use efficiency gene, a water use efficiency gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a transcribable DNA sequence that encodes a non-coding RNA molecule, which may target a GA oxidase gene, such as a GA3 oxidase or GA20 oxidase gene, for suppression. A donor template may comprise a promoter, such as a tissue-specific or tissue-preferred promoter, a constitutive promoter, or an inducible promoter. A donor template may comprise a leader, enhancer, promoter, transcriptional start site, 5′-UTR, one or more exon(s), one or more intron(s), transcriptional termination site, region or sequence, 3’-UTR, and/or polyadenylation signal. The leader, enhancer, and/or promoter may be operably linked to a gene or transcribable DNA sequence encoding a non-coding RNA, a guide RNA, an mRNA and/or protein.

As used herein, a “vascular promoter” refers to a plant-expressible promoter that drives, causes or initiates expression of a transcribable DNA sequence or transgene operably linked to such promoter in one or more vascular tissue(s) of the plant, even if the promoter is also expressed in other non-vascular plant cell(s) or tissue(s). Such vascular tissue(s) may comprise one or more of the phloem, vascular parenchymal, and/or bundle sheath cell(s) or tissue(s) of the plant. A “vascular promoter” is distinguished from a constitutive promoter in that it has a regulated and relatively more limited pattern of expression that includes one or more vascular tissue(s) of the plant. A vascular promoter includes both vascular-specific promoters and vascular-preferred promoters.

As used herein, a “leaf promoter” refers to a plant-expressible promoter that drives, causes or initiates expression of a transcribable DNA sequence or transgene operably linked to such promoter in one or more leaf tissue(s) of the plant, even if the promoter is also expressed in other non-leaf plant cell(s) or tissue(s). A leaf promoter includes both leaf-specific promoters and leaf-preferred promoters. A “leaf promoter” is distinguished from a vascular promoter in that it is expressed more predominantly or exclusively in leaf tissue(s) of the plant relative to other plant tissues, whereas a vascular promoter is expressed in vascular tissue(s) more generally including vascular tissue(s) outside of the leaf, such as the vascular tissue(s) of the stem, or stem and leaves, of the plant.

### DESCRIPTION

The co-pending PCT Application No. PCT/US2017/047405 and U.S. application Ser. No. 15/679,699, both filed on Aug. 17, 2017, are incorporated herein by reference in their entirety.

Most grain producing grasses, such as wheat, rice and sorghum, produce both male and female structures within each floret of the panicle (i.e., they have a single reproductive structure). However, corn or maize is unique among the grain-producing grasses in that it forms separate male (tassel) and female (ear) inflorescences. Corn produces completely sexually dimorphic reproductive structures by selective abortion of male organs (anthers) in florets of the ear, and female organs (ovules) in the florets of the tassel within early stages of development. Precisely regulated gibberellin synthesis and signaling is critical to regulation of this selective abortion process, with the female reproductive ear being most sensitive to disruptions in the GA pathway. Indeed, the “anther ear” phenotype is the most common reproductive phenotype in GA corn mutants.

In contrast to corn, mutations in the gibberellin synthesis or signaling pathways that led to the “Green Revolution” in wheat, rice and sorghum had little impact on their reproductive structures because these crop species do not undergo the selective abortion process of the grain bearing panicle during development, and thus are not sensitive to disruptions in GA levels. The same mutations have not been utilized in corn because disruption of the GA synthesis and signaling pathway has repeatedly led to dramatic distortion and masculinization of the ear (“anther ear”) and sterility (disrupted anther and microspore development) in the tassel, in addition to extreme dwarfing in some cases. See, e.g., Chen, Y. et al., “The Maize DWARF1 Encodes a Gibberellin 3-Oxidase and Is Dual Localized to the Nucleus and Cytosol,” Plant Physiology 166: 2028-2039 (2014). These GA mutant phenotypes (off-types) in corn led to significant reductions in kernel production and a reduction in yield. Furthermore, production of anthers within the ear increases the likelihood of fungal or insect infections, which reduces the quality of the grain that is produced on those mutant ears. Forward breeding to develop semi-dwarf lines of corn has not been successful, and the reproductive off-types (as well as the extreme dwarfing) of GA mutants have been challenging to overcome. Thus, the same mutations in the GA pathway that led to the Green Revolution in other grasses have not yet been successful in corn.

Despite these prior difficulties in achieving higher grain yields in corn through manipulation of the GA pathway, the present inventors have discovered a way to manipulate GA levels in corn plants in a manner that reduces overall plant height and stem internode length and increases resistance to lodging, but does not cause the reproductive off-types previously associated with mutations of the GA pathway in corn. Further evidence indicates that these short stature or semi-dwarf corn plants may also have one or more additional traits, including increased stem diameter, reduced green snap, deeper roots, increased leaf area, earlier canopy closure, higher stomatal conductance, lower ear height, increased foliar water content, improved drought tolerance, increased nitrogen use efficiency, increased water use efficiency, reduced anthocyanin content and area in leaves under normal or nitrogen or water limiting stress conditions, increased ear weight, increased kernel number, increased kernel weight, increased yield, and/or increased harvest index.

Without being bound by theory, it is proposed that incomplete suppression of GA20 or GA3 oxidase gene(s) and/or targeting of a subset of one or more GA oxidase gene(s) may be effective in achieving a short stature, semi-dwarf phenotype with increased resistance to lodging, but without reproductive off-types in the ear. It is further proposed, without being limited by theory, that restricting the suppression of GA20 and/or GA3 oxidase gene(s) to certain active GA-producing tissues, such as the vascular and/or leaf tissues of the plant, may be sufficient to produce a short-stature plant with increased lodging resistance, but without significant off-types in reproductive tissues. Expression of a GA20 or GA3 oxidase suppression element in a tissue-specific or tissue-preferred manner may be sufficient and effective at producing plants with the short stature phenotype, while avoiding potential off-types in reproductive tissues that were previously observed with GA mutants in corn (e.g., by avoiding or limiting the suppression of the GA20 oxidase gene(s) in those reproductive tissues). For example, GA20 and/or GA3 oxidase gene(s) may be targeted for suppression using a vascular promoter, such as a rice tungro bacilliform virus (RTBV) promoter, that drives expression in vascular tissues of plants. As supported in the Examples below, the expression pattern of the RTBV promoter is enriched in vascular tissues of corn plants relative to non-vascular tissues, which is sufficient to produce a semi-dwarf phenotype in corn plants when operably linked to a suppression element targeting GA20 and GA3 oxidase gene(s). Lowering of active GA levels in tissue(s) of a corn or cereal plant that produce active GAs may reduce plant height and increase lodging resistance, and off-types may be avoided in those plants if active GA levels are not also significantly impacted or lowered in reproductive tissues, such as the developing female organ or ear of the plant. If active GA levels could be reduced in the stalk, stem, or internode(s) of corn or cereal plants without significantly affecting GA levels in reproductive tissues (e.g., the female or male reproductive organs or inflorescences), then corn or cereal plants having reduced plant height and increased lodging resistance could be created without off-types in the reproductive tissues of the plant.

Thus, recombinant DNA constructs and transgenic plants are provided herein comprising a GA20 or GA3 oxidase suppression element or sequence operably linked to a plant expressible promoter, which may be a tissue-specific or tissue-preferred promoter. Such a tissue-specific or tissue-preferred promoter may drive expression of its associated GA oxidase suppression element or sequence in one or more active GA-producing tissue(s) of the plant to suppress or reduce the level of active GAs produced in those tissue(s). Such a tissue-specific or tissue-preferred promoter may drive expression of its associated GA oxidase suppression construct or transgene during one or more vegetative stage(s) of development. Such a tissue-specific or tissue-preferred promoter may also have little or no expression in one or more cell(s) or tissue(s) of the developing female organ or ear of the plant to avoid the possibility of off-types in those reproductive tissues. According to some embodiments, the tissue-specific or tissue-preferred promoter is a vascular promoter, such as the RTBV promoter. The sequence of the RTBV promoter is provided herein as SEQ ID NO: 65, and a truncated version of the RTBV promoter is further provided herein as SEQ ID NO: 66.

Active or bioactive gibberellic acids (i.e., “active gibberellins” or “active GAs”) are known in the art for a given plant species, as distinguished from inactive GAs. For example, active GAs in corn and higher plants include the following: GA1, GA3, GA4, and GA7. Thus, an “active GA-producing tissue” is a plant tissue that produces one or more active GAs.

In addition to suppressing GA20 oxidase genes in active GA-producing tissues of the plant with a vascular tissue promoter, it was surprisingly found that suppression of the same GA20 oxidase genes with various constitutive promoters could also cause the short, semi-dwarf stature phenotypes in corn, but without any visible off-types in the ear. Given that mutations in the GA pathway have previously been shown to cause off-types in reproductive tissues, it was surprising that constitutive suppression of GA20 oxidase did not cause similar reproductive phenotypes in the ear. Thus, it is further proposed that suppression of one or more GA20 oxidase genes could be carried out using a constitutive promoter to create a short stature, lodging-resistant corn or cereal plant without any significant or observable reproductive off-types in the plant. Other surprising observations were made when the same GA20 oxidase suppression construct was expressed in the stem, leaf or reproductive tissues. As described further below, targeted suppression of the same GA20 oxidase genes in the stem or ear tissues of corn plants did not cause the short stature, semi-dwarf phenotype. Moreover, directed expression of the GA20 oxidase suppression construct directly in reproductive tissues of the developing ear of corn plants with a female reproductive tissue (ear) promoter did not cause any significant or observable off-types in the ear. However, expression of the same GA20 oxidase suppression construct in leaf tissues was sufficient to cause a moderate short stature phenotype without significant or observable reproductive off-types in the plant.

Without being limited by theory, it is proposed that short stature, semi-dwarf phenotypes in corn and other cereal plants may result from a sufficient level of expression of a suppression construct targeting certain GA oxidase gene(s) in active GA-producing tissue(s) of the plant. At least for targeted suppression of certain GA20 oxidase genes in corn, restricting the pattern of expression to avoid reproductive ear tissues may not be necessary to avoid reproductive off-types in the developing ear. However, expression of the GA20 oxidase suppression construct at low levels, and/or in a limited number of plant tissues, may be insufficient to cause a significant short stature, semi-dwarf phenotype. Given that the observed semi-dwarf phenotype with targeted GA20 oxidase suppression is the result of shortening the stem internodes of the plant, it is surprising that suppression of GA20 oxidase genes in at least some stem tissues was not sufficient to cause shortening of the internodes and reduced plant height. Without being bound by theory, it is proposed that suppression of certain GA oxidase gene(s) in tissue(s) and/or cell(s) of the plant where active GAs are produced, and not necessarily in stem or internode tissue(s), may be sufficient to produce semi-dwarf plants, even though the short stature trait is due to shortening of the stem internodes. Given that GAs can migrate through the vasculature of the plant, it is proposed that manipulating GA oxidase genes in plant tissue(s) where active GAs are produced may result in a short stature, semi-dwarf plant, even though this may be largely achieved by suppressing the level of active GAs produced in non-stem tissues (i.e., away from the site of action in the stem where reduced internode elongation leads to the semi-dwarf phenotype). Indeed, suppression of certain GA20 oxidase genes in leaf tissues was found to cause a moderate semi-dwarf phenotype in corn plants. Given that expression of a GA20 oxidase suppression construct with several different “stem” promoters did not produce the semi-dwarf phenotype in corn, it is noteworthy that expression of the same GA20 oxidase suppression construct with a vascular promoter was effective at consistently producing the semi-dwarf phenotype with a high degree of penetrance across events and germplasms. This semi-dwarf phenotype was also observed with expression of the same GA20 oxidase suppression construct using other vascular promoters.

According to embodiments of the present disclosure, modified cereal or corn plants are provided that have at least one beneficial agronomic trait and at least one female reproductive organ or ear that is substantially or completely free of off-types. The beneficial agronomic trait may include, for example, shorter plant height, shorter internode length in one or more internode(s), larger (thicker) stem or stalk diameter, increased lodging resistance, improved drought tolerance, increased nitrogen use efficiency, increased water use efficiency, deeper roots, larger leaf area, earlier canopy closure, and/or increased harvestable yield. Off-types may include male (tassel or anther) sterility, reduced kernel or seed number, and/or the presence of one or more masculinized or male (or male-like) reproductive structures in the female organ or ear (e.g., anther ear) of the plant. A modified cereal or corn plant is provided herein that lacks significant off-types in the reproductive tissues of the plant. Such a modified cereal or corn plant may have a female reproductive organ or ear that appears normal relative to a control or wild-type plant. Indeed, modified cereal or corn plants are provided that comprise at least one reproductive organ or ear that does not have or exhibit, or is substantially or completely free of, off-types including male sterility, reduced kernel or seed number, and/or masculinized structure(s) in one or more female organs or ears. As used herein, a female organ or ear of a plant, such as corn, is “substantially free” of male reproductive structures if male reproductive structures are absent or nearly absent in the female organ or ear of the plant based on visual inspection of the female organ or ear at later reproductive stages. A female organ or ear of a plant, such as corn, is “completely free” of mature male reproductive structures if male reproductive structures are absent or not observed or observable in the female organ or ear of the plant, such as a corn plant, by visual inspection of the female organ or ear at later reproductive stages. A female organ or ear of a plant, such as corn, without significant off-types and substantially free of male reproductive structures in the ear may have a number of kernels or seeds per female organ or ear of the plant that is at least 90%, at least 95%, at least 96%, at least 97%, at least 98%, at least 99%, at least 99.5%, at least 99.6%, at least 99.7%, at least 99.8%, or at least 99.9% of the number of kernels or seeds per female organ or ear of a wild-type or control plant. Likewise, a female organ or ear of a plant, such as corn, without significant off-types and substantially free of male reproductive structures in the ear may have an average kernel or seed weight per female organ or ear of the plant that is at least 90%, at least 95%, at least 96%, at least 97%, at least 98%, at least 99%, at least 99.5%, at least 99.6%, at least 99.7%, at least 99.8%, or at least 99.9% of the average kernel or seed weight per female organ or ear of a wild-type or control plant. A female organ or ear of a plant, such as corn, that is completely free of mature male reproductive structures may have a number of kernels or seeds per female organ or ear of the plant that is about the same as a wild-type or control plant. In other words, the reproductive development of the female organ or ear of the plant may be normal or substantially normal. However, the number of seeds or kernels per female organ or ear may depend on other factors that affect resource utilization and development of the plant. Indeed, the number of kernels or seeds per female organ or ear of the plant, and/or the kernel or seed weight per female organ or ear of the plant, may be about the same or greater than a wild-type or control plant.

The plant hormone gibberellin plays an important role in a number of plant developmental processes including germination, cell elongation, flowering, embryogenesis and seed development. Certain biosynthetic enzymes (e.g., GA20 oxidase and GA3 oxidase) and catabolic enzymes (e.g., GA2 oxidase) in the GA pathway are critical to affecting active GA levels in plant tissues. Thus, in addition to suppression of certain GA20 oxidase genes, it is further proposed that suppression of a GA3 oxidase gene in a constitutive or tissue-specific or tissue-preferred manner may also produce corn plants having a short stature phenotype and increased lodging resistance, with possible increased yield, but without off-types in the ear. Thus, according to some embodiments, constructs and transgenes are provided comprising a GA3 oxidase suppression element or sequence operably linked to a constitutive or tissue-specific or tissue-preferred promoter, such as a vascular or leaf promoter. According to some embodiments, the tissue-specific or tissue-preferred promoter is a vascular promoter, such as the RTBV promoter. However, other types of tissue-specific or tissue preferred promoters may potentially be used for GA3 oxidase suppression in active GA-producing tissues of a corn or cereal plant to produce a semi-dwarf phenotype without significant off-types.

Any method known in the art for suppression of a target gene may be used to suppress GA oxidase gene(s) according to embodiments of the present invention including expression of antisense RNAs, double stranded RNAs (dsRNAs) or inverted repeat RNA sequences, or via co-suppression or RNA interference (RNAi) through expression of small interfering RNAs (siRNAs), short hairpin RNAs (shRNAs), trans-acting siRNAs (ta-siRNAs), or micro RNAs (miRNAs). Furthermore, sense and/or antisense RNA molecules may be used that target the coding and/or non-coding genomic sequences or regions within or near a GA oxidase gene to cause silencing of the gene. Accordingly, any of these methods may be used for the targeted suppression of an endogenous GA20 oxidase or GA3 oxidase gene(s) in a tissue-specific or tissue-preferred manner. See, e.g., U.S. Patent Application Publication Nos. 2009/0070898, 2011/0296555, and 2011/0035839, the contents and disclosures of which are incorporated herein by reference.

The term “suppression” as used herein, refers to a lowering, reduction or elimination of the expression level of a mRNA and/or protein encoded by a target gene in a plant, plant cell or plant tissue at one or more stage(s) of plant development, as compared to the expression level of such target mRNA and/or protein in a wild-type or control plant, cell or tissue at the same stage(s) of plant development. According to some embodiments, a modified or transgenic plant is provided having a GA20 oxidase gene expression level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified or transgenic plant is provided having a GA3 oxidase gene expression level that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified or transgenic plant is provided having a GA20 oxidase gene expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a modified or transgenic plant is provided having a GA3 oxidase gene expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to these embodiments, the at least one tissue of a modified or transgenic plant having a reduced expression level of a GA20 oxidase and/or GA3 oxidase gene(s) includes one or more active GA producing tissue(s) of the plant, such as the vascular and/or leaf tissue(s) of the plant, during one or more vegetative stage(s) of development.

In some embodiments, suppression of an endogenous GA20 oxidase gene or a GA3 oxidase gene is tissue-specific (e.g., only in leaf and/or vascular tissue). Suppression of a GA20 oxidase gene may be constitutive and/or vascular or leaf tissue specific or preferred. In other embodiments, suppression of a GA20 oxidase gene or a GA3 oxidase gene is constitutive and not tissue-specific. According to some embodiments, expression of an endogenous GA20 oxidase gene and/or a GA3 oxidase gene is reduced in one or more tissue types (e.g., in leaf and/or vascular tissue(s)) of a modified or transgenic plant as compared to the same tissue(s) of a control plant.

According to embodiments of the present disclosure, a recombinant DNA molecule, construct or vector is provided comprising a suppression element targeting GA20 oxidase or GA3 oxidase gene(s) that is operably linked to a plant-expressible constitutive or tissue-specific or tissue-preferred promoter. The suppression element may comprise a transcribable DNA sequence of at least 19 nucleotides in length, such as from about 19 nucleotides in length to about 27 nucleotides in length, or 19, 20, 21, 22, 23, 24, 25, 26, or 27 nucleotides in length, wherein the transcribable DNA sequence corresponds to at least a portion of the target GA oxidase gene to be suppressed, and/or to a DNA sequence complementary thereto. The suppression element may be 19-30, 19-50, 19-100, 19-200, 19-300, 19-500, 19-1000, 19-1500, 19-2000, 19-3000, 19-4000, or 19-5000 nucleotides in length. The suppression element may be at least 19, at least 20, at least 21, at least 22, or at least 23 nucleotides or more in length (e.g., at least 25, at least 30, at least 50, at least 100, at least 200, at least 300, at least 500, at least 1000, at least 1500, at least 2000, at least 3000, at least 4000, or at least 5000 nucleotides in length). Depending on the length and sequence of a suppression element, one or more sequence mismatches or non-complementary bases, such as 1, 2, 3, 4, 5, 6, 7, 8 or more mismatches, may be tolerated without a loss of suppression if the non-coding RNA molecule encoded by the suppression element is still able to sufficiently hybridize and bind to the target mRNA molecule of the GA20 oxidase or GA3 oxidase gene(s). Indeed, even shorter RNAi suppression elements ranging from about 19 nucleotides to about 27 nucleotides in length may have one or more mismatches or non-complementary bases, yet still be effective at suppressing a target GA oxidase gene. Accordingly, a sense or anti-sense suppression element sequence may be at least 80%, at least 85%, at least 90%, at least 95%, at least 96%, at least 97%, at least 98%, at least 99%, at least 99.5% or 100% identical to a corresponding sequence of at least a segment or portion of the targeted GA oxidase gene, or its complementary sequence, respectively.

A suppression element or transcribable DNA sequence of the present invention for targeted suppression of GA oxidase gene(s) may include one or more of the following: (a) a DNA sequence that includes at least one anti-sense DNA sequence that is anti-sense or complementary to at least one segment or portion of the targeted GA oxidase gene; (b) a DNA sequence that includes multiple copies of at least one anti-sense DNA sequence that is anti-sense or complementary to at least one segment or portion of the targeted GA oxidase gene; (c) a DNA sequence that includes at least one sense DNA sequence that comprises at least one segment or portion of the targeted GA oxidase gene; (d) a DNA sequence that includes multiple copies of at least one sense DNA sequence that each comprise at least one segment or portion of the targeted GA oxidase gene; (e) a DNA sequence that includes an inverted repeat of a segment or portion of a targeted GA oxidase gene and/or transcribes into RNA for suppressing the targeted GA oxidase gene by forming double-stranded RNA, wherein the transcribed RNA includes at least one anti-sense DNA sequence that is anti-sense or complementary to at least one segment or portion of the targeted GA oxidase gene and at least one sense DNA sequence that comprises at least one segment or portion of the targeted GA oxidase gene; (f) a DNA sequence that is transcribed into RNA for suppressing the targeted GA oxidase gene by forming a single double-stranded RNA and includes multiple serial anti-sense DNA sequences that are each anti-sense or complementary to at least one segment or portion of the targeted GA oxidase gene and multiple serial sense DNA sequences that each comprise at least one segment or portion of the targeted GA oxidase gene; (g) a DNA sequence that is transcribed into RNA for suppressing the targeted GA oxidase gene by forming multiple double strands of RNA and includes multiple anti-sense DNA sequences that are each anti-sense or complementary to at least one segment or portion of the targeted GA oxidase gene and multiple sense DNA sequences that each comprise at least one segment or portion of the targeted GA oxidase gene, wherein the multiple anti-sense DNA segments and multiple sense DNA segments are arranged in a series of inverted repeats; (h) a DNA sequence that includes nucleotides derived from a miRNA, preferably a plant miRNA; (i) a DNA sequence that includes a miRNA precursor that encodes an artificial miRNA complementary to at least one segment or portion of the targeted GA oxidase gene; (j) a DNA sequence that includes nucleotides of a siRNA; (k) a DNA sequence that is transcribed into an RNA aptamer capable of binding to a ligand; and (1) a DNA sequence that is transcribed into an RNA aptamer capable of binding to a ligand and DNA that transcribes into a regulatory RNA capable of regulating expression of the targeted GA oxidase gene, wherein the regulation of the targeted GA oxidase gene is dependent on the conformation of the regulatory RNA, and the conformation of the regulatory RNA is allosterically affected by the binding state of the RNA aptamer by the ligand. Any of these gene suppression elements, whether transcribed into a single stranded or double-stranded RNA, may be designed to suppress more than one GA oxidase target gene, depending on the number and sequence of the suppression element(s).

Multiple sense and/or anti-sense suppression elements for more than one GA oxidase target may be arranged serially in tandem or arranged in tandem segments or repeats, such as tandem inverted repeats, which may also be interrupted by one or more spacer sequence(s), and the sequence of each suppression element may target one or more GA oxidase gene(s). Furthermore, the sense or anti-sense sequence of the suppression element may not be perfectly matched or complementary to the targeted GA oxidase gene sequence, depending on the sequence and length of the suppression element. Even shorter RNAi suppression elements from about 19 nucleotides to about 27 nucleotides in length may have one or more mismatches or non-complementary bases, yet still be effective at suppressing the target GA oxidase gene. Accordingly, a sense or anti-sense suppression element sequence may be at least 80%, at least 85%, at least 90%, at least 95%, at least 96%, at least 97%, at least 98%, at least 99%, at least 99.5% or 100% identical to a corresponding sequence of at least a segment or portion of the targeted GA oxidase gene, or its complementary sequence, respectively.

For anti-sense suppression, the transcribable DNA sequence or suppression element comprises a sequence that is anti-sense or complementary to at least a portion or segment of the targeted GA oxidase gene. The suppression element may comprise multiple anti-sense sequences that are complementary to one or more portions or segments of the targeted GA oxidase gene(s), or multiple copies of an anti-sense sequence that is complementary to a targeted GA oxidase gene. The anti-sense suppression element sequence may be at least 80%, at least 85%, at least 90%, at least 95%, at least 96%, at least 97%, at least 98%, at least 99%, at least 99.5% or 100% identical to a DNA sequence that is complementary to at least a segment or portion of the targeted GA oxidase gene. In other words, the anti-sense suppression element sequence may be at least 80%, at least 85%, at least 90%, at least 95%, at least 96%, at least 97%, at least 98%, at least 99%, at least 99.5% or 100% complementary to the targeted GA oxidase gene.

For suppression of GA oxidase gene(s) using an inverted repeat or a transcribed dsRNA, a transcribable DNA sequence or suppression element may comprise a sense sequence that comprises a segment or portion of a targeted GA oxidase gene and an anti-sense sequence that is complementary to a segment or portion of the targeted GA oxidase gene, wherein the sense and anti-sense DNA sequences are arranged in tandem. The sense and/or anti-sense sequences, respectively, may each be less than 100% identical or complementary to a segment or portion of the targeted GA oxidase gene as described above. The sense and anti-sense sequences may be separated by a spacer sequence, such that the RNA molecule transcribed from the suppression element forms a stem, loop or stem-loop structure between the sense and anti-sense sequences. The suppression element may instead comprise multiple sense and anti-sense sequences that are arranged in tandem, which may also be separated by one or more spacer sequences. Such suppression elements comprising multiple sense and anti-sense sequences may be arranged as a series of sense sequences followed by a series of anti-sense sequences, or as a series of tandemly arranged sense and anti-sense sequences. Alternatively, one or more sense DNA sequences may be expressed separately from the one or more anti-sense sequences (i.e., one or more sense DNA sequences may be expressed from a first transcribable DNA sequence, and one or more anti-sense DNA sequences may be expressed from a second transcribable DNA sequence, wherein the first and second transcribable DNA sequences are expressed as separate transcripts).

For suppression of GA oxidase gene(s) using a microRNA (miRNA), the transcribable DNA sequence or suppression element may comprise a DNA sequence derived from a miRNA sequence native to a virus or eukaryote, such as an animal or plant, or modified or derived from such a native miRNA sequence. Such native or native-derived miRNA sequences may form a fold back structure and serve as a scaffold for the precursor miRNA (pre-miRNA), and may correspond to the stem region of a native miRNA precursor sequence, such as from a native (or native-derived) primary-miRNA (pri-miRNA) or pre-miRNA sequence. However, in addition to these native or native-derived miRNA scaffold or preprocessed sequences, engineered or synthetic miRNAs of the present embodiments further comprise a sequence corresponding to a segment or portion of the targeted GA oxidase gene(s). Thus, in addition to the pre-processed or scaffold miRNA sequences, the suppression element may further comprise a sense and/or anti-sense sequence that corresponds to a segment or portion of a targeted GA oxidase gene, and/or a sequence that is complementary thereto, although one or more sequence mismatches may be tolerated.

Engineered miRNAs are useful for targeted gene suppression with increased specificity. See, e.g., Parizotto et al., Genes Dev. 18:2237-2242 (2004), and U.S. Patent Application Publication Nos. 2004/0053411, 2004/0268441, 2005/0144669, and 2005/0037988, the contents and disclosures of which are incorporated herein by reference. miRNAs are non-protein coding RNAs. When a miRNA precursor molecule is cleaved, a mature miRNA is formed that is typically from about 19 to about 25 nucleotides in length (commonly from about 20 to about 24 nucleotides in length in plants), such as 19, 20, 21, 22, 23, 24, or 25 nucleotides in length, and has a sequence corresponding to the gene targeted for suppression and/or its complement. The mature miRNA hybridizes to target mRNA transcripts and guides the binding of a complex of proteins to the target transcripts, which may function to inhibit translation and/or result in degradation of the transcript, thus negatively regulating or suppressing expression of the targeted gene. miRNA precursors are also useful in plants for directing in-phase production of siRNAs, trans-acting siRNAs (ta-siRNAs), in a process that requires a RNA-dependent RNA polymerase to cause suppression of a target gene. See, e.g., Allen et al., Cell 121:207-221 (2005), Vaucheret Science STKE, 2005:pe43 (2005), and Yoshikawa et al. Genes Dev., 19:2164-2175 (2005), the contents and disclosures of which are incorporated herein by reference.

Plant miRNAs regulate their target genes by recognizing and binding to a complementary or near-perfectly complementary sequence (miRNA recognition site) in the target mRNA transcript, followed by cleavage of the transcript by RNase III enzymes, such as ARGONAUTE1. In plants, certain mismatches between a given miRNA recognition site and the corresponding mature miRNA are typically not tolerated, particularly mismatched nucleotides at positions 10 and 11 of the mature miRNA. Positions within the mature miRNA are given in the 5′ to 3′ direction. Perfect complementarity between a given miRNA recognition site and the corresponding mature miRNA is usually required at positions 10 and 11 of the mature miRNA. See, for example, Franco-Zorrilla et al. (2007) Nature Genetics, 39:1033-1037; and Axtell et al. (2006) Cell, 127:565-577.

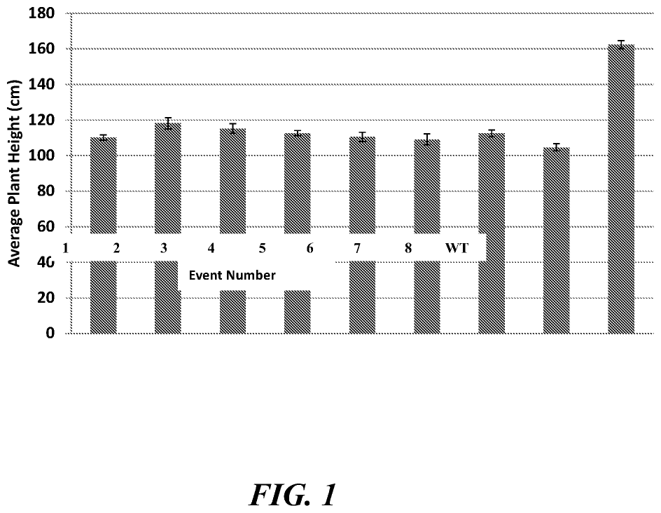

Many microRNA genes (MIR genes) have been identified and made publicly available in a database (“miRBase”, available on line at microrna.sanger.ac.uk/sequences; also see Griffiths-Jones et al. (2003) Nucleic Acids Res., 31:439-441). MIR genes have been reported to occur in intergenic regions, both isolated and in clusters in the genome, but can also be located entirely or partially within introns of other genes (both protein-coding and non-protein-coding). For a review of miRNA biogenesis, see Kim (2005) Nature Rev. Mol. Cell. Biol., 6:376-385. Transcription of MIR genes can be, at least in some cases, under promotional control of a MIR gene's own promoter. The primary transcript, termed a “pri-miRNA”, can be quite large (several kilobases) and can be polycistronic, containing one or more pre-miRNAs (fold-back structures containing a stem-loop arrangement that is processed to the mature miRNA) as well as the usual 5′ “cap” and polyadenylated tail of an mRNA. See, for example, FIG. 1 in Kim (2005) Nature Rev. Mol. Cell. Biol., 6:376-385.

Transgenic expression of miRNAs (whether a naturally occurring sequence or an artificial sequence) can be employed to regulate expression of the miRNA's target gene or genes. Recognition sites of miRNAs have been validated in all regions of a mRNA, including the 5′ untranslated region, coding region, intron region, and 3′ untranslated region, indicating that the position of the miRNA target or recognition site relative to the coding sequence may not necessarily affect suppression (see, e.g., Jones-Rhoades and Bartel (2004). Mol. Cell, 14:787-799, Rhoades et al. (2002) Cell, 110:513-520, Allen et al. (2004) Nat. Genet., 36:1282-1290, Sunkar and Zhu (2004) Plant Cell, 16:2001-2019). miRNAs are important regulatory elements in eukaryotes, and transgenic suppression with miRNAs is a useful tool for manipulating biological pathways and responses. A description of native miRNAs, their precursors, recognition sites, and promoters is provided in U.S. Patent Application Publication No. 2006/0200878, the contents and disclosures of which are incorporated herein by reference.

Designing an artificial miRNA sequence can be achieved by substituting nucleotides in the stem region of a miRNA precursor with a sequence that is complementary to the intended target, as demonstrated, for example, by Zeng et al. (2002) Mol. Cell, 9:1327-1333. According to many embodiments, the target may be a sequence of a GA20 oxidase gene or a GA3 oxidase gene. One non-limiting example of a general method for determining nucleotide changes in a native miRNA sequence to produce an engineered miRNA precursor for a target of interest includes the following steps: (a) Selecting a unique target sequence of at least 18 nucleotides specific to the target gene, e.g., by using sequence alignment tools such as BLAST (see, for example, Altschul et al. (1990) J. Mol. Biol., 215:403-410; Altschul et al. (1997) Nucleic Acids Res., 25:3389-3402); cDNA and/or genomic DNA sequences may be used to identify target transcript orthologues and any potential matches to unrelated genes, thereby avoiding unintentional silencing or suppression of non-target sequences; (b) Analyzing the target gene for undesirable sequences (e.g., matches to sequences from non-target species), and score each potential target sequence for GC content, Reynolds score (see Reynolds et al. (2004) Nature Biotechnol., 22:326-330), and functional asymmetry characterized by a negative difference in free energy (“ΔΔG”) (see Khvorova et al. (2003) Cell, 115:209-216). Preferably, target sequences (e.g., 19-mers) may be selected that have all or most of the following characteristics: (1) a Reynolds score >4, (2) a GC content between about 40% to about 60%, (3) a negative ΔΔG, (4) a terminal adenosine, (5) lack of a consecutive run of 4 or more of the same nucleotide; (6) a location near the 3′ terminus of the target gene; (7) minimal differences from the miRNA precursor transcript. In one aspect, a non-coding RNA molecule used herein to suppress a target gene (e.g., a GA20 or GA3 oxidase gene) is designed to have a target sequence exhibiting one or more, two or more, three or more, four or more, or five or more of the foregoing characteristics. Positions at every third nucleotide of a suppression element may be important in influencing RNAi efficacy; for example, an algorithm, “siExplorer” is publicly available at rna.chem.t.u-tokyo.ac.jp/siexplorer.htm (see Katoh and Suzuki (2007) Nucleic Acids Res., 10.1093/nar/gkl1120); (c) Determining a reverse complement of the selected target sequence (e.g., 19-mer) to use in making a modified mature miRNA. Relative to a 19-mer sequence, an additional nucleotide at position 20 may be matched to the selected target or recognition sequence, and the nucleotide at position 21 may be chosen to either be unpaired to prevent spreading of silencing on the target transcript or paired to the target sequence to promote spreading of silencing on the target transcript; and (d) Transforming the artificial miRNA into a plant.

According to embodiments of the present disclosure, a recombinant DNA molecule, construct or vector is provided comprising a transcribable DNA sequence or suppression element encoding a miRNA or precursor miRNA molecule for targeted suppression of a GA oxidase gene(s). Such a transcribable DNA sequence and suppression element may comprise a sequence of at least 19 nucleotides in length that corresponds to one or more GA oxidase gene(s) and/or a sequence complementary to one or more GA oxidase gene(s), although one or more sequence mismatches or non-base-paired nucleotides may be tolerated.

GA oxidase gene(s) may also be suppressed using one or more small interfering RNAs (siRNAs). The siRNA pathway involves the non-phased cleavage of a longer double-stranded RNA intermediate (“RNA duplex”) into small interfering RNAs (siRNAs). The size or length of siRNAs ranges from about 19 to about 25 nucleotides or base pairs, but common classes of siRNAs include those containing 21 or 24 base pairs. Thus, a transcribable DNA sequence or suppression element may encode a RNA molecule that is at least about 19 to about 25 nucleotides (or more) in length, such as at least 19, 20, 21, 22, 23, 24, or 25 nucleotides in length. For siRNA suppression, a recombinant DNA molecule, construct or vector is thus provided comprising a transcribable DNA sequence and suppression element encoding a siRNA molecule for targeted suppression of a GA oxidase gene(s). Such a transcribable DNA sequence and suppression element may be at least 19 nucleotides in length and have a sequence corresponding to one or more GA oxidase gene(s), and/or a sequence complementary to one or more GA oxidase gene(s).

GA oxidase gene(s) may also be suppressed using one or more trans-acting small interfering RNAs (ta-siRNAs). In the to-siRNA pathway, miRNAs serve to guide in-phase processing of siRNA primary transcripts in a process that requires an RNA-dependent RNA polymerase for production of a double-stranded RNA precursor. ta-siRNAs are defined by lack of secondary structure, a miRNA target site that initiates production of double-stranded RNA, requirements of DCL4 and an RNA-dependent RNA polymerase (RDR6), and production of multiple perfectly phased ˜21-nt small RNAs with perfectly matched duplexes with 2-nucleotide 3′ overhangs (see Allen et al. (2005) Cell, 121:207-221). The size or length of ta-siRNAs ranges from about 20 to about 22 nucleotides or base pairs, but are mostly commonly 21 base pairs. Thus, a transcribable DNA sequence or suppression element of the present invention may encode a RNA molecule that is at least about 20 to about 22 nucleotides in length, such as 20, 21, or 22 nucleotides in length. For to-siRNA suppression, a recombinant DNA molecule, construct or vector is thus provided comprising a transcribable DNA sequence or suppression element encoding a to-siRNA molecule for targeted suppression of a GA oxidase gene(s). Such a transcribable DNA sequence and suppression element may be at least 20 nucleotides in length and have a sequence corresponding to one or more GA oxidase gene(s) and/or a sequence complementary to one or more GA oxidase gene(s). For methods of constructing suitable to-siRNA scaffolds, see, e.g., U.S. Pat. No. 9,309,512, which is incorporated herein by reference in its entirety.

According to embodiments of the present invention, a recombinant DNA molecule, vector or construct is provided comprising a transcribable DNA sequence encoding a non-coding RNA molecule that binds or hybridizes to a target mRNA in a plant cell, wherein the target mRNA molecule encodes a GA20 or GA3 oxidase gene, and wherein the transcribable DNA sequence is operably linked to a constitutive or tissue-specific or tissue-preferred promoter. In addition to targeting a mature mRNA sequence, a non-coding RNA molecule may instead target an intronic sequence of a GA oxidase gene or mRNA transcript, or a GA oxidase mRNA sequence overlapping coding and non-coding sequences. According to other embodiments, a recombinant DNA molecule, vector or construct is provided comprising a transcribable DNA sequence encoding a non-coding RNA (precursor) molecule that is cleaved or processed into a mature non-coding RNA molecule that binds or hybridizes to a target mRNA in a plant cell, wherein the target mRNA molecule encodes a GA20 or GA3 oxidase protein, and wherein the transcribable DNA sequence is operably linked to a constitutive or tissue-specific or tissue-preferred promoter. For purposes of the present disclosure, a “non-coding RNA molecule” is a RNA molecule that does not encode a protein. Non-limiting examples of a non-coding RNA molecule include a microRNA (miRNA), a miRNA precursor, a small interfering RNA (siRNA), a siRNA precursor, a small RNA (18-26 nt in length) and precursors encoding the same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single-guide RNA (sgRNA).

According to embodiments of the present disclosure, suitable tissue-specific or tissue preferred promoters for expression of a GA20 oxidase or GA3 oxidase suppression element may include those promoters that drive or cause expression of its associated suppression element or sequence at least in the vascular and/or leaf tissue(s) of a corn or cereal plant, or possibly other tissues in the case of GA3 oxidase. Expression of the GA oxidase suppression element or construct with a tissue-specific or tissue-preferred promoter may also occur in other tissues of the cereal or corn plant outside of the vascular and leaf tissues, but active GA levels in the developing reproductive tissues of the plant (particularly in the female reproductive organ or ear) are preferably not significantly reduced or impacted (relative to wild type or control plants), such that development of the female organ or ear may proceed normally in the transgenic plant without off-types in the ear and a loss in yield potential.

Any vascular promoters known in the art may potentially be used as the tissue-specific or tissue-preferred promoter. Examples of vascular promoters include the RTBV promoter (see, e.g., SEQ ID NO: 65), a known sucrose synthase gene promoter, such as a corn sucrose synthase-1 (Sus1 or Sh1) promoter (see, e.g., SEQ ID NO: 67), a corn Sh1 gene paralog promoter, a barley sucrose synthase promoter (Ss1) promoter, a rice sucrose synthase-1 (RSs1) promoter (see, e.g., SEQ ID NO: 68), or a rice sucrose synthase-2 (RSs2) promoter (see, e.g., SEQ ID NO: 69), a known sucrose transporter gene promoter, such as a rice sucrose transporter promoter (SUT1) (see, e.g., SEQ ID NO: 70), or various known viral promoters, such as a Commelina yellow mottle virus (CoYMV) promoter, a wheat dwarf geminivirus (WDV) large intergenic region (LIR) promoter, a maize streak geminivirus (MSV) coat protein (CP) promoter, or a rice yellow stripe 1 (YS1)-like or OsYSL2 promoter (SEQ ID NO: 71), and any functional sequence portion or truncation of any of the foregoing promoters with a similar pattern of expression, such as a truncated RTBV promoter (see, e.g., SEQ ID NO: 66).

Any leaf promoters known in the art may potentially be used as the tissue-specific or tissue-preferred promoter. Examples of leaf promoters include a corn pyruvate phosphate dikinase or PPDK promoter (see, e.g., SEQ ID NO: 72), a corn fructose 1,6 bisphosphate aldolase or FDA promoter (see, e.g., SEQ ID NO: 73), and a rice Nadh-Gogat promoter (see, e.g., SEQ ID NO: 74), and any functional sequence portion or truncation of any of the foregoing promoters with a similar pattern of expression. Other examples of leaf promoters from monocot plant genes include a ribulose biphosphate carboxylase (RuBisCO) or RuBisCO small subunit (RBCS) promoter, a chlorophyll a/b binding protein gene promoter, a phosphoenolpyruvate carboxylase (PEPC) promoter, and a Myb gene promoter, and any functional sequence portion or truncation of any of these promoters with a similar pattern of expression.

Any other vascular and/or leaf promoters known in the art may also be used, including promoter sequences from related genes (e.g., sucrose synthase, sucrose transporter, and viral gene promoter sequences) from the same or different plant species or virus that have a similar pattern of expression. Further provided are promoter sequences with a high degree of homology to any of the foregoing. For example, a vascular promoter may comprise a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 65, 66, 67, 68, 69, 70, and 71, any functional sequence portion or truncation thereof, and/or any sequence complementary to any of the foregoing sequences; a leaf promoter may comprise, for example, a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 72, 73, and 74, any functional sequence portion or truncation thereof, and/or any sequence complementary to any of the foregoing sequences; and a constitutive promoter may comprise a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 75, 76, 77, 78, 79, 80, 81, 82, and 83, any functional sequence portion or truncation thereof, and/or any sequence complementary to any of the foregoing sequences. Examples of vascular and/or leaf promoters may further include other known, engineered and/or later-identified promoter sequences shown to have a pattern of expression in vascular and/or leaf tissue(s) of a cereal or corn plant. Furthermore, any known or later-identified constitutive promoter may also be used for expression of a GA20 oxidase or GA3 oxidase suppression element. Common examples of constitutive promoters are provided below.

As understood in the art, the term “promoter” may generally refer to a DNA sequence that contains an RNA polymerase binding site, transcription start site, and/or TATA box and assists or promotes the transcription and expression of an associated transcribable polynucleotide sequence and/or gene (or transgene). A promoter may be synthetic or artificial and/or engineered, varied or derived from a known or naturally occurring promoter sequence. A promoter may be a chimeric promoter comprising a combination of two or more heterologous sequences. A promoter of the present invention may thus include variants of promoter sequences that are similar in composition, but not identical to, other promoter sequence(s) known or provided herein. A promoter may be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or nearly all tissues of the plant are referred to as “constitutive” promoters. However, the expression level with a “constitutive promoter” is not necessarily uniform across different tissue types and cells. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential or predominant expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. A tissue-specific or tissue-preferred promoter may also be defined in terms of the specific or preferred tissue(s) in which it drives expression of its associated transcribable DNA sequence or suppression element. For example, a promoter that causes specific expression in vascular tissues may be referred to as a “vascular-specific promoter”, whereas a promoter that causes preferential or predominant expression in vascular tissues may be referred to as a “vascular-preferred promoter”. Likewise, a promoter that causes specific expression in leaf tissues may be referred to as a “leaf-specific promoter”, whereas a promoter that causes preferential or predominant expression in leaf tissues may be referred to as a “leaf-preferred promoter”. An “inducible” promoter is a promoter that initiates transcription in response to an environmental stimulus such as cold, drought or light, or other stimuli, such as wounding or chemical application. A promoter may also be classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed, as defined above.

Several of the GA oxidases in cereal plants consist of a family of related GA oxidase genes. For example, corn has a family of at least nine GA20 oxidase genes that includes GA20 oxidase_1, GA20 oxidase_2, GA20 oxidase_3, GA20 oxidase_4, GA20 oxidase_5, GA20 oxidase_6, GA20 oxidase_7, GA20 oxidase_8, and GA20 oxidase_9. However, there are only two GA3 oxidases in corn, GA3 oxidase_1 and GA3 oxidase_2. The DNA and protein sequences by SEQ ID NOs for each of these GA20 oxidase genes are provided in Table 1, and the DNA and protein sequences by SEQ ID NOs for each of these GA3 oxidase genes are provided in Table 2.

TABLE 1
DNA and protein sequences by sequence identifier for GA20
oxidase genes in corn.

| | | Coding Sequence | |
|---|---|---|---|
| GA20 oxidase Gene | cDNA | (CDS) | Protein |
| GA20 oxidase_1 | SEQ ID NO: 1 | SEQ ID NO: 2 | SEQ ID NO: 3 |
| GA20 oxidase_2 | SEQ ID NO: 4 | SEQ ID NO: 5 | SEQ ID NO: 6 |
| GA20 oxidase_3 | SEQ ID NO: 7 | SEQ ID NO: 8 | SEQ ID NO: 9 |
| GA20 oxidase_4 | SEQ ID NO: 10 | SEQ ID NO: 11 | SEQ ID NO: 12 |
| GA20 oxidase_5 | SEQ ID NO: 13 | SEQ ID NO: 14 | SEQ ID NO: 15 |
| GA20 oxidase_6 | SEQ ID NO: 16 | SEQ ID NO: 17 | SEQ ID NO: 18 |
| GA20 oxidase_7 | SEQ ID NO: 19 | SEQ ID NO: 20 | SEQ ID NO: 21 |
| GA20 oxidase_8 | SEQ ID NO: 22 | SEQ ID NO: 23 | SEQ ID NO: 24 |
| GA20 oxidase_9 | SEQ ID NO: 25 | SEQ ID NO: 26 | SEQ ID NO: 27 |

TABLE 2
DNA and protein sequences by sequence identifier for
GA3 oxidase genes in corn.

| | | Coding Sequence | |
|---|---|---|---|
| GA3 oxidase Gene | cDNA | (CDS) | Protein |
| GA3 oxidase_1 | SEQ ID NO: 28 | SEQ ID NO: 29 | SEQ ID NO: 30 |
| GA3 oxidase_2 | SEQ ID NO: 31 | SEQ ID NO: 32 | SEQ ID NO: 33 |

The genomic DNA sequence of GA20 oxidase_3 is provided in SEQ ID NO: 34, and the genomic DNA sequence of GA20 oxidase_5 is provided in SEQ ID NO: 35. For the GA20 oxidase_3 gene, SEQ ID NO: 34 provides 3000 nucleotides upstream of the GA20 oxidase_3 5′-UTR; nucleotides 3001-3096 correspond to the 5′-UTR; nucleotides 3097-3665 correspond to the first exon; nucleotides 3666-3775 correspond to the first intron; nucleotides 3776-4097 correspond to the second exon; nucleotides 4098-5314 correspond to the second intron; nucleotides 5315-5584 correspond to the third exon; and nucleotides 5585-5800 correspond to the 3′-UTR. SEQ ID NO: 34 also provides 3000 nucleotides downstream of the end of the 3′-UTR (nucleotides 5801-8800). For the GA20 oxidase_5 gene, SEQ ID NO: 35 provides 3000 nucleotides upstream of the GA20 oxidase_5 start codon (nucleotides 1-3000); nucleotides 3001-3791 correspond to the first exon; nucleotides 3792-3906 correspond to the first intron; nucleotides 3907-4475 correspond to the second exon; nucleotides 4476-5197 correspond to the second intron; nucleotides 5198-5473 correspond to the third exon; and nucleotides 5474-5859 correspond to the 3′-UTR. SEQ ID NO: 35 also provides 3000 nucleotides downstream of the end of the 3′-UTR (nucleotides 5860-8859).

The genomic DNA sequence of GA3 oxidase_1 is provided in SEQ ID NO: 36, and the genomic DNA sequence of GA3 oxidase_2 is provided in SEQ ID NO: 37. For the GA3 oxidase_1 gene, nucleotides 1-29 of SEQ ID NO: 36 correspond to the 5’-UTR; nucleotides 30-514 of SEQ ID NO: 36 correspond to the first exon; nucleotides 515-879 of SEQ ID NO: 36 correspond to the first intron; nucleotides 880-1038 of SEQ ID NO: 36 correspond to the second exon; nucleotides 1039-1158 of SEQ ID NO: 36 correspond to the second intron; nucleotides 1159-1663 of SEQ ID NO: 36 correspond to the third exon; and nucleotides 1664-1788 of SEQ ID NO: 36 correspond to the 3′-UTR. For the GA3 oxidase_2 gene, nucleotides 1-38 of SEQ ID NO: 37 correspond to the 5-UTR; nucleotides 39-532 of SEQ ID NO: 37 correspond to the first exon; nucleotides 533-692 of SEQ ID NO: 37 correspond to the first intron; nucleotides 693-851 of SEQ ID NO: 37 correspond to the second exon; nucleotides 852-982 of SEQ ID NO: 37 correspond to the second intron; nucleotides 983-1445 of SEQ ID NO: 37 correspond to the third exon; and nucleotides 1446-1698 of SEQ ID NO: 37 correspond to the 3′-UTR.

In addition to phenotypic observations with targeting the GA20 oxidase_3 and/or GA20 oxidase_5 gene(s), or the GA3 oxidase_1 and/or GA3 oxidase_2 gene(s), for suppression, a semi-dwarf phenotype is also observed with suppression of the GA20 oxidase_4 gene. The genomic DNA sequence of GA20 oxidase_4 is provided in SEQ ID NO: 38. For the GA oxidase_4 gene, SEQ ID NO: 38 provides nucleotides 1-1416 upstream of the 5′-UTR; nucleotides 1417-1543 of SEQ ID NO: 38 correspond to the 5′-UTR; nucleotides 1544-1995 of SEQ ID NO: 38 correspond to the first exon; nucleotides 1996-2083 of SEQ ID NO: 38 correspond to the first intron; nucleotides 2084-2411 of SEQ ID NO: 38 correspond to the second exon; nucleotides 2412-2516 of SEQ ID NO: 38 correspond to the second intron; nucleotides 2517-2852 of SEQ ID NO: 38 correspond to the third exon; nucleotides 2853-3066 of SEQ ID NO: 38 correspond to the 3′-UTR; and nucleotides 3067-4465 of SEQ ID NO: 38 corresponds to genomic sequence downstream of to the 3′-UTR.

According to embodiments of the present disclosure, a recombinant DNA molecule, vector or construct is provided comprising a transcribable DNA sequence encoding a non-coding RNA molecule, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least a segment or portion of a mRNA molecule (i) expressed from an endogenous GA oxidase gene and/or (ii) encoding an endogenous GA oxidase protein in the plant, wherein the transcribable DNA sequence is operably linked to a plant-expressible promoter, and wherein the plant is a cereal or corn plant.

According to some embodiments, a non-coding RNA molecule targets GA20 oxidase gene(s), such as GA20 oxidase_3 and/or GA20 oxidase_5 gene(s), for suppression and comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of one or more of SEQ ID NOs: 7, 8, 13 and 14. According to some embodiments, a non-coding RNA molecule is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to one or both of SEQ ID NOs: 9 and 15. According to further embodiments, a non-coding RNA molecule may comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% similar to one or both of SEQ ID NOs: 9 and 15. In addition to targeting a mature mRNA sequence (including either or both of the untranslated or exonic sequences), a non-coding RNA molecule may further target the intronic sequences of a GA20 oxidase gene or transcript.

According to some embodiments, a non-coding RNA molecule targets GA3 oxidase gene(s) for suppression and comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of one or more of SEQ ID NOs: 28, 29, 31 and 32. According to other embodiments, a non-coding RNA molecule is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to one or both of SEQ ID NOs: 30 and 33. According to further embodiments, a non-coding RNA molecule may comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% similar to one or both of SEQ ID NOs: 30 and 33. In addition to targeting a mature mRNA sequence (including either or both of the untranslated or exonic sequences), a non-coding RNA molecule may further target the intronic sequences of a GA3 oxidase gene or transcript.

According to some embodiments, a non-coding RNA molecule targets GA20 oxidase_4 gene for suppression and comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of one or both of SEQ ID NOs: 10 and 11. According to other embodiments, a non-coding RNA molecule is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to one or both of SEQ ID NO: 12. According to further embodiments, a non-coding RNA molecule may comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% similar to SEQ ID NOs: 12. In addition to targeting a mature mRNA sequence (including either or both of the untranslated or exonic sequences), a non-coding RNA molecule may further target the intronic sequences of a GA20 oxidase gene or transcript.

According to many embodiments, the non-coding RNA molecule encoded by the transcribable DNA sequence of the recombinant DNA molecule, vector or construct may be a precursor miRNA or siRNA that is processed or cleaved in a plant cell to form a mature miRNA or siRNA that targets a GA20 oxidase or GA3 oxidase gene.

According to embodiments of the present invention, GA levels may be reduced in the stalk or stem of a cereal or corn plant by targeting only a limited subset of genes within a GA oxidase family for suppression. Without being bound by theory, it is proposed that targeting of a limited number of genes within a GA oxidase family for suppression may produce the short stature phenotype and resistance to lodging in transgenic plants, but without off-types in the reproductive or ear tissues of the plant due to differential expression among GA oxidase genes, sufficient compensation for the suppressed GA oxidase gene(s) by other GA oxidase gene(s) in those reproductive tissues, and/or incomplete suppression of the targeted GA oxidase gene(s). Thus, not only may off-types be avoided by limiting expression or suppression of GA oxidase gene(s) with a tissue-specific or tissue preferred promoter, it is proposed that a limited subset of GA oxidase genes (e.g., a limited number of GA20 oxidase genes) may be targeted for suppression, such that the other GA oxidase genes within the same gene family (e.g., other GA20 oxidase genes) may compensate for loss of expression of the suppressed GA oxidase gene(s) in those tissues. Incomplete suppression of the targeted GA oxidase gene(s) may also allow for a sufficient level of expression of the targeted GA oxidase gene(s) in one or more tissues to avoid off-types or undesirable traits in the plant that would negatively affect crop yield, such as reproductive off-types or excessive shortening of plant height. Unlike complete loss-of-function mutations in a gene, suppression may allow for partial activity of the targeted gene to persist. Since the different GA20 oxidase genes have different patterns of expression in plants, targeting of a limited subset of GA20 oxidase genes for suppression may allow for modification of certain traits while avoiding off-types previously associated with GA mutants in cereal plants. In other words, the growth, developmental and reproductive traits or off-types previously associated with GA mutants in corn and other cereal crops may be decoupled by targeting only a limited number or subset (i.e., one or more, but not all) of the GA20 or GA3 oxidase genes and/or by incomplete suppression of a targeted GA oxidase gene. By transgenically targeting a subset of one or more endogenous GA3 or GA20 oxidase genes for suppression within a plant, a more pervasive pattern of expression (e.g., with a constitutive promoter) may be used to produce semi-dwarf plants without significant reproductive off-types and/or other undesirable traits in the plant, even with expression of the transgenic construct in reproductive tissue(s). Indeed, suppression elements and constructs are provided herein that selectively target the GA20 oxidase_3 and/or GA20 oxidase_5 genes (identified in Table 1 above) for suppression, which may be operably linked to a vascular, leaf and/or constitutive promoter.

With a suppression construct that only targets a limited subset of GA20 oxidase genes, such as the GA20 oxidase_3, GA20 oxidase_4, and/or GA20 oxidase_5 gene(s), or which targets the GA3 oxidase_1 and/or GA3 oxidase_2 gene(s), restricting the pattern of expression of the suppression element may be less crucial for obtaining normal reproductive development of the cereal or corn plant and avoidance of off-types in the female organ or ear due to compensation, etc., from the other GA20 and/or GA3 oxidase genes. Therefore, expression of a suppression construct and element, selectively or preferentially targeting, for instance, the GA20 oxidase_3 and/or GA20 oxidase_5 gene(s), the GA20 oxidase_4 gene, and/or the GA3 oxidase_1 and/or GA3 oxidase_2 gene(s) in corn, or similar genes and homologs in other cereal plants, may be driven by a variety of different plant-expressible promoter types including constitutive and tissue-specific or tissue-preferred promoters, such as a vascular or leaf promoter, which may include, for example, the RTBV promoter introduced above (e.g., a promoter comprising the RTBV (SEQ ID NO: 65) or truncated RTBV (SEQ ID NO: 66) sequence), and any other promoters that drive expression in tissues encompassing much or all of the vascular and/or leaf tissue(s) of a plant. Any known or later-identified constitutive promoter with a sufficiently high level of expression may also be used for expression of a suppression construct targeting a subset of GA20 and/or GA3 oxidase genes in corn, particularly the GA20 oxidase_3 and/or GA20 oxidase_5 gene(s), the GA20 oxidase_4 gene, and/or the GA3 oxidase_1 and/or GA3 oxidase_2 gene(s), or similar genes and homologs in other cereal plants.

Examples of constitutive promoters that may be used in monocot plants, such as cereal or corn plants, include, for example, various actin gene promoters, such as a rice Actin 1 promoter (see, e.g., U.S. Pat. No. 5,641,876; see also SEQ ID NO: 75 or SEQ ID NO: 76) and a rice Actin 2 promoter (see, e.g., U.S. Pat. No. 6,429,357; see also, e.g., SEQ ID NO: 77 or SEQ ID NO: 78), a CaMV 35S or 19S promoter (see, e.g., U.S. Pat. No. 5,352,605; see also, e.g., SEQ ID NO: 79 for CaMV 35S), a maize ubiquitin promoter (see, e.g., U.S. Pat. No. 5,510,474), a Coix lacryma-jobi polyubiquitin promoter (see, e.g., SEQ ID NO: 80), a rice or maize Gos2 promoter (see, e.g., Pater et al., The Plant Journal, 2(6): 837-44 1992; see also, e.g., SEQ ID NO: 81 for the rice Gos2 promoter), a FMV 35S promoter (see, e.g., U.S. Pat. No. 6,372,211), a dual enhanced CMV promoter (see, e.g., U.S. Pat. No. 5,322,938), a MMV promoter (see, e.g., U.S. Pat. No. 6,420,547; see also, e.g., SEQ ID NO: 82), a PCLSV promoter (see, e.g., U.S. Pat. No. 5,850,019; see also, e.g., SEQ ID NO: 83), an Emu promoter (see, e.g., Last et al., Theor. Appl. Genet. 81:581 (1991); and Mcelroy et al., Mol. Gen. Genet. 231:150 (1991)), a tubulin promoter from maize, rice or other species, a nopaline synthase (nos) promoter, an octopine synthase (ocs) promoter, a mannopine synthase (mas) promoter, or a plant alcohol dehydrogenase (e.g., maize Adhl) promoter, any other promoters including viral promoters known or later-identified in the art to provide constitutive expression in a cereal or corn plant, any other constitutive promoters known in the art that may be used in monocot or cereal plants, and any functional sequence portion or truncation of any of the foregoing promoters.

A sufficient level of expression of a transcribable DNA sequence encoding a non-coding RNA molecule targeting a GA oxidase gene for suppression may be necessary to produce a short stature, semi-dwarf phenotype that resists lodging, since lower levels of expression may be insufficient to lower active GA levels in the plant to a sufficient extent to cause a significant phenotype. Thus, tissue-specific and tissue-preferred promoters that drive, etc., a moderate or strong level of expression of their associated transcribable DNA sequence in active GA-producing tissue(s) of a plant may be preferred. Furthermore, such tissue-specific and tissue-preferred should drive, etc., expression of their associated transcribable DNA sequence during one or more vegetative stage(s) of plant development when the plant is growing and/or elongating including one or more of the following vegetative stage(s): V_{E}, V1, V2, V3, V4, V5, V6, V7, V8, V9, V10, V11, V12, V13, V14, Vn, V_{T}, such as expression at least during V3-V12, V4-V12, V5-V12, V6-V12, V7-V12, V8-V12, V3-V14, V5-V14, V6-V14, V7-V14, V8-V14, V9-V14, V10-V14, etc., or during any other range of vegetative stages when growth and/or elongation of the plant is occurring.

According to many embodiments, the plant-expressible promoter may preferably drive expression constitutively or in at least a portion of the vascular and/or leaf tissues of the plant. Different promoters driving expression of a suppression element targeting the endogenous GA20 oxidase_3 and/or GA20 oxidase_5 gene(s), the GA20 oxidase_4 gene, the GA3 oxidase_1 and/or GA3 oxidase_2 gene(s) in corn, or similar genes and homologs in other cereal plants, may be effective at reducing plant height and increasing lodging resistance to varying degrees depending on their particular pattern and strength of expression in the plant. However, some tissue-specific and tissue-preferred promoters driving expression of a GA20 or GA3 oxidase suppression element in a plant may not produce a significant short stature or anti-lodging phenotypes due to the spatial-temporal pattern of expression of the promoter during plant development, and/or the amount or strength of expression of the promoter being too low or weak. Furthermore, some suppression constructs may only reduce and not eliminate expression of the targeted GA20 or GA3 oxidase gene(s) when expressed in a plant, and thus depending on the pattern and strength of expression with a given promoter, the pattern and level of expression of the GA20 or GA3 oxidase suppression construct with such a promoter may not be sufficient to produce an observable plant height and lodging resistance phenotype in plants.

According to present embodiments, a recombinant DNA molecule, vector or construct for suppression of one or more endogenous GA20 or GA3 oxidase gene(s) in a plant is provided comprising a transcribable DNA sequence encoding a non-coding RNA molecule, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least a segment or portion of a mRNA molecule expressed from an endogenous GA oxidase gene and encoding an endogenous GA oxidase protein in the plant, wherein the transcribable DNA sequence is operably linked to a plant-expressible promoter, and wherein the plant is a cereal or corn plant. As stated above, in addition to targeting a mature mRNA sequence, a non-coding RNA molecule may further target the intronic sequence(s) of a GA oxidase gene or transcript. According to many embodiments, a non-coding RNA molecule may target a GA20 oxidase_3 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7 or SEQ ID NO: 8. According to some embodiments, a non-coding RNA molecule targeting a GA20 oxidase_3 gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 7 or SEQ ID NO: 8. According to some embodiments, a non-coding RNA molecule may target a GA20 oxidase gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 9. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein that is at least 80%, at least 85%, at least 90%, at least 95%, at least 96%, at least 97%, at least 98%, at least 99%, at least 99.5%, or 100% similar to SEQ ID NO: 9.

As mentioned above, a non-coding RNA molecule may target an intron sequence of a GA oxidase gene instead of, or in addition to, an exonic, 5′ UTR or 3′ UTR of the GA oxidase gene. Thus, a non-coding RNA molecule targeting the GA20 oxidase_3 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 34, and/or of nucleotides 3666-3775 or 4098-5314 of SEQ ID NO: 34. It is important to note that the sequences provided herein for the GA20 oxidase_3 gene may vary across the diversity of corn plants, lines and germplasms due to polymorphisms and/or the presence of different alleles of the gene.

Furthermore, a GA20 oxidase_3 gene may be expressed as alternatively spliced isoforms that may give rise to different mRNA, cDNA and coding sequences that can affect the design of a suppression construct and non-coding RNA molecule. Thus, a non-coding RNA molecule targeting a GA20 oxidase_3 gene for suppression may be more broadly defined as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 34.

According to embodiments of the present disclosure, a recombinant DNA molecule, vector or construct for suppression of an endogenous GA20 oxidase_5 gene in a plant is provided comprising a transcribable DNA sequence encoding a non-coding RNA molecule, wherein the non-coding RNA molecule targeting the GA20 oxidase_5 gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 13 or SEQ ID NO: 14. According to some embodiments, a non-coding RNA molecule targeting the GA20 oxidase_5 gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 13 or SEQ ID NO: 14. According to some embodiments, a non-coding RNA molecule may target a GA20 oxidase gene for suppression comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 15. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein that is at least 80%, at least 85%, at least 90%, at least 95%, at least 96%, at least 97%, at least 98%, at least 99%, at least 99.5%, or 100% similar to SEQ ID NO: 15.

As mentioned above, a non-coding RNA molecule may target an intron sequence of a GA oxidase gene instead of, or in addition to, an exonic or untranslated region of the mature mRNA of the GA oxidase gene. Thus, a non-coding RNA molecule targeting the GA20 oxidase_5 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 35, and/or of nucleotides 3792-3906 or 4476-5197 of SEQ ID NO: 35. The sequences provided herein for GA20 oxidase_5 may vary across the diversity of corn plants, lines and germplasms due to polymorphisms and/or the presence of different alleles of the gene. Furthermore, a GA20 oxidase_5 gene may be expressed as alternatively spliced isoforms that may give rise to different mRNA, cDNA and coding sequences that can affect the design of a suppression construct and non-coding RNA molecule. Thus, a non-coding RNA molecule targeting a GA20 oxidase_3 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 35.

According to further embodiments, a recombinant DNA molecule, vector or construct for joint suppression of endogenous GA20 oxidase_3 and GA20 oxidase_5 genes in a plant is provided comprising a transcribable DNA sequence encoding a non-coding RNA molecule, wherein the non-coding RNA molecule targeting the GA20 oxidase_3 and GA20 oxidase_5 genes for suppression comprises a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7 and/or SEQ ID NO: 8, and (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 13 and/or SEQ ID NO: 14. According to some of these embodiments, the non-coding RNA molecule jointly targeting the GA20 oxidase_3 and GA20 oxidase_5 genes for suppression may be complementary to at least 19 consecutive nucleotides, but no more than 27 consecutive nucleotides, such as complementary to 19, 20, 21, 22, 23, 24, 25, 26, or 27 consecutive nucleotides, of (i) SEQ ID NO: 7 (and/or SEQ ID NO: 8) and (ii) SEQ ID NO: 13 (and/or SEQ ID NO: 14). According to many embodiments, the non-coding RNA molecule jointly targeting the GA20 oxidase_3 and GA20 oxidase_5 genes for suppression comprises a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 9, and (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 15. As mentioned above, the non-coding RNA molecule may target an intron sequence of a GA oxidase gene. Thus, the non-coding RNA molecule may target an intron sequence(s) of one or both of the GA20 oxidase_3 and/or GA20 oxidase_5 gene(s) as identified above.

According to particular embodiments, the non-coding RNA molecule encoded by a transcribable DNA sequence comprises (i) a sequence that is at least 95%, at least 96%, at least 97%, at least 98%, at least 99%, at least 99.5%, or 100% complementary to SEQ ID NO: 39, 41, 43 or 45, and/or (ii) a sequence or suppression element encoding a non-coding RNA molecule comprising a sequence that is at least 95%, at least 96%, at least 97%, at least 98%, at least 99%, at least 99.5%, or 100% identical to SEQ ID NO: 40, 42, 44 or 46. According to some embodiments, the non-coding RNA molecule encoded by a transcribable DNA sequence may comprise a sequence with one or more mismatches, such as 1, 2, 3, 4, 5 or more complementary mismatches, relative to the sequence of a target or recognition site of a targeted GA20 oxidase gene mRNA, such as a sequence that is nearly complementary to SEQ ID NO: 40 but with one or more complementary mismatches relative to SEQ ID NO: 40. According to a particular embodiment, the non-coding RNA molecule encoded by the transcribable DNA sequence comprises a sequence that is 100% identical to SEQ ID NO: 40, which is 100% complementary to a target sequence within the cDNA and coding sequences of the GA20 oxidase_3 (i.e., SEQ ID NOs: 7 and 8, respectively), and/or to a corresponding sequence of a mRNA encoded by an endogenous GA20 oxidase_3 gene. However, the sequence of a non-coding RNA molecule encoded by a transcribable DNA sequence that is 100% identical to SEQ ID NO: 40, 42, 44 or 46 may not be perfectly complementary to a target sequence within the cDNA and coding sequences of the GA20 oxidase_5 gene (i.e., SEQ ID NOs: 13 and 14, respectively), and/or to a corresponding sequence of a mRNA encoded by an endogenous GA20 oxidase_5 gene. For example, the closest complementary match between the non-coding RNA molecule or miRNA sequence in SEQ ID NO: 40 and the cDNA and coding sequences of the GA20 oxidase_5 gene may include one mismatch at the first position of SEQ ID NO: 39 (i.e., the “C” at the first position of SEQ ID NO: 39 is replaced with a “G”; i.e., GTCCATCATGCGGTGCAACTA). However, the non-coding RNA molecule or miRNA sequence in SEQ ID NO: 40 may still bind and hybridize to the mRNA encoded by the endogenous GA20 oxidase_5 gene despite this slight mismatch.

According to embodiments of the present disclosure, a recombinant DNA molecule, vector or construct for suppression of one or more endogenous GA3 oxidase gene(s) in a plant is provided comprising a transcribable DNA sequence encoding a non-coding RNA molecule, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least a segment or portion of a mRNA molecule expressed from an endogenous GA3 oxidase gene and encoding an endogenous GA3 oxidase protein in the plant, wherein the transcribable DNA sequence is operably linked to a plant-expressible promoter, and wherein the plant is a cereal or corn plant. In addition to targeting a mature mRNA sequence, a non-coding RNA molecule may further target the intronic sequences of a GA3 oxidase gene or transcript.

According to some embodiments, a non-coding RNA molecule may target a GA3 oxidase_1 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 28 or SEQ ID NO: 29. According to some embodiments, a non-coding RNA molecule targeting a GA3 oxidase gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 28 or SEQ ID NO: 29. According to some embodiments, a non-coding RNA molecule targeting a GA3 oxidase gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 30. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein that is at least 80%, at least 85%, at least 90%, at least 95%, at least 96%, at least 97%, at least 98%, at least 99%, at least 99.5%, or 100% similar to SEQ ID NO:

30.

As mentioned above, a non-coding RNA molecule may target an intron sequence of a GA3 oxidase gene instead of, or in addition to, an exonic, 5′ UTR or 3′ UTR of the GA oxidase gene. Thus, a non-coding RNA molecule targeting the GA3 oxidase_1 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 36, and/or of nucleotides 515-879 or 1039-1158 of SEQ ID NO: 36. The sequences provided herein for GA3 oxidase_1 may vary across the diversity of corn plants, lines and germplasms due to polymorphisms and/or the presence of different alleles of the gene. Furthermore, a GA3 oxidase_1 gene may be expressed as alternatively spliced isoforms that may give rise to different mRNA, cDNA and coding sequences that can affect the design of a suppression construct and non-coding RNA molecule. Thus, a non-coding RNA molecule targeting a GA3 oxidase_1 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 36.

According to some embodiments, a non-coding RNA molecule may target a GA3 oxidase_2 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 31 or SEQ ID NO: 32. According to some embodiments, a non-coding RNA molecule targeting the GA3 oxidase gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 31 or SEQ ID NO: 32. According to some embodiments, a non-coding RNA molecule targeting the GA3 oxidase gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 33. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein that is at least 80%, at least 85%, at least 90%, at least 95%, at least 96%, at least 97%, at least 98%, at least 99%, at least 99.5%, or 100% similar to SEQ ID NO: 33.

As mentioned above, a non-coding RNA molecule may target an intron sequence of a GA3 oxidase gene instead of, or in addition to, an exonic, 5′ UTR or 3′ UTR of the GA3 oxidase gene. Thus, a non-coding RNA molecule targeting the GA3 oxidase_2 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 37, and/or of nucleotides 533-692 or 852-982 of SEQ ID NO: 37. The sequences provided herein for GA3 oxidase_2 may vary across the diversity of corn plants, lines and germplasms due to polymorphisms and/or the presence of different alleles of the gene. Furthermore, a GA3 oxidase_2 gene may be expressed as alternatively spliced isoforms that may give rise to different mRNA, cDNA and coding sequences that can affect the design of a suppression construct and non-coding RNA molecule. Thus, a non-coding RNA molecule targeting a GA3 oxidase_2 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 37.

According to particular embodiments, a non-coding RNA molecule encoded by a transcribable DNA sequence for targeting a GA3 oxidase gene comprises (i) a sequence that is at least 95%, at least 96%, at least 97%, at least 98%, at least 99%, at least 99.5%, or 100% complementary to SEQ ID NO: 57 or 59, and/or (ii) a sequence or suppression element encoding a non-coding RNA molecule comprising a sequence that is at least 95%, at least 96%, at least 97%, at least 98%, at least 99%, at least 99.5%, or 100% identical to SEQ ID NO: 58 or 60. According to some embodiments, the non-coding RNA molecule encoded by a transcribable DNA sequence may comprise a sequence with one or more mismatches, such as 1, 2, 3, 4, 5 or more complementary mismatches, relative to the sequence of a target or recognition site of a targeted GA3 oxidase gene mRNA, such as a sequence that is nearly complementary to SEQ ID NO: 57 or 59 but with one or more complementary mismatches relative to SEQ ID NO: 57 or 59. According to a particular embodiment, the non-coding RNA molecule encoded by the transcribable DNA sequence comprises a sequence that is 100% identical to SEQ ID NO: 58 or 60, which is 100% complementary to a target sequence within the cDNA and coding sequences of a GA3 oxidase_1 or GA3 oxidase_2 gene in corn (i.e., SEQ ID NOs: 28, 29, 31 and/or 32), and/or to a corresponding sequence of a mRNA encoded by an endogenous GA3 oxidase_1 or GA3 oxidase_2 gene.

According to some embodiments, a non-coding RNA molecule may target a GA20 oxidase_4 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 10 or SEQ ID NO: 11. According to some embodiments, a non-coding RNA molecule targeting a GA20 oxidase_4 gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 10 or SEQ ID NO: 11. According to some embodiments, a non-coding RNA molecule targeting the GA20 oxidase gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 12. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein that is at least 80%, at least 85%, at least 90%, at least 95%, at least 96%, at least 97%, at least 98%, at least 99%, at least 99.5%, or 100% similar to SEQ ID NO: 12.

As mentioned above, a non-coding RNA molecule may target an intron sequence of a GA20 oxidase gene instead of, or in addition to, an exonic, 5′ UTR or 3′ UTR of the GA20 oxidase gene. Thus, a non-coding RNA molecule targeting a GA20 oxidase_4 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 38, and/or of nucleotides 1996-2083 or 2412-2516 of SEQ ID NO: 38. The sequences provided herein for GA20 oxidase_4 may vary across the diversity of corn plants, lines and germplasms due to polymorphisms and/or the presence of different alleles of the gene. Furthermore, a GA20 oxidase_4 gene may be expressed as alternatively spliced isoforms that may give rise to different mRNA, cDNA and coding sequences that can affect the design of a suppression construct and non-coding RNA molecule. Thus, a non-coding RNA molecule targeting a GA20 oxidase_4 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 38.

According to particular embodiments, a non-coding RNA molecule encoded by a transcribable DNA sequence for targeting a GA20 oxidase_4 gene comprises (i) a sequence that is at least 95%, at least 96%, at least 97%, at least 98%, at least 99%, at least 99.5%, or 100% complementary to SEQ ID NO: 61, and/or (ii) a sequence or suppression element encoding a non-coding RNA molecule comprising a sequence that is at least 95%, at least 96%, at least 97%, at least 98%, at least 99%, at least 99.5%, or 100% identical to SEQ ID NO: 62. According to some embodiments, the non-coding RNA molecule encoded by a transcribable DNA sequence may comprise a sequence with one or more mismatches, such as 1, 2, 3, 4, 5 or more complementary mismatches, relative to the sequence of a target or recognition site of a targeted GA20 oxidase gene mRNA, such as a sequence that is nearly complementary to SEQ ID NO: 61 but with one or more complementary mismatches relative to SEQ ID NO: 61. According to a particular embodiment, the non-coding RNA molecule encoded by the transcribable DNA sequence comprises a sequence that is 100% identical to SEQ ID NO: 62, which is 100% complementary to a target sequence within the cDNA and coding sequences of a GA20 oxidase_4 gene in corn (i.e., SEQ ID NO: 10 or 11), and/or to a corresponding sequence of a mRNA encoded by an endogenous GA20 oxidase_4 gene.

According to embodiments of the present disclosure, a recombinant DNA construct is provided comprising a transcribable DNA sequence encoding a non-coding RNA molecule targeting an endogenous GA20 oxidase_3 and/or the GA20 oxidase_5 gene(s) for suppression, wherein the transcribable DNA sequence is operably linked to a constitutive, tissue-specific or tissue-preferred promoter, and wherein the transcribable DNA sequence causes the expression level of an endogenous GA20 oxidase_3 and/or the GA20 oxidase_5 gene(s) to become reduced or lowered in one or more tissue(s) of a plant transformed with the transcribable DNA sequence.

Such a non-coding RNA molecule encoded by the transcribable DNA sequence may comprise a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 9, and/or (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 15.

According to embodiments of the present disclosure, a recombinant DNA construct is provided comprising a transcribable DNA sequence encoding a non-coding RNA molecule targeting an endogenous GA3 oxidase_1 and/or the GA3 oxidase_2 gene(s) for suppression, wherein the transcribable DNA sequence is operably linked to a constitutive, tissue-specific or tissue-preferred promoter, and wherein the transcribable DNA sequence causes the expression level of an endogenous GA3 oxidase_1 and/or the GA3 oxidase_2 gene(s) to become reduced or lowered in one or more tissue(s) of a plant transformed with the transcribable DNA sequence. Such a non-coding RNA molecule encoded by the transcribable DNA sequence may comprise a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 30, and/or (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 33.

According to embodiments of the present disclosure, a recombinant DNA construct is provided comprising a transcribable DNA sequence encoding a non-coding RNA molecule targeting an endogenous GA20 oxidase_4 gene for suppression, wherein the transcribable DNA sequence is operably linked to a constitutive, tissue-specific or tissue-preferred promoter, and wherein the transcribable DNA sequence causes the expression level of an endogenous GA20 oxidase_4 gene to become reduced or lowered in one or more tissue(s) of a plant transformed with the transcribable DNA sequence. Such a non-coding RNA molecule encoded by the transcribable DNA sequence may comprise a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 12.

According to many embodiments, a modified or transgenic plant is provided that is transformed with a recombinant DNA construct comprising a transcribable DNA sequence encoding a non-coding RNA molecule targeting an endogenous GA20 oxidase_3 and/or GA20 oxidase_5 gene(s) for suppression, and/or has an endogenous GA20 oxidase_3 and/or GA20 oxidase_5 gene(s) edited through targeted genome editing techniques, as provided herein, wherein the transcribable DNA sequence is operably linked to a constitutive promoter or a tissue-specific or tissue-preferred promoter, such as a vascular promoter or a leaf promoter, and wherein the expression level of the endogenous GA20 oxidase_3 and/or GA20 oxidase_5 gene(s) is eliminated, reduced or lowered in one or more plant tissue(s), such as one or more vascular and/or leaf tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant. According to many embodiments, in a modified or transgenic plant is provided that is transformed with a recombinant DNA construct comprising a transcribable DNA sequence encoding a non-coding RNA molecule targeting an endogenous GA20 oxidase_3 and/or GA20 oxidase_5 gene(s) for suppression, and/or has an endogenous GA20 oxidase_3 and/or GA20 oxidase_5 gene(s) edited through targeted genome editing techniques to reduce or eliminate its level of expression and/or activity, wherein the transcribable DNA sequence is operably linked to a constitutive promoter or a tissue-specific or tissue-preferred promoter, such as a vascular promoter or a leaf promoter, and wherein the level of one or more active GAs, such as GA1, GA3, GA4, and/or GA7, is reduced or lowered in one or more plant tissue(s), such as one or more stem, internode, vascular and/or leaf tissue(s) or one or more stem and/or internode tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant.

According to many embodiments, a modified or transgenic plant is provided that is transformed with a recombinant DNA construct comprising a transcribable DNA sequence encoding a non-coding RNA molecule targeting an endogenous GA3 oxidase_1 and/or GA3 oxidase_2 gene(s) for suppression, and/or has an endogenous GA3 oxidase_1 or GA3 oxidase_2 gene edited through targeted genome editing techniques, as provided herein, wherein the transcribable DNA sequence is operably linked to a constitutive promoter or a tissue-specific or tissue-preferred promoter, such as a vascular promoter or a leaf promoter, and wherein the expression level of the endogenous GA3 oxidase_1 and/or GA3 oxidase_2 gene(s) is eliminated, reduced or lowered in one or more plant tissue(s), such as one or more vascular and/or leaf tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant. According to many embodiments, a modified or transgenic plant is provided that is transformed with a recombinant DNA construct comprising a transcribable DNA sequence encoding a non-coding RNA molecule targeting an endogenous GA3 oxidase_1 and/or GA3 oxidase_2 gene(s) for suppression, and/or has an endogenous GA3 oxidase_1 and/or GA3 oxidase_2 gene edited through targeted genome editing techniques to reduce or eliminate its level of expression and/or activity, wherein the transcribable DNA sequence is operably linked to a constitutive promoter or a tissue-specific or tissue-preferred promoter, such as a vascular promoter or a leaf promoter, and wherein the level of one or more active GAs, such as GA1, GA3, GA4, and/or GA7, is reduced or lowered in one or more plant tissue(s), such as one or more stem, internode, vascular and/or leaf tissue(s) or one or in more stem and/or internode tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant.

According to many embodiments, a modified or transgenic plant is provided that is transformed with a recombinant DNA construct comprising a transcribable DNA sequence encoding a non-coding RNA molecule targeting an endogenous GA20 oxidase_4 gene for suppression, and/or has an endogenous GA20 oxidase_4 gene edited through targeted genome editing techniques, as provided herein, wherein the transcribable DNA sequence is operably linked to a constitutive promoter or a tissue-specific or tissue-preferred promoter, such as a vascular promoter or a leaf promoter, and wherein the expression level of the endogenous GA20 oxidase_4 gene(s) is eliminated, reduced or lowered in one or more plant tissue(s), such as one or more vascular and/or leaf tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant. According to many embodiments, a modified or transgenic plant is provided that is transformed with a recombinant DNA construct comprising a transcribable DNA sequence encoding a non-coding RNA molecule targeting an endogenous GA20 oxidase_4 gene(s) for suppression, and/or has an endogenous GA20 oxidase_4 gene edited through targeted genome editing techniques to reduce or eliminate its level of expression and/or activity, wherein the transcribable DNA sequence is operably linked to a constitutive promoter or a tissue-specific or tissue-preferred promoter, such as a vascular promoter or a leaf promoter, and wherein the level of one or more active GAs, such as GA1, GA3, GA4, and/or GA7, is reduced or lowered in one or more plant tissue(s), such as one or more stem, internode, vascular and/or leaf tissue(s) or one or more stem and/or internode tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant.

According to many embodiments, a modified or transgenic plant is provided that is transformed with a recombinant DNA construct comprising a transcribable DNA sequence encoding a non-coding RNA molecule targeting an endogenous GA20 oxidase_3, GA20 oxidase_4, and/or GA20 oxidase_5 gene(s) for suppression, is transformed with a recombinant DNA construct comprising a transcribable DNA sequence encoding a non-coding RNA molecule targeting an endogenous GA3 oxidase_1 and/or the GA3 oxidase_2 gene(s) for suppression, and/or has an endogenous GA20 oxidase_3, GA20 oxidase_4, and/or the GA20 oxidase_5 gene edited through targeted genome editing techniques, to reduce or eliminate its level of expression and/or activity, as provided herein, wherein the transcribable DNA sequence is operably linked to a constitutive promoter or a tissue-specific or tissue-preferred promoter, such as a vascular promoter or a leaf promoter, and wherein the modified or transgenic plant has one or more of the following traits: a semi-dwarf or reduced plant height or stature, decreased stem internode length, increased lodging resistance, and/or increased stem or stalk diameter. Such a modified or transgenic plant may not have any significant reproductive off-types. A modified or transgenic plant may have one or more of the following additional traits: reduced green snap, deeper roots, increased leaf area, earlier canopy closure, higher stomatal conductance, lower ear height, increased foliar water content, improved drought tolerance, increased nitrogen use efficiency, increased water use efficiency, reduced anthocyanin content and anthocyanin area in leaves under normal and/or nitrogen or water limiting stress conditions, increased ear weight, increased kernel number, increased kernel weight, increased yield, and/or increased harvest index. According to many of these embodiments, the level of expression and/or activity of an endogenous GA20 oxidase_3, GA20 oxidase_4, and/or GA20 oxidase_5 gene(s), or an endogenous GA3 oxidase_1 and/or GA3 oxidase_2 gene(s), may be eliminated, reduced or lowered in one or more plant tissue(s), such as one or more vascular and/or leaf tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant, and/or the level of one or more active GAs, such as GA1, GA3, GA4, and/or GA7, is reduced or lowered in one or more plant tissue(s), such as one or more stem, internode, vascular and/or leaf tissue(s), or one or more stem and/or internode tissue(s), of the modified or transgenic plant by at least 5%, at least 10%, at least 20%, at least 25%, at least 30%, at least 40%, at least 50%, at least 60%, at least 70%, at least 75%, at least 80%, at least 90%, or 100% as compared to a wild type or control plant.

According to many of the embodiments described in the above paragraphs, the non-coding RNA molecule encoded by the transcribable DNA sequence of the recombinant DNA molecule, vector or construct may be a precursor miRNA or siRNA that may be subsequently processed or cleaved in a plant cell to form a mature miRNA or siRNA.

A recombinant DNA molecule, construct or vector of the present disclosure may comprise a transcribable DNA sequence encoding a non-coding RNA molecule that targets an endogenous GA oxidase gene for suppression, wherein the transcribable DNA sequence is operatively linked to a plant-expressible promoter, such as a constitutive or vascular and/or leaf promoter. For purposes of the present disclosure, a non-coding RNA molecule encoded by a transcribable DNA sequence that targets an endogenous GA oxidase gene for suppression may include a mature non-coding RNA molecule that targets an endogenous GA oxidase gene for suppression, and/or a precursor RNA molecule that may become processed in a plant cell into a mature non-coding RNA molecule, such as a miRNA or siRNA, that targets an endogenous GA oxidase gene for suppression. In addition to its associated promoter, a transcribable DNA sequence encoding a non-coding RNA molecule for suppression of an endogenous GA oxidase gene may also be operatively linked to one or more additional regulatory element(s), such as an enhancer(s), leader, transcription start site (TSS), linker, 5′ and 3′ untranslated region(s) (UTRs), intron(s), polyadenylation signal, termination region or sequence, etc., that are suitable, necessary or preferred for strengthening, regulating or allowing expression of the transcribable DNA sequence in a plant cell. Such additional regulatory element(s) may be optional and/or used to enhance or optimize expression of the transgene or transcribable DNA sequence. As provided herein, an “enhancer” may be distinguished from a “promoter” in that an enhancer typically lacks a transcription start site, TATA box, or equivalent sequence and is thus insufficient alone to drive transcription. As used herein, a “leader” may be defined generally as the DNA sequence of the 5′-UTR of a gene (or transgene) between the transcription start site (TSS) and 5′ end of the transcribable DNA sequence or protein coding sequence start site of the transgene.

According to further embodiments, methods are provided for transforming a plant cell, tissue or explant with a recombinant DNA molecule or construct comprising a transcribable DNA sequence or transgene operably linked to a plant-expressible promoter to produce a transgenic plant. The transcribable DNA sequence may encode a non-coding RNA molecule that targets a GA oxidase gene(s) for suppression, or a RNA precursor that is processed into a mature RNA molecule, such as a miRNA or siRNA, that targets one or more GA oxidase gene(s) for suppression. Numerous methods for transforming chromosomes or plastids in a plant cell with a recombinant DNA molecule or construct are known in the art, which may be used according to method embodiments of the present invention to produce a transgenic plant cell and plant. Any suitable method or technique for transformation of a plant cell known in the art may be used according to present methods. Effective methods for transformation of plants include bacterially mediated transformation, such as Agrobacterium-mediated or Rhizobium-mediated transformation, and microprojectile or particle bombardment-mediated transformation. A variety of methods are known in the art for transforming explants with a transformation vector via bacterially mediated transformation or microprojectile or particle bombardment and then subsequently culturing, etc., those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art.

Methods of transforming plant cells and explants are well known by persons of ordinary skill in the art. Methods for transforming plant cells by microprojectile bombardment with particles coated with recombinant DNA are provided, for example, in U.S. Pat. Nos. 5,550,318; 5,538,880 6,160,208; 6,399,861; and 6,153,812, and Agrobacterium-mediated transformation is described, for example, in U.S. Pat. Nos. 5,159,135; 5,824,877; 5,591,616; 6,384,301; 5,750,871; 5,463,174; and 5,188,958, all of which are incorporated herein by reference.

Additional methods for transforming plants can be found in, for example, Compendium of Transgenic Crop Plants (2009) Blackwell Publishing. Any suitable method of plant transformation known or later developed in the art can be used to transform a plant cell or explant with any of the nucleic acid molecules, constructs or vectors provided herein.

Transgenic plants produced by transformation methods may be chimeric or non-chimeric for the transformation event depending on the methods and explants used. Methods are further provided for expressing a non-coding RNA molecule that targets an endogenous GA oxidase gene for suppression in one or more plant cells or tissues under the control of a plant-expressible promoter, such as a constitutive, tissue-specific, tissue-preferred, vascular and/or leaf promoter as provided herein. Such methods may be used to create transgenic cereal or corn plants having a shorter, semi-dwarf stature, reduced internode length, increased stalk/stem diameter, and/or improved lodging resistance. Such transgenic cereal or corn plants may further have other traits that may be beneficial for yield, such as reduced green snap, deeper roots, increased leaf area, earlier canopy closure, improved drought tolerance, increased nitrogen use efficiency, increased water use efficiency, higher stomatal conductance, lower ear height, increased foliar water content, reduced anthocyanin content and/or area in leaves under normal or nitrogen or water limiting stress conditions, increased ear weight, increased seed or kernel number, increased seed or kernel weight, increased yield, and/or increased harvest index, relative to a wild type or control plant. As used herein, “harvest index” refers to the mass of the harvested grain divided by the total mass of the above-ground biomass of the plant over a harvested area.

Transgenic plants expressing a GA oxidase transgene or non-coding RNA molecule that targets an endogenous GA oxidase gene for suppression may have an earlier canopy closure (e.g., approximately one day earlier, or 12-48 hours, 12-36 hours, 18-36 hours, or about 24 hours earlier canopy closure) than a wild type or control plant. Although transgenic plants expressing a GA oxidase transgene or non-coding RNA molecule that targets an endogenous GA oxidase gene for suppression may have a lower ear height than a wild type or control plant, the height of the ear may generally be at least 18 inches above the ground. Transgenic plants expressing a non-coding RNA molecule that targets an endogenous GA oxidase gene for suppression may have greater biomass and/or leaf area during one or more late vegetative stages (e.g., V8-V12) than a wild type or control plant. Transgenic plants expressing a GA oxidase transgene or non-coding RNA molecule that targets an endogenous GA oxidase gene for suppression may have deeper roots during later vegetative stages when grown in the field, than a wild type or control plant, which may be due to an increased root front velocity. These transgenic plants may reach a depth 90 cm below ground sooner (e.g., 10-25 days sooner, 15-25 days sooner, or about 20 days sooner) than a wild type or control plant, which may occur by the vegetative to reproductive transition of the plant (e.g., by V16/R1 at about 50 days after planting as opposed at about 70 days after planting for control plants).

Recipient cell(s) or explant or cellular targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, a callus cell, a chloroplast, a stomatal cell, a trichome cell, a root hair cell, a storage root cell, or a vascular tissue cell, a seed, embryo, meristem, cotyledon, hypocotyl, endosperm, root, shoot, stem, node, callus, cell suspension, protoplast, flower, leaf, pollen, anther, ovary, ovule, pericarp, bud, and/or vascular tissue, or any transformable portion of any of the foregoing. For plant transformation, any target cell(s), tissue(s), explant(s), etc., that may be used to receive a recombinant DNA transformation vector or molecule of the present disclosure may be collectively be referred to as an “explant” for transformation. Preferably, a transformable or transformed explant cell or tissue may be further developed or regenerated into a plant. Any cell or explant from which a fertile plant can be grown or regenerated is contemplated as a useful recipient cell or explant for practice of this disclosure (i.e., as a target explant for transformation). Callus can be initiated or created from various tissue sources, including, but not limited to, embryos or parts of embryos, non-embryonic seed tissues, seedling apical meristems, microspores, and the like. Any cells that are capable of proliferating as callus may serve as recipient cells for transformation. Transformation methods and materials for making transgenic plants (e.g., various media and recipient target cells or explants and methods of transformation and subsequent regeneration of into transgenic plants) are known in the art.

Transformation of a target plant material or explant may be practiced in tissue culture on nutrient media, for example a mixture of nutrients that allow cells to grow in vitro or cell culture. Transformed explants, cells or tissues may be subjected to additional culturing steps, such as callus induction, selection, regeneration, etc., as known in the art. Transformation may also be carried out without creation or use of a callus tissue. Transformed cells, tissues or explants containing a recombinant DNA sequence insertion or event may be grown, developed or regenerated into transgenic plants in culture, plugs, or soil according to methods known in the art. Transgenic plants may be further crossed to themselves or other plants to produce transgenic seeds and progeny. A transgenic plant may also be prepared by crossing a first plant comprising the recombinant DNA sequence or transformation event with a second plant lacking the insertion. For example, a recombinant DNA construct or sequence may be introduced into a first plant line that is amenable to transformation, which may then be crossed with a second plant line to introgress the recombinant DNA construct or sequence into the second plant line. Progeny of these crosses can be further back crossed into the more desirable line multiple times, such as through 6 to 8 generations or back crosses, to produce a progeny plant with substantially the same genotype as the original parental line, but for the introduction of the recombinant DNA construct or sequence.

A transgenic or modified plant, plant part, cell, or explant provided herein may be of an elite variety or an elite line. An elite variety or an elite line refers to a variety that has resulted from breeding and selection for superior agronomic performance. A transgenic or edited plant, cell, or explant provided herein may be a hybrid plant, cell, or explant. As used herein, a “hybrid” is created by crossing two plants from different varieties, lines, inbreds, or species, such that the progeny comprises genetic material from each parent. Skilled artisans recognize that higher order hybrids can be generated as well. For example, a first hybrid can be made by crossing Variety A with Variety B to create a A×B hybrid, and a second hybrid can be made by crossing Variety C with Variety D to create an C×D hybrid. The first and second hybrids can be further crossed to create the higher order hybrid (A×B)×(C×D) comprising genetic information from all four parent varieties.

According to embodiments of the present disclosure, a modified plant is provided comprising a GA oxidase suppression element that targets two or more GA oxidase genes for suppression, or a combination of two or more GA oxidase suppression element(s) and/or gene edit(s). A recombinant DNA construct or vector may comprise a single cassette or suppression element comprising a transcribable DNA sequence designed or chosen to encode a non-coding RNA molecule that is complementary to mRNA recognition or target sequences of two or more GA oxidase genes including at least a first GA oxidase gene and a second GA oxidase gene—i.e., the mRNAs of the targeted GA oxidase genes share an identical or nearly identical (or similar) sequence such that a single suppression element and encoded non-coding RNA molecule can target each of the targeted GA oxidase genes for suppression. For example, an expression cassette and suppression construct is provided herein comprising a transcribable DNA sequence that encodes a single non-coding RNA molecule that targets both the GA20 oxidase_3 and GA20 oxidase_5 genes for suppression.

According to other embodiments, a recombinant DNA construct or vector may comprise two or more suppression elements or sequences that may be stacked together in a construct or vector either in tandem in a single expression cassette or separately in two or more expression cassettes. A recombinant DNA construct or vector may comprise a single expression cassette or suppression element comprising a transcribable DNA sequence that encodes a non-coding RNA molecule comprising two or more targeting sequences arranged in tandem, including at least a first targeting sequence and a second targeting sequence, wherein the first targeting sequence is complementary to a mRNA recognition or target site of a first GA oxidase gene, and the second targeting sequence is complementary to a mRNA recognition or target site of a second GA oxidase gene, and wherein the transcribable DNA sequence is operably linked to a plant-expressible promoter. The plant-expressible promoter may be a constitutive promoter, or a tissue-specific or tissue-preferred promoter, as provided herein. The non-coding RNA molecule may be expressed as a pre-miRNA that becomes processed into two or more mature miRNAs including at least a first mature miRNA and a second miRNA, wherein the first miRNA comprises a targeting sequence that is complementary to the mRNA recognition or target site of the first GA oxidase gene, and the second miRNA comprises a targeting sequence that is complementary to the mRNA recognition or target site of the second GA oxidase gene.

According to other embodiments, a recombinant DNA construct or vector may comprise two or more expression cassettes including a first expression cassette and a second expression cassette, wherein the first expression cassette comprises a first transcribable DNA sequence operably linked to a first plant-expressible promoter, and the second expression cassette comprises a second transcribable DNA sequence operably linked to a second plant-expressible promoter, wherein the first transcribable DNA sequence encodes a first non-coding RNA molecule comprising a targeting sequence that is complementary to a mRNA recognition or target site of a first GA oxidase gene, and the second transcribable DNA sequence encodes a second non-coding RNA molecule comprising a targeting sequence that is complementary to a mRNA recognition or target site of a second GA oxidase gene. The first and second plant-expressible promoters may each be a constitutive promoter, or a tissue-specific or tissue-preferred promoter, as provided herein, and the first and second plant-expressible promoters may be the same or different promoters.

According to other embodiments, two or more suppression elements or constructs targeting GA oxidase gene(s) and/or GA oxidase gene edit(s) may be combined in a modified plant by crossing two or more plants together in one or more generations to produce a modified plant having a desired combination of suppression element(s) and/or gene edit(s). According to these embodiments, a first modified plant comprising a suppression element or construct targeting a GA oxidase gene(s) (or a GA oxidase gene edit) may be crossed to a second modified plant comprising a suppression element or construct targeting a GA oxidase gene(s) (or a GA oxidase gene edit), such that a modified progeny plant may be made comprising a first suppression element or construct and a second suppression element or construct, a suppression element or construct and a GA oxidase gene edit, or a first GA oxidase gene edit and a second GA oxidase gene edit. Alternatively, a modified plant comprising two or more suppression elements or constructs targeting GA oxidase gene(s) and/or GA oxidase gene edit(s) may be made by (i) co-transforming a first suppression element or construct and a second suppression element or construct (each targeting a GA oxidase gene for suppression), (ii) transforming a modified plant with a second suppression element or construct, wherein the modified plant already comprises a first suppression element or construct, (iii) transforming a modified plant with a suppression element or construct, wherein the modified plant already comprises an edited GA oxidase gene, (iv) transforming a modified plant with a construct(s) for making one or more edits in GA oxidase gene(s), wherein the modified plant already comprises a suppression element or construct, or (v) transforming with construct(s) for making two or more edits in GA oxidase gene(s).

According to embodiments of the present disclosure, modified plants are provided comprising two or more constructs targeting GA oxidase gene(s) for suppression including a first recombinant DNA construct and a second recombinant DNA construct, wherein the first recombinant DNA construct comprises a first transcribable DNA sequence encoding a first non-coding RNA molecule that is complementary to a mRNA recognition or target sequence of a first GA oxidase gene, and the second recombinant DNA construct comprises a second transcribable DNA sequence encoding a second non-coding RNA molecule that is complementary to a mRNA recognition or target sequence of a second GA oxidase gene. The first and second recombinant DNA constructs may be stacked in a single vector and transformed into a plant as a single event, or present in separate vectors or constructs that may be transformed as separate events. According to these embodiments, the first GA oxidase gene may be a GA20 oxidase_3, GA20 oxidase_5, GA20 oxidase_4, GA3 oxidase_1, or GA3 oxidase_2 gene, the first non-coding RNA molecule is complementary to a recognition or target sequence of an mRNA expressed from such GA oxidase gene, and the second GA oxidase gene may be a GA20 oxidase_3, GA20 oxidase_5, GA20 oxidase_4, GA3 oxidase_1, or GA3 oxidase_2 gene. According to some embodiments, the first and second GA oxidase genes may be the same or different GA oxidase gene(s). Alternatively, the second GA oxidase gene may be another GA oxidase gene, such as a GA20 oxidase_1, GA20 oxidase_2, GA20 oxidase_6, GA20 oxidase_7, GA20 oxidase_8, or GA20 oxidase_9 gene, and the second non-coding RNA molecule is complementary to a recognition or target sequence of an mRNA expressed from such GA oxidase gene.

According to embodiments of the present disclosure, modified plants are provided comprising a recombinant DNA construct targeting GA oxidase genes for suppression comprising a transcribable DNA sequence encoding a non-coding RNA molecule that comprises two or more targeting sequences arranged in tandem including at least a first targeting sequence that is complementary to a mRNA recognition or target sequence of a first GA oxidase gene and a second targeting sequence that is complementary to a mRNA recognition or target sequence of a second GA oxidase gene. The non-coding RNA molecule may be expressed as a pre-miRNA that becomes processed into two or more mature miRNAs including at least a first mature miRNA and a second miRNA, wherein the first miRNA comprises the first targeting sequence that is complementary to the mRNA recognition or target site of the first GA oxidase gene, and the second miRNA comprises the second targeting sequence that is complementary to the mRNA recognition or target site of the second GA oxidase gene. According to these embodiments, the first GA oxidase gene may be a GA20 oxidase_3, GA20 oxidase_5, GA20 oxidase_4, GA3 oxidase_1, or GA3 oxidase_2 gene, the first non-coding RNA molecule is complementary to a recognition or target sequence of an mRNA expressed from such GA oxidase gene, and the second GA oxidase gene may be a GA20 oxidase_3, GA20 oxidase_5, GA20 oxidase_4, GA3 oxidase_1, or GA3 oxidase_2 gene. According to some embodiments, the first and second GA oxidase genes may be the same or different GA oxidase gene(s). Alternatively, the second GA oxidase gene may be another GA oxidase gene, such as a GA20 oxidase_1, GA20 oxidase_2, GA20 oxidase_6, GA20 oxidase_7, GA20 oxidase_8, or GA20 oxidase_9 gene, and the second non-coding RNA molecule is complementary to a recognition or target sequence of an mRNA expressed from such GA oxidase gene.

In the above stacking scenarios, and regardless of whether the targeting sequences are stacked in tandem in a single transcribable DNA sequence (or expression cassette) or in separate transcribable DNA sequences (or expression cassettes), the second GA oxidase gene may be a GA oxidase gene other than a GA20 oxidase_3, GA20 oxidase_5, GA20 oxidase_4, GA3 oxidase_1, or GA3 oxidase_2 gene, such as a GA20 oxidase_1, GA20 oxidase_2, GA20 oxidase_6, GA20 oxidase_7, GA20 oxidase_8, or GA20 oxidase_9 gene. According to these embodiments, the second targeting sequence of a non-coding RNA molecule may be at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of any one or more of SEQ ID NOs: 1, 2, 4, 5, 16, 17, 19, 20, 22, 23, 25, and/or 26. According to some embodiments, the second targeting sequence of a non-coding RNA molecule may be complementary to at least 19 consecutive nucleotides, but no more than 27 consecutive nucleotides, such as complementary to 19, 20, 21, 22, 23, 24, 25, 26, or 27 consecutive nucleotides, of any one or more of SEQ ID NOs: 1, 2, 4, 5, 16, 17, 19, 20, 22, 23, 25, and/or 26. According to some embodiments, the second targeting sequence of a non-coding RNA molecule may be at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein in the plant that is at least 80%, at least 85%, at least 90%, at least 95%, at least 96%, at least 97%, at least 98%, at least 99%, at least 99.5%, or 100% identical to any one or more of SEQ ID NOs: 3, 6, 18, 21, 24, and/or 27. According to further embodiments, the second targeting sequence of a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein that is at least 80%, at least 85%, at least 90%, at least 95%, at least 96%, at least 97%, at least 98%, at least 99%, at least 99.5%, or 100% similar to any one or more of SEQ ID NO: 3, 6, 18, 21, 24, and/or 27.

A recombinant DNA molecule or construct of the present disclosure may comprise or be included within a DNA transformation vector for use in transformation of a target plant cell, tissue or explant. Such a transformation vector may generally comprise sequences or elements necessary or beneficial for effective transformation in addition to at least one transgene, expression cassette and/or transcribable DNA sequence encoding a GA oxidase gene or a non-coding RNA molecule targeting an endogenous GA oxidase gene for suppression. For Agrobacterium-mediated, Rhizobia-mediated or other bacteria-mediated transformation, the transformation vector may comprise an engineered transfer DNA (or T-DNA) segment or region having two border sequences, a left border (LB) and a right border (RB), flanking at least a transcribable DNA sequence or transgene, such that insertion of the T-DNA into the plant genome will create a transformation event for the transcribable DNA sequence, transgene or expression cassette. Thus, a transcribable DNA sequence, transgene or expression cassette encoding a non-coding RNA molecule targeting an endogenous GA oxidase gene for suppression may be located between the left and right borders of the T-DNA, perhaps along with an additional transgene(s) or expression cassette(s), such as a plant selectable marker transgene and/or other gene(s) of agronomic interest that may confer a trait or phenotype of agronomic interest to a plant. According to alternative embodiments, the transcribable DNA sequence, transgene or expression cassette encoding a non-coding RNA molecule targeting an endogenous GA oxidase gene for suppression and the plant selectable marker transgene (or other gene of agronomic interest) may be present in separate T-DNA segments on the same or different recombinant DNA molecule(s), such as for co-transformation. A transformation vector or construct may further comprise prokaryotic maintenance elements, which may be located in the vector outside of the T-DNA region(s).

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. In some embodiments, a vector or polynucleotide provided herein comprises at least one selectable marker gene selected from the group consisting of nptII, aph IV, aadA, aac3, aacC4, bar, pat, DMO, EPSPS, aroA, GFP, and GUS. Plant transformation may also be carried out in the absence of selection during one or more steps or stages of culturing, developing or regenerating transformed explants, tissues, plants and/or plant parts.

According to present embodiments, methods for transforming a plant cell, tissue or explant with a recombinant DNA molecule or construct may further include site-directed or targeted integration. According to these methods, a portion of a recombinant DNA donor template molecule (i.e., an insertion sequence) may be inserted or integrated at a desired site or locus within the plant genome. The insertion sequence of the donor template may comprise a transgene or construct, such as a transgene or transcribable DNA sequence encoding a non-coding RNA molecule that targets an endogenous GA oxidase gene for suppression. The donor template may also have one or two homology arms flanking the insertion sequence to promote the targeted insertion event through homologous recombination and/or homology-directed repair. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a monocot or cereal plant. Thus, a recombinant DNA molecule of the present disclosure may comprise a donor template for site-directed or targeted integration of a transgene or construct, such as a transgene or transcribable DNA sequence encoding a non-coding RNA molecule that targets an endogenous GA oxidase gene for suppression, into the genome of a plant.

Any site or locus within the genome of a plant may potentially be chosen for site-directed integration of a transgene, construct or transcribable DNA sequence provided herein. For site-directed integration, a double-strand break (DSB) or nick may first be made at a selected genomic locus with a site-specific nuclease, such as, for example, a zinc-finger nuclease, an engineered or native meganuclease, a TALE-endonuclease, or an RNA-guided endonuclease (e.g., Cas9 or Cpf1). Any method known in the art for site-directed integration may be used. In the presence of a donor template molecule with an insertion sequence, the DSB or nick may then be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, construct or sequence may be achieved.

The introduction of a DSB or nick may also be used to introduce targeted mutations in the genome of a plant. According to this approach, mutations, such as deletions, insertions, inversions and/or substitutions may be introduced at a target site via imperfect repair of the DSB or nick to produce a knock-out or knock-down of a GA oxidase gene. Such mutations may be generated by imperfect repair of the targeted locus even without the use of a donor template molecule. A “knock-out” of a GA oxidase gene may be achieved by inducing a DSB or nick at or near the endogenous locus of the GA oxidase gene that results in non-expression of the GA oxidase protein or expression of a non-functional protein, whereas a “knock-down” of a GA oxidase gene may be achieved in a similar manner by inducing a DSB or nick at or near the endogenous locus of the GA oxidase gene that is repaired imperfectly at a site that does not affect the coding sequence of the GA oxidase gene in a manner that would eliminate the function of the encoded GA oxidase protein. For example, the site of the DSB or nick within the endogenous locus may be in the upstream or 5′ region of the GA oxidase gene (e.g., a promoter and/or enhancer sequence) to affect or reduce its level of expression. Similarly, such targeted knock-out or knock-down mutations of a GA oxidase gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out mutations of a GA oxidase gene may be achieved by deleting or inverting at least a portion of the gene or by introducing a frame shift or premature stop codon into the coding sequence of the gene. A deletion of a portion of a GA oxidase gene may also be introduced by generating DSBs or nicks at two target sites and causing a deletion of the intervening target region flanked by the target sites.

A site-specific nuclease provided herein may be selected from the group consisting of a zinc-finger nuclease (ZFN), a meganuclease, an RNA-guided endonuclease, a TALE-endonuclease (TALEN), a recombinase, a transposase, or any combination thereof. See, e.g., Khandagale, K. et al., “Genome editing for targeted improvement in plants,” Plant Biotechnol Rep 10: 327-343 (2016); and Gaj, T. et al., “ZFN, TALEN and CRISPR/Cas-based methods for genome engineering,” Trends Biotechnol. 31(7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp1 recombinase. According to some embodiments, a Cre recombinase or a Gin recombinase provided herein is tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator.

According to embodiments of the present disclosure, an RNA-guided endonuclease may be selected from the group consisting of Cast, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cpf1 enzyme.

In an aspect, a site-specific nuclease provided herein is selected from the group consisting of a zinc-finger nuclease, a meganuclease, an RNA-guided nuclease, a TALE-nuclease, a recombinase, a transposase, or any combination thereof. In another aspect, a site-specific nuclease provided herein is selected from the group consisting of a Cas9 or a Cpf1. In another aspect, a site-specific nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, an RNA-guided nuclease provided herein is selected from the group consisting of a Cas9 or a Cpf1. In another aspect, an RNA guided nuclease provided herein is selected from the group consisting of a Cas1, a Cas1B, a Cas2, a Cas3, a Cas4, a Cas5, a Cas6, a Cas7, a Cas8, a Cas9, a Cas10, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx15, a Csf1, a Csf2, a Csf3, a Csf4, a Cpf1, CasX, CasY, a homolog thereof, or a modified version thereof. In another aspect, a method and/or a composition provided herein comprises at least one, at least two, at least three, at least four, at least five, at least six, at least seven, at least eight, at least nine, or at least ten site-specific nucleases. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific nucleases.

For RNA-guided endonucleases, a guide RNA (gRNA) molecule is further provided to direct the endonuclease to a target site in the genome of the plant via base-pairing or hybridization to cause a DSB or nick at or near the target site. The gRNA may be transformed or introduced into a plant cell or tissue (perhaps along with a nuclease, or nuclease-encoding DNA molecule, construct or vector) as a gRNA molecule, or as a recombinant DNA molecule, construct or vector comprising a transcribable DNA sequence encoding the guide RNA operably linked to a plant-expressible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a GA oxidase gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al., “Target specificity of the CRISPR-Cas9 system,” Quant Biol. 2(2): 59-70 (2014), the content and disclosure of which is incorporated herein by reference. The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

For genome editing at or near the GA20 oxidase_3 gene with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 34 or a sequence complementary thereto (e.g., 12, 13, 14, 15, 16, 17, 18, 19, 20, 21, 22, 23, 24, 25 or more consecutive nucleotides of SEQ ID NO: 34 or a sequence complementary thereto). For genome editing at or near the GA20 oxidase_5 gene with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 35 or a sequence complementary thereto (e.g., 10, 11, 12, 13, 14, 15, 16, 17, 18, 19, 20, 21, 22, 23, 24, 25 or more consecutive nucleotides of SEQ ID NO: 35 or a sequence complementary thereto). As used herein, the term “consecutive” in reference to a polynucleotide or protein sequence means without deletions or gaps in the sequence.

For knockdown (and possibly knockout) mutations through genome editing, an RNA-guided endonuclease may be targeted to an upstream or downstream sequence, such as a promoter and/or enhancer sequence, or an intron, 5′UTR, and/or 3′UTR sequence of a GA20 oxidase_3 or GA20 oxidase_5 gene to mutate one or more promoter and/or regulatory sequences of the gene and affect or reduce its level of expression. For knockdown (and possibly knockout) of the GA20 oxidase_3 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-3096 of SEQ ID NO: 34, the nucleotide sequence range 3666-3775 of SEQ ID NO: 34, the nucleotide sequence range 4098-5314 of SEQ ID NO: 34, the nucleotide sequence range 5585-5800 of SEQ ID NO: 34, or the nucleotide sequence range 5801-8800 of SEQ ID NO: 34, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-3096, 3666-3775, 4098-5314, 5585-5800, 5801-8800, or 5585-8800 of SEQ ID NO: 34, or a sequence complementary thereto).

For knockdown (and possibly knockout) of the GA20 oxidase_5 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-3000 of SEQ ID NO: 35, the nucleotide sequence range 1-3000 of SEQ ID NO: 35, the nucleotide sequence range 3792-3906 of SEQ ID NO: 35, the nucleotide sequence range 4476-5197 of SEQ ID NO: 35, or the nucleotide sequence range 5860-8859 of SEQ ID NO: 35, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-3000, 3792-3906, 4476-5197, or 5860-8859 of SEQ ID NO: 35, or a sequence complementary thereto).

For knockout (and possibly knockdown) mutations through genome editing, an RNA-guided endonuclease may be targeted to a coding and/or intron sequence of a GA20 oxidase_3 or GA20 oxidase_5 gene to potentially eliminate expression and/or activity of a functional GA oxidase protein from the gene. However, a knockout of a GA oxidase gene expression may also be achieved in some cases by targeting the upstream and/or 5′UTR sequence(s) of the gene, or other sequences at or near the genomic locus of the gene. Thus, a knockout of a GA oxidase gene expression may be achieved by targeting a genomic sequence at or near the site or locus of a targeted GA20 oxidase_3 or GA20 oxidase_5 gene, an upstream or downstream sequence, such as a promoter and/or enhancer sequence, or an intron, 5′UTR, and/or 3′UTR sequence, of a GA20 oxidase_3 or GA20 oxidase_5 gene, as described above for knockdown of a GA20 oxidase_3 or GA20 oxidase_5 gene.

For knockout (and possibly knockdown) of the GA20 oxidase_3 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 3097-5584 of SEQ ID NO: 34, the nucleotide sequence range 3097-3665 of SEQ ID NO: 34, the nucleotide sequence range 3776-4097 of SEQ ID NO: 34, or the nucleotide sequence range 5315-5584 of SEQ ID NO: 34, or a sequence complementary thereto (e.g., 10, 11, 12, 13, 14, 15, 16, 17, 18, 19, 20, 21, 22, 23, 24, 25 or more consecutive nucleotides within the nucleotide sequence range 3097-5584, 3097-3665, 3097-3775, 3665-4097, 3776-4097, 3776-5314, 4098-5584, or 5315-5584 of SEQ ID NO: 34, or a sequence complementary thereto).

For knockout (and possibly knockdown) of the GA20 oxidase_5 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 3001-5473 of SEQ ID NO: 35, the nucleotide sequence range 3001-3791 of SEQ ID NO: 35, the nucleotide sequence range 3907-4475 of SEQ ID NO: 35, or the nucleotide sequence range 5198-5473 of SEQ ID NO: 35, or a sequence complementary thereto (e.g., 10, 11, 12, 13, 14, 15, 16, 17, 18, 19, 20, 21, 22, 23, 24, 25 or more consecutive nucleotides within the nucleotide sequence range 3001-5473, 3001-3791, 3001-3906, 3792-4475, 3907-4475, 3907-5197, 4476-5473, or 5198-5473 of SEQ ID NO: 35, or a sequence complementary thereto).

According to some embodiments, a guide RNA for targeting an endogenous GA20 oxidase_3 and/or GA20 oxidase_5 gene is provided, which may comprise a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, or at least 21 consecutive nucleotides of any one or more of SEQ ID NOs: 138-167. According to some embodiments, a guide RNA for targeting both of the endogenous GA20 oxidase_3 and GA20 oxidase_5 genes is provided, which may comprise a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, or at least 21 consecutive nucleotides of SEQ ID NO: 34, and at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, or at least 21 consecutive nucleotides of SEQ ID NOs: 34 and 35. According to some embodiments, a guide RNA for targeting both of the endogenous GA20 oxidase_3 and GA20 oxidase_5 genes is provided, which may comprise a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, or at least 21 consecutive nucleotides of any one or more of SEQ ID NOs: 158-167.

For genome editing at or near the GA20 oxidase_4 gene with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 38 or a sequence complementary thereto (e.g., 12, 13, 14, 15, 16, 17, 18, 19, 20, 21, 22, 23, 24, 25 or more consecutive nucleotides of SEQ ID NO: 38 or a sequence complementary thereto).

For knockout (and possibly knockdown) mutations through genome editing, an RNA-guided endonuclease may be targeted to a coding and/or intron sequence of a GA20 oxidase_4 gene to potentially eliminate expression and/or activity of a functional GA20 oxidase_4 protein from the gene. For the GA20 oxidase_4 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1544-2852 of SEQ ID NO: 38, the nucleotide sequence range 1544-1995 of SEQ ID NO: 38, the nucleotide sequence range 2084-2411 of SEQ ID NO: 38, or the nucleotide sequence range 2517-2852 of SEQ ID NO: 38, or a sequence complementary thereto (e.g., 10, 11, 12, 13, 14, 15, 16, 17, 18, 19, 20, 21, 22, 23, 24, 25 or more consecutive nucleotides within the nucleotide sequence range 1544-2852, 1544-1995, 1544-2083, 1996-2411, 2084-2411, 2084-2516, 2412-2852, or 2517-2852 of SEQ ID NO: 38, or a sequence complementary thereto).

For knockdown (and possibly knockout) mutations through genome editing, an RNA-guided endonuclease may be targeted to an upstream or downstream sequence, such as a promoter and/or enhancer sequence, or an intron, 5′UTR, and/or 3′UTR sequence of a GA20 oxidase_4 gene to mutate one or more promoter and/or regulatory sequences of the gene and affect or reduce its level of expression. For knockdown of the GA20 oxidase_3 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-1416 of SEQ ID NO: 38, the nucleotide sequence range 1417-1543 of SEQ ID NO: 38, the nucleotide sequence range 1996-2083 of SEQ ID NO: 38, the nucleotide sequence range 2412-2516 of SEQ ID NO: 38, the nucleotide sequence range 2853-3066 of SEQ ID NO: 38, or the nucleotide sequence range 3067-4465 of SEQ ID NO: 38, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-1416, 1417-1543, 1-1543, 1996-2083, 2412-2516, 2853-3066, 3067-4465 or 2853-4465 of SEQ ID NO: 38, or a sequence complementary thereto).

In addition to the guide sequence, a guide RNA may further comprise one or more other structural or scaffold sequence(s), which may bind or interact with an RNA-guided endonuclease.

Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

According to some embodiments, recombinant DNA constructs and vectors are provided comprising a polynucleotide sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA constructs and vectors are further provided comprising a polynucleotide sequence encoding a guide RNA, wherein the guide RNA comprises a guide sequence of sufficient length having a percent identity or complementarity to a target site within the genome of a plant, such as at or near a targeted GA oxidase gene. According to some embodiments, a polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc.

In an aspect, the present disclosure provides a modified corn plant, or plant part thereof, comprising a mutant allele at the GA20 oxidase_3 locus and a mutant allele at the GA20 oxidase_5 locus, wherein at least one of the GA20 oxidase_3 and GA20 oxidase_5 loci comprises homozygous mutant alleles. In another aspect, a modified corn plant comprises homozygous alleles at the GA20 oxidase_3 locus. In another aspect, a modified corn plant comprises homozygous alleles at the GA20 oxidase_5 locus. In a further aspect, only one of the GA20 oxidase_3 and GA20 oxidase_5 loci comprises homozygous alleles in a modified corn plant. In another aspect, a modified corn plant comprises homozygous alleles at both of the GA20 oxidase_3 and GA20 oxidase_5 loci. In another aspect, one or both of the GA20 oxidase_3 and GA20 oxidase_5 loci comprise a heteroallelic combination or two identical mutant alleles in a modified plant. In an aspect, a modified plant comprises a homozygous GA20 oxidase_3 locus comprising a heteroallelic combination of mutant alleles and a homozygous GA20 oxidase_5 locus comprising a heteroallelic combination of mutant alleles. In another aspect, a modified plant comprises a homozygous GA20 oxidase_3 locus comprising a heteroallelic combination of mutant alleles and a homozygous GA20 oxidase_5 locus comprising two identical mutant alleles. In an aspect, a modified plant comprises a homozygous GA20 oxidase_3 locus comprising two identical mutant alleles and a homozygous GA20 oxidase_5 locus comprising a heteroallelic combination of mutant alleles. In another aspect, a modified plant comprises a homozygous GA20 oxidase_3 locus comprising two identical mutant alleles and a homozygous GA20 oxidase_5 locus comprising two identical mutant alleles. In an aspect, a modified plant comprises a homozygous GA20 oxidase_3 locus comprising a heteroallelic combination of mutant alleles and a heterozygous GA20 oxidase_5 locus. In another aspect, a modified plant comprises a homozygous GA20 oxidase_3 locus comprising a heteroallelic combination of mutant alleles and a heterozygous GA20 oxidase_5 locus. In an aspect, a modified plant comprises a heterozygous GA20 oxidase_3 locus and a homozygous GA20 oxidase_5 locus comprising a heteroallelic combination of mutant alleles. In another aspect, a modified plant comprises a heterozygous GA20 oxidase_3 locus and a homozygous GA20 oxidase_5 locus comprising two identical mutant alleles.

In another aspect, the present disclosure provides a modified corn plant, or plant part thereof, comprising a first homozygous mutation in one of GA20 oxidase_3 and GA20 oxidase_5 genes and further comprising a second heterozygous or homozygous mutation in the other one of the GA20 oxidase_3 and GA20 oxidase_5 genes. In an aspect, a first homozygous mutation is in GA20 oxidase_3. In another aspect, a second mutation is heterozygous in GA20 oxidase_5. In an aspect, a first homozygous mutation is in GA20 oxidase_5. In another aspect, a second mutation is heterozygous in GA20 oxidase_3. In a further aspect, a first homozygous mutation comprises a heteroallelic combination of mutations, or two identical mutant alleles, in one of the GA20 oxidase_3 and GA20 oxidase_5 genes.

In an aspect, a GA20 oxidase_3 locus or gene comprises a sequence sharing at least 80%, at least 85%, at least 90%, at least 95%, at least 99%, or at least 99.5% sequence identity to SEQ ID No. 34 or 168. In an aspect, a GA20 oxidase_5 locus or gene comprises a sequence sharing at least 80%, at least 85%, at least 90%, at least 95%, at least 99%, or at least 99.5% sequence identity to SEQ ID No. 35 or 169.

In an aspect, a GA20 oxidase_3 or GA20 oxidase_5 mutation (mutant gene or mutant allele) comprises a mutation type selected from the group consisting of a nonsense mutation, a missense mutation, a frameshift mutation, and a splice-site mutation. In an aspect, a GA20 oxidase_3 or GA20 oxidase_5 mutation (or mutant allele) results in a truncated mRNA or polypeptide, or results in a non-translatable mRNA molecule. A missense mutation is a change in one DNA base pair that results in the substitution of one amino acid for another in the protein made by a gene. A nonsense mutation is also a change in one DNA base pair. Instead of substituting one amino acid for another, however, the altered DNA sequence prematurely signals the cell to stop building a protein. This type of mutation results in a shortened protein that may function improperly or not at all. A frameshift mutation occurs when the addition or loss of DNA bases changes a gene's reading frame. A frameshift mutation shifts the grouping of these bases and changes the code for amino acids. The resulting protein, even if made, is usually nonfunctional. Insertions, deletions, and duplications can all be frameshift mutations. In another aspect, a GA20 oxidase_3 or GA20 oxidase_5 mutation (mutant gene or mutant allele) can comprise a silent mutation which does not change an encoded amino acid sequence, but can affect mRNA transcript expression, stability or protein translation efficiency, or otherwise contribute to reduced enzyme activity, relative to a corresponding wild type GA20 oxidase_3 or GA20 oxidase_5 gene. In a further aspect, a GA20 oxidase_3 or GA20 oxidase_5 mutation (mutant gene or mutant allele) can comprise a mutation or edit at or around the TATA box or other promoter elements that affect gene transcription. In an aspect, a GA20 oxidase_3 mutation or allele in a modified corn plant is a recessive mutation or allele. In an aspect, a GA20 oxidase_3 mutation or allele in a modified corn plant is a dominant mutation or allele. In an aspect, a GA20 oxidase_5 mutation or allele in a modified corn plant is a recessive mutation or allele. In an aspect, a GA20 oxidase_5 mutation or allele in a modified corn plant is a dominant mutation or allele.

In an aspect, a GA20 oxidase_3 or GA20 oxidase_5 mutation (or mutant allele) comprises a mutation in a GA20 oxidase_3 or GA20 oxidase_5 sequence region selected from the group consisting of a promoter, 5′ UTR, first exon, first intron, second exon, second intron, third exon, 3′ UTR, and terminator. In an aspect, a GA20 oxidase_3 or GA20 oxidase_5 mutation (or mutant allele) comprises a mutation in the first or second exon of the GA20 oxidase_3 or GA20 oxidase_5 gene.

In an aspect, a mutant GA20 oxidase_3 or GA20 oxidase_5 allele exhibits an at least 30%, at least 40%, at least 50%, at least 60%, at least 70%, at least 80%, at least 90%, at least 95%, or 100% reduction of expression or enzymatic activity relative to an unmodified, wild-type GA20 oxidase_3 or GA20 oxidase_5 gene allele. In another aspect, a mutant GA20 oxidase_3 or GA20 oxidase_5 allele comprises a mutation in a sequence region selected from the group consisting of a promoter, 5′ UTR, first exon, first intron, second exon, second intron, third exon, 3′ UTR, terminator, and any combination thereof. In another aspect, a mutant GA20 oxidase_3 or GA20 oxidase_5 allele comprises one or more mutation types selected from the group consisting of a nonsense mutation, a missense mutation, a frameshift mutation, a splice-site mutation, and any combination thereof. In another aspect, a mutant GA20 oxidase_3 or GA20 oxidase_5 allele results in one or more of the following: a protein truncation, a non-translatable transcript, a non-functional protein, a premature stop codon, and any combination thereof. In another aspect, a mutant GA20 oxidase_3 or GA20 oxidase_5 allele comprises a mutation selected from the group consisting of a substitution, a deletion, an insertion, a duplication, and an inversion of one or more nucleotides relative to a wild-type GA20 oxidase_3 gene. In another aspect, a mutant GA20 oxidase_3 or GA20 oxidase_5 allele comprises one or more mutations in the first exon. In another aspect, a mutant GA20 oxidase_3 or GA20 oxidase_5 allele comprises one or more mutations in the second exon.

In an aspect, a modified corn plant, or plant part thereof, comprises a first mutation comprising one or more alleles, as a pair of two identical alleles or a heteroallelic combination, selected from the group consisting of: a deletion of 13 bases starting at 536; a deletion of base 542; an insertion of CC at base 542; a deletion of base 541; a deletion of 3 nt starting at base 540; a deletion of 2 bases starting at base 422; an insertion of an A at base 422; an insertion of a T at base 422; a deletion of base 564; an insertion of an A at base 564; an insertion of a C at base 565; and an insertion of a C at base 63; wherein the base numbering is based on SEQ ID No. 168 and counted from the first nucleotide of SEQ ID NO: 168 in the 5′ to 3′ direction. In another aspect, a modified corn plant, or plant part thereof, comprises a first mutation comprising one or more alleles, as a pair of two identical alleles or a heteroallelic combination, selected from the group consisting of: a deletion of base 644; a deletion of 2 bases starting at base 644; an insertion of a T at base 644; a deletion of base 372; a deletion of base 786; a deletion of 5 bases starting at base 786; a deletion of 2 bases starting at base 101; an insertion of a T at base base 102; a deletion of 3 bases starting at base 99; an insertion of an A at base 282; and an insertion of a C at base 282; wherein the base numbering is based on SEQ ID No. 169 and counted from the first nucleotide of SEQ ID NO: 169 in the 5′ to 3′ direction. In an aspect, a modified corn plant, or plant part thereof, comprises a first mutation identified by one or more of SEQ ID Nos.: 170 to 193 and 206 to 217 relative to the corresponding reference sequence in SEQ ID No: 168. In an aspect, a modified corn plant, or plant part thereof, comprises a first mutation identified by one or more of SEQ ID Nos.: 218 to 239 and 251 to 261 relative to the corresponding reference sequence in SEQ ID No: 169. In an aspect, the present disclosure provides a progeny plant of one or more plants listed in Table 18 or 19. In another aspect, also provided is a progeny plant of any one of plant Nos. 17 to 31 in Table 19. In a further aspect, a plant is provided from a cross or hybridization of one or more plants listed in Table 18 or 19.

In another aspect, the present disclosure provides a modified corn plant, or plant part thereof, comprising a homozygous mutant GA20 oxidase_3 gene and a homozygous mutant GA20 oxidase_5 gene. In an aspect, a homozygous mutant GA20 oxidase_3 gene, a homozygous mutant GA20 oxidase_5 gene, or both comprise a heteroallelic combination of mutant alleles or two identical mutant alleles. In an aspect, a homozygous mutant GA20 oxidase_3 gene, a homozygous mutant GA20 oxidase_5 gene, or both comprise a mutation in a sequence region selected from the group consisting of promoter, 5′ UTR, first exon, first intron, second exon, second intron, third exon, 3′ UTR, terminator, and any combination thereof. In an aspect, a homozygous mutant GA20 oxidase_3 gene, a homozygous mutant GA20 oxidase_5 gene, or both comprise one or more mutation types selected from the group consisting of a nonsense mutation, a missense mutation, a frameshift mutation, a splice-site mutation, and any combination thereof. In an aspect, a homozygous mutant GA20 oxidase_3 gene, a homozygous mutant GA20 oxidase_5 gene, or both result in one or more of the following: a protein truncation, a non-translatable transcript, a non-functional protein, a premature stop codon, and any combination thereof. In an aspect, a homozygous mutant GA20 oxidase_3 gene, a homozygous mutant GA20 oxidase_5 gene, or both comprise a mutation selected from the group consisting of a substitution, a deletion, an insertion, a duplication, and an inversion of one or more nucleotides relative to a wild-type GA20 oxidase_3 gene. In an aspect, a mutant GA20 oxidase_3 gene, a homozygous mutant GA20 oxidase_5 gene, or both comprise a null allele.

In another aspect, the present disclosure provides a modified corn plant, or plant part thereof, comprising homozygous mutant alleles at an endogenous GA20 oxidase_3 locus and homozygous mutant alleles at an endogenous GA20 oxidase_5 locus. In an aspect, homozygous mutant alleles at the endogenous GA20 oxidase_3 locus, homozygous mutant alleles at the endogenous GA20 oxidase_5 locus, or both comprise a heteroallelic combination or two identical mutant alleles.

In an aspect, a modified corn plant described here has a shorter plant height and/or improved lodging resistance relative to an unmodified control plant. In an aspect, a modified corn plant is at least 10%, at least 20%, at least 25%, at least 30%, at least 35%, or at least 40% shorter than an unmodified control plant. In another aspect, a modified corn plant has a stalk or stem diameter at one or more stem internodes is at least 5%, at least 10%, at least 15%, at least 20%, at least 25%, at least 30%, at least 35%, or at least 40% greater than the stalk or stem diameter at the same one or more internodes of an unmodified control plant. In an aspect, a modified corn plant has a stalk or stem diameter at one or more of the first, second, third, and/or fourth internode below the ear is at least 5%, at least 10%, at least 15%, at least 20%, at least 25%, at least 30%, at least 35%, or at least 40% greater than the same internode of an unmodified control plant. In another aspect, the level of one or more active GAs in at least one internode tissue of the stem or stalk of a modified corn plant is at least 5%, at least 10%, at least 15%, at least 20%, at least 25%, at least 30%, at least 35%, or at least 40% lower than the same internode tissue of an unmodified control plant. In an aspect, the level of one or more active GAs in at least one internode tissue of the stem or stalk of a modified corn plant is lower than the same internode tissue of an unmodified control plant.

In another aspect, a modified corn plant does not have any significant off-types in at least one female organ or ear. In an aspect, a modified corn plant exhibits essentially no reproductive abnormality. In a further aspect, an off-type or reproductive abnormality is selected from the group consisting of male (tassel or anther) sterility, reduced kernel or seed number, and the presence of one or more masculinized or male (or male-like) reproductive structures in the female organ or ear (e.g., anther ear).

In another aspect, a modified corn plant comprises one or more traits, relative to an unmodified control plant, selected from the group consisting of shorter plant height, increased stalk/stem diameter, improved lodging resistance, reduced green snap, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and increased prolificacy.

In an aspect, the present disclosure provides a modified corn plant, or plant part thereof, comprising homozygous mutant alleles at GA20 oxidase_3 locus, and exhibiting an at least 2.5%, at least 5%, at least 7.5%, at least 10%, at least 15%, or at least 20% reduction in plant height at maturity relative to a unmodified control plant. In another aspect, the present disclosure provides a modified corn plant, or plant part thereof, comprising homozygous mutant alleles at GA20 oxidase_5 locus, and exhibiting an at least 2.5%, at least 5%, at least 7.5%, at least 10%, at least 15%, or at least 20% reduction in plant height at maturity relative to a unmodified control plant. In a further aspect, a plant height reduction is between 5% and 30%, between 7.5% and 25%, between 10 and 20%, 5% and 7.5%, between 7.5% and 10%, between 10 and 15%, or between 15% to 20%.

In an aspect, a modified corn plant is an inbred. In another aspect, a modified corn plant is a hybrid. In an aspect, a modified corn plant is a plant modified by a targeted genome editing technique.

In an aspect, the present disclosure provides a modified corn plant, or plant part thereof, comprising a mutant allele at a GA20 oxidase_3 locus, a mutant allele at a GA20 oxidase_5 locus, or both, and further comprising an expression cassette comprising a transcribable DNA sequence encoding a non-coding RNA for suppressing one or more of the GA20 oxidase_3 gene, the GA20 oxidase_4 gene, and the GA20 oxidase_5 gene. Any modified or edited mutant allele described here can be combined with an expression cassette encoding a non-coding RNA. Also the mutant allele at the GA20 oxidase_3 locus, a mutant allele at the GA20 oxidase_5 locus can be in any zygosity status. In one aspect, the GA20 oxidase_3 locus, the GA20 oxidase_5 locus, or both comprise homozygous mutant alleles. In another aspect, the GA20 oxidase_3 locus, the GA20 oxidase_5 locus, or both comprise heterozygous alleles. In an aspect, the GA20 oxidase_3 locus comprises homozygous mutant alleles, and said GA20 oxidase_5 locus comprises heterozygous alleles. In another aspect, the GA20 oxidase_5 locus comprises homozygous mutant alleles, and said GA20 oxidase_3 locus comprises heterozygous alleles. In an aspect, an expression cassette is a recombinant DNA expression cassette, and a transcribable DNA sequence is operably linked to a plant-expressible promoter. In an aspect, a non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein being at least 95%, at least 96%, at least 97%, at least 98%, at least 99%, at least 99.5%, or 100% identical to SEQ ID NO: 9, 12, or 15. In another aspect, a non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7, 8, 10, 11, 13, or 14. In a further aspect, a non-coding RNA molecule encoded by the transcribable DNA sequence is a precursor miRNA or siRNA that is processed or cleaved in a plant cell to form a mature miRNA or siRNA.

According to some embodiments, a recombinant DNA construct or vector may comprise a first polynucleotide sequence encoding a site-specific nuclease and a second polynucleotide sequence encoding a guide RNA that may be introduced into a plant cell together via plant transformation techniques. Alternatively, two recombinant DNA constructs or vectors may be provided including a first recombinant DNA construct or vector and a second DNA construct or vector that may be introduced into a plant cell together or sequentially via plant transformation techniques, wherein the first recombinant DNA construct or vector comprises a polynucleotide sequence encoding a site-specific nuclease and the second recombinant DNA construct or vector comprises a polynucleotide sequence encoding a guide RNA. According to some embodiments, a recombinant DNA construct or vector comprising a polynucleotide sequence encoding a site-specific nuclease may be introduced via plant transformation techniques into a plant cell that already comprises (or is transformed with) a recombinant DNA construct or vector comprising a polynucleotide sequence encoding a guide RNA. Alternatively, a recombinant DNA construct or vector comprising a polynucleotide sequence encoding a guide RNA may be introduced via plant transformation techniques into a plant cell that already comprises (or is transformed with) a recombinant DNA construct or vector comprising a polynucleotide sequence encoding a site-specific nuclease. According to yet further embodiments, a first plant comprising (or transformed with) a recombinant DNA construct or vector comprising a polynucleotide sequence encoding a site-specific nuclease may be crossed with a second plant comprising (or transformed with) a recombinant DNA construct or vector comprising a polynucleotide sequence encoding a guide RNA. Such recombinant DNA constructs or vectors may be transiently transformed into a plant cell or stably transformed or integrated into the genome of a plant cell.

In an aspect, vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, vectors comprising polynucleotides encoding a Cas9 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous GA oxidase gene of a corn or cereal plant, such as the GA20 oxidase_3 gene or the GA20 oxidase_5 gene in corn, to create a DSB or nick at such genomic locus to knockout or knockdown expression of the GA oxidase gene via repair of the DSB or nick. For example, an engineered site-specific nuclease, such as a recombinase, zinc finger nuclease (ZFN), meganuclease, or TALEN, may be designed to target and bind to (i) a target site within the genome of a plant corresponding to a sequence within SEQ ID NO: 34, or its complementary sequence, to create a DSB or nick at the genomic locus for the GA20 oxidase_3 gene, (ii) a target site within the genome of a plant corresponding to a sequence within SEQ ID NO: 35, or its complementary sequence, to create a DSB or nick at the genomic locus for the GA20 oxidase_5 gene, and/or (iii) a target site within the genome of a plant corresponding to a sequence within SEQ ID NO: 38, or its complementary sequence, to create a DSB or nick at the genomic locus for the GA20 oxidase_4 gene, which may then lead to the creation of a mutation or insertion of a sequence at the site of the DSB or nick, through cellular repair mechanisms, which may be guided by a donor molecule or template.

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp1 recombinase. In an aspect, a Cre recombinase or a Gin recombinase provided herein may be tethered to a zinc-finger DNA binding domain. The Flp-FRT site-directed recombination system may come from the 2p, plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger a-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., US Patent App. Nos. 2005/0064474, 2009/0117617, and 2012/0142062, the contents and disclosures of which are incorporated herein by reference. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory, because the DNA-binding specificities of zinc finger domains can be re-engineered using one of various methods, customized ZFNs can theoretically be constructed to target nearly any target sequence (e.g., at or near a GA oxidase gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick. In an aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection, or Agrobacterium-mediated transformation). The ZFNs may be introduced as ZFN proteins, as polynucleotides encoding ZFN proteins, and/or as combinations of proteins and protein-encoding polynucleotides.

Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-Msol, I-SceI, I-Anil, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a GA oxidase gene. In an aspect, a method and/or composition provided herein comprises one or more, two or more, three or more, four or more, or five or more meganucleases. In another aspect, a meganuclease provided herein is capable of generating a targeted DSB. In an aspect, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

TALENs are artificial restriction enzymes generated by fusing the transcription activator-like effector (TALE) DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN

DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also refers to one or both members of a pair of TALENs that work together to cleave DNA at the same site.

Transcription activator-like effectors (TALEs) can be engineered to bind practically any DNA sequence, such as at or near the genomic locus of a GA oxidase gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122; Cermak et al., Nucleic Acids Research (2011). 39:e82; and tale-nt.cac.cornell.edu/about. In an aspect, a method and/or composition provided herein comprises one or more, two or more, three or more, four or more, or five or more TALENs. In another aspect, a TALEN provided herein is capable of generating a targeted DSB. In an aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation). See, e.g., US Patent App. Nos. 2011/0145940, 2011/0301073, and 2013/0117869, the contents and disclosures of which are incorporated herein by reference.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” also encompasses the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution or insertion, whereas “edits” or “genomic edits” refers to two or more targeted mutation(s), deletion(s), inversion(s), substitution(s) and/or insertion(s), with each “edit” being introduced via a targeted genome editing technique.

Given that suppression of GA20 oxidase_3, GA20 oxidase_4, and/or GA20 oxidase_5 genes in corn produces plants having a shorter plant height and internode length in addition to other beneficial traits, it is proposed that expression of one or more of these genes may be reduced or eliminated through genome editing one or more of these gene(s) to provide similar beneficial traits to corn plants. Given further that constitutive expression of suppression constructs targeting these GA20 oxidase genes produces corn plants having the beneficial short height traits without off-types in the ear, and that expression directly in reproductive ear tissues also does not give rise to reproductive off-types, it is proposed that one or more of these gene loci may be edited to knock-down or knock-out their expression to produce similar effects in corn plants. Targeted gene editing approaches could be used to modify the sequence of the promoter and/or regulatory region(s) of one or more of the GA20 oxidase_3, GA20 oxidase_4, and/or GA20 oxidase_5 genes to knock-down or knock-out expression of these gene(s), such as through targeted deletions, insertions, mutations, or other sequence changes. Indeed, the promoter and/or regulatory region(s) or sequence(s), or the 5′-UTR, 3′UTR, and/or intron sequence(s), of one or more of the GA20 oxidase_3, GA20 oxidase_4, and/or GA20 oxidase_5 genes may be largely deleted or mutated. Alternatively, all or a portion of the coding (exon), 5-UTR, 3′UTR, and/or intron sequence(s) of one or more of the GA20 oxidase_3, GA20 oxidase_4, and/or GA20 oxidase_5 genes may be edited, deleted, mutated, or otherwise modified to knock-down or knock-out expression or activity of these gene(s). Such targeted modifications to the GA20 oxidase_3, GA20 oxidase_4, and/or GA20 oxidase_5 gene loci may be achieved using any suitable genome editing technology known in the art, such as via repair of a double strand break (DSB) or nick introduced by a site-specific nuclease, such as, for example, a zinc-finger nuclease, an engineered or native meganuclease, a TALE-endonuclease, or an RNA-guided endonuclease (e.g., Cas9 or Cpf1). Such repair of the DSB or nick may introduce spontaneous or stochastic deletions, additions, mutations, etc., at the targeted site where the DSB or nick was introduced, or repair of the site may involve the use of a donor template molecule to direct or cause a preferred or specific deletion, addition, mutation, etc., at the targeted site. In an aspect, the present disclosure provides a modified plant comprising a transgene-based suppression construct for GA20 oxidase_4 in combination with transgene-based suppression of GA20 oxidase_3, GA20 oxidase_5, or both. In another aspect, a modified plant is provided comprising a transgene-based suppression construct for GA20 oxidase_4 in combination with targeted modification(s) of GA20 oxidase_3, GA20 oxidase_5, or both. In another aspect, a modified plant is provided comprising a targeted modification of GA20 oxidase_4 in combination with transgene-based suppression of GA20 oxidase_3, GA20 oxidase_5, or both. In a further aspect, a modified plant is provided comprising a targeted modification of GA20 oxidase_4 in combination with targeted modification(s) of GA20 oxidase_3, GA20 oxidase_5, or both.

As provided herein, a plant transformed with a recombinant DNA molecule or transformation vector comprising a transgene encoding a transcribable DNA sequence encoding a non-coding RNA molecule that targets an endogenous GA oxidase gene for suppression may include a variety of monocot or cereal plants, such as maize/corn and other monocot or cereal plants that have separate male and female flowers (similarly to corn) and may thus be susceptible to off-types in female reproductive organs, structures or tissues with mutations to the GA pathway.

The present compositions and methods may be further applicable to other cereal plants that would benefit from a reduced plant height and/or increased resistance to lodging. Such plants may be transformed with recombinant DNA molecules or constructs to suppress one or more endogenous GA20 and/or GA3 oxidase genes in the plant according to the methods and approaches provided herein to produce a cereal plant that may be shorter and/or resistant to lodging. Indeed, a cereal plant ectopically expressing a transcribable DNA sequence encoding a non-coding RNA molecule that targets an endogenous GA oxidase gene for suppression may have a variety of beneficial traits, such as shorter stature or plant height, shorter internode length, increased stalk/stem diameter, improved lodging resistance, in addition to other improved yield-related and/or drought tolerant traits as provided herein, relative to a wild-type or control plant not having the transgene or transcribable DNA sequence. As described further below, cereal crop plants that have already been modified to have increased yield and resist lodging through mutations in the GA pathway, such as wheat, rice, millet, barley and sorghum, may instead be transformed with a recombinant DNA molecule or construct as provided herein. Unlike many of the GA pathway mutations in these crops which may be recessive, transgenic constructs expressing a suppression element targeting an endogenous biosynthetic GA oxidase gene in those crops may be dominant even when hemizygous or present in the plant as a single copy. Thus, plants that may be transformed with a recombinant DNA molecule or construct expressing a suppression construct may potentially include a variety of monocot or cereal crops. Having a dominant transgenic locus that causes a semi-dwarf, lodging resistant phenotype may be advantageous and preferred over a recessive mutant allele for the same phenotype due to benefits in breeding and trait integration.

According to embodiments of the present disclosure, it is further proposed that GA oxidase genes in other cereal plants having the greatest sequence identity/similarity to the GA20 oxidase_3, GA20 oxidase_4, GA20 oxidase_5, GA3 oxidase_1, and/or GA3 oxidase_2 genes in corn that are shown herein to produce a short stature, semi-dwarf phenotype and other beneficial traits when suppressed with a recombinant DNA suppression construct, may also be targets for suppression to produce transgenic cereal plants having similar semi-dwarf and/or lodging resistance phenotypes. Table 3 provides a list of GA oxidase genes from other cereal plants (sorghum—Sorghum bicolor; rice—Oryza sativa; foxtail millet—Setaria italica; wheat—Triticum aestivum; and barley—Hordeum vulgare) having a high degree of sequence identity with one of the GA oxidase genes in corn that when suppressed produces a short stature, semi-dwarf phenotype.

TABLE 3
Homologs of corn GA oxidase genes from other cereal crop plants.

| | Cereal | Corn | cDNA | CDS | Protein | Genomic |
|---|---|---|---|---|---|---|
| Gene Name | Species | Homolog | (SEQ ID NO) | (SEQ ID NO) | (SEQ ID NO) | (SEQ ID NO) |
| GA20 | Sorghum | GA20 Ox_3/ | 84 | 85 | 86 | 87 |
| oxidase 2 | bicolor | GA20 Ox_5 | | | | |
| GA20 | Setaria | GA20 Ox_3/ | 88 | 89 | 90 | 91 |
| oxidase 2-like | italica | GA20 Ox_5 | | | | |
| GA20 | Oryza | GA20 Ox_3/ | 92 | 93 | 94 | 95 |
| oxidase 2 | sativa | GA20 Ox_5 | | | | |
| GA20 | Triticum | GA20 Ox_3/ | — | 96 | 97 | 98 |
| oxidase-D2 | aestivum | GA20 Ox_5 | | | | |
| Fe2OG | Hordeum | GA20 Ox_3/ | 99 | 100 | 101 | — |
| dioxygenase | vulgare | GA20 Ox_5 | | | | |
| Probable 2-ODD | Sorghum | GA20 Ox_4 | 102 | 103 | 104 | 105 |
| | bicolor | | | | | |
| flavonol | Setaria | GA20 Ox_4 | 106 | 107 | 108 | 109 |
| synthase/flavanone | italica | | | | | |
| 3-hydroxylase-like | | | | | | |
| naringenin, | Oryza | GA20 Ox_4 | 110 | 111 | 112 | 113 |
| 2-oxoglutarate | sativa | | | | | |
| 3-dioxygenase | | | | | | |
| Fe2OG | Triticum | GA20 Ox_4 | 114 | 115 | 116 | 117 |
| dioxygenase | aestivum | | | | | |
| Fe2OG | Hordeum | GA20 Ox_4 | — | — | 118 | — |
| dioxygenase | vulgare | | | | | |
| GA3-beta- | Sorghum | GA3 Ox_1/ | 119 | 120 | 121 | 122 |
| dioxygenase 2-2 | bicolor | GA3 Ox_2 | | | | |
| GA3-beta- | Setaria | GA3 Ox_1/ | 123 | 124 | 125 | 126 |
| dioxygenase | italica | GA3 Ox_2 | | | | |
| 2-2-like | | | | | | |
| GA3-beta- | Oryza | GA3 Ox_1/ | 127 | 128 | 129 | 130 |
| dioxygenase 2-3 | sativa | GA3 Ox_2 | | | | |
| GA3-beta- | Hordeum | GA3 Ox_1/ | 131 | 132 | 133 | — |
| hydroxylase | vulgare | GA3 Ox_2 | | | | |
| GA3ox- | Triticum | GA3 Ox_1/ | 134 | 135 | 136 | 137 |
| D2 protein | aestivum | GA3 Ox_2 | | | | |

According to another aspect of the present disclosure, a recombinant DNA molecule, vector or construct is provided for suppression of an endogenous GA oxidase (or GA oxidase-like) gene in a cereal plant, the recombinant DNA molecule, vector or construct comprising a transcribable DNA sequence encoding a non-coding RNA molecule, wherein the non-coding RNA molecule comprises a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of any one or more of SEQ ID NO: 84, 85, 87, 88, 89, 91, 92, 93, 95, 96, 98, 99, 100, 102, 103, 105, 106, 107, 109, 110, 111, 113, 114, 115, 119, 120, 122, 123, 124, 126, 127, 128, 130, 131, 132, 134, 135, and/or 137, and/or (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding a protein in the cereal plant that is at least 80%, at least 85%, at least 90%, at least 95%, at least 96%, at least 97%, at least 98%, at least 99%, at least 99.5%, or 100% identical to any one or more of SEQ ID NO: 86, 90, 94, 97, 101, 104, 108, 112, 116, 118, 121, 125, 129, 133, and/or 136. Likewise, a non-coding RNA molecule may target an endogenous GA oxidase (or GA oxidase-like) gene in a cereal plant having a percent identity to the GA oxidase gene(s) shown to affect plant height in corn. Thus, a non-coding RNA molecule is further provided comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous protein in a cereal plant that is at least 80%, at least 85%, at least 90%, at least 95%, at least 96%, at least 97%, at least 98%, at least 99%, at least 99.5%, or 100% identical to any one or more of SEQ ID NO: 9, 12, 15, 30, and/or 33. As mentioned above, the non-coding RNA molecule may target an exon, intron and/or UTR sequence of a GA oxidase (or GA oxidase-like) gene.

Further provided are methods for introducing or transforming into a cereal plant, plant part, or plant cell any of the foregoing constructs, vectors, or constructs, according to any of the methods described herein, which may be constructed in any suitable manner described herein including different stacking or joint targeting arrangements, as well as modified cereal plants, plant parts, plant tissues, and plant cells made thereby and/or comprising any such recombinant DNA molecule, vector or construct. Since a non-coding RNA molecule expressed from the above constructs would be designed to target an endogenous GA oxidase gene, the cereal plant transformed with such recombinant DNA molecules, vectors or constructs should preferably correspond to the species of origin for the target sequence, or closely related species, strains, germplasms, lines, etc. For example, a suppression construct complementary to SEQ ID NO: 84 should be used to transform a sorghum plant, such as a Sorghum bicolor plant, or perhaps related sorghum species, strains, etc., that would be expected to have a closely related or similar GA oxidase (or GA oxidase-like) gene sequence.

The genomic sequences for each of the above identified genes from cereal plants are further provided in Table 3, which may be used to target those genes for genome editing according to any known technique. Any site-specific nuclease and method may be used as described herein to generate a DSB or nick at or near the genomic locus for the gene, which may be repaired imperfectly or via template-mediated recombination to create mutations, etc., at, near or within the gene. Suitable nucleases may be selected from the group consisting of a zinc-finger nuclease (ZFN), a meganuclease, an RNA-guided endonuclease, a TALE-endonuclease (TALEN), a recombinase, a transposase, or any combination thereof. For an RNA-guided endonuclease, a recombinant DNA construct or vector is provided comprising a guide RNA may be used to direct the nuclease to the target site. Accordingly, a guide RNA for editing a GA oxidase (or GA-oxidase-like) gene in a cereal crop may comprise a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of any one or more of SEQ ID NO: 84, 85, 87, 88, 89, 91, 92, 93, 95, 96, 98, 99, 100, 102, 103, 105, 106, 107, 109, 110, 111, 113, 114, 115, 119, 120, 122, 123, 124, 126, 127, 128, 130, 131, 132, 134, 135, and/or 137. For site-specific nucleases that are not RNA-guided, such as a zinc-finger nuclease (ZFN), a meganuclease, a TALE-endonuclease (TALEN), a recombinase, and/or a transposase, the genomic target specificity for editing is determined by its protein structure, particularly its DNA binding domain. Such site-specific nucleases may be chosen, designed or engineered to bind and cut a desired target site at or near any of the above GA oxidase (or GA oxidase-like) genes within the genome of a cereal plant. Similar to transformation with a suppression construct, a cereal plant transformed with a particular guide RNA, or a recombinant DNA molecule, vector or construct encoding a guide RNA, should preferably be the species in which the targeted genomic sequence exists, or a closely related species, strain, germplasm, line, etc., such that the guide RNA is able to recognize and bind to the desired target cut site.

Further provided are methods for introducing or transforming into a cereal plant, plant part, or plant cell any guide RNA described above, or any construct, vector, or construct encoding such a guide RNA, perhaps in addition to an RNA-guided nuclease, according to any of the methods described herein, as well as modified cereal plants, plant parts, plant tissues, and plant cells made thereby and/or comprising any such recombinant DNA molecule, vector or construct and/or an edited GA oxidase (or GA oxidase-like) gene. Modified cereal plants having an edited GA oxidase (or GA oxidase-like) gene, and/or a suppression element targeting a GA oxidase (or GA oxidase-like) gene, may have one or more beneficial traits provided herein, such as a shorter plant height, shorter internode length, increased stalk/stem diameter, improved lodging resistance, and/or drought tolerance, relative to a wild-type or control plant not having any such edit or suppression element. In addition to genome editing, mutations in a GA oxidase (or GA oxidase-like) gene may be introduced through other mutagenesis techniques as described herein

According to another aspect of the present disclosure, a transgenic plant(s), plant cell(s), seed(s), and plant part(s) are provided comprising a transformation event or insertion into the genome of at least one plant cell thereof, wherein the transformation event or insertion comprises a recombinant DNA sequence, construct or expression cassette comprising a transcribable DNA sequence encoding a non-coding RNA molecule that targets an endogenous GA oxidase gene for suppression, wherein the transcribable DNA sequence is operably linked to a plant-expressible promoter, such as a constitutive, vascular and/or leaf promoter. Such a transgenic plant may be produced by any suitable transformation method as provided above, to produce a transgenic R₀ plant, which may then be selfed or crossed to other plants to generate R₁ seed and subsequent progeny generations and seed through additional crosses, etc. Embodiments of the present disclosure further include a plant cell, tissue, explant, plant part, etc., comprising one or more transgenic cells having a transformation event or genomic insertion of a recombinant DNA or polynucleotide sequence comprising a transcribable DNA sequence encoding a non-coding RNA molecule that targets an endogenous GA oxidase gene for suppression.

Transgenic plants, plant cells, seeds, and plant parts of the present disclosure may be homozygous or hemizygous for a transgenic event or insertion of a transcribable DNA sequence for suppression of a GA oxidase gene into the genome of at least one plant cell thereof, or a targeted genome editing event, and plants, plant cells, seeds, and plant parts of the present embodiments may contain any number of copies of such transgenic event(s), insertion(s) and/or edit(s). The dosage or amount of expression of a transgene or transcribable DNA sequence may be altered by its zygosity and/or number of copies, which may affect the degree or extent of phenotypic changes in the transgenic plant, etc. As introduced above, transgenic plants provided herein may include a variety of monocot or cereal plants, and even crop plants, such as wheat, rice and sorghum, already having increased yield and/or lodging resistance due to prior breeding efforts and mutations of the GA pathway in these plants. Advantages of using a transgene or transcribable DNA sequence to express a suppression element targeting a biosynthetic GA oxidase gene include not only the ability to limit expression in a tissue-specific or tissue-preferred manner, but also the potential dominance (e.g., dominant negative effects) of a single or hemizygous copy of the transcribable DNA sequence to cause the beneficial short-stature, semi-dwarf traits or phenotypes in crop plants. Thus, recombinant DNA molecules or constructs of the present disclosure may be used to create beneficial traits in a variety of monocot or cereal plants without off-types using only a single copy of the transgenic event, insertion or construct. Unlike previously described mutations or alleles in the GA pathway that are recessive and require plants to be homozygous for the mutant allele, plants transformed with the GA-modifying transgenes and suppression constructs of the present disclosure may improve traits, yield and crop breeding efforts by facilitating the production of hybrid cereal plants since they only require a single or hemizygous copy of the transgene or suppression construct.

According to some embodiments, a transgenic or modified cereal or corn plant comprising a GA oxidase transgene or transcribable DNA sequence for suppression of an endogenous GA oxidase gene, or a genome edited GA oxidase gene, may be further characterized as having one or more beneficial traits, such as a shorter stature or semi-dwarf plant height, reduced internode length, increased stalk/stem diameter, improved lodging resistance, reduced green snap, deeper roots, increased leaf area, earlier canopy closure, increased foliar water content and/or higher stomatal conductance under water limiting conditions, reduced anthocyanin content and/or area in leaves under normal or nitrogen or water limiting stress conditions, improved yield-related traits including a larger female reproductive organ or ear, an increase in ear weight, harvest index, yield, seed or kernel number, and/or seed or kernel weight, relative to a wild type or control plant. Such a transgenic cereal or corn plant may further have increased stress tolerance, such as increased drought tolerance, nitrogen utilization, and/or tolerance to high density planting.

For purposes of the present disclosure, a “plant” includes an explant, plant part, seedling, plantlet or whole plant at any stage of regeneration or development. As used herein, a “transgenic plant” refers to a plant whose genome has been altered by the integration or insertion of a recombinant DNA molecule, construct or sequence. A transgenic plant includes an R₀ plant developed or regenerated from an originally transformed plant cell(s) as well as progeny transgenic plants in later generations or crosses from the R₀ transgenic plant. As used herein, a “plant part” may refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed (e.g., embryo, endosperm, and seed coat), fruit (e.g., the mature ovary), propagule, or other plant tissues (e.g., vascular tissue, dermal tissue, ground tissue, and the like), or any portion thereof. Plant parts of the present disclosure may be viable, nonviable, regenerable, and/or non-regenerable. A “propagule” may include any plant part that can grow into an entire plant.

According to present embodiments, a plant cell transformed with a construct or molecule comprising a transcribable DNA sequence for suppression of an endogenous GA oxidase gene, or with a construct used for genome editing, may include any plant cell that is competent for transformation as understood in the art based on the method of transformation, such as a meristem cell, an embryonic cell, a callus cell, etc. As used herein, a “transgenic plant cell” simply refers to any plant cell that is transformed with a stably-integrated recombinant DNA molecule, construct or sequence. A transgenic plant cell may include an originally-transformed plant cell, a transgenic plant cell of a regenerated or developed R₀ plant, a transgenic plant cell cultured from another transgenic plant cell, or a transgenic plant cell from any progeny plant or offspring of the transformed R₀ plant, including cell(s) of a plant seed or embryo, or a cultured plant cell, callus cell, etc.

Embodiments of the present disclosure further include methods for making or producing transgenic or modified plants, such as by transformation, genome editing, mutating, crossing, etc., wherein the method comprises introducing a recombinant DNA molecule, construct or sequence comprising a GA oxidase transgene or a transcribable DNA sequence for suppression of an endogenous GA oxidase gene into a plant cell, or editing or mutating the genomic locus of an endogenous GA oxidase gene, and then regenerating or developing the transgenic or modified plant from the transformed or edited plant cell, which may be performed under selection pressure favoring a transgenic event. Such methods may comprise transforming a plant cell with a recombinant DNA molecule, construct or sequence comprising the transcribable DNA sequence for suppression of an endogenous GA oxidase gene, and selecting for a plant having one or more altered phenotypes or traits, such as one or more of the following traits at one or more stages of development: shorter or semi-dwarf stature or plant height, shorter internode length in one or more internode(s), increased stalk/stem diameter, improved lodging resistance, reduced green snap, deeper roots, increased leaf area, earlier canopy closure, increased foliar water content and/or higher stomatal conductance under water limiting conditions, reduced anthocyanin content and/or area in leaves under normal or nitrogen or water limiting stress conditions, improved yield-related traits including a larger female reproductive organ or ear, an increase in ear weight, harvest index, yield, seed or kernel number, and/or seed or kernel weight, increased stress tolerance, such as increased drought tolerance, increased nitrogen utilization, and/or increased tolerance to high density planting, as compared to a wild type or control plant.

According to another aspect of the present disclosure, methods are provided for planting a modified or transgenic plant(s) provided herein at a normal/standard or high density in field. According to some embodiments, the yield of a crop plant per acre (or per land area) may be increased by planting a modified or transgenic plant(s) of the present disclosure at a higher density in the field. As described herein, modified or transgenic plants expressing a transcribable DNA sequence that encodes a non-coding RNA molecule targeting an endogenous GA oxidase gene for suppression, or having a genome-edited GA oxidase gene, may have reduced plant height, shorter internode(s), increased stalk/stem diameter, and/or increased lodging resistance. It is proposed that modified or transgenic plants may tolerate high density planting conditions since an increase in stem diameter may resist lodging and the shorter plant height may allow for increased light penetrance to the lower leaves under high density planting conditions. Thus, modified or transgenic plants provided herein may be planted at a higher density to increase the yield per acre (or land area) in the field. For row crops, higher density may be achieved by planting a greater number of seeds/plants per row length and/or by decreasing the spacing between rows.

According to some embodiments, a modified or transgenic crop plant may be planted at a density in the field (plants per land/field area) that is at least 5%, 10%, 15%, 20%, 25%, 50%, 75%, 100%, 125%, 150%, 175%, 200%, 225%, or 250% higher than the normal planting density for that crop plant according to standard agronomic practices. A modified or transgenic crop plant may be planted at a density in the field of at least 38,000 plants per acre, at least 40,000 plants per acre, at least 42,000 plants per acre, at least 44,000 plants per acre, at least 45,000 plants per acre, at least 46,000 plants per acre, at least 48,000 plants per acre, 50,000 plants per acre, at least 52,000 plants per acre, at least 54,000 per acre, or at least 56,000 plants per acre. As an example, corn plants may be planted at a higher density, such as in a range from about 38,000 plants per acre to about 60,000 plants per acre, or about 40,000 plants per acre to about 58,000 plants per acre, or about 42,000 plants per acre to about 58,000 plants per acre, or about 40,000 plants per acre to about 45,000 plants per acre, or about 45,000 plants per acre to about 50,000 plants per acre, or about 50,000 plants per acre to about 58,000 plants per acre, or about 52,000 plants per acre to about 56,000 plants per acre, or about 38,000 plants per acre, about 42,000 plant per acre, about 46,000 plant per acre, or about 48,000 plants per acre, about 50,000 plants per acre, or about 52,000 plants per acre, or about 54,000 plant per acre, as opposed to a standard density range, such as about 18,000 plants per acre to about 38,000 plants per acre.

According to embodiments of the present disclosure, a modified corn plant(s) is/are provided that comprise (i) a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and/or (ii) an average stem or stalk diameter of at least 18 mm, at least 18.5 mm, at least 19 mm, at least 19.5 mm, at least 20 mm, at least 20.5 mm, at least 21 mm, at least 21.5 mm, or at least 22 mm. Stated a different way, a modified corn plant(s) is/are provided that comprise a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and/or an average stem or stalk diameter that is greater than 18 mm, greater than 18.5 mm, greater than 19 mm, greater than 19.5 mm, greater than 20 mm, greater than 20.5 mm, greater than 21 mm, greater than 21.5 mm, or greater than 22 mm. Any such plant height trait or range that is expressed in millimeters (mm) may be converted into a different unit of measurement based on known conversions (e.g., one inch is equal to 2.54 cm or 25.4 millimeters, and millimeters (mm), centimeters (cm) and meters (m) only differ by one or more powers of ten). Thus, any measurement provided herein is further described in terms of any other comparable units of measurement according to known and established conversions. However, the exact plant height and/or stem diameter of a modified corn plant may depend on the environment and genetic background. Thus, the change in plant height and/or stem diameter of a modified corn plant may instead be described in terms of a minimum difference or percent change relative to a control plant. A modified corn plant may further comprise at least one ear that is substantially free of male reproductive tissues or structures or other off-types.

According to embodiments of the present disclosure, modified corn plants are provided that comprise a plant height during late vegetative and/or reproductive stages of development (e.g., at R3 stage) of between 1000 mm and 1800 mm, between 1000 mm and 1700 mm, between 1050 mm and 1700 mm, between 1100 mm and 1700 mm, between 1150 mm and 1700 mm, between 1200 mm and 1700 mm, between 1250 mm and 1700 mm, between 1300 mm and 1700 mm, between 1350 mm and 1700 mm, between 1400 mm and 1700 mm, between 1450 mm and 1700 mm, between 1000 mm and 1500 mm, between 1050 mm and 1500 mm, between 1100 mm and 1500 mm, between 1150 mm and 1500 mm, between 1200 mm and 1500 mm, between 1250 mm and 1500 mm, between 1300 mm and 1500 mm, between 1350 mm and 1500 mm, between 1400 mm and 1500 mm, between 1450 mm and 1500 mm, between 1000 mm and 1600 mm, between 1100 mm and 1600 mm, between 1200 mm and 1600 mm, between 1300 mm and 1600 mm, between 1350 mm and 1600 mm, between 1400 mm and 1600 mm, between 1450 mm and 1600 mm, of between 1000 mm and 2000 mm, between 1200 mm and 2000 mm, between 1200 mm and 1800 mm, between 1300 mm and 1700 mm, between 1400 mm and 1700 mm, between 1400 mm and 1600 mm, between 1400 mm and 1700 mm, between 1400 mm and 1800 mm, between 1400 mm and 1900 mm, between 1400 mm and 2000 mm, or between 1200 mm and 2500 mm, and/or an average stem diameter of between 17.5 mm and 22 mm, between 18 mm and 22 mm, between 18.5 and 22 mm, between 19 mm and 22 mm, between 19.5 mm and 22 mm, between 20 mm and 22 mm, between 20.5 mm and 22 mm, between 21 mm and 22 mm, between 21.5 mm and 22 mm, between 17.5 mm and 21 mm, between 17.5 mm and 20 mm, between 17.5 mm and 19 mm, between 17.5 mm and 18 mm, between 18 mm and 21 mm, between 18 mm and 20 mm, or between 18 mm and 19 mm. A modified corn plant may be substantially free of off-types, such as male reproductive tissues or structures in one or more ears of the modified corn plant.

According to embodiments of the present disclosure, modified corn plants are provided that have (i) a plant height that is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the height of a wild-type or control plant, and/or (ii) a stem or stalk diameter that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the stem diameter of the wild-type or control plant. According to embodiments of the present disclosure, a modified corn plant may have a reduced plant height that is no more than 20%, 25%, 30%, 35%, 40%, 45%, 50%, 55%, or 60% shorter than the height of a wild-type or control plant, and/or a stem or stalk diameter that is less than (or not more than) 10%, 15%, 20%, 25%, 30%, 35%, 40%, 45%, 50%, 55%, 60%, 65%, 70%, 75%, 80%, 85%, 90%, 95%, or 100% greater than the stem or stalk diameter of a wild-type or control plant. For example, a modified plant may have (i) a plant height that is at least 10%, at least 15%, or at least 20% less or shorter (i.e., greater than or equal to 10%, 15%, or 20% shorter), but not greater or more than 50% shorter, than a wild type or control plant, and/or (ii) a stem or stalk diameter that is that is at least 5%, at least 10%, or at least 15% greater, but not more than 30%, 35%, or 40% greater, than a wild type or control plant. For clarity, the phrases “at least 20% shorter” and “greater than or equal to 20% shorter” would exclude, for example, 10% shorter. Likewise for clarity, the phrases “not greater than 50% shorter”, “no more than 50% shorter” and “not more than 50% shorter” would exclude 60% shorter; the phrase “at least 5% greater” would exclude 2% greater; and the phrases “not more than 30% greater” and “no more than 30% greater” would exclude 40% greater.

According to embodiments of the present disclosure, modified corn plants are provided that comprise a height between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the height of a wild-type or control plant, and/or a stem or stalk diameter that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, between 50% and 75%, between 8% and 20%, or between 8% and 15% greater than the stem or stalk diameter of the wild-type or control plant.

According to embodiments of the present disclosure, modified corn plants are provided that comprise an average internode length (or a minus-2 internode length and/or minus-4 internode length relative to the position of the ear) that is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% less than the same or average internode length of a wild-type or control plant. The “minus-2 internode” of a corn plant refers to the second internode below the ear of the plant, and the “minus-4 internode” of a corn plant refers to the fourth internode below the ear of the plant According to many embodiments, modified corn plants are provided that have an average internode length (or a minus-2 internode length and/or minus-4 internode length relative to the position of the ear) that is between 5% and 75%, between 5% and 50%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 10% and 10%, between 10% and 75%, between 25% and 75%, between 10% and 50%, between 20% and 50%, between 25% and 50%, between 30% and 75%, between 30% and 50%, between 25% and 50%, between 15% and 50%, between 20% and 50%, between 25% and 45%, or between 30% and 45% less than the same or average internode length of a wild-type or control plant.

According to embodiments of the present disclosure, modified corn plants are provided that comprise an ear weight (individually or on average) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the ear weight of a wild-type or control plant. A modified corn plant provided herein may comprise an ear weight that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 25% and 75%, between 25% and 50%, or between 50% and 75% greater than the ear weight of a wild-type or control plant.

According to embodiments of the present disclosure, modified corn or cereal plants are provided that have a harvest index of at least 0.57, at least 0.58, at least 0.59, at least 0.60, at least 0.61, at least 0.62, at least 0.63, at least 0.64, or at least 0.65 (or greater). A modified corn plant may comprise a harvest index of between 0.57 and 0.65, between 0.57 and 0.64, between 0.57 and 0.63, between 0.57 and 0.62, between 0.57 and 0.61, between 0.57 and 0.60, between 0.57 and 0.59, between 0.57 and 0.58, between 0.58 and 0.65, between 0.59 and 0.65, or between 0.60 and 0.65. A modified corn plant may have a harvest index that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, or at least 50% greater than the harvest index of a wild-type or control plant. A modified corn plant may have a harvest index that is between 1% and 45%, between 1% and 40%, between 1% and 35%, between 1% and 30%, between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 30%, or between 5% and 40% greater than the harvest index of a wild-type or control plant.

According to embodiments of the present disclosure, modified corn or cereal plants are provided that have an increase in harvestable yield of at least 1 bushel per acre, at least 2 bushels per acre, at least 3 bushels per acre, at least 4 bushels per acre, at least 5 bushels per acre, at least 6 bushels per acre, at least 7 bushels per acre, at least 8 bushels per acre, at least 9 bushels per acre, or at least 10 bushels per acre, relative to a wild-type or control plant. A modified corn plant may have an increase in harvestable yield between 1 and 10, between 1 and 8, between 2 and 8, between 2 and 6, between 2 and 5, between 2.5 and 4.5, or between 3 and 4 bushels per acre. A modified corn plant may have an increase in harvestable yield that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, or at least 25% greater than the harvestable yield of a wild-type or control plant. A modified corn plant may have a harvestable yield that is between 1% and 25%, between 1% and 20%, between 1% and 15%, between 1% and 14%, between 1% and 13%, between 1% and 12%, between 1% and 11%, between 1% and 10%, between 1% and 9%, between 1% and 8%, between 1% and 7%, between 1% and 6%, between 1% and 5%, between 1% and 4%, between 1% and 3%, between 1% and 2%, between 5% and 15%, between 5% and 20%, between 5% and 25%, between 2% and 10%, between 2% and 9%, between 2% and 8%, between 2% and 7%, between 2% and 6%, between 2% and 5%, or between 2% and 4% greater than the harvestable yield of a wild-type or control plant.

According to embodiments of the present disclosure, a modified cereal or corn plant is provided that has a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a wild-type or control plant. A modified cereal or corn plant may have a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a wild-type or control plant. Further provided are populations of cereal or corn plants having increased lodging resistance and a reduced lodging frequency. Populations of modified cereal or corn plants are provided having a lodging frequency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% less or lower than a population of wild-type or control plants. A population of modified corn plants may comprise a lodging frequency that is between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 100%, between 10% and 75%, between 10% and 50%, between 10% and 40%, between 10% and 30%, between 10% and 20%, between 25% and 75%, between 25% and 50%, or between 50% and 75% less or lower than a population of wild-type or control plants, which may be expressed as an average over a specified number of plants or crop area of equal density.

According to embodiments of the present disclosure, modified corn plants are provided having a significantly reduced or decreased plant height (e.g., 2000 mm or less) and a significantly increased stem diameter (e.g., 18 mm or more), relative to a wild-type or control plant. According to these embodiments, the decrease or reduction in plant height and increase in stem diameter may be within any of the height, diameter or percentage ranges recited herein. Such modified corn plants having a reduced plant height and increased stem diameter relative to a wild-type or control plant may be transformed with a transcribable DNA sequence encoding a non-coding RNA molecule that targets at least one GA20 oxidase gene and/or at least one GA3 oxidase gene for suppression. Modified corn plants having a significantly reduced plant height and/or a significantly increased stem diameter relative to a wild-type or control plant may further have at least one ear that is substantially free of male reproductive tissues or structures and/or other off-types. Modified corn plants having a significantly reduced plant height and/or an increased stem diameter relative to a wild-type or control plant may have reduced activity of one or more GA20 oxidase and/or GA3 oxidase gene(s) in one or more tissue(s) of the plant, such as one or more vascular and/or leaf tissue(s) of the plant, relative to the same tissue(s) of the wild-type or control plant. According to many embodiments, modified corn plants may comprise at least one polynucleotide or transcribable DNA sequence encoding a non-coding RNA molecule operably linked to a promoter, which may be a constitutive, tissue-specific or tissue-preferred promoter, wherein the non-coding RNA molecule targets at least one GA20 oxidase and/or GA3 oxidase gene(s) for suppression as provided herein. The non-coding RNA molecule may be a miRNA, siRNA, or miRNA or siRNA precursor molecule. According to some embodiments, modified corn plants having a significantly reduced plant height and/or an increased stem diameter relative to a wild-type or control plant may further have an increased harvest index and/or increased lodging resistance relative to the wild-type or control plant.

Modified corn or cereal plants having a significantly reduced plant height and/or a significantly increased stem diameter relative to a wild-type or control plant may comprise a mutation (e.g., an insertion, deletion, substitution, etc.) in a GA oxidase gene introduced through a gene editing technology or other mutagenesis technique, wherein expression of the GA oxidase gene is reduced or eliminated in one or more tissues of the modified plant. Such modified corn plants having a reduced plant height and/or an increased stem diameter relative to a wild-type or control plant may further have an increased harvest index and/or increased lodging resistance relative to the wild-type or control plant. Such modified corn plants may be substantially free of off-types, such as male reproductive tissues or structures and/or other off-types in at least one ear of the modified plants. Plant mutagenesis techniques (excluding genome editing) may include chemical mutagenesis (i.e., treatment with a chemical mutagen, such as an azide, hydroxylamine, nitrous acid, acridine, nucleotide base analog, or alkylating agent—e.g., EMS (ethylmethane sulfonate), MNU (N-methyl-N-nitrosourea), etc.), physical mutagenesis (e.g., gamma rays, X-rays, UV, ion beam, other forms of radiation, etc.), and insertional mutagenesis (e.g., transposon or T-DNA insertion). Plants or various plant parts, plant tissues or plant cells may be subjected to mutagenesis. Treated plants may be reproduced to collect seeds or produce a progeny plant, and treated plant parts, plant tissues or plant cells may be developed or regenerated into plants or other plant tissues. Mutations generated with chemical or physical mutagenesis techniques may include a frameshift, missense or nonsense mutation leading to loss of function or expression of a targeted gene, such as a GA3 or GA20 oxidase gene.

One method for mutagenesis of a gene is called “TILLING” (for targeting induced local lesions in genomes), in which mutations are created in a plant cell or tissue, preferably in the seed, reproductive tissue or germline of a plant, for example, using a mutagen, such as an EMS treatment. The resulting plants are grown and self-fertilized, and the progeny are used to prepare DNA samples. PCR amplification and sequencing of a nucleic acid sequence of a GA oxidase gene may be used to identify whether a mutated plant has a mutation in the GA oxidase gene. Plants having mutations in the GA oxidase gene may then be tested for an altered trait, such as reduced plant height. Alternatively, mutagenized plants may be tested for an altered trait, such as reduced plant height, and then PCR amplification and sequencing of a nucleic acid sequence of a GA oxidase gene may be used to determine whether a plant having the altered trait also has a mutation in the GA oxidase gene. See, e.g., Colbert et al., 2001, Plant Physiol 126:480-484; and McCallum et al., 2000, Nature Biotechnology 18:455-457. TILLING can be used to identify mutations that alter the expression a gene or the activity of proteins encoded by a gene, which may be used to introduce and select for a targeted mutation in a GA oxidase gene of a corn or cereal plant.

Corn or cereal plants that have been subjected to a mutagenesis or genome editing treatment may be screened and selected based on an observable phenotype (e.g., any phenotype described herein, such as shorter plant height, increased stem/stalk diameter, etc.), or using a selection agent with a selectable marker (e.g., herbicide, etc.), a screenable marker, or a molecular technique (e.g., lower GA levels, lower GA oxidase transcript or protein levels, presence of transgene or transcribable sequence, etc.). Such screening and/or selecting techniques may be used to identify and select plants having a mutation in a GA oxidase gene that leads to a desirable plant phenotype.

According to embodiments of the present disclosure, a population of modified corn or cereal plants are provided, wherein the population of modified corn or cereal plants have an average plant height that is significantly less, and/or an average stem or stalk diameter that is significantly more, than a population of wild-type or control plants. The population of modified corn or cereal plants may share ancestry with a single modified corn or cereal plant and/or have a single transgenic GA oxidase suppression construct insertion, event or edit in common. Modified corn plants within a population of modified corn plants may generally comprise at least one ear that is substantially free of male reproductive tissues or structures and/or other off-types. A population of modified corn or cereal plants may have increased lodging resistance on average or per number of plants or field area than a population of wild-type or control plants. The population of modified corn or cereal plants may have a lodging frequency that is at least 5%, at least 10%, at least 20%, at least 30%, at least 40%, at least 50%, at least 60%, at least 70% at least 80%, at least 90%, or 100% less (or lower) than a population of control corn or cereal plants. A population of modified corn plants may have a harvest index of at least 0.57 or greater.

According to embodiments of the present invention, modified corn or cereal plants are provided having a reduced gibberellin content (in active form) in at least the stem and internode tissue(s), such as the stem, internode, leaf and/or vascular tissue(s), as compared to the same tissue(s) of wild-type or control plants. According to many embodiments, modified corn or cereal plants are provided having a significantly reduced plant height and/or a significantly increased stem diameter relative to wild-type or control plants, wherein the modified corn or cereal plants further have significantly reduced or decreased level(s) of active gibberellins or active GAs (e.g., one or more of GA1, GA3, GA4, and/or GA7) in one or more stem, internode, leaf and/or vascular tissue(s), relative to the same tissue(s) of the wild-type or control plants. For example, the level of one or more active GAs in the stem, internode, leaf and/or vascular tissue(s) of a modified corn or cereal plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less or lower than in the same tissue(s) of a wild-type or control corn plant.

According to some embodiments, a modified corn or cereal plant may comprise an active gibberellin (GA) level(s) (e.g., one or more of GA1, GA3, GA4, and/or GA7) in one or more stem, internode, leaf and/or vascular tissue(s) that is between 5% and 50%, between 10% and 100%, between 20% and 100%, between 30% and 100%, between 40% and 100%, between 50% and 100%, between 60% and 100%, between 70% and 100%, between 80% and 100%, between 80% and 90%, between 10% and 90%, between 10% and 80%, between 10% and 70%, between 10% and 60%, between 10% and 50%, between 10% and 40%, between 10% and 30%, between 10% and 20%, between 50% and 100%, between 20% and 90%, between 20% and 80%, between 20% and 70%, between 20% and 60%, between 20% and 50%, between 20% and 40%, between 20% and 40%, between 20% and 30%, between 30% and 90%, between 30% and 80%, between 30% and 70%, between 30% and 60%, between 30% and 50%, between 30% and 40%, between 40% and 90% between 40% and 80%, between 40% and 70%, between 40% and 60%, between 40% and 50%, between 50% and 90%, between 50% and 80%, between 50% and 70%, between 50% and 60%, between 60% and 90%, between 60% and 80%, between 60% and 70%, between 70% and 90%, or between 70% and 80% less or (or lower) than in the same tissue(s) of a wild-type or control corn plant. A modified corn or cereal plant having a reduced active gibberellin (GA) level(s) in one or more stem, internode, leaf and/or vascular tissue(s) may further be substantially free of off-types, such as male reproductive tissues or structures and/or other off-types in at least one ear of a modified corn plant.

According to embodiments of the present disclosure, modified corn or cereal plants are provided having a significantly reduced or eliminated expression level of one or more GA3 oxidase and/or GA20 oxidase gene transcript(s) and/or protein(s) in one or more tissue(s), such as one or more stem, internode, leaf and/or vascular tissue(s), of the modified plants, as compared to the same tissue(s) of wild-type or control plants. According to many embodiments, a modified corn or cereal plant is provided comprising a significantly reduced plant height and/or a significantly increased stem diameter relative to wild-type or control plants, wherein the modified corn or cereal plant has a significantly reduced or eliminated expression level of one or more GA20 oxidase and/or GA3 oxidase gene transcript(s) and/or protein(s) in one or more tissues, such as one or more stem, internode, leaf and/or vascular tissue(s), of the modified plant, as compared to the same tissue(s) of a wild-type or control corn plant. For example, a modified corn or cereal plant has a significantly reduced or eliminated expression level of a GA20 oxidase_3 and/or GA20 oxidase_5 gene transcript(s) and/or protein(s), and/or a significantly reduced or eliminated expression level of a GA3 oxidase_1 and/or GA3 oxidase_2 gene transcript(s) and/or protein(s), in the whole modified plant, or in one or more stem, internode, leaf and/or vascular tissue(s) of the modified plant, as compared to the same tissue(s) of a wild-type or control plant. For example, the level of one or more GA3 oxidase and/or GA20 oxidase gene transcript(s) and/or protein(s), or one or more GA oxidase (or GA oxidase-like) gene transcript(s) and/or protein(s), in one or more stem, internode, leaf and/or vascular tissue(s) of a modified corn plant may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% less or lower than in the same tissue(s) of a wild-type or control corn or cereal plant.

According to some embodiments, a modified corn or cereal plant may comprise level(s) of one or more GA3 oxidase and/or GA20 oxidase gene transcript(s) and/or protein(s), or one or more GA oxidase (or GA oxidase-like) gene transcript(s) and/or protein(s), in the whole plant, or in one or more stem, internode, leaf and/or vascular tissue(s), that is between 5% and 50%, between 10% and 100%, between 20% and 100%, between 30% and 100%, between 40% and 100%, between 50% and 100%, between 60% and 100%, between 70% and 100%, between 80% and 100%, between 80% and 90%, between 10% and 90%, between 10% and 80%, between 10% and 70%, between 10% and 60%, between 10% and 50%, between 10% and 40%, between 10% and 30%, between 10% and 20%, between 50% and 100%, between 20% and 90%, between 20% and 80%, between 20% and 70%, between 20% and 60%, between 20% and 50%, between 20% and 40%, between 20% and 40%, between 20% and 30%, between 30% and 90%, between 30% and 80%, between 30% and 70%, between 30% and 60%, between 30% and 50%, between 30% and 40%, between 40% and 90% between 40% and 80%, between 40% and 70%, between 40% and 60%, between 40% and 50%, between 50% and 90%, between 50% and 80%, between 50% and 70%, between 50% and 60%, between 60% and 90%, between 60% and 80%, between 60% and 70%, between 70% and 90%, or between 70% and 80% less or lower than in the same tissue(s) of a wild-type or control corn or cereal plant. A modified corn or cereal plant having a reduced or eliminated expression level of at least one GA20 oxidase and/or GA3 oxidase gene(s) in one or more tissue(s), may also be substantially free of off-types, such as male reproductive tissues or structures and/or other off-types in at least one ear of the modified corn plant.

According to some embodiments, methods are provided comprising reducing or eliminating the expression of at least one GA20 oxidase gene and/or at least one GA3 oxidase gene in a crop plant, such as in one or more stem, internode, vascular and/or leaf tissue of the crop plant, wherein the expression of the at least one GA20 oxidase gene and/or at least one GA3 oxidase gene(s) is/are not significantly altered or changed in at least one reproductive tissue of the plant, and/or wherein the level(s) of one or more active GAs is/are not significantly altered or changed in at least one reproductive tissue of the plant, as compared to a wild-type or control plant. According to many embodiments, the expression level(s) of at least one GA20 oxidase or GA3 oxidase gene is reduced or eliminated in at least one tissue of a modified plant with a recombinant DNA construct comprising a transcribable DNA sequence encoding a suppression element for the GA20 oxidase or GA3 oxidase gene, such as at least one mature miRNA or miRNA precursor that is processed into a mature miRNA, wherein the miRNA is able to reduce or suppress the expression level of the at least one GA20 oxidase or GA3 oxidase gene, and wherein the transcribable DNA sequence is operably linked to a constitutive, tissue-specific or tissue-preferred promoter.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. Nucleic acids can be isolated and detected using techniques known in the art. For example, nucleic acids can be isolated and detected using, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis. Any method known in the art may be used to screen for, and/or identify, cells, plants, etc., having a transgene or genome edit in its genome, which may be based on any suitable form of visual observation, selection, molecular technique, etc.

In some embodiments, methods are provided for detecting recombinant nucleic acids and/or polypeptides in plant cells. For example, nucleic acids may be detected using hybridization probes or through production of amplicons using PCR with primers as known in the art.

Hybridization between nucleic acids is discussed in Sambrook et al. (1989, Molecular Cloning: A Laboratory Manual, 2nd Ed., Cold Spring Harbor Laboratory Press, Cold Spring Harbor, N.Y.). Polypeptides can be detected using antibodies. Techniques for detecting polypeptides using antibodies include enzyme linked immunosorbent assays (ELISAs), Western blots, immunoprecipitations, immunofluorescence, and the like. An antibody provided herein may be a polyclonal antibody or a monoclonal antibody. An antibody having specific binding affinity for a polypeptide provided herein can be generated using methods known in the art. An antibody or hybridization probe may be attached to a solid support, such as a tube, plate or well, using methods known in the art.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials.

The screening and selection of modified, edited or transgenic plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

### EMBODIMENTS

The Following Paragraphs List a First Subset of Exemplary Embodiments.

Embodiment 1. A recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in a cereal plant or plant cell, the endogenous GA20 oxidase protein being at least 80%, at least 85%, at least 90%, at least 95%, at least 96%, at least 97%, at least 98%, at least 99%, at least 99.5%, or 100% identical to SEQ ID NO: 9, and wherein the transcribable DNA sequence is operably linked to a plant-expressible promoter.

Embodiment 2. The recombinant DNA construct of Embodiment 1, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7 or SEQ ID NO: 8.

Embodiment 3. The recombinant DNA construct of Embodiment 1, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in a monocot or cereal plant or plant cell, the endogenous GA20 oxidase protein being at least 80%, at least 85%, at least 90%, at least 95%, at least 96%, at least 97%, at least 98%, at least 99%, at least 99.5%, or 100% identical to SEQ ID NO: 15.

Embodiment 4. The recombinant DNA construct of Embodiment 3, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 13 or SEQ ID NO: 14.

Embodiment 5. The recombinant DNA construct of Embodiment 1, wherein the plant-expressible promoter is a vascular promoter.

Embodiment 6. The recombinant DNA construct of Embodiment 5, wherein the vascular promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

Embodiment 7. The recombinant DNA construct of Embodiment 5, wherein the vascular promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 67, SEQ ID NO: 68, SEQ ID NO: 69, SEQ ID NO: 70, or SEQ ID NO:

71, or a functional portion thereof.

Embodiment 8. The recombinant DNA construct of Embodiment 1, wherein the plant-expressible promoter is a RTBV promoter.

Embodiment 9. The recombinant DNA construct of Embodiment 8, wherein the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 65 or SEQ ID NO: 66, or a functional portion thereof.

Embodiment 10. The recombinant DNA construct of Embodiment 1, wherein the plant-expressible promoter is a leaf promoter.

Embodiment 11. The recombinant DNA construct of Embodiment 10, wherein the leaf promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

Embodiment 12. The recombinant DNA construct of Embodiment 10, wherein the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 72, SEQ ID NO: 73 or SEQ ID NO: 74, or a functional portion thereof.

Embodiment 13. The recombinant DNA construct of Embodiment 1, wherein the plant-expressible promoter is a constitutive promoter.

Embodiment 14. The recombinant DNA construct of Embodiment 13, wherein the constitutive promoter is selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof.

Embodiment 15. The recombinant DNA construct of Embodiment 13, wherein the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 75, SEQ ID NO: 76, SEQ ID NO: 77, SEQ ID NO: 78, SEQ ID NO: 79, SEQ ID NO: 80, SEQ ID NO: 81, SEQ ID NO: 82 or SEQ ID NO: 83, or a functional portion thereof.

Embodiment 16. The recombinant DNA construct of Embodiment 1, wherein the non-coding RNA molecule encoded by the transcribable DNA sequence is a precursor miRNA or siRNA that is processed or cleaved in a plant cell to form a mature miRNA or siRNA.

Embodiment 17. A transformation vector comprising the recombinant DNA construct of Embodiment 1.

Embodiment 18. A transgenic cereal plant, plant part or plant cell comprising the recombinant DNA construct of Embodiment 1.

Embodiment 19. The transgenic cereal plant of Embodiment 18, wherein the transgenic plant has one or more of the following traits relative to a control plant: shorter plant height, increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy.

Embodiment 20. The transgenic cereal plant of Embodiment 18, wherein the transgenic plant has a shorter plant height and/or improved lodging resistance.

Embodiment 21. The transgenic cereal plant of Embodiment 18, wherein the height of the transgenic plant is at least 10%, at least 20%, at least 25%, at least 30%, at least 35%, or at least 40% shorter than a wild-type control plant.

Embodiment 22. The transgenic cereal plant of Embodiment 18, wherein the stalk or stem diameter of the transgenic plant at one or more stem internodes is at least 5%, at least 10%, at least 15%, at least 20%, at least 25%, at least 30%, at least 35%, or at least 40% greater than the stalk or stem diameter at the same one or more internodes of a wild-type control plant.

Embodiment 23. The transgenic cereal plant of any one of Embodiments 18, wherein the transgenic cereal plant is a corn plant, and wherein the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same internode of a wild-type control plant.

Embodiment 24. The transgenic cereal plant of Embodiment 18, wherein the level of one or more active GAs in at least one internode tissue of the stem or stalk of the transgenic plant is lower than the same internode tissue of a wild-type control plant.

Embodiment 25. The transgenic cereal plant of Embodiment 18, wherein the level of one or more active GAs in at least one internode tissue of the stem or stalk of the transgenic plant is at least 5%, at least 10%, at least 15%, at least 20%, at least 25%, at least 30%, at least 35%, or at least 40% lower than the same internode tissue of a wild-type control plant.

Embodiment 26. A transgenic corn plant, plant part or plant cell comprising the recombinant DNA construct of Embodiment 1.

Embodiment 27. A method for producing a transgenic cereal plant, comprising: (a) transforming at least one cell of an explant with the recombinant DNA construct of Embodiment 1, and (b) regenerating or developing the transgenic cereal plant from the transformed explant.

Embodiment 28. The method of Embodiment 25, wherein the cereal plant is transformed via Agrobacterium mediated transformation or particle bombardment.

Embodiment 29. A recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in a monocot or cereal plant or plant cell, the endogenous GA20 oxidase protein being at least 80%, at least 85%, at least 90%, at least 95%, at least 96%, at least 97%, at least 98%, at least 99%, at least 99.5%, or 100% identical to SEQ ID NO: 15, and wherein the transcribable DNA sequence is operably linked to a plant-expressible promoter.

Embodiment 30. The recombinant DNA construct of Embodiment 29, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 13 or SEQ ID NO: 14.

Embodiment 31. The recombinant DNA construct of Embodiment 29, wherein the plant-expressible promoter is a vascular promoter.

Embodiment 32. The recombinant DNA construct of Embodiment 31, wherein the vascular promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

Embodiment 33. The recombinant DNA construct of Embodiment 31, wherein the vascular promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 67, SEQ ID NO: 68, SEQ ID NO: 69, SEQ ID NO: 70, or SEQ ID NO: 71, or a functional portion thereof.

Embodiment 34. The recombinant DNA construct of Embodiment 29, wherein the plant-expressible promoter is a RTBV promoter.

Embodiment 35. The recombinant DNA construct of Embodiment 34, wherein the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 65 or SEQ ID NO: 66, or a functional portion thereof.

Embodiment 36. The recombinant DNA construct of Embodiment 29, wherein the plant-expressible promoter is a leaf promoter.

Embodiment 37. The recombinant DNA construct of Embodiment 36, wherein the leaf promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

Embodiment 38. The recombinant DNA construct of Embodiment 36, wherein the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 72, SEQ ID NO: 73 or SEQ ID NO: 74, or a functional portion thereof.

Embodiment 39. The recombinant DNA construct of Embodiment 29, wherein the plant-expressible promoter is a constitutive promoter.

Embodiment 40. The recombinant DNA construct of Embodiment 39, wherein the constitutive promoter is selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof.

Embodiment 41. The recombinant DNA construct of Embodiment 39, wherein the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 75, SEQ ID NO: 76, SEQ ID NO: 77, SEQ ID NO: 78, SEQ ID NO: 79, SEQ ID NO: 80, SEQ ID NO: 81, SEQ ID NO: 82 or SEQ ID NO: 83, or a functional portion thereof.

Embodiment 42. The recombinant DNA construct of Embodiment 29, wherein the non-coding RNA molecule encoded by the transcribable DNA sequence is a precursor miRNA or siRNA that is processed or cleaved in a plant cell to form a mature miRNA or siRNA.

Embodiment 43. A transformation vector comprising the recombinant DNA construct of Embodiment 29.

Embodiment 44. A transgenic cereal plant, plant part or plant cell comprising the recombinant DNA construct of Embodiment 29.

Embodiment 45. The transgenic cereal plant of Embodiment 44, wherein the transgenic plant has one or more of the following traits relative to a control plant: shorter plant height, increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy.

Embodiment 46. The transgenic cereal plant of Embodiment 44, wherein the transgenic plant has a shorter plant height and/or improved lodging resistance.

Embodiment 47. The transgenic cereal plant of Embodiment 44, wherein the height of the transgenic plant is at least 10%, at least 20%, at least 25%, at least 30%, at least 35%, or at least 40% shorter than a wild-type control plant.

Embodiment 48. The transgenic cereal plant of Embodiment 44, wherein the stalk or stem diameter of the transgenic plant at one or more stem internodes is at least 5%, at least 10%, at least 15%, at least 20%, at least 25%, at least 30%, at least 35%, or at least 40% greater than the stalk or stem diameter at the same one or more internodes of a wild-type control plant.

Embodiment 49. The transgenic cereal plant of any one of Embodiments 44, wherein the transgenic cereal plant is a corn plant, and wherein the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same internode of a wild-type control plant.

Embodiment 50. The transgenic cereal plant of Embodiment 44, wherein the level of one or more active GAs in at least one internode tissue of the stem or stalk of the transgenic plant is lower than the same internode tissue of a wild-type control plant.

Embodiment 51. The transgenic cereal plant of Embodiment 44, wherein the level of one or more active GAs in at least one internode tissue of the stem or stalk of the transgenic plant is at least 5%, at least 10%, at least 15%, at least 20%, at least 25%, at least 30%, at least 35%, or at least 40% lower than the same internode tissue of a wild-type control plant.

Embodiment 52. A transgenic corn plant, plant part or plant cell comprising the recombinant DNA construct of Embodiment 29.

Embodiment 53. A method for producing a transgenic cereal plant, comprising: (a) transforming at least one cell of an explant with the recombinant DNA construct of Embodiment 29, and (b) regenerating or developing the transgenic cereal plant from the transformed explant.

Embodiment 54. The method of Embodiment 29, wherein the cereal plant is transformed via Agrobacterium mediated transformation or particle bombardment.

Embodiment 55. A recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in a monocot or cereal plant or plant cell, the endogenous GA3 oxidase protein being at least 80%, at least 85%, at least 90%, at least 95%, at least 96%, at least 97%, at least 98%, at least 99%, at least 99.5%, or 100% identical to SEQ ID NO: 30 or 33, and wherein the transcribable DNA sequence is operably linked to a plant-expressible promoter.

Embodiment 56. The recombinant DNA construct of Embodiment 55, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 28, 29, 31 or 32.

Embodiment 57. The recombinant DNA construct of Embodiment 55, wherein the plant-expressible promoter is a vascular promoter.

Embodiment 58. The recombinant DNA construct of Embodiment 57, wherein the vascular promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

Embodiment 59. The recombinant DNA construct of Embodiment 57, wherein the vascular promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 67, SEQ ID NO: 68, SEQ ID NO: 69, SEQ ID NO: 70, or SEQ ID NO:

71, or a functional portion thereof.

Embodiment 60. The recombinant DNA construct of Embodiment 55, wherein the plant-expressible promoter is a RTBV promoter.

Embodiment 61. The recombinant DNA construct of Embodiment 60, wherein the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 65 or SEQ ID NO: 66, or a functional portion thereof.

Embodiment 62. The recombinant DNA construct of Embodiment 55, wherein the plant-expressible promoter is a leaf promoter.

Embodiment 63. The recombinant DNA construct of Embodiment 62, wherein the leaf promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

Embodiment 64. The recombinant DNA construct of Embodiment 62, wherein the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 72, SEQ ID NO: 73 or SEQ ID NO: 74, or a functional portion thereof.

Embodiment 65. The recombinant DNA construct of Embodiment 55, wherein the plant-expressible promoter is a constitutive promoter.

Embodiment 66. The recombinant DNA construct of Embodiment 65, wherein the constitutive promoter is selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof.

Embodiment 67. The recombinant DNA construct of Embodiment 65, wherein the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 75, SEQ ID NO: 76, SEQ ID NO: 77, SEQ ID NO: 78, SEQ ID NO: 79, SEQ ID NO: 80, SEQ ID NO: 81, SEQ ID NO: 82 or SEQ ID NO: 83, or a functional portion thereof.

Embodiment 68. The recombinant DNA construct of Embodiment 55, wherein the non-coding RNA molecule encoded by the transcribable DNA sequence is a precursor miRNA or siRNA that is processed or cleaved in a plant cell to form a mature miRNA or siRNA.

Embodiment 69. A transformation vector comprising the recombinant DNA construct of Embodiment 55.

Embodiment 70. A transgenic cereal plant, plant part or plant cell comprising the recombinant DNA construct of Embodiment 55.

Embodiment 71. The transgenic cereal plant of Embodiment 70, wherein the transgenic plant has one or more of the following traits relative to a control plant: shorter plant height, increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy.

Embodiment 72. The transgenic cereal plant of Embodiment 70, wherein the transgenic plant has a shorter plant height and/or improved lodging resistance.

Embodiment 73. The transgenic cereal plant of Embodiment 70, wherein the height of the transgenic plant is at least 10%, at least 20%, at least 25%, at least 30%, at least 35%, or at least 40% shorter than a wild-type control plant.

Embodiment 74. The transgenic cereal plant of Embodiment 70, wherein the stalk or stem diameter of the transgenic plant at one or more stem internodes is at least 5%, at least 10%, at least 15%, at least 20%, at least 25%, at least 30%, at least 35%, or at least 40% greater than the stalk or stem diameter at the same one or more internodes of a wild-type control plant.

Embodiment 75. The transgenic cereal plant of any one of Embodiments 70, wherein the transgenic cereal plant is a corn plant, and wherein the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same internode of a wild-type control plant.

Embodiment 76. The transgenic cereal plant of Embodiment 70, wherein the level of one or more active GAs in at least one internode tissue of the stem or stalk of the transgenic plant is lower than the same internode tissue of a wild-type control plant.

Embodiment 77. The transgenic cereal plant of Embodiment 70, wherein the level of one or more active GAs in at least one internode tissue of the stem or stalk of the transgenic plant is at least 5%, at least 10%, at least 15%, at least 20%, at least 25%, at least 30%, at least 35%, or at least 40% lower than the same internode tissue of a wild-type control plant.

Embodiment 78. A transgenic corn plant, plant part or plant cell comprising the recombinant DNA construct of Embodiment 55.

Embodiment 79. A method for producing a transgenic cereal plant, comprising: (a) transforming at least one cell of an explant with the recombinant DNA construct of Embodiment 55, and (b) regenerating or developing the transgenic cereal plant from the transformed explant.

Embodiment 80. The method of Embodiment 79, wherein the cereal plant is transformed via Agrobacterium mediated transformation or particle bombardment.

Embodiment 81. A recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in a monocot or cereal plant or plant cell, the endogenous GA20 oxidase protein being at least 80%, at least 85%, at least 90%, at least 95%, at least 96%, at least 97%, at least 98%, at least 99%, at least 99.5%, or 100% identical to SEQ ID NO: 12, and wherein the transcribable DNA sequence is operably linked to a plant-expressible promoter.

Embodiment 82. The recombinant DNA construct of Embodiment 81, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 10 or 11.

Embodiment 83. The recombinant DNA construct of Embodiment 81, wherein the plant-expressible promoter is a vascular promoter.

Embodiment 84. The recombinant DNA construct of Embodiment 83, wherein the vascular promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

Embodiment 85. The recombinant DNA construct of Embodiment 83, wherein the vascular promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 67, SEQ ID NO: 68, SEQ ID NO: 69, SEQ ID NO: 70, or SEQ ID NO: 71, or a functional portion thereof.

Embodiment 86. The recombinant DNA construct of Embodiment 81, wherein the plant-expressible promoter is a RTBV promoter.

Embodiment 87. The recombinant DNA construct of Embodiment 86, wherein the plant-expressible promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 65 or SEQ ID NO: 66, or a functional portion thereof.

Embodiment 88. The recombinant DNA construct of Embodiment 81, wherein the plant-expressible promoter is a leaf promoter.

Embodiment 89. The recombinant DNA construct of Embodiment 88, wherein the leaf promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

Embodiment 90. The recombinant DNA construct of Embodiment 88, wherein the leaf promoter comprises a DNA sequence that is at least 80%, at least 85%, at least 90%, at least 95%, at least 96%, at least 97%, at least 98%, at least 99%, at least 99.5% or 100% identical to one or more of SEQ ID NO: 72, SEQ ID NO: 73 or SEQ ID NO: 74, or a functional portion thereof.

Embodiment 91. The recombinant DNA construct of Embodiment 81, wherein the plant-expressible promoter is a constitutive promoter.

Embodiment 92. The recombinant DNA construct of Embodiment 91, wherein the constitutive promoter is selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof.

Embodiment 93. The recombinant DNA construct of Embodiment 91, wherein the constitutive promoter comprises a DNA sequence that is at least 80%, at least 85%, at least 90%, at least 95%, at least 96%, at least 97%, at least 98%, at least 99%, at least 99.5% or 100% identical to one or more of SEQ ID NOs: 75, SEQ ID NO: 76, SEQ ID NO: 77, SEQ ID NO: 78, SEQ ID NO: 79, SEQ ID NO: 80, SEQ ID NO: 81, SEQ ID NO: 82 or SEQ ID NO: 83, or a functional portion thereof.

Embodiment 94. The recombinant DNA construct of Embodiment 81, wherein the non-coding RNA molecule encoded by the transcribable DNA sequence is a precursor miRNA or siRNA that is processed or cleaved in a plant cell to form a mature miRNA or siRNA.

Embodiment 95. A transformation vector comprising the recombinant DNA construct of Embodiment 81.

Embodiment 96. A transgenic cereal plant, plant part or plant cell comprising the recombinant DNA construct of Embodiment 81.

Embodiment 97. The transgenic cereal plant of Embodiment 96, wherein the transgenic plant has one or more of the following traits relative to a control plant: shorter plant height, increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and/or increased prolificacy.

Embodiment 98. The transgenic cereal plant of Embodiment 96, wherein the transgenic plant has a shorter plant height and/or improved lodging resistance.

Embodiment 99. The transgenic cereal plant of Embodiment 96, wherein the height of the transgenic plant is at least 10%, at least 20%, at least 25%, at least 30%, at least 35%, or at least 40% shorter than a wild-type control plant.

Embodiment 100. The transgenic cereal plant of Embodiment 96, wherein the stalk or stem diameter of the transgenic plant at one or more stem internodes is at least 5%, at least 10%, at least 15%, at least 20%, at least 25%, at least 30%, at least 35%, or at least 40% greater than the stalk or stem diameter at the same one or more internodes of a wild-type control plant.

Embodiment 101. The transgenic cereal plant of any one of Embodiments 96, wherein the transgenic cereal plant is a corn plant, and wherein the stalk or stem diameter of the transgenic corn plant at one or more of the first, second, third, and/or fourth internode below the ear is at least 5%, at least 10%, at least 15%, at least 20%, at least 25%, at least 30%, at least 35%, or at least 40% greater than the same internode of a wild-type control plant.

Embodiment 102. The transgenic cereal plant of Embodiment 96, wherein the level of one or more active GAs in at least one internode tissue of the stem or stalk of the transgenic plant is lower than the same internode tissue of a wild-type control plant.

Embodiment 103. The transgenic cereal plant of Embodiment 96, wherein the level of one or more active GAs in at least one internode tissue of the stem or stalk of the transgenic plant is at least 5%, at least 10%, at least 15%, at least 20%, at least 25%, at least 30%, at least 35%, or at least 40% lower than the same internode tissue of a wild-type control plant.

Embodiment 104. A transgenic corn plant, plant part or plant cell comprising the recombinant DNA construct of Embodiment 81.

Embodiment 105. A method for producing a transgenic cereal plant, comprising: (a) transforming at least one cell of an explant with the recombinant DNA construct of Embodiment 81, and (b) regenerating or developing the transgenic cereal plant from the transformed explant.

Embodiment 106. The method of Embodiment 105, wherein the cereal plant is transformed via Agrobacterium mediated transformation or particle bombardment.

Embodiment 107. The recombinant DNA construct of Embodiment 1, 29, 55 or 81, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein in a monocot or cereal plant or plant cell, the endogenous GA oxidase protein being at least 80%, at least 85%, at least 90%, at least 95%, at least 96%, at least 97%, at least 98%, at least 99%, at least 99.5%, or 100% identical to one or more of SEQ ID NOs: 3, 6, 9, 12, 15, 18, 21, 24, 27, 30 and 33.

Embodiment 108. The recombinant DNA construct of Embodiment 107, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of one or more of SEQ ID NOs: 1, 2, 4, 5, 7, 8, 10, 11, 13, 14, 16, 17, 19, 20, 22, 23, 25, 26, 28, 29, 31, and 32.

Embodiment 109. A recombinant DNA construct comprising a transcribable DNA sequence encoding a non-coding RNA molecule, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous protein in a monocot or cereal plant or plant cell, the endogenous protein being at least 80%, at least 85%, at least 90%, at least 95%, at least 96%, at least 97%, at least 98%, at least 99%, at least 99.5%, or 100% identical to SEQ ID NO: 86, 90, 94, 97, 101, 104, 108, 112, 116, 118, 121, 125, 129, 133, or 136, and wherein the transcribable DNA sequence is operably linked to a plant-expressible promoter.

Embodiment 110. The recombinant DNA construct of Embodiment 109, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 84, 85, 87, 88, 89, 91, 92, 93, 95, 96, 98, 99, 100, 102, 103, 105, 106, 107, 109, 110, 111, 113, 114, 115, 119, 120, 122, 123, 124, 126, 127, 128, 130, 131, 132, 134, 135, or 137.

Embodiment 111. The recombinant DNA construct of Embodiment 109, wherein the plant-expressible promoter is a vascular promoter.

Embodiment 112. The recombinant DNA construct of Embodiment 109, wherein the plant-expressible promoter is a RTBV promoter.

Embodiment 113. The recombinant DNA construct of Embodiment 109, wherein the plant-expressible promoter is a leaf promoter.

Embodiment 114. The recombinant DNA construct of Embodiment 109, wherein the plant-expressible promoter is a constitutive promoter.

Embodiment 115. A transformation vector comprising the recombinant DNA construct of Embodiment 81.

Embodiment 116. A transgenic cereal plant, plant part or plant cell comprising the recombinant DNA construct of Embodiment 109.

Embodiment 117. The transgenic cereal plant of Embodiment 116, wherein the transgenic plant has a shorter plant height and/or improved lodging resistance.

Embodiment 118. The transgenic cereal plant of Embodiment 116, wherein the level of one or more active GAs in at least one internode tissue of the stem or stalk of the transgenic plant is lower than the same internode tissue of a wild-type control plant.

Embodiment 119. A method for producing a transgenic cereal plant, comprising: (a) transforming at least one cell of an explant with the recombinant DNA construct of Embodiment 116, and (b) regenerating or developing the transgenic cereal plant from the transformed explant.

Embodiment 120. The method of Embodiment 119, wherein the cereal plant is transformed via Agrobacterium mediated transformation or particle bombardment.

Embodiment 121. A method for lowering the level of at least one active GA molecule in the stem or stalk of a corn or cereal plant comprising: suppressing one or more GA3 oxidase or GA20 oxidase genes with a recombinant DNA construct in one or more tissues of the transgenic cereal or corn plant.

Embodiment 122. The method of Embodiment 121, wherein the recombinant DNA construct encodes a non-coding RNA molecule that targets one or more GA3 or GA20 oxidase genes for suppression, wherein the transcribable DNA sequence is operably linked to a plant-expressible promoter.

Embodiment 123. The method of Embodiment 122, wherein the plant-expressible promoter is a vascular promoter.

Embodiment 124. The method of Embodiment 122, wherein the plant-expressible promoter is a RTBV promoter.

Embodiment 125. The method of Embodiment 122, wherein the plant-expressible promoter is a constitutive promoter.

Embodiment 126. The method of Embodiment 122, wherein the plant-expressible promoter is a leaf promoter.

Embodiment 127. The method of Embodiment 121, wherein the transgenic corn or cereal plant is a corn plant.

Embodiment 128. A transgenic corn or cereal plant comprising a recombinant DNA construct, wherein the recombinant DNA construct comprises a transcribable DNA sequence encoding a non-coding RNA molecule that targets at least one endogenous GA20 or GA3 oxidase gene for suppression, the transcribable DNA sequence being operably linked to a plant-expressible promoter, and wherein the transgenic monocot or cereal plant has a shorter plant height relative to a wild-type control plant.

Embodiment 129. The transgenic corn or cereal plant of Embodiment 128, wherein the transgenic plant has one or more of the following additional traits relative to the control plant: increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen or water limiting stress conditions, increased ear weight, increased harvest index, increased yield, increased seed number, increased seed weight, and increased prolificacy.

Embodiment 130. The transgenic corn or cereal plant of Embodiment 128, wherein the height of the transgenic plant is at least 10%, at least 20%, at least 25%, at least 30%, at least 35%, or at least 40% shorter than the control plant.

Embodiment 131. The transgenic corn or cereal plant of Embodiment 128, wherein the stalk or stem diameter of the transgenic plant at one or more stem internodes is at least 5%, at least 10%, at least 15%, at least 20%, at least 25%, at least 30%, at least 35%, or at least 40% greater than the control plant.

Embodiment 132. The transgenic corn or cereal plant of any one of Embodiments 128, wherein the level of one or more active GAs in at least one internode tissue of the stem or stalk of the transgenic plant is lower than the same internode tissue of the control plant.

Embodiment 133. The transgenic corn or cereal plant of any one of Embodiments 128, wherein the level of one or more active GAs in at least one internode tissue of the stem or stalk of the transgenic plant is at least 5%, at least 10%, at least 15%, at least 20%, at least 25%, at least 30%, at least 35%, or at least 40% lower than the same internode tissue of the control plant.

Embodiment 134. The transgenic corn or cereal plant of any one of Embodiments 128, wherein the transgenic plant does not have any significant off-types in at least one female organ or ear.

Embodiment 135. The transgenic corn or cereal plant of any one of Embodiments 128, wherein the transgenic cereal plant is a corn plant, and wherein the non-coding RNA molecule targets the endogenous GA20 oxidase_3 and/or GA20 oxidase_5 gene(s) for suppression.

Embodiment 136. The transgenic corn or cereal plant of Embodiment 128, wherein the plant-expressible promoter is a vascular promoter.

Embodiment 137. The transgenic corn or cereal plant of Embodiment 128, wherein the plant-expressible promoter is a RTBV promoter.

Embodiment 138. The transgenic corn or cereal plant of Embodiment 128, wherein the plant-expressible promoter is a constitutive promoter.

Embodiment 139. The transgenic corn or cereal plant of Embodiment 128, wherein the plant-expressible promoter is a leaf promoter.

Embodiment 140. The transgenic corn or cereal plant of Embodiment 128, wherein the transgenic plant has one or more of the following additional traits relative to the control plant: increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen or water limiting stress conditions, increased ear weight, increased harvest index, increased yield, increased seed number, increased seed weight, and increased prolificacy.

Embodiment 141. A cereal plant comprising a mutation at or near an endogenous GA oxidase gene introduced by a mutagenesis technique, wherein the expression level of the endogenous GA oxidase gene is reduced or eliminated in the cereal plant, and wherein the cereal plant has a shorter plant height relative to a wild-type control plant.

Embodiment 142. The cereal plant of Embodiment 141, wherein the cereal plant comprising the mutation has one or more of the following additional traits relative to the control plant: increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen or water limiting stress conditions, increased ear weight, increased harvest index, increased yield, increased seed number, increased seed weight, and increased prolificacy.

Embodiment 143. The cereal plant of Embodiment 141, wherein the height of the cereal plant is at least 10%, at least 20%, at least 25%, at least 30%, at least 35%, or at least 40% shorter than the control plant.

Embodiment 144. The cereal plant of Embodiment 141, wherein the stalk or stem diameter of the cereal plant at one or more stem internodes is at least 5%, at least 10%, at least 15%, at least 20%, at least 25%, at least 30%, at least 35%, or at least 40% greater than the control plant.

Embodiment 145. The cereal plant of Embodiment 141, wherein the level of one or more active GAs in at least one internode tissue of the stem or stalk of the cereal plant is lower than the same internode tissue of the control plant.

Embodiment 146. The cereal plant of Embodiment 141, wherein the level of one or more active GAs in at least one internode tissue of the stem or stalk of the cereal plant is at least 5%, at least 10%, at least 15%, at least 20%, at least 25%, at least 30%, at least 35%, or at least 40% lower than the same internode tissue of the control plant.

Embodiment 147. The cereal plant of Embodiment 141, wherein the cereal plant does not have any significant off-types in at least one female organ or ear.

Embodiment 148. The cereal plant of Embodiment 141, wherein the cereal plant is a corn plant.

Embodiment 149. A corn or cereal plant comprising a genomic edit introduced via a targeted genome editing technique at or near the locus of an endogenous GA oxidase gene, wherein the expression level of the endogenous GA oxidase gene is reduced or eliminated relative to a control plant, and wherein the edited cereal plant has a shorter plant height relative to the control plant.

Embodiment 150. The edited corn or cereal plant of Embodiment 149, wherein the edited plant has one or more of the following additional traits relative to the control plant: increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen or water limiting stress conditions, increased ear weight, increased harvest index, increased yield, increased seed number, increased seed weight, and increased prolificacy.

Embodiment 151. The edited corn or cereal plant of Embodiment 149, wherein the height of the edited plant is at least 10%, at least 20%, at least 25%, at least 30%, at least 35%, or at least 40% shorter than the control plant.

Embodiment 152. The edited corn or cereal plant of Embodiment 149, wherein the stalk or stem diameter of the edited plant at one or more stem internodes is at least 5%, at least 10%, at least 15%, at least 20%, at least 25%, at least 30%, at least 35%, or at least 40% greater than the control plant.

Embodiment 153. The edited corn or cereal plant of Embodiment 149, wherein the level of one or more active GAs in at least one internode tissue of the stem or stalk of the edited plant is lower than the same internode tissue of the control plant.

Embodiment 154. The edited corn or cereal plant of Embodiment 149, wherein the level of one or more active GAs in at least one internode tissue of the stem or stalk of the edited plant is at least 5%, at least 10%, at least 15%, at least 20%, at least 25%, at least 30%, at least 35%, or at least 40% lower than the same internode tissue of the control plant.

Embodiment 155. The edited corn or cereal plant of Embodiment 149, wherein the edited plant does not have any significant off-types in at least one female organ or ear.

Embodiment 156. The edited corn or cereal plant of Embodiment 149, wherein the genomic edit is introduced using a meganuclease, a zinc-finger nuclease (ZFN), a RNA-guided endonuclease, a TALE-endonuclease (TALEN), a recombinase, or a transposase.

Embodiment 157. The edited corn or cereal plant of Embodiment 149, wherein the genomic edit comprises a substitution, deletion, insertion, or inversion of one or more nucleotides relative to the sequence of the endogenous GA oxidase gene in the control plant.

Embodiment 158. A composition comprising a guide RNA, wherein the guide RNA comprises a guide sequence that is at least 95%, at least 96%, at least 97%, at least 99%, or 100% identical or complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of a target DNA sequence at or near the genomic locus of an endogenous GA oxidase gene of a cereal plant.

Embodiment 159. The composition of Embodiment 158, wherein the guide RNA molecule comprises a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of SEQ ID NO: 34, 35 or 38, or a sequence complementary thereto.

Embodiment 160. The composition of Embodiment 158, wherein the guide RNA molecule comprises a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of SEQ ID NO: 87, 91, 95, 98, 105, 109, 113, 117, 122, 126, 130 or 137, or a sequence complementary thereto.

Embodiment 161. The composition of Embodiment 158, further comprising an RNA-guided endonuclease.

Embodiment 162. The composition of Embodiment 161, wherein the RNA-guided endonuclease in the presence of the guide RNA molecule causes a double strand break or nick at or near the target DNA sequence in the genome of the cereal plant.

Embodiment 163. The composition of Embodiment 161, wherein the RNA-guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn1, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx12, Csx15, Csf1, Csf2, Csf3, Csf4, Cpf1, CasX, CasY, Argonaute, and any homologs or modified versions thereof having RNA-guided endonuclease activity.

Embodiment 164. The composition of Embodiment 158, further comprising a recombinant DNA donor template comprising at least one homology sequence or homology arm, wherein the at least one homology sequence or homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence, wherein the target DNA sequence is a genomic sequence at or near the genomic locus of the endogenous GA oxidase gene of a corn or cereal plant.

Embodiment 165. A recombinant DNA construct comprising a transcribable DNA sequence encoding a non-coding guide RNA molecule, wherein the guide RNA molecule comprises a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of a target DNA sequence at or near the genomic locus of an endogenous GA oxidase gene of a corn or cereal plant.

Embodiment 166. The recombinant DNA construct of Embodiment 165, wherein the guide RNA comprises a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of SEQ ID NO: 34, 35 or 38, or a sequence complementary thereto.

Embodiment 167. The recombinant DNA construct of Embodiment 165, wherein the guide RNA molecule comprises a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of SEQ ID NO: 87, 91, 95, 98, 105, 109, 113, 117, 122, 126, 130 or 137, or a sequence complementary thereto.

Embodiment 168. The recombinant DNA construct of Embodiment 165, wherein the transcribable DNA sequence is operably linked to a plant-expressible promoter.

Embodiment 169. The recombinant DNA construct of Embodiment 165, wherein the guide RNA molecule is a CRISPR RNA (crRNA) or a single-chain guide RNA (sgRNA).

Embodiment 170. The recombinant DNA construct of Embodiment 165, wherein the guide RNA comprises a sequence complementary to a protospacer adjacent motif (PAM) sequence present in the genome of the cereal plant immediately adjacent to the target DNA sequence at or near the genomic locus of the endogenous GA oxidase gene.

Embodiment 171. The recombinant DNA construct of any one of Embodiment 165, wherein the PAM sequence comprises a canonical 5′-NGG-3′ sequence.

Embodiment 172. The recombinant DNA construct of Embodiment 165, wherein the endogenous GA oxidase gene encodes a protein that is at least 80%, at least 85%, at least 90%, at least 95%, at least 96%, at least 97%, at least 98%, at least 99%, at least 99.5%, or 100% identical to SEQ ID NO: 9, 12 or 15.

Embodiment 173. A DNA molecule comprising the recombinant DNA construct of Embodiment 165.

Embodiment 174. A transformation vector comprising the recombinant DNA construct of Embodiment 165.

Embodiment 175. A bacterial cell comprising the recombinant DNA construct of Embodiment 165.

Embodiment 176. A corn or cereal plant, plant part or plant cell comprising the recombinant DNA construct of Embodiment 165.

Embodiment 177. A composition comprising the recombinant DNA construct of Embodiment 165.

Embodiment 178. The composition of Embodiment 177, further comprising a RNA-guided endonuclease.

Embodiment 179. The composition of Embodiment 177, wherein the RNA-guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, Argonaute, and homologs or modified versions thereof having RNA-guided endonuclease activity.

Embodiment 180. The composition of Embodiment 177, further comprising a second recombinant DNA construct comprising a second transcribable DNA sequence encoding the RNA-guided endonuclease.

Embodiment 181. The composition of Embodiment 177, comprising a DNA molecule or vector comprising the recombinant DNA construct and the second recombinant DNA construct.

Embodiment 182. The composition of Embodiment 177, comprising a first DNA molecule or vector and a second DNA molecule or vector, wherein the first DNA molecule or vector comprises the recombinant DNA construct encoding the guide RNA molecule, and the second DNA molecule or vector comprises the second recombinant DNA construct encoding the RNA-guided endonuclease.

Embodiment 183. The composition of Embodiment 177, further comprising a recombinant DNA donor template comprising at least one homology sequence or homology arm, wherein the at least one homology sequence or homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence, wherein the target DNA sequence is a genomic sequence at or near the genomic locus of an endogenous GA oxidase gene of a corn or cereal plant.

Embodiment 184. A recombinant DNA donor template comprising at least one homology sequence, wherein the at least one homology sequence is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence, wherein the target DNA sequence is a genomic sequence at or near the genomic locus of an endogenous GA oxidase gene of a corn or cereal plant.

Embodiment 185. The recombinant DNA donor template of Embodiment 184, wherein the at least one homology sequence comprises at least one mutation relative to the complementary strand of the target DNA sequence at or near the genomic locus of the endogenous GA oxidase gene.

Embodiment 186. The recombinant DNA donor template of Embodiment 185, wherein the at least one mutation comprises a substitution, deletion, insertion, or inversion of one or more nucleotides relative to the complementary strand of the target DNA sequence.

Embodiment 187. The recombinant DNA donor template of Embodiment 184, wherein the at least one homology sequence is at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of SEQ ID NO: 34, 35 or 38, or a sequence complementary thereto.

Embodiment 188. The recombinant DNA donor template of Embodiment 184, wherein the at least one homology sequence is at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of SEQ ID NO: 87, 91, 95, 98, 105, 109, 113, 117, 122, 126, 130 or 137, or a sequence complementary thereto.

Embodiment 189. A recombinant DNA donor template comprising two homology arms including a first homology arm and a second homology arm, wherein the first homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a first flanking DNA sequence, wherein the second homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a second flanking DNA sequence, and wherein the first flanking DNA sequence and the second flanking DNA sequence are genomic sequences at or near the genomic locus of an endogenous GA oxidase gene of a corn or cereal plant.

Embodiment 190. The recombinant DNA donor template of Embodiment 189, further comprising an insertion sequence located between the first homology arm and the second homology arm.

Embodiment 191. The recombinant DNA donor template of Embodiment 189, wherein the insertion sequence comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 750, at least 1000, at least 2500, or at least 5000 nucleotides.

Embodiment 192. The recombinant DNA donor template of Embodiment 189, wherein each homology arm is at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of SEQ ID NO: 34, 35 or 38, or a sequence complementary thereto.

Embodiment 193. The recombinant DNA donor template of Embodiment 189, wherein each homology arm is at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of SEQ ID NO: 87, 91, 95, 98, 105, 109, 113, 117, 122, 126, 130 or 137, or a sequence complementary thereto.

Embodiment 194. The recombinant DNA donor template of Embodiment 189, wherein one or more nucleotides present in the genome of the monocot or cereal plant between the first flanking DNA sequence and the second flanking DNA sequence are absent in the recombinant DNA donor template molecule between the first homology arm and the second homology arm.

Embodiment 195. The recombinant DNA donor template of Embodiment 194, wherein at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 750, at least 1000, at least 2500, or at least 5000 nucleotides present in the genome of the monocot or cereal plant between the first and second flanking DNA sequences are absent in the recombinant DNA donor template molecule between the first and second homology arms.

Embodiment 196. A DNA molecule or vector comprising the recombinant DNA donor template of Embodiment 189.

Embodiment 197. A bacterial or host cell comprising the recombinant DNA donor template of Embodiment 189.

Embodiment 198. A corn or cereal plant, plant part or plant cell comprising the recombinant DNA construct of Embodiment 189.

Embodiment 199. An engineered site-specific nuclease that binds to a target site at or in near the genomic locus of an endogenous GA oxidase gene of a corn or cereal plant and causes a double-strand break or nick at the target site.

Embodiment 200. The engineered site-specific nuclease of Embodiment 199, wherein the site-specific nuclease is a meganuclease or homing endonuclease.

Embodiment 201. The engineered site-specific nuclease of Embodiment 200, wherein the engineered meganuclease or homing endonuclease comprises a scaffold or base enzyme selected from the group consisting of I-CreI, I-CeuI, I-Msol, I-SceI, I-Anil, and I-DmoI.

Embodiment 202. The engineered site-specific nuclease of Embodiment 199, wherein the site-specific nuclease is a zinc finger nuclease (ZFN) comprising a DNA binding domain and a cleavage domain.

Embodiment 203. The engineered zinc finger nuclease of Embodiment 202, wherein the cleavage domain is a FokI nuclease domain.

Embodiment 204. The engineered site-specific nuclease of Embodiment 199, wherein the site-specific nuclease is a transcription activator-like effector nuclease (TALEN) comprising a DNA binding domain and a cleavage domain.

Embodiment 205. The engineered TALEN of Embodiment 204, wherein the cleavage domain is selected from the group consisting of a PvuII nuclease domain, a MutH nuclease domain, a TevI nuclease domain, a FokI nuclease domain, an AlwI nuclease domain, a MlyI nuclease domain, a SbfI nuclease domain, a SdaI nuclease domain, a StsI nuclease domain, a CleDORF nuclease domain, a Clo051 nuclease domain, and a Pept071 nuclease domain.

Embodiment 206. The engineered site-specific nuclease of Embodiment 199, wherein the target site bound by the site-specific nuclease is at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of SEQ ID NO: 34, 35 or 38, or a sequence complementary thereto.

Embodiment 207. The engineered site-specific nuclease of Embodiment 199, wherein the target site bound by the site-specific nuclease is at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of SEQ ID NO: 87, 91, 95, 98, 105, 109, 113, 117, 122, 126, 130 or 137, or a sequence complementary thereto.

Embodiment 208. A recombinant DNA construct comprising a transgene encoding a site-specific nuclease, wherein the site-specific nuclease binds to a target site at or near the genomic locus of an endogenous GA oxidase gene of a monocot or cereal plant and causes a double-strand break or nick at the target site.

Embodiment 209. The recombinant DNA construct of Embodiment 208, wherein the transgene is operably linked to a plant-expressible promoter.

Embodiment 210. The recombinant DNA construct of Embodiment 208, wherein the site-specific nuclease is a meganuclease or homing endonuclease, a zinc finger nuclease, or a transcription activator-like effector nuclease (TALEN).

Embodiment 211. A DNA molecule or vector comprising the recombinant DNA construct of Embodiment 208.

Embodiment 212. A bacterial or host cell comprising the recombinant DNA construct of Embodiment 208.

Embodiment 213. A corn or cereal plant, plant part or plant cell comprising the recombinant DNA construct of Embodiment 208.

Embodiment 214. A recombinant DNA donor template comprising at least one homology arm and an insertion sequence, wherein the at least one homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a genomic DNA sequence of a corn or cereal plant, and wherein the insertion sequence comprises a recombinant DNA construct comprising a transcribable DNA sequence encoding a non-coding RNA molecule, wherein the non-coding RNA molecule targets for suppression one or more endogenous GA20 or GA3 oxidase genes in a monocot or cereal plant or plant cell, and wherein the transcribable DNA sequence is operably linked to a plant-expressible promoter.

Embodiment 215. The recombinant DNA donor template of Embodiment 214, wherein the at least one homology arm comprises two homology arms including a first homology arm and a second homology arm, wherein the first homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a first flanking DNA sequence, and the second homology arm comprises a sequence that is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a second flanking DNA sequence, wherein the first flanking DNA sequence and the second flanking DNA sequence are genomic sequences at or near the same genomic locus of a monocot or cereal plant, and wherein the insertion sequence is located between the first homology arm and the second homology arm and comprises a recombinant DNA construct comprising a transcribable DNA sequence encoding a non-coding RNA molecule.

Embodiment 216. The recombinant DNA donor template of Embodiment 215, wherein the transcribable DNA sequence is operably linked to a plant-expressible promoter.

Embodiment 217. The recombinant DNA donor template of Embodiment 215, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding a GA oxidase protein that is at least 80%, at least 85%, at least 90%, at least 95%, at least 96%, at least 97%, at least 98%, at least 99%, at least 99.5%, or 100% identical to SEQ ID NO: 9, 12, 15, 30 or 33.

Embodiment 218. The recombinant DNA donor template of Embodiment 215, wherein the non-coding RNA molecule comprises a sequence that is at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding a GA oxidase protein that is at least 80%, at least 85%, at least 90%, at least 95%, at least 96%, at least 97%, at least 98%, at least 99%, at least 99.5%, or 100% identical to SEQ ID NO: 86, 90, 94, 97, 101, 104, 108, 112, 116, 118, 121, 125, 129, 133, or 136.

Embodiment 219. A composition comprising the recombinant DNA donor template of Embodiment 214.

Embodiment 220. A bacterial or host cell comprising the recombinant DNA donor template of Embodiment 214.

Embodiment 221. A transgenic corn or cereal plant, plant part or plant cell comprising the insertion sequence of the recombinant DNA donor template of Embodiment 214.

Embodiment 222. The transgenic corn or cereal plant of Embodiment 214, wherein the transgenic plant has one or more of the following traits relative to a control plant: shorter plant height, increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen or water limiting stress conditions, increased ear weight, increased harvest index, increased yield, increased seed number, increased seed weight, and/or increased prolificacy.

Embodiment 223. The transgenic corn or cereal plant of Embodiment 222, wherein the transgenic plant has a shorter plant height and/or improved lodging resistance.

Embodiment 224. The transgenic corn or cereal plant of Embodiments 222, wherein the height of the transgenic plant is at least 10%, at least 20%, at least 25%, at least 30%, at least 35%, or at least 40% shorter than a control plant.

Embodiment 225. The transgenic corn or cereal plant of Embodiments 222, wherein the level of one or more active GAs in at least one internode tissue of the stem or stalk of the transgenic plant is lower than the same internode tissue of a control plant.

Embodiment 226. A method for producing a transgenic corn or cereal plant, comprising: (a) transforming at least one cell of an explant with the recombinant DNA donor template of Embodiment 215, and (b) regenerating or developing the transgenic corn or cereal plant from the transformed explant, wherein the transgenic corn or cereal plant comprises the insertion sequence of the recombinant DNA donor template.

Embodiment 227. The method of Embodiment 226, wherein the monocot or cereal plant is transformed via Agrobacterium mediated transformation or particle bombardment.

Embodiment 228. A method for producing a corn or cereal plant having a genomic edit at or near an endogenous GA oxidase gene, comprising: (a) introducing into at least one cell of an explant of the corn or cereal plant a site-specific nuclease or a recombinant DNA molecule comprising a transgene encoding the site-specific nuclease, wherein the site-specific nuclease binds to a target site at or near the genomic locus of the endogenous GA oxidase gene and causes a double-strand break or nick at the target site, and (b) regenerating or developing an edited corn or cereal plant from the at least one explant cell comprising the genomic edit at or near the endogenous GA oxidase gene of the edited corn or cereal plant.

Embodiment 229. The method of Embodiment 228, wherein the introducing step (a) further comprises introducing a DNA donor template comprising at least one homology sequence or homology arm, wherein the at least one homology sequence or homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence, wherein the target DNA sequence is a genomic sequence at or near the genomic locus of the endogenous GA oxidase gene of the corn or cereal plant.

Embodiment 230. The method of Embodiment 228, further comprising: (c) selecting the edited corn or cereal plant.

Embodiment 231. The method of Embodiment 230, wherein the selecting step (c) comprises determining if the endogenous GA oxidase gene locus was edited using a molecular assay.

Embodiment 232. The method of Embodiment 230, wherein the selecting step (c) comprises determining if the endogenous GA oxidase gene was edited by observing a plant phenotype.

Embodiment 233. The method of Embodiment 231, wherein the plant phenotype is a decrease in plant height relative to a control plant.

Embodiment 234. The method of Embodiment 228, wherein the introducing step (a) creates at least one mutation at or near the genomic locus of the endogenous GA oxidase gene, and wherein the mutation comprises a substitution, deletion, insertion, or inversion of one or more nucleotides relative to the genomic DNA sequence of a control plant.

Embodiment 235. A modified corn plant having a plant height of less than 2000 mm, less than 1950 mm, less than 1900 mm, less than 1850 mm, less than 1800 mm, less than 1750 mm, less than 1700 mm, less than 1650 mm, less than 1600 mm, less than 1550 mm, less than 1500 mm, less than 1450 mm, less than 1400 mm, less than 1350 mm, less than 1300 mm, less than 1250 mm, less than 1200 mm, less than 1150 mm, less than 1100 mm, less than 1050 mm, or less than 1000 mm, and either (i) an average stem or stalk diameter of greater than 18 mm, greater than 18.5 mm, greater than 19 mm, greater than 19.5 mm, greater than 20 mm, greater than 20.5 mm, greater than 21 mm, greater than 21.5 mm, or greater than 22 mm, (ii) improved lodging resistance relative to a wild type control plant, or (iii) improved drought tolerance relative to a wild type control plant.

Embodiment 236. The modified corn plant of Embodiment 235, wherein the corn plant has one or more of the following traits relative to a wild type control plant: increased stalk/stem diameter, improved lodging resistance, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen or water limiting stress conditions, increased ear weight, increased harvest index, increased yield, increased seed number, increased seed weight, and/or increased prolificacy.

Embodiment 237. The modified corn plant of Embodiment 235, wherein the level of one or more active GAs in at least one internode tissue of the stem or stalk of the corn plant is lower than the same internode tissue of a wild type control plant.

Embodiment 238. A modified cereal plant having a reduced plant height relative to a wild type control plant, and (i) an increased stem or stalk diameter relative to a wild type control plant, (ii) improved lodging resistance relative to a wild type control plant, or (iii) improved drought tolerance relative to a wild type control plant.

Embodiment 239. The modified cereal plant of Embodiment 238, wherein the level of one or more active GAs in the stem or stalk of the cereal plant is lower than in a wild type control plant.

Embodiment 240. A modified corn plant, or plant part thereof, comprising a mutant allele at GA20 oxidase_3 locus and a mutant allele at GA20 oxidase_5 locus, wherein at least one of the GA20 oxidase_3 and GA20 oxidase_5 loci comprises homozygous mutant alleles.

Embodiment 241. A modified corn plant, or plant part thereof, comprising a first homozygous mutation in one of GA20 oxidase_3 and GA20 oxidase_5 genes and further comprising a second heterozygous or homozygous mutation in the other one of the GA20 oxidase_3 and GA20 oxidase_5 genes.

Embodiment 242. A modified corn plant, or plant part thereof, comprising a homozygous mutant GA20 oxidase_3 gene and a homozygous mutant GA20 oxidase_5 gene.

Embodiment 243. A modified corn plant, or plant part thereof, comprising homozygous mutant alleles at the endogenous GA20 oxidase_3 locus and homozygous mutant alleles at the endogenous GA20 oxidase_5 locus.

Embodiment 244. A modified corn plant, or plant part thereof, comprising homozygous mutant alleles at the GA20 oxidase_3 locus, and exhibiting an at least 2.5%, at least 5%, at least 7.5%, at least 10%, at least 15%, or at least 20% reduction in plant height at maturity relative to an unmodified control plant.

Embodiment 245. A modified corn plant, or plant part thereof, comprising homozygous mutant alleles at the GA20 oxidase_5 locus, and exhibiting an at least 2.5%, at least 5%, at least 7.5%, at least 10%, at least 15%, or at least 20% reduction in plant height at maturity relative to an unmodified control plant.

The Following Paragraphs List a Second Subset of Exemplary Embodiments.

Embodiment 1. A modified corn plant, or plant part thereof, comprising a mutant allele at GA20 oxidase_3 locus and a mutant allele at GA20 oxidase_5 locus, wherein at least one of said GA20 oxidase_3 and GA20 oxidase_5 loci comprises homozygous mutant alleles.

Embodiment 2. The modified corn plant, or plant part thereof, of Embodiment 1, wherein said GA20 oxidase_3 locus comprises homozygous mutant alleles.

Embodiment 3. The modified corn plant, or plant part thereof, of Embodiment 1, wherein only one of said GA20 oxidase_3 and GA20 oxidase_5 loci comprises homozygous mutant alleles.

Embodiment 4. The modified corn plant, or plant part thereof, of Embodiment 1, wherein said GA20 oxidase_5 locus comprises homozygous mutant alleles.

Embodiment 5. The modified corn plant, or plant part thereof, of Embodiment 1, wherein both of said GA20 oxidase_3 and GA20 oxidase_5 loci comprise homozygous mutant alleles.

Embodiment 6. The modified corn plant, or plant part thereof, of any one of Embodiments 1 to 5, wherein one or both of said GA20 oxidase_3 and GA20 oxidase_5 loci comprise a heteroallelic combination or two identical mutant alleles.

Embodiment 7. The modified corn plant, or plant part thereof, of any one of Embodiments 1 to 5, wherein said mutant allele exhibits an at least 30%, at least 40%, at least 50%, at least 60%, at least 70%, at least 80%, at least 90%, at least 95%, or 100% reduction of expression or enzymatic activity relative to an unmodified, wild-type GA20 oxidase_3 or GA20 oxidase_5 gene allele.

Embodiment 8. The modified corn plant, or plant part thereof, of any one of Embodiments 1 to 7, wherein said mutant allele at the GA20 oxidase_3 locus comprises a mutation in a sequence region selected from the group consisting of a promoter, 5′ UTR, first exon, first intron, second exon, second intron, third exon, 3′ UTR, terminator, and any combination thereof.

Embodiment 9. The modified corn plant, or plant part thereof, of any one of Embodiments 1 to 8, wherein said mutant allele at the GA20 oxidase_3 locus comprises one or more mutation types selected from the group consisting of a nonsense mutation, a missense mutation, a frameshift mutation, a splice-site mutation, and any combination thereof.

Embodiment 10. The modified corn plant, or plant part thereof, of any one of Embodiments 1 to 9, wherein said mutant allele at the GA20 oxidase_3 locus results in one or more of the following: a GA20 oxidase_3 protein truncation, a non-translatable GA20 oxidase_3 gene transcript, a non-functional GA20 oxidase_3 protein, a premature stop codon in the GA20 oxidase_3 gene, and any combination thereof.

Embodiment 11. The modified corn plant, or plant part thereof, of any one of Embodiments 1 to 10, wherein each mutant allele at the GA20 oxidase_3 locus comprises a mutation selected from the group consisting of a substitution, a deletion, an insertion, a duplication, and an inversion of one or more nucleotides relative to a wild-type GA20 oxidase_3 gene.

Embodiment 12. The modified corn plant, or plant part thereof, of any one of Embodiments 1 to 11, wherein said mutant allele at the GA20 oxidase_3 locus comprises one or more mutations in the first exon of the GA20 oxidase_3 gene.

Embodiment 13. The modified corn plant, or plant part thereof, of any one of Embodiments 1 to 12, wherein said mutant allele at the GA20 oxidase_3 locus comprises one or more mutations in the second exon of the GA20 oxidase_3 gene.

Embodiment 14. The modified corn plant, or plant part thereof, of any one of Embodiments 1 to 13, wherein said mutant allele at the GA20 oxidase_5 locus comprises a mutation in a sequence region selected from the group consisting of a promoter, 5′ UTR, first exon, first intron, second exon, second intron, third exon, 3′ UTR, terminator, and any combination thereof.

Embodiment 15. The modified corn plant, or plant part thereof, of any one of Embodiments 1 to 14, wherein said mutant allele at the GA20 oxidase_5 locus comprises one or more mutation types selected from the group consisting of a nonsense mutation, a missense mutation, a frameshift mutation, a splice-site mutation, and any combination thereof.

Embodiment 16. The modified corn plant, or plant part thereof, of any one of Embodiments 1 to 15, wherein said mutant allele at the GA20 oxidase_5 locus results in one or more of the following: a GA20 oxidase_5 protein truncation, a non-translatable GA20 oxidase_5 gene transcript, a non-functional GA20 oxidase_5 protein, a premature stop codon in the GA20 oxidase_5 gene, and any combination thereof.

Embodiment 17. The modified corn plant, or plant part thereof, of any one of Embodiments 1 to 16, wherein each mutant allele at the GA20 oxidase_5 locus comprises a mutation selected from the group consisting of a substitution, a deletion, an insertion, a duplication, and an inversion of one or more nucleotides relative to a wild-type GA20 oxidase_5 gene.

Embodiment 18. The modified corn plant, or plant part thereof, of any one of Embodiments 1 to 17, wherein said mutant allele at the GA20 oxidase_5 locus comprises one or more mutations in the first exon of the GA20 oxidase_5 gene.

Embodiment 19. The modified corn plant, or plant part thereof, of any one of Embodiments 1 to 18, wherein said mutant allele at the GA20 oxidase_5 locus comprises one or more mutations in the second exon of the GA20 oxidase_5 gene.

Embodiment 20. A modified corn plant, or plant part thereof, comprising a first homozygous mutation in one of GA20 oxidase_3 and GA20 oxidase_5 genes and further comprising a second heterozygous or homozygous mutation in the other one of said GA20 oxidase_3 and GA20 oxidase_5 genes.

Embodiment 21. The modified corn plant, or plant part thereof, of Embodiment 20, wherein said first homozygous mutation is in the GA20 oxidase_3 gene.

Embodiment 22. The modified corn plant, or plant part thereof, of Embodiment 21, wherein said second mutation is heterozygous in the GA20 oxidase_5 gene.

Embodiment 23. The modified corn plant, or plant part thereof, of Embodiment 20, wherein said first homozygous mutation is in the GA20 oxidase_5 gene.

Embodiment 24. The modified corn plant, or plant part thereof, of Embodiment 23, wherein said second mutation is heterozygous in the GA20 oxidase_3 gene.

Embodiment 25. The modified corn plant, or plant part thereof, of Embodiment 20, wherein said first homozygous mutation comprises a heteroallelic combination of mutations or two identical mutant alleles in one of the GA20 oxidase_3 and GA20 oxidase_5 genes.

Embodiment 26. The modified corn plant, or plant part thereof, of any one of Embodiments 20 to 25, wherein said first homozygous mutation results in the modified corn plant having an at least 30%, at least 40%, at least 50%, at least 60%, at least 70%, at least 80%, at least 90%, at least 95%, or 100% reduction of expression or enzymatic activity of the GA20 oxidase_3 relative to an unmodified, wild-type GA20 oxidase_3 gene allele.

Embodiment 27. The modified corn plant, or plant part thereof, of any one of Embodiments 20 to 25, wherein said first homozygous mutation or said second heterozygous or homozygous mutation results in the modified corn plant having an at least 30%, at least 40%, at least 50%, at least 60%, at least 70%, at least 80%, at least 90%, at least 95%, or 100% reduction of expression or enzymatic activity of the GA20 oxidase_5 relative to an unmodified, wild-type GA20 oxidase_5 gene allele.

Embodiment 28. The modified corn plant, or plant part thereof, of any one of Embodiments 20 to 27, wherein said mutant allele at the GA20 oxidase_3 locus comprises a mutation in a sequence region selected from the group consisting of a promoter, 5′ UTR, first exon, first intron, second exon, second intron, third exon, 3′ UTR, terminator, and any combination thereof.

Embodiment 29. The modified corn plant, or plant part thereof, of any one of Embodiments 20 to 28, wherein said mutant allele at the GA20 oxidase_3 locus comprises one or more mutation types selected from the group consisting of a nonsense mutation, a missense mutation, a frameshift mutation, a splice-site mutation, and any combination thereof.

Embodiment 30. The modified corn plant, or plant part thereof, of any one of Embodiments 20 to 29, wherein said mutant allele at the GA20 oxidase_3 locus results in one or more of the following: a GA20 oxidase_3 protein truncation, a non-translatable GA20 oxidase_3 gene transcript, a non-functional GA20 oxidase_3 protein, a premature stop codon in the GA20 oxidase_3 gene, and any combination thereof.

Embodiment 31. The modified corn plant, or plant part thereof, of any one of Embodiments 20 to 30, wherein each mutant allele at the GA20 oxidase_3 locus comprises a mutation selected from the group consisting of a substitution, a deletion, an insertion, a duplication, and an inversion of one or more nucleotides relative to a wild-type GA20 oxidase_3 gene.

Embodiment 32. The modified corn plant, or plant part thereof, of any one of Embodiments 20 to 31, wherein said mutant allele at the GA20 oxidase_3 locus comprises one or more mutations in the first exon of the GA20 oxidase_3 gene.

Embodiment 33. The modified corn plant, or plant part thereof, of any one of Embodiments 20 to 32, wherein said mutant allele at the GA20 oxidase_3 locus comprises one or more mutations in the second exon of the GA20 oxidase_3 gene.

Embodiment 34. The modified corn plant, or plant part thereof, of any one of Embodiments 20 to 33, wherein said mutant allele at the GA20 oxidase_5 locus comprises a mutation in a sequence region selected from the group consisting of a promoter, 5′ UTR, first exon, first intron, second exon, second intron, third exon, 3′ UTR, terminator, and any combination thereof.

Embodiment 35. The modified corn plant, or plant part thereof, of any one of Embodiments 20 to 34, wherein said mutant allele at the GA20 oxidase_5 locus comprises one or more mutation types selected from the group consisting of a nonsense mutation, a missense mutation, a frameshift mutation, a splice-site mutation, and any combination thereof.

Embodiment 36. The modified corn plant, or plant part thereof, of any one of Embodiments 20 to 35, wherein said mutant allele at the GA20 oxidase_5 locus results in one or more of the following: a GA20 oxidase_5 protein truncation, a non-translatable GA20 oxidase_5 gene transcript, a non-functional GA20 oxidase_5 protein, a premature stop codon in the GA20 oxidase_5 gene, and any combination thereof.

Embodiment 37. The modified corn plant, or plant part thereof, of any one of Embodiments 20 to 36, wherein each mutant allele at the GA20 oxidase_5 locus comprises a mutation selected from the group consisting of a substitution, a deletion, an insertion, a duplication, and an inversion of one or more nucleotides relative to a wild-type GA20 oxidase_5 gene.

Embodiment 38. The modified corn plant, or plant part thereof, of any one of Embodiments 20 to 37, wherein said mutant allele at the GA20 oxidase_5 locus comprises one or more mutations in the first exon of the GA20 oxidase_5 gene.

Embodiment 39. The modified corn plant, or plant part thereof, of any one of Embodiments 20 to 38, wherein said mutant allele at the GA20 oxidase_5 locus comprises one or more mutations in the second exon of the GA20 oxidase_5 gene.

Embodiment 40. The modified corn plant, or plant part thereof, of Embodiment 20, wherein said first homozygous mutation comprises one or more alleles, as a pair of two identical alleles or a heteroallelic combination, selected from the group consisting of:

a. a deletion of 13 bases starting at 536;
b. a deletion of base 542;
c. an insertion of CC at base 542;
d. a deletion of base 541;
e. a deletion of 3 nt starting at base 540;
f. a deletion of 2 bases starting at base 422;
g. an insertion of an A at base 422;
h. an insertion of a T at base 422;
i. a deletion of base 564;
j. an insertion of an A at base 564;
k. an insertion of a C at base 565; and
l. an insertion of a C at base 63;

wherein said base numbering is based on SEQ ID No. 168 and counted from the first nucleotide of SEQ ID NO: 168 in the 5′ to 3′ direction.

Embodiment 41. The modified corn plant, or plant part thereof, of Embodiment 20, wherein said first homozygous mutation is identified by one or more of SEQ ID Nos: 170 to 193 and 206 to 217 relative to the corresponding reference sequence in SEQ ID No: 168.

Embodiment 42. The modified corn plant, or plant part thereof, of Embodiment 20, wherein said first homozygous mutation comprises one or more alleles, as a pair of two identical alleles or a heteroallelic combination, selected from the group consisting of:

a. a deletion of base 644;
b. a deletion of 2 bases starting at base 644;
c. an insertion of a T at base 644;
d. a deletion of base 372;
e. a deletion of base 786;
f. a deletion of 5 bases starting at base 786;
g. a deletion of 2 bases starting at base 101;
h. an insertion of a T at base base 102;
i. a deletion of 3 bases starting at base 99;
j. an insertion of an A at base 282; and
k. an insertion of a C at base 282;

wherein said base numbering is based on SEQ ID No. 169, and counted from the first nucleotide of SEQ ID NO: 169 in the 5′ to 3′ direction.

Embodiment 43. The modified corn plant, or plant part thereof, of Embodiment 20, wherein said first homozygous mutation is identified by one or more of SEQ ID Nos.: 218 to 239 and 251 to 261 relative to the corresponding reference sequence in SEQ ID No: 169.

Embodiment 44. A modified corn plant, or plant part thereof, comprising a homozygous mutant GA20 oxidase_3 gene and a homozygous mutant GA20 oxidase_5 gene.

Embodiment 45. The modified corn plant, or plant part thereof, of Embodiment 44, wherein said homozygous mutant GA20 oxidase_3 gene, said homozygous mutant GA20 oxidase_5 gene, or both comprise a heteroallelic combination of mutant alleles or two identical mutant alleles.

Embodiment 46. The modified corn plant, or plant part thereof, of Embodiment 44 or 45, wherein said mutant GA20 oxidase_3 gene comprises a mutation in a sequence region selected from the group consisting of a promoter, 5′ UTR, first exon, first intron, second exon, second intron, third exon, 3′ UTR, terminator, and any combination thereof.

Embodiment 47. The modified corn plant, or plant part thereof, of Embodiment 44 or 45, wherein said mutant GA20 oxidase_3 gene comprises one or more mutation types selected from the group consisting of a nonsense mutation, a missense mutation, a frameshift mutation, a splice-site mutation, and any combination thereof.

Embodiment 48. The modified corn plant, or plant part thereof, of Embodiment 44 or 45, wherein said mutant GA20 oxidase_3 gene results in one or more of the following: a GA20 oxidase_3 protein truncation, a non-translatable GA20 oxidase_3 gene transcript, a non-functional GA20 oxidase_3 protein, a premature stop codon in the GA20 oxidase_3 gene, and any combination thereof.

Embodiment 49. The modified corn plant, or plant part thereof, of Embodiment 44 or 45, wherein said mutant GA20 oxidase_3 gene comprises a mutation selected from the group consisting of a substitution, a deletion, an insertion, a duplication, and an inversion of one or more nucleotides relative to a wild-type GA20 oxidase_3 gene.

Embodiment 50. The modified corn plant, or plant part thereof, of any one of Embodiments 44 to 49, wherein said mutant GA20 oxidase_5 gene comprises a mutation in a sequence region selected from the group consisting of a promoter, 5′ UTR, first exon, first intron, second exon, second intron, third exon, 3′ UTR, terminator, and any combination thereof.

Embodiment 51. The modified corn plant, or plant part thereof, of any one of Embodiments 44 to 49, wherein said mutant GA20 oxidase_5 gene comprises one or more mutation types selected from the group consisting of a nonsense mutation, a missense mutation, a frameshift mutation, a splice-site mutation, and any combination thereof.

Embodiment 52. The modified corn plant, or plant part thereof, of Embodiment 44 or 45, wherein said mutant GA20 oxidase_5 gene results in one or more of the following: a GA20 oxidase_5 protein truncation, a non-translatable GA20 oxidase_5 gene transcript, a non-functional GA20 oxidase_5 protein, a premature stop codon in the GA20 oxidase_5 gene, and any combination thereof.

Embodiment 53. The modified corn plant, or plant part thereof, of any one of Embodiments 44 to 49, wherein said mutant GA20 oxidase_5 gene comprises a mutation selected from the group consisting of a substitution, a deletion, an insertion, a duplication, and an inversion of one or more nucleotides relative to a wild-type GA20 oxidase_5 gene.

Embodiment 54. The modified corn plant, or plant part thereof, of any one of Embodiments 44 to 49, wherein said homozygous mutant GA20 oxidase_3 gene, said homozygous mutant GA20 oxidase_5 gene, or both comprises one or more mutations in the first exon of the corresponding gene.

Embodiment 55. The modified corn plant, or plant part thereof, of any one of Embodiments 44 to 49, wherein said homozygous mutant GA20 oxidase_3 gene, said homozygous mutant GA20 oxidase_5 gene, or both comprises one or more mutations in the second exon of the corresponding gene.

Embodiment 56. The modified corn plant, or plant part thereof, of any one of Embodiments 44 to 55, wherein said homozygous mutant GA20 oxidase_3 gene, said homozygous mutant GA20 oxidase_5 gene, or both comprise a null allele.

Embodiment 57. The modified corn plant, or plant part thereof, of any one of Embodiments 44 to 55, wherein said homozygous mutant GA20 oxidase_3 gene or said homozygous mutant GA20 oxidase_5 gene exhibits an at least 30%, at least 40%, at least 50%, at least 60%, at least 70%, at least 80%, at least 90%, at least 95%, or 100% reduction of expression or enzymatic activity relative to a corresponding wild-type gene.

Embodiment 58. The modified corn plant, or plant part thereof, of any one of Embodiments 44 to 55, wherein said homozygous mutant GA20 oxidase_3 gene comprises a sequence selected from the group consisting of SEQ ID Nos: 170 to 181.

Embodiment 59. The modified corn plant, or plant part thereof, of any one of Embodiments 44 to 55, wherein said homozygous mutant GA20 oxidase_3 gene comprises a sequence selected from the group consisting of SEQ ID Nos: 182 to 193.

Embodiment 60. The modified corn plant, or plant part thereof, of any one of Embodiments 44 to 55, wherein said homozygous mutant GA20 oxidase_3 gene comprises a mutation identified by one or more of SEQ ID Nos: 182 to 193 relative to the corresponding reference sequence in SEQ ID Nos: 194 to 205.

Embodiment 61. The modified corn plant, or plant part thereof, of any one of Embodiments 44 to 55, wherein said homozygous mutant GA20 oxidase_3 gene comprises a sequence selected from the group consisting of SEQ ID Nos: 206 to 217.

Embodiment 62. The modified corn plant, or plant part thereof, of any one of Embodiments 44 to 61, wherein said homozygous mutant GA20 oxidase_5 gene comprises a sequence selected from the group consisting of SEQ ID Nos: 218 to 228.

Embodiment 63. The modified corn plant, or plant part thereof, of any one of Embodiments 44 to 61, wherein said homozygous mutant GA20 oxidase_5 gene comprises a sequence selected from the group consisting of SEQ ID Nos: 229 to 239.

Embodiment 64. The modified corn plant, or plant part thereof, of any one of Embodiments 44 to 61, wherein said homozygous mutant GA20 oxidase_5 gene comprises a mutation identified by one or more of SEQ ID Nos: 229 to 239 relative to the corresponding reference sequence in SEQ ID Nos: 240 to 250.

Embodiment 65. The modified corn plant, or plant part thereof, of any one of Embodiments 44 to 61, wherein said homozygous mutant GA20 oxidase_5 gene comprises a sequence selected from the group consisting of SEQ ID Nos: 251 to 261.

Embodiment 66. A modified corn plant, or plant part thereof, comprising homozygous mutant alleles at an endogenous GA20 oxidase_3 locus and homozygous mutant alleles at an endogenous GA20 oxidase_5 locus.

Embodiment 67. The modified corn plant or plant part thereof of Embodiment 66, wherein said homozygous mutant alleles at the endogenous GA20 oxidase_3 locus, said homozygous mutant alleles at the endogenous GA20 oxidase_5 locus, or both comprise a heteroallelic combination or two identical mutant alleles.

Embodiment 68. The modified corn plant, or plant part thereof, of Embodiment 66 or 67, wherein said mutant alleles exhibit an at least 30%, at least 40%, at least 50%, at least 60%, at least 70%, at least 80%, at least 90%, at least 95%, or 100% reduction of expression or enzymatic activity relative to a corresponding wild-type GA20 oxidase_3 or GA20 oxidase_5 gene allele.

Embodiment 69. The modified corn plant, or plant part thereof, of any one of Embodiments 66 to 68, wherein said homozygous mutant alleles at the endogenous GA20 oxidase_3 locus comprise a mutation in a sequence region selected from the group consisting of a promoter, 5′ UTR, first exon, first intron, second exon, second intron, third exon, 3′ UTR, terminator, and any combination thereof.

Embodiment 70. The modified corn plant, or plant part thereof, of any one of Embodiments 66 to 69, wherein said homozygous mutant alleles at the endogenous GA20 oxidase_3 locus comprise one or more mutation types selected from the group consisting of a nonsense mutation, a missense mutation, a frameshift mutation, a splice-site mutation, and any combination thereof.

Embodiment 71. The modified corn plant, or plant part thereof, of any one of Embodiments 66 to 70, wherein said homozygous mutant alleles at the endogenous GA20 oxidase_3 locus result in of the following: a GA20 oxidase_3 protein truncation, a non-translatable GA20 oxidase_3 gene transcript, a non-functional GA20 oxidase_3 protein, a premature stop codon in the GA20 oxidase_3 gene, and any combination thereof.

Embodiment 72. The modified corn plant, or plant part thereof, of any one of Embodiments 66 to 71, wherein said homozygous mutant alleles at the endogenous GA20 oxidase_3 locus comprise a mutation selected from the group consisting of a substitution, a deletion, an insertion, a duplication, and an inversion of one or more nucleotides relative to a wild-type GA20 oxidase_3 gene.

Embodiment 73. The modified corn plant, or plant part thereof, of any one of Embodiments 66 to 72, wherein said homozygous mutant alleles at the endogenous GA20 oxidase_3 locus comprise one or more mutations in the first exon of the GA20 oxidase_3 gene.

Embodiment 74. The modified corn plant, or plant part thereof, of any one of Embodiments 66 to 73, wherein said homozygous mutant alleles at the endogenous GA20 oxidase_3 locus comprise one or more mutations in the second exon of the GA20 oxidase_3 gene.

Embodiment 75. The modified corn plant, or plant part thereof, of any one of Embodiments 66 to 74, wherein said homozygous mutant alleles at the endogenous GA20 oxidase_5 locus comprise a mutation in a sequence region selected from the group consisting of a promoter, 5′ UTR, first exon, first intron, second exon, second intron, third exon, 3′ UTR, terminator, and any combination thereof.

Embodiment 76. The modified corn plant, or plant part thereof, of any one of Embodiments 66 to 75, wherein said homozygous mutant alleles at the endogenous GA20 oxidase_5 locus comprise one or more mutation types selected from the group consisting of a nonsense mutation, a missense mutation, a frameshift mutation, a splice-site mutation, and any combination thereof.

Embodiment 77. The modified corn plant, or plant part thereof, of any one of Embodiments 66 to 76, wherein said homozygous mutant alleles at the endogenous GA20 oxidase_5 locus result in one or more of the following: a GA20 oxidase_5 protein truncation, a non-translatable GA20 oxidase_5 gene transcript, a non-functional GA20 oxidase_5 protein, a premature stop codon in the GA20 oxidase_5 gene, and any combination thereof.

Embodiment 78. The modified corn plant, or plant part thereof, of any one of Embodiments 66 to 77, wherein said homozygous mutant alleles at the endogenous GA20 oxidase_5 locus comprise a mutation selected from the group consisting of a substitution, a deletion, an insertion, a duplication, and an inversion of one or more nucleotides relative to a wild-type GA20 oxidase_5 gene.

Embodiment 79. The modified corn plant, or plant part thereof, of any one of Embodiments 66 to 78, wherein said homozygous mutant alleles at the endogenous GA20 oxidase_5 locus comprise one or more mutations in the first exon of the GA20 oxidase_5 gene.

Embodiment 80. The modified corn plant, or plant part thereof, of any one of Embodiments 66 to 79, wherein said homozygous mutant alleles at the endogenous GA20 oxidase_5 locus comprise one or more mutations in the second exon of the GA20 oxidase_5 gene.

Embodiment 81. The modified corn plant, or plant part thereof, of any one of Embodiments 1 to 80, wherein said modified corn plant has a shorter plant height and/or improved lodging resistance relative to an unmodified control plant.

Embodiment 82. The modified corn plant, or plant part thereof, of any one of Embodiments 1 to 80, wherein said modified corn plant is at least 10%, at least 20%, at least 25%, at least 30%, at least 35%, or at least 40% shorter than an unmodified control plant.

Embodiment 83. The modified corn plant, or plant part thereof, of any one of Embodiments 1 to 80, wherein the stalk or stem diameter of said modified corn plant at one or more stem internodes is at least 5%, at least 10%, at least 15%, at least 20%, at least 25%, at least 30%, at least 35%, or at least 40% greater than the stalk or stem diameter at the same one or more internodes of an unmodified control plant.

Embodiment 84. The modified corn plant, or plant part thereof, of any one of Embodiments 1 to 80, wherein the stalk or stem diameter of said modified corn plant at one or more of the first, second, third, and/or fourth internode below the ear is at least 5%, at least 10%, at least 15%, at least 20%, at least 25%, at least 30%, at least 35%, or at least 40% greater than the same internode of an unmodified control plant.

Embodiment 85. The modified corn plant, or plant part thereof, of any one of Embodiments 1 to 80, wherein the level of one or more active GAs in at least one internode tissue of the stem or stalk of said modified corn plant is at least 5%, at least 10%, at least 15%, at least 20%, at least 25%, at least 30%, at least 35%, or at least 40% lower than the same internode tissue of an unmodified control plant.

Embodiment 86. The modified corn plant, or plant part thereof, of any one of Embodiments 1 to 80, wherein the level of one or more active GAs in at least one internode tissue of the stem or stalk of said modified corn plant is lower than the same internode tissue of an unmodified control plant.

Embodiment 87. The modified corn plant, or plant part thereof, of any one of Embodiments 1 to 80, wherein said modified corn plant does not have any significant off-types in at least one female organ or ear.

Embodiment 88. The modified corn plant, or plant part thereof, of any one of Embodiments 1 to 80, wherein said modified corn plant exhibits essentially no reproductive abnormality.

Embodiment 89. The modified corn plant, or plant part thereof, of any one of Embodiments 1 to 80, wherein said modified corn plant comprises one or more traits, relative to an unmodified control plant, selected from the group consisting of shorter plant height, increased stalk/stem diameter, improved lodging resistance, reduced green snap, deeper roots, increased leaf area, earlier canopy closure, higher stomatal conductance, lower ear height, increased foliar water content, improved drought tolerance, improved nitrogen use efficiency, reduced anthocyanin content and area in leaves under normal or nitrogen-limiting or water-limiting stress conditions, increased ear weight, increased harvest index, increased yield, increased seed number, increased seed weight, and increased prolificacy.

Embodiment 90. The modified corn plant or plant part thereof of any one of Embodiments 66 to 80, wherein said homozygous mutant alleles at the endogenous GA20 oxidase_3 locus comprise a sequence selected from the group consisting of SEQ ID Nos: 170 to 181.

Embodiment 91. The modified corn plant or plant part thereof of any one of Embodiments 66 to 80, wherein said homozygous mutant alleles at the endogenous GA20 oxidase_3 locus comprise a sequence selected from the group consisting of SEQ ID Nos: 182 to 193.

Embodiment 92. The modified corn plant or plant part thereof of any one of Embodiments 66 to 80, wherein said homozygous mutant alleles at the endogenous GA20 oxidase_3 locus comprise one or more alleles identified by one or more of SEQ ID Nos: 182 to 193 relative to the corresponding reference sequence in SEQ ID Nos: 194 to 205.

Embodiment 93. The modified corn plant or plant part thereof of any one of Embodiments 66 to 80, wherein said homozygous mutant alleles at the endogenous GA20 oxidase_3 locus comprise a sequence selected from the group consisting of SEQ ID Nos: 206 to 217.

Embodiment 94. The modified corn plant or plant part thereof of any one of Embodiments 66 to 93, wherein said homozygous mutant alleles at the endogenous GA20 oxidase_5 locus comprise a sequence selected from the group consisting of SEQ ID Nos: 218 to 228.

Embodiment 95. The modified corn plant or plant part thereof of any one of Embodiments 66 to 93, wherein said homozygous mutant alleles at the endogenous GA20 oxidase_5 locus comprise a sequence selected from the group consisting of SEQ ID Nos: 229 to 239.

Embodiment 96. The modified corn plant or plant part thereof of any one of Embodiments 66 to 93, wherein said homozygous mutant alleles at the endogenous GA20 oxidase_5 locus comprise one or more alleles identified by one or more of SEQ ID Nos: 229 to 239 relative to the corresponding reference sequence in SEQ ID Nos: 240 to 250.

Embodiment 97. The modified corn plant or plant part thereof of any one of Embodiments 66 to 93, wherein said homozygous mutant alleles at the endogenous GA20 oxidase_5 locus comprise a sequence selected from the group consisting of SEQ ID Nos: 251 to 261.

Embodiment 98. A modified corn plant, or plant part thereof, comprising homozygous mutant alleles at a GA20 oxidase_3 locus, and exhibiting an at least 2.5%, at least 5%, at least 7.5%, at least 10%, at least 15%, or at least 20% reduction in plant height at maturity relative to an unmodified control plant.

Embodiment 99. The modified corn plant, or plant part thereof, of Embodiment 98, wherein said plant height reduction is between 5% and 30%, between 7.5% and 25%, between 10 and 20%, 5% and 7.5%, between 7.5% and 10%, between 10 and 15%, or between 15% to 20%.

Embodiment 100. The modified corn plant, or plant part thereof, of Embodiment 98, wherein said homozygous mutant alleles comprise a heteroallelic combination or two identical mutant alleles.

Embodiment 101. A modified corn plant, or plant part thereof, comprising homozygous mutant alleles at a GA20 oxidase_5 locus, and exhibiting an at least 2.5%, at least 5%, at least 7.5%, at least 10%, at least 15%, or at least 20% reduction in plant height at maturity relative to an unmodified control plant.

Embodiment 102. The modified corn plant, or plant part thereof, of Embodiment 101, wherein said plant height reduction is between 5% and 30%, between 7.5% and 25%, between 10 and 20%, 5% and 7.5%, between 7.5% and 10%, between 10 and 15%, or between 15% to 20%.

Embodiment 103. The modified corn plant, or plant part thereof, of Embodiment 101, wherein said homozygous mutant alleles comprise a heteroallelic combination or two identical mutant alleles.

Embodiment 104. The modified corn plant or plant part thereof of any one of the preceding Embodiments, wherein said modified corn plant is an inbred.

Embodiment 105. The modified corn plant or plant part thereof of any one of the preceding Embodiments, wherein said modified corn plant is a hybrid.

Embodiment 106. A modified corn plant, or plant part thereof, comprising a mutant allele at GA20 oxidase_3 locus, a mutant allele at GA20 oxidase_5 locus, or both, and further comprising an expression cassette comprising a transcribable DNA sequence encoding a non-coding RNA for suppressing one or more of the GA20 oxidase_3 gene, the GA20 oxidase_4 gene, and the GA20 oxidase_5 gene.

Embodiment 107. The modified corn plant, or plant part thereof, Embodiment 106, wherein said GA20 oxidase_3 locus, said GA20 oxidase_5 locus, or both comprise homozygous mutant alleles.

Embodiment 108. The modified corn plant, or plant part thereof, Embodiment 106, wherein said GA20 oxidase_3 locus, said GA20 oxidase_5 locus, or both comprise heterozygous alleles.

Embodiment 109. The modified corn plant, or plant part thereof, Embodiment 106, wherein said GA20 oxidase_3 locus comprises homozygous mutant alleles, and said GA20 oxidase_5 locus comprises heterozygous alleles.

Embodiment 110. The modified corn plant, or plant part thereof, Embodiment 106, wherein said GA20 oxidase_5 locus comprises homozygous mutant alleles, and said GA20 oxidase_3 locus comprises heterozygous alleles

Embodiment 111. The modified corn plant, or plant part thereof, of any one of Embodiments 106 to 110, wherein one or both of said GA20 oxidase_3 and GA20 oxidase_5 loci comprise a heteroallelic combination or two identical mutant alleles.

Embodiment 112. The modified corn plant, or plant part thereof, of any one of Embodiments 106 to 110, wherein said mutant allele at the GA20 oxidase_3 locus comprises a mutation in a sequence region selected from the group consisting of a promoter, 5′ UTR, first exon, first intron, second exon, second intron, third exon, 3′ UTR, terminator, and any combination thereof.

Embodiment 113. The modified corn plant, or plant part thereof, of any one of Embodiments 106 to 110, wherein said mutant allele at the GA20 oxidase_3 locus comprises one or more mutation types selected from the group consisting of a nonsense mutation, a missense mutation, a frameshift mutation, a splice-site mutation, and any combination thereof.

Embodiment 114. The modified corn plant, or plant part thereof, of any one of Embodiments 106 to 110, wherein said mutant allele at the GA20 oxidase_3 locus results in one or more of the following: a GA20 oxidase_3 protein truncation, a non-translatable GA20 oxidase_3 gene transcript, a non-functional GA20 oxidase_3 protein, a premature stop codon in the GA20 oxidase_3 gene, and any combination thereof.

Embodiment 115. The modified corn plant, or plant part thereof, of any one of Embodiments 106 to 110, wherein each mutant allele at the GA20 oxidase_3 locus comprises a mutation selected from the group consisting of a substitution, a deletion, an insertion, a duplication, and an inversion of one or more nucleotides relative to a wild-type GA20 oxidase_3 gene.

Embodiment 116. The modified corn plant, or plant part thereof, of any one of Embodiments 106 to 110, wherein said mutant allele at the GA20 oxidase_3 locus comprises one or more mutations in the first exon of the GA20 oxidase_3 gene.

Embodiment 117. The modified corn plant, or plant part thereof, of any one of Embodiments 106 to 110, wherein said mutant allele at the GA20 oxidase_3 locus comprises one or more mutations in the second exon of the GA20 oxidase_3 gene.

Embodiment 118. The modified corn plant, or plant part thereof, of any one of Embodiments 106 to 110, wherein said mutant allele at the GA20 oxidase_5 locus comprises a mutation in a sequence region selected from the group consisting of a promoter, 5′ UTR, first exon, first intron, second exon, second intron, third exon, 3′ UTR, terminator, and any combination thereof.

Embodiment 119. The modified corn plant, or plant part thereof, of any one of Embodiments 106 to 110, wherein said mutant allele at the GA20 oxidase_5 locus comprises one or more mutation types selected from the group consisting of a nonsense mutation, a missense mutation, a frameshift mutation, a splice-site mutation, and any combination thereof.

Embodiment 120. The modified corn plant, or plant part thereof, of any one of Embodiments 106 to 110, wherein said mutant allele at the GA20 oxidase_5 locus results in one or more of the following: a GA20 oxidase_5 protein truncation, a non-translatable GA20 oxidase_5 gene transcript, a non-functional GA20 oxidase_5 protein, a premature stop codon in the GA20 oxidase_5 gene, and any combination thereof.

Embodiment 121. The modified corn plant, or plant part thereof, of any one of Embodiments 106 to 110, wherein each mutant allele at the GA20 oxidase_5 locus comprises a mutation selected from the group consisting of a substitution, a deletion, an insertion, a duplication, and an inversion of one or more nucleotides relative to a wild-type GA20 oxidase_5 gene.

Embodiment 122. The modified corn plant, or plant part thereof, of any one of Embodiments 106 to 110, wherein said mutant allele at the GA20 oxidase_5 locus comprises one or more mutations in the first exon of the GA20 oxidase_5 gene.

Embodiment 123. The modified corn plant, or plant part thereof, of any one of Embodiments 106 to 110, wherein said mutant allele at the GA20 oxidase_5 locus comprises one or more mutations in the second exon of the GA20 oxidase_5 gene.

Embodiment 124. The modified corn plant, or plant part thereof, of any one of Embodiments 106 to 110, wherein said expression cassette is a recombinant DNA expression cassette, and wherein the transcribable DNA sequence is operably linked to a plant-expressible promoter.

Embodiment 125. The modified corn plant, or plant part thereof, of Embodiment 124, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein being at least 95%, at least 96%, at least 97%, at least 98%, at least 99%, at least 99.5%, or 100% identical to SEQ ID NO: 9, 12, or 15.

Embodiment 126. The modified corn plant, or plant part thereof, of Embodiment 124, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7, 8, 10, 11, 13, or 14.

Embodiment 127. The modified corn plant, or plant part thereof, of Embodiment 124, wherein the plant-expressible promoter is a vascular promoter.

Embodiment 128. The modified corn plant, or plant part thereof, of Embodiment 124, wherein the plant-expressible promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

Embodiment 129. The modified corn plant, or plant part thereof, of Embodiment 124, wherein the plant-expressible promoter is a RTBV promoter.

Embodiment 130. The modified corn plant, or plant part thereof, of Embodiment 124, wherein the plant-expressible promoter is a leaf promoter.

Embodiment 131. The modified corn plant, or plant part thereof, of Embodiment 124, wherein the plant-expressible promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

Embodiment 132. The modified corn plant, or plant part thereof, of Embodiment 124, wherein the plant-expressible promoter is a constitutive promoter.

Embodiment 133. The modified corn plant, or plant part thereof, of Embodiment 124, wherein the plant-expressible promoter comprises one or more constitutive promoters selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof.

Embodiment 134. The modified corn plant, or plant part thereof, of Embodiment 124, wherein the non-coding RNA molecule encoded by the transcribable DNA sequence is a precursor miRNA or siRNA that is processed or cleaved in a plant cell to form a mature miRNA or siRNA.

The Following Paragraphs List a Third Subset of Exemplary Embodiments.

Embodiment 55. A method of making a modified corn plant, or plant part thereof, comprising:

(a) crossing a first corn plant comprising a mutant allele of the GA20 oxidase_3 locus with a second plant comprising a mutant allele of the GA20 oxidase_5 locus; and
(b) selecting a progeny corn plant, or plant part thereof, from the cross in step (a) that is homozygous for one or more mutant alleles of the GA20 oxidase_3 locus and homozygous for one or more mutant alleles of the GA20 oxidase_5 locus.

Embodiment 56. The method of Embodiment 55, wherein the first corn plant is homozygous for one or more mutant alleles of the GA20 oxidase_3 locus, and the second corn plant is heterozygous for a mutant allele of the GA20 oxidase_5 locus.

Embodiment 57. The method of Embodiment 55, wherein the first corn plant is heterozygous for a mutant allele of the GA20 oxidase_3 locus, and the second corn plant is homozygous for one or more mutant alleles of the GA20 oxidase_5 locus.

Embodiment 58. The method of Embodiment 55, wherein the first corn plant is homozygous for one or more mutant alleles of the GA20 oxidase_3 locus, and the second corn plant is homozygous for one or more mutant alleles of the GA20 oxidase_5 locus.

Embodiment 59. The method of any one of Embodiments 55 to 56, wherein the first corn plant is heterozygous for a mutant allele of the GA20 oxidase_5 locus.

Embodiment 60. The method of Embodiment 59, wherein the second corn plant is homozygous for a wild type allele of the GA20 oxidase_3 locus.

Embodiment 61. The method of Embodiment 59, wherein the second corn plant is heterozygous for a mutant allele of the GA20 oxidase_3 locus.

Embodiment 62. The method of Embodiment 59, wherein the second corn plant is homozygous for one or more mutant alleles of the GA20 oxidase_3 locus.

Embodiment 63. The method of any one of Embodiments 55 to 58, wherein the first corn plant is homozygous for one or more mutant alleles of the GA20 oxidase_5 locus.

Embodiment 64. The method of Embodiment 63, wherein the second corn plant is homozygous for a wild type allele of the GA20 oxidase_3 locus.

Embodiment 65. The method of Embodiment 63, wherein the second corn plant is heterozygous for a mutant allele of the GA20 oxidase_3 locus.

Embodiment 66. The method of Embodiment 63, wherein the second corn plant is homozygous for one or more mutant alleles of the GA20 oxidase_3 locus.

Embodiment 67. The method of any one of Embodiments 55 to 58, wherein the first corn plant is homozygous for a wild type allele of the GA20 oxidase_5 locus.

Embodiment 68. The method of Embodiment 67, wherein the second corn plant is homozygous for a wild-type allele of the GA20 oxidase_3 locus.

Embodiment 69. The method of Embodiment 67, wherein the second corn plant is homozygous for one or more mutant alleles of the GA20 oxidase_3 locus.

Embodiment 70. The method of Embodiment 67, wherein the second corn plant is heterozygous for a mutant allele of the GA20 oxidase_3 locus.

Embodiment 71. The method of any one of Embodiments 55 to 70, wherein the progeny corn plant is an F₁ progeny corn plant.

Embodiment 72. A method of making a modified corn plant, or plant part thereof, comprising:

(a) crossing a first corn plant comprising a mutant allele of the GA20 oxidase_3 locus and a mutant allele of the GA20 oxidase_5 locus with a second plant; and
(b) selecting a progeny corn plant, or plant part thereof, from the cross in step (a) that is homozygous for one or more mutant alleles of the GA20 oxidase_3 locus and homozygous for one or more mutant alleles of the GA20 oxidase_5 locus.

Embodiment 73. The method of Embodiment 72, wherein the first corn plant is heterozygous for a mutant allele of the GA20 oxidase_3 locus and is heterozygous for a mutant allele of the GA20 oxidase_5 locus.

Embodiment 74. The method of Embodiment 72, wherein the first corn plant is homozygous for one or more mutant alleles of the GA20 oxidase_3 locus and is heterozygous for a mutant allele of the GA20 oxidase_5 locus.

Embodiment 75. The method of Embodiment 72, wherein the first corn plant is homozygous for one or more mutant alleles of the GA20 oxidase_3 locus and is homozygous for one or more mutant alleles of the GA20 oxidase_5 locus.

Embodiment 76. The method of Embodiment 72, wherein the first corn plant is homozygous for one or more mutant alleles of the GA20 oxidase_3 locus, and the second corn plant is heterozygous for a mutant allele of the GA20 oxidase_5 locus.

Embodiment 77. The method of Embodiment 72, wherein the first corn plant is heterozygous for a mutant allele of the GA20 oxidase_3 locus, and the second corn plant is homozygous for one or more mutant alleles of the GA20 oxidase_5 locus.

Embodiment 78. The method of Embodiment 72, wherein the first corn plant is homozygous for one or more mutant alleles of the GA20 oxidase_3 locus, and the second corn plant is homozygous for one or more mutant alleles of the GA20 oxidase_5 locus.

Embodiment 79. The method of Embodiment 72, wherein the first corn plant is homozygous for one or more mutant alleles of the GA20 oxidase_3 locus, and the second corn plant is homozygous for one or more mutant alleles of the GA20 oxidase_5 locus.

Embodiment 80. The method of any one of Embodiments 76 to 79, wherein the first corn plant is heterozygous for a mutant allele of the GA20 oxidase_5 locus.

Embodiment 81. The method of Embodiment 80, wherein the second corn plant is homozygous for a wild type allele of the GA20 oxidase_3 locus.

Embodiment 82. The method of Embodiment 80, wherein the second corn plant is heterozygous for a mutant allele of the GA20 oxidase_3 locus.

Embodiment 83. The method of Embodiment 80, wherein the second corn plant is homozygous for one or more mutant alleles of the GA20 oxidase_3 locus.

Embodiment 84. The method of any one of Embodiments 76 to 79, wherein the first corn plant is homozygous for one or more mutant alleles of the GA20 oxidase_5 locus.

Embodiment 85. The method of Embodiment 84, wherein the second corn plant is homozygous for a wild type allele of the GA20 oxidase_3 locus.

Embodiment 86. The method of Embodiment 84, wherein the second corn plant is heterozygous for a mutant allele of the GA20 oxidase_3 locus.

Embodiment 87. The method of Embodiment 84, wherein the second corn plant is homozygous for one or more mutant alleles of the GA20 oxidase_3 locus.

Embodiment 88. The method of any one of Embodiments 76 to 79, wherein the second corn plant is heterozygous for a mutant allele of the GA20 oxidase_3 locus.

Embodiment 89. The method of Embodiment 88, wherein the first corn plant is heterozygous for a mutant allele of the GA20 oxidase_3 locus.

Embodiment 90. The method of Embodiment 88, wherein the first corn plant is homozygous for one or more mutant alleles of the GA20 oxidase_5 locus.

Embodiment 91. The method of any one of Embodiments 76 to 79, wherein the second corn plant is homozygous for one or more mutant alleles of the GA20 oxidase_3 locus.

Embodiment 92. The method of Embodiment 91, wherein the first corn plant is heterozygous for a mutant allele of the GA20 oxidase_5 locus.

Embodiment 93. The method of Embodiment 91, wherein the first corn plant is homozygous for one or more mutant alleles of the GA20 oxidase_5 locus.

Embodiment 94. The method of any one of Embodiments 72 to 93, wherein the progeny corn plant is an F₁ progeny corn plant.

The Following Paragraphs List a Fourth Subset of Exemplary Embodiments.

Embodiment 53. A method of making a modified corn plant, or plant part thereof, comprising:

(a) crossing a first corn plant comprising a mutant allele of the GA20 oxidase_3 locus with a second plant comprising a mutant allele of the GA20 oxidase_5 locus; and
(b) selecting a progeny corn plant, or plant part thereof, from the cross in step (a) that is
(i) homozygous for one or more mutant alleles of the GA20 oxidase_3 locus and heterozygous for a mutant allele of the GA20 oxidase_5 locus, or
(ii) heterozygous for a mutant allele of the GA20 oxidase_3 locus and homozygous for one or more mutant alleles of the GA20 oxidase_5 locus.

Embodiment 54. The method of Embodiment 53, wherein the first corn plant is homozygous for one or more mutant alleles of the GA20 oxidase_3 locus, and the second corn plant is heterozygous for a mutant allele of the GA20 oxidase_5 locus.

Embodiment 55. The method of Embodiment 53, wherein the first corn plant is heterozygous for a mutant allele of the GA20 oxidase_3 locus, and the second corn plant is homozygous for one or more mutant alleles of the GA20 oxidase_5 locus.

Embodiment 56. The method of Embodiment 53, wherein the first corn plant is homozygous for one or more mutant alleles of the GA20 oxidase_3 locus, and the second corn plant is homozygous for one or more mutant alleles of the GA20 oxidase_5 locus.

Embodiment 57. The method of any one of Embodiments 53 to 56, wherein the first corn plant is heterozygous for a mutant allele of the GA20 oxidase_5 locus.

Embodiment 58. The method of Embodiment 57, wherein the second corn plant is homozygous for a wild type allele of the GA20 oxidase_3 locus.

Embodiment 59. The method of Embodiment 57, wherein the second corn plant is heterozygous for a mutant allele of the GA20 oxidase_3 locus.

Embodiment 60. The method of Embodiment 57, wherein the second corn plant is homozygous for one or more mutant alleles of the GA20 oxidase_3 locus.

Embodiment 61. The method of any one of Embodiments 53 to 56, wherein the first corn plant is homozygous for one or more mutant alleles of the GA20 oxidase_5 locus.

Embodiment 62. The method of Embodiment 61, wherein the second corn plant is homozygous for a wild type allele of the GA20 oxidase_3 locus.

Embodiment 63. The method of Embodiment 61, wherein the second corn plant is heterozygous for a mutant allele of the GA20 oxidase_3 locus.

Embodiment 64. The method of Embodiment 61, wherein the second corn plant is homozygous for one or more mutant alleles of the GA20 oxidase_3 locus.

Embodiment 65. The method of any one of Embodiments 53 to 56, wherein the first corn plant is homozygous for a wild type allele of the GA20 oxidase_5 locus.

Embodiment 66. The method of Embodiment 64, wherein the second corn plant is homozygous for a wild-type allele of the GA20 oxidase_3 locus.

Embodiment 67. The method of Embodiment 64, wherein the second corn plant is homozygous for one or more mutant alleles of the GA20 oxidase_3 locus.

Embodiment 68. The method of Embodiment 64, wherein the second corn plant is heterozygous for a mutant allele of the GA20 oxidase_3 locus.

Embodiment 69. The method of any one of Embodiments 53 to 68, wherein the progeny corn plant is an F₁ progeny corn plant.

Embodiment 70. A method of making a modified corn plant, or plant part thereof, comprising:

(a) crossing a first corn plant comprising a mutant allele of the GA20 oxidase_3 locus and a mutant allele of the GA20 oxidase_5 locus with a second plant; and
(b) selecting a progeny corn plant, or plant part thereof, from the cross in step (a) that is
(i) homozygous for one or more mutant alleles of the GA20 oxidase_3 locus and heterozygous for a mutant allele of the GA20 oxidase_5 locus, or
(ii) heterozygous for a mutant allele of the GA20 oxidase_3 locus and homozygous for one or more mutant alleles of the GA20 oxidase_5 locus.

Embodiment 71. The method of Embodiment 70, wherein the first corn plant is heterozygous for a mutant allele of the GA20 oxidase_3 locus and is heterozygous for a mutant allele of the GA20 oxidase_5 locus.

Embodiment 72. The method of Embodiment 70, wherein the first corn plant is homozygous for one or more mutant alleles of the GA20 oxidase_3 locus and is heterozygous for a mutant allele of the GA20 oxidase_5 locus.

Embodiment 73. The method of Embodiment 70, wherein the first corn plant is homozygous for one or more mutant alleles of the GA20 oxidase_3 locus and is homozygous for one or more mutant alleles of the GA20 oxidase_5 locus.

Embodiment 74. The method of Embodiment 70, wherein the first corn plant is homozygous for one or more mutant alleles of the GA20 oxidase_3 locus, and the second corn plant is heterozygous for a mutant allele of the GA20 oxidase_5 locus.

Embodiment 75. The method of Embodiment 70, wherein the first corn plant is heterozygous for a mutant allele of the GA20 oxidase_3 locus, and the second corn plant is homozygous for one or more mutant alleles of the GA20 oxidase_5 locus.

Embodiment 76. The method of Embodiment 70, wherein the first corn plant is homozygous for one or more mutant alleles of the GA20 oxidase_3 locus, and the second corn plant is homozygous for one or more mutant alleles of the GA20 oxidase_5 locus.

Embodiment 77. The method of Embodiment 70, wherein the first corn plant is homozygous for one or more mutant alleles of the GA20 oxidase_3 locus, and the second corn plant is homozygous for one or more mutant alleles of the GA20 oxidase_5 locus.

Embodiment 78. The method of any one of Embodiments 74 to 77, wherein the first corn plant is heterozygous for a mutant allele of the GA20 oxidase_5 locus.

Embodiment 79. The method of Embodiment 78, wherein the second corn plant is homozygous for a wild type allele of the GA20 oxidase_3 locus.

Embodiment 80. The method of Embodiment 78, wherein the second corn plant is heterozygous for a mutant allele of the GA20 oxidase_3 locus.

Embodiment 81. The method of Embodiment 78, wherein the second corn plant is homozygous for one or more mutant alleles of the GA20 oxidase_3 locus.

Embodiment 82. The method of any one of Embodiments 74 to 77, wherein the first corn plant is homozygous for one or more mutant alleles of the GA20 oxidase_5 locus.

Embodiment 83. The method of Embodiment 82, wherein the second corn plant is homozygous for a wild type allele of the GA20 oxidase_3 locus.

Embodiment 84. The method of Embodiment 82, wherein the second corn plant is heterozygous for a mutant allele of the GA20 oxidase_3 locus.

Embodiment 85. The method of Embodiment 82, wherein the second corn plant is homozygous for one or more mutant alleles of the GA20 oxidase_3 locus.

Embodiment 86. The method of any one of Embodiments 74 to 77, wherein the second corn plant is heterozygous for a mutant allele of the GA20 oxidase_3 locus.

Embodiment 87. The method of Embodiment 86, wherein the first corn plant is heterozygous for a mutant allele of the GA20 oxidase_3 locus.

Embodiment 88. The method of Embodiment 86, wherein the first corn plant is homozygous for one or more mutant alleles of the GA20 oxidase_5 locus.

Embodiment 89. The method of any one of Embodiments 74 to 77, wherein the second corn plant is homozygous for one or more mutant alleles of the GA20 oxidase_3 locus.

Embodiment 90. The method of Embodiment 89, wherein the first corn plant is heterozygous for a mutant allele of the GA20 oxidase_5 locus.

Embodiment 91. The method of Embodiment 89, wherein the first corn plant is homozygous for one or more mutant alleles of the GA20 oxidase_5 locus.

Embodiment 92. The method of any one of Embodiments 70 to 91, wherein the progeny corn plant is an F₁ progeny corn plant.

### EXAMPLES

### Example 1. Reduced Plant Height in Inbred Corn Lines Across Transformation Events for the GA20 Oxidase Suppression Element

An inbred corn plant line was transformed via Agrobacterium mediated transformation with a transformation vector having an expression construct comprising a transcribable DNA sequence with a sequence (SEQ ID NO: 39) encoding a targeting sequence (SEQ ID NO: 40) of a miRNA under the control of a rice tungro bacilliform virus (RTBV) promoter (SEQ ID NO: 65) that is known to cause expression in vascular tissues of plants. The miRNA encoded by the construct comprises a RNA sequence that targets the GA20 oxidase_3 and GA20 oxidase_5 genes in corn plants for suppression. Several transformation events were generated with this construct, and these transformants were tested in the greenhouse to determine if they had reduced plant height relative to non-transgenic wild type control plants. As can be seen in FIG. 1, a significant reduction in plant height was consistently observed in transgenic plants expressing the suppression construct across several transformation events (see Events 1-8) relative to wild type (WT) control plants. Plant height for each of the transformation events was calculated as an average among approximately 10 plants for each event and compared to the average height for control plants. Standard errors were calculated for each event and the control plants, which are represented as error bars in FIG. 1. Furthermore, ear development in each of these transformants appeared normal.

As can be seen from the results of this experiment, average plant height in plants expressing the miRNA targeting the GA20 oxidase_3 and GA20 oxidase_5 genes for suppression had consistently reduced plant heights of up to 35% relative to control plants across multiple events. This data supports the conclusion that the effects seen with this suppression construct are not due to insertion of the construct at any one locus within the plant genome.

This data further indicates that expression of this GA20 oxidase suppression construct using the RTBV vascular promoter is effective at causing these plant height phenotypes. In addition, early data in R0 corn plants constitutively expressing the same GA20 oxidase suppression construct under the control of different constitutive promoters also produce short stature plants (see Example 15 below). Thus, expression of the targeted GA20 oxidase suppression construct may be effective at reducing plant height and providing the other beneficial anti-lodging and yield-related traits described herein given that different expression patterns including vascular and constitutive expression provide similar plant height phenotypes without apparent off-types in the ear.

### Example 2. Reduced Plant Height in Hybrid Corn Plants Expressing the GA20 Oxidase Suppression Element

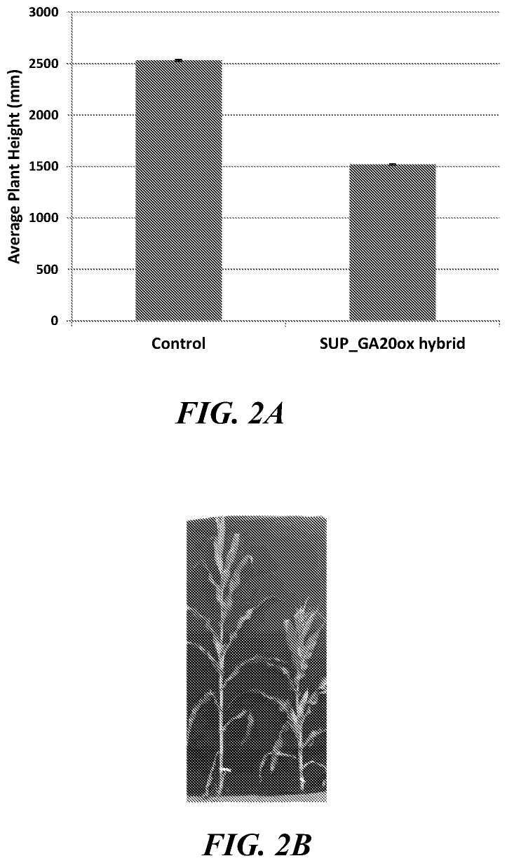

Hybrid corn plants carrying the GA20 oxidase suppression construct described in Example 1 also showed reduced plant height relative to wild type control plants when grown under field conditions. Average plant height of transgenic hybrid corn plants expressing the GA20 oxidase suppression element in 10 microplots was calculated and compared to average plant height of (non-transgenic) wild type control hybrid corn plants in 32 microplots. Each microplot for the transgenic and non-transgenic control included approximately 6 plants, although the actual number of plants per plot may vary depending on the number of plants that germinate and develop into plants having ears. As can be seen in FIG. 2A, a significant reduction in average plant height was observed in transgenic hybrid plants expressing the suppression construct (SUP-GA20ox hybrid), relative to wild type hybrid corn plants (Control). Standard errors were calculated for the transgenic hybrid and control plants, which are represented as error bars in FIG. 2A. An image of a hybrid control plant (left) next to a transgenic hybrid plant expressing the GA20 oxidase suppression element (right) is further shown in FIG. 2B.

In this experiment, average plant height of field grown hybrid corn plants expressing the miRNA targeting the GA20 oxidase_3 and GA20 oxidase_5 genes was reduced by about 40% relative to wild type hybrid control plants. This data shows that the plant height phenotype is present in hybrid corn plants in addition to inbred lines. However, overall biomass in this experiment appeared neutral in the semi-dwarf corn plants compared to controls.

### Example 3. Increased Stem Diameter in Hybrid Corn Plants Expressing the GA20 Oxidase Suppression Element

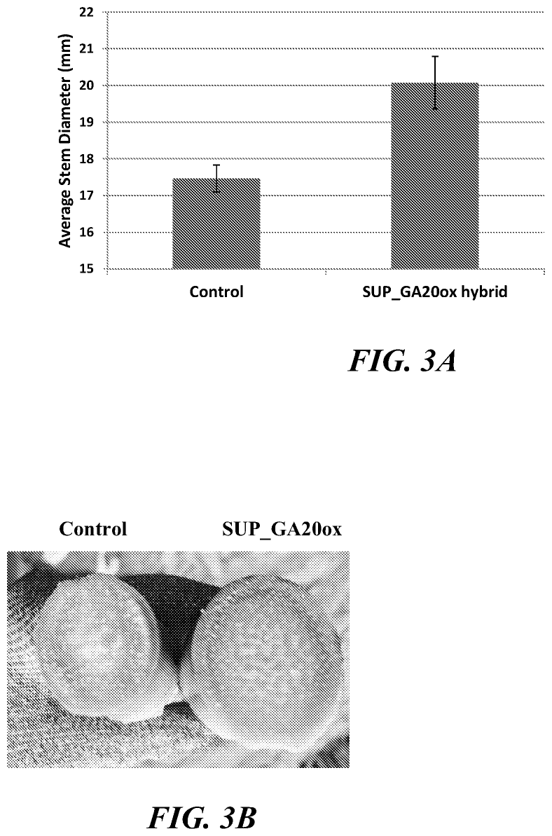

Hybrid corn plants carrying the GA20 oxidase suppression construct described in Example 1 also showed increased stem diameter relative to wild type control plants when grown under field conditions. Stem diameter was measured on the second internode below the primary ear. Average stem diameter of transgenic hybrid corn plants expressing the GA20 oxidase suppression element in 8 microplots was calculated and compared to the average stem diameter of (non-transgenic) wild type control hybrid corn plants in 8 microplots. Each microplot included approximately 6 plants. As can be seen in FIG. 3A, a significant increase in average stem diameter was observed in transgenic hybrid plants expressing the suppression construct (SUP-GA20ox hybrid), relative to wild type hybrid corn plants (Control). Standard errors were calculated for the transgenic hybrid and control plants, which are represented as error bars in FIG. 3A. An image of the cross-section of a stalk from a hybrid control plant (Control; left) is shown next to the cross-section of a stalk from a transgenic hybrid plant expressing the GA20 oxidase suppression element (SUP GA20ox; right) is further shown in FIG. 3B.

In this experiment, average stem diameter of field grown hybrid corn plants expressing the miRNA targeting the GA20 oxidase_3 and GA20 oxidase_5 genes was increased about 13% relative to wild type hybrid control plants. This data shows that hybrid corn plants expressing the GA20 oxidase miRNA may have thicker stalks in addition to the reduced plant height phenotype.

### Example 4. Hybrid Corn Plants Expressing the GA20 Oxidase Suppression Element had an Increase in Fresh Ear Weight

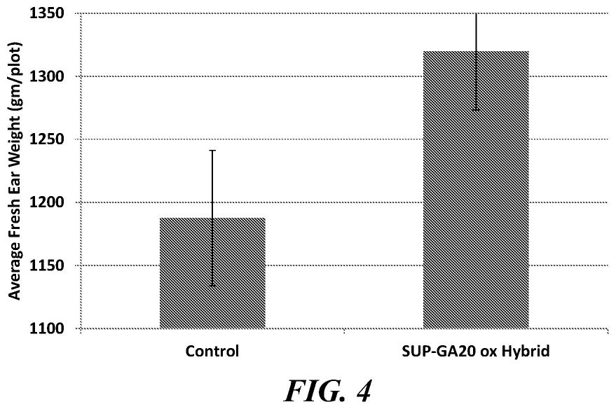

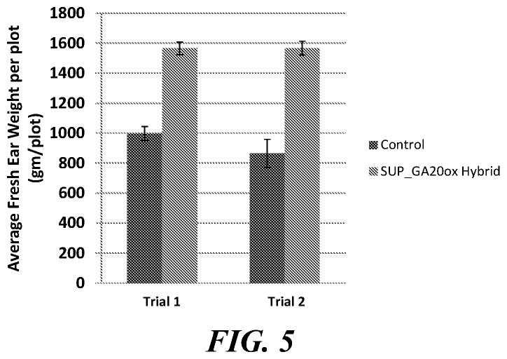

Hybrid corn plants carrying the GA20 oxidase suppression construct described in Example 1 also showed an increase in fresh ear weight relative to wild type control plants when grown under field conditions. Average fresh ear weight per plot of transgenic hybrid corn plants expressing the GA20 oxidase suppression element in 24 microplots was calculated and compared to the average fresh ear weight of (non-transgenic) hybrid corn control plants in 8 microplots. Again, each microplot included about 6 plants. As can be seen in FIG. 4, an increase in average fresh ear weight per plot was observed in transgenic hybrid plants expressing the suppression construct (SUP-GA20ox hybrid), relative to wild type hybrid corn plants (Control), and ear and kernel development appeared normal. Standard deviations for this experiment were calculated for the transgenic hybrid and control plants, which are represented as error bars in FIG. 4. As shown in FIG. 5, similar results were obtained at another field testing site that also experienced wind damage.

In this experiment, average fresh ear weight of field grown hybrid corn plants expressing the miRNA targeting the GA20 oxidase_3 and GA20 oxidase_5 genes was increased relative to wild type hybrid control plants, indicating that these transgenic plants may further have improved yield-related traits. However, these results are based on observational data without a large-scale statistical comparison to controls, and yield performance should be tested under broad acre conditions.

### Example 5. Hybrid Corn Plants Expressing the GA20 Oxidase Suppression Element Displayed Increased Resistance to Lodging

At a field testing location, wind damage to pre-flowering hybrid corn plants demonstrated an increased lodging resistance with plants expressing the GA20 oxidase suppression construct described in Example 1, relative to wild type hybrid control plants. While the wild type (non-transgenic) hybrid control plants were visually lodged in response to this high wind event, transgenic hybrid corn plants expressing the GA20 oxidase suppression element in a neighboring field location resisted lodging damage. To evaluate the effects of lodging resistance by hybrid corn plants expressing the GA20 oxidase suppression construct, average fresh ear weights per plot of transgenic GA20 oxidase-suppressing hybrid corn plants across two field trial locations experiencing the lodging damage, were compared to average fresh ear weights of wild type hybrid control plants. Data collected from these two trials indicated that the hybrid control plants had average fresh ear weights that were reduced by about 57% and 81%, respectively in the two trials, relative to hybrid plants expressing the GA20 oxidase suppression construct.

The visual observation that transgenic GA20 oxidase-suppressing hybrid corn plants had increased lodging resistance than non-transgenic control plants, along with the increase in average fresh ear weight in these trials with the transgenic GA20 oxidase-suppressing plants, indicate that increased lodging resistance may translate into an increase in average fresh ear weight. Thus, increased lodging resistance in GA20 oxidase-suppressing plants may further increase the yield potential/stability of these transgenic corn plants by resisting the effects of lodging during severe weather events.

### Example 6. Hybrid Corn Plants Expressing the GA20 Oxidase Suppression Element had an Increase in Harvest Index

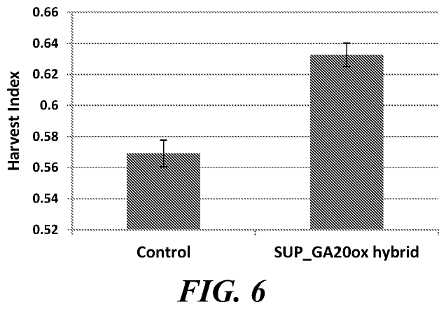

Hybrid corn plants carrying the GA20 oxidase suppression construct described in Example 1 further showed an increase in harvest index relative to wild type control plants when grown under field conditions. The harvest index of transgenic hybrid corn plants expressing the GA20 oxidase suppression element was determined from plants grown in 8 microplots and compared to non-transgenic hybrid corn control plants. Each microplot included approximately 6 plants. As can be seen in FIG. 6, a significant increase in harvest index was observed in the transgenic hybrid plants expressing the suppression construct (SUP-GA20ox hybrid), relative to wild type hybrid corn plants (Control). Standard errors were calculated for the transgenic hybrid and control plants, which are represented as error bars in FIG. 6.

In this experiment, the harvest index of field grown hybrid corn plants expressing the miRNA targeting the GA20 oxidase_3 and GA20 oxidase_5 genes was increased about 11% relative to wild type hybrid control plants. This increase in harvest index was further associated with a reduction in stover weight as compared to wild type control plants, but no difference in total dry biomass weight was observed in the transgenic plants.

### Example 7. Hybrid Corn Plants Expressing the GA20 Oxidase Suppression Element had an Increase in Average Grain Yield Estimate

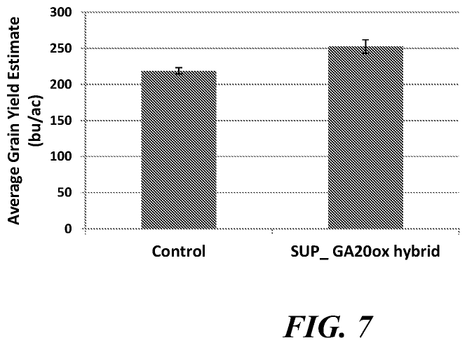

The average grain yield estimate for hybrid corn plants expressing the GA20 oxidase suppression element (identified in Example 1) was calculated from 16 microplots in the field (with approximately 6 plants per plot). The calculated average grain yield estimate for these transgenic hybrid corn plants suppressing GA20 oxidase was increased by about 15% over corn hybrid control plants (FIG. 7). Grain yield estimate is a metric that provides a general estimation of expected yield based on the ear trait metrics. Grain yield estimate is derived from hand harvested ears on small plots, and units are kg/ha (instead of bu/ac). Grain yield estimate (kg/ha) is calculated by the formula (Kernel number per unit area (kernels/m²) x Single Kernel Weight (mg)×15.5%/100).

### Example 8. Hybrid Corn Plants Expressing the GA20 Oxidase Suppression Element had an Increase in Average Prolificacy Score

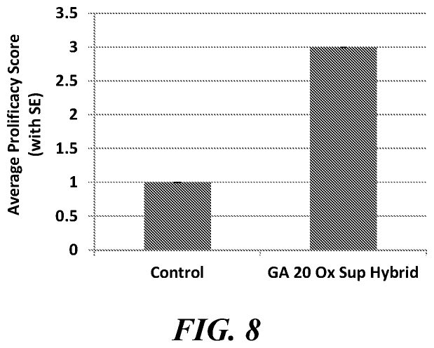

Hybrid corn plants expressing the GA20 oxidase suppression element (identified in Example 1) was also shown in a microplot experiment to have increased prolificacy and secondary ears as compared to non-transgenic hybrid control plants. The prolificacy score was determined from 10 microplots of the transgenic hybrid corn plants in the field (with approximately 6 plants per plot). As shown in FIG. 8, the average prolificacy score of transgenic hybrid corn plants suppressing the GA 20 oxidase was 3, whereas the average prolificacy score of control plants was 1. To determine the prolificacy score, plants were assayed for the development of secondary ears at the R1 stage of development. Plants were rated on the following scale: 1=Little or no secondary ear formation; 2=Silks are prominent on the secondary ear; 3=Developed secondary ear emerged from the ear leaf sheath; and 4=Good secondary ear development similar to the primary ear. End-of-season harvest further indicated at least some secondary ears were productive with normally developed kernels.

### Example 9. Broad-Acre Yield and Trait Trials in the Field with Hybrid Corn Plants Transformed with the GA20 Oxidase Suppression Construct

The GA20 oxidase suppression construct described in Example 1 was transformed into a female commercial corn inbred line, and a number of transformation events were created. The transformed plants were grown and self-crossed to bulk up sufficient seeds, and then crossed to various male commercial corn inbred lines to produce hybrid corn plants. Each distinct male inbred line used to produce the male-female hybrid is called a tester. The hybrid corn plants with different testers were then grown on broad acres in the field according to standard agronomic practice (SAP). The planting density for SAP was 34,000 plants per acres with 30″ row spacing.

For yield trials, four different transformation events expressing the GA20 oxidase suppression construct were crossed to 2 different commercial tester lines. The hybrid corn plants were then tested in 16 geographic locations across 6 US Midwest states. Yield of transgenic hybrid corn plants across these locations was calculated and compared to the yield of non-transgenic hybrid corn control plants. Table 4 provides the yield difference in bushels/acre between the transgenic hybrid corn plants for each event as compared to a non-transgenic control. A negative number indicates a yield decrease. Yield differences with a statistical p-value of less than 0.2 are indicated in Table 4 with bold and italic font. This notation is also used to indicate statistical significance for the remaining tables in these Examples, unless otherwise noted. As shown in Table 4 under the SAP heading, a significant increase in yield was observed in transgenic hybrid corn plants expressing the suppression construct (transgenic plants) under SAP conditions, relative to wild type hybrid corn plants (Control). The significant increase in yield was observed across 4 transgenic events, and 2 tester lines.

A comparable broad-acre yield trial was conducted under high density (HD) planting conditions with 42,000 plants per acre and 30″ row spacing, and compared to standard agronomic practice (SAP) density. The differences in yield under HD conditions are provided in Table 4 under the HD heading. Mixed results were obtained under these high density conditions with yield varying across events and testers. However, an increase in yield was observed for two events with one of the two testers, and the possibility remains for higher yield across a greater number of germplasms under different high density conditions.

TABLE 4
Broad-acre yield difference between transgenic
plants and control, under SAP and HD

| | SAP | | | HD | | |
|---|---|---|---|---|---|---|
| | | | Across | | | Across |
| | Tester-1 | Tester-2 | Testers | Tester-1 | Tester-2 | Testers |
| Across Events | 3.7 | 3.9 | 4 | 3.5 | [symbol] | −3.9 |
| Event-1 | [symbol] | 2.7 | [symbol] | [symbol] | −4.3 | 1.3 |
| Event-2 | 3.2 | [symbol] | [symbol] | −5.1 | [symbol] | [symbol] |
| Event-3 | 2.3 | 1.8 | 2 | [symbol] | [symbol] | −0.7 |
| Event-4 | 1.7 | 4.6 | 3.4 | 3.1 | [symbol] | [symbol] |

Trait trials were also conducted in the field to measure a number of developmental and reproductive traits. These trials were conducted under normal density (SAP) as described above and ultra high density (UHD) planting conditions of 54,000 plants per acre with 20″ row spacing. The trials were conducted in hybrid corn plants with 7 transformation events and 3 testers, and the data for each tester was pooled over the 7 events.

Table 5 summarizes the trait trial results in hybrid corn plants. The measurement is either a percent difference, or a difference of days or number of leaves, between the transgenic plants and the control. Where appropriate, the development stage, such as R3, etc., at which the measurement was taken, is indicated in parenthesis under the column “Trait Name”. Pollen shedding is measured in terms of the number of days from germination to 50% of plants shedding pollen. Silking emergence is measured in terms of the number of days from germination to 50% of plants silking. Pollen-silk interval is a measure of the number of days from 50% of plants shedding pollen to silking. Stalk strength is a measure of the amount of force at which the stalk segment breaks laterally, using a stalk breaker instrument. Leaf area index (LAI) is a dimensionless quantity that characterizes the extent of the plant canopy, defined as the one-sided green leaf area per unit ground surface area within a broadleaf canopy space.

TABLE 5
Trait differences between transgenic and control plants under SAP and UHD.

| | | 30″ SAP | | | 20″ UHD | | |
|---|---|---|---|---|---|---|---|
| Measurement | Trait Name | Tester-1 | Tester-2 | Tester-3 | Tester-1 | Tester-2 | Tester-3 |
| % Delta | Plant height (R3) | [symbol] | [symbol] | [symbol] | [symbol] | [symbol] | [symbol] |
| | Plant height below 6 ft | YES | YES | YES | YES | YES | YES |
| | Ear height (R3) | [symbol] | [symbol] | [symbol] | [symbol] | [symbol] | [symbol] |
| | Ear height above 18 | YES | YES | YES | YES | YES | YES |
| | inches | | | | | | |
| % Delta | Internode length (ear | [symbol] | [symbol] | [symbol] | [symbol] | [symbol] | [symbol] |
| | minus 2) (R3) | | | | | | |
| | Internode length (ear | [symbol] | [symbol] | [symbol] | [symbol] | [symbol] | [symbol] |
| | minus 4) (R3) | | | | | | |
| | Stalk Diameter (2 | 4.4 | 5.8 | 4.5 | [symbol] | [symbol] | [symbol] |
| | nodes below ear) (R3) | | | | | | |
| | Stalk Diameter (4 | 3.9 | −1.6 | 1 | [symbol] | [symbol] | [symbol] |
| | nodes below ear) (R3) | | | | | | |
| | Stalk strength 2nd | 10.2 | 0.1 | 0.7 | [symbol] | [symbol] | N/A |
| | node below ear (R5) | | | | | | |
| | Stalk strength 4th | −13.6 | [symbol] | −11.5 | [symbol] | [symbol] | N/A |
| | node below ear (R5) | | | | | | |
| Days | Pollen-silk interval | [symbol] | [symbol] | [symbol] | [symbol] | [symbol] | [symbol] |
| | Pollen shedding | [symbol] | [symbol] | [symbol] | [symbol] | [symbol] | [symbol] |
| | Silking emergence | [symbol] | −0.25 | [symbol] | [symbol] | [symbol] | [symbol] |
| Number | Green leaf # (R4) | [symbol] | [symbol] | [symbol] | [symbol] | [symbol] | [symbol] |
| | Green leaf # (R5) | [symbol] | [symbol] | [symbol] | [symbol] | [symbol] | [symbol] |
| | Green leaf # (7 days | −0.5 | [symbol] | −0.3 | [symbol] | −0.3 | [symbol] |
| | after R5) | | | | | | |
| | Green leaf # (14 days | [symbol] | −0.4 | −0.2 | [symbol] | −0.3 | [symbol] |
| | after R5) | | | | | | |
| % Delta | Leaf area index (V6) | [symbol] | [symbol] | [symbol] | [symbol] | [symbol] | −16.6 |
| | Leaf area index (V8) | [symbol] | −1.4 | 15.4 | [symbol] | [symbol] | [symbol] |
| | Leaf area index (V10) | 10.7 | 2 | 8.5 | [symbol] | [symbol] | −7.7 |
| | Leaf area index (V12) | 2.3 | −5.4 | 3.6 | [symbol] | [symbol] | [symbol] |

As shown in Table 5, a significant decrease in plant height, ear height, and internode length was observed in transgenic plants relative to the control. The transgenic plants consistently exhibited plant heights below 6 feet, and ear heights above 18 inches, allowing harvesting by combine without modification to the machinery. In this experiment, increased stalk diameter was observed particularly under higher density planting conditions.

Table 6 summarizes the ear trait trial results for hybrid corn. The trials were conducted in hybrid corn plants with 7 transformation events and 3 testers, and the data for each tester was pooled over the 7 events. The measurements are the percent delta difference between the transgenic plants and the control. Where appropriate, the development stage, such as R3, etc., at which the measurement was taken, is indicated in parenthesis under the column “Trait Name”. Ear area is a measure of the plot average size of an ear in terms of area from a 2-dimensional view taken by imaging the ear, including kernels and void. Ear diameter is the plot average of the ear diameter measured as the maximal “wide” axis of the ear over its widest section. Ear length is a measure of the plot average of the length of ear measured from the tip of the ear in a straight line to the base of the ear node. Ear tip void pct is a measure of the plot average of the area percentage of void at the top 30% area of the ear, from a 2-dimensional view taken by imaging the ear, including kernels and void. Ear void measures the plot average of the area percentage of void on an ear, from a 2-dimensional view, is measured by imaging the ear, including kernels and void. Grain yield estimate is defined in Example 7. Kernels per unit area is measured as the plot average of the number of kernels per unit area of the field. Ears were collected from a set row length, typically one meter, and shelled and combined to count the kernels, and the count was then converted to the total kernels per unit area of the field. Single kernel weight measures the plot average of weight per kernel. It is calculated as the ratio of (sample kernel weight adjusted to 15.5% moisture)/(sample kernel number). Kernels per ear is a measure of the plot average of the number of kernels per ear. It is calculated as (total kernel weight/(Single Kernel Weight*total ear count), where total kernel weight and total ear count are measured from ear samples over an area between 0.19 to 10 square meters.

TABLE 6
Ear trait differences between transgenic and control plants, under SAP and UHD.

| | 30″ SAP | | | 20″ UHD | | |
|---|---|---|---|---|---|---|
| Trait Name | Tester-1 | Tester-2 | Tester-3 | Tester-1 | Tester-2 | Tester-3 |
| Ear area (R6) (cm²) | [symbol] | [symbol] | [symbol] | [symbol] | [symbol] | [symbol] |
| Ear diameter (R6) (mm) | [symbol] | −0.7 | [symbol] | −1.7 | 1.3 | −1.8 |
| Ear length (R6) (cm) | [symbol] | [symbol] | [symbol] | [symbol] | [symbol] | [symbol] |
| Ear tip void_pct (R6) (%) | −9.1 | −1.1 | 7.7 | −5.8 | 24 | 11.1 |
| Ear void (R6) (%) | −3.3 | 1.9 | 9.5 | −6.7 | 10 | 16.4 |
| Grain yield estimate (R6) | 2.8 | −4.6 | −5.0 | 0.2 | 19.2 | 0.9 |
| (kg/hectare) | | | | | | |
| Kernels per unit area (R6) | −0.7 | −9.8 | −6.7 | [symbol] | [symbol] | 9 |
| (kernels/m²) | | | | | | |
| Kernels per ear (R6) (count) | −3.2 | 0.5 | −3.5 | [symbol] | [symbol] | 6.5 |
| Single kernel weight (R6) | 1.8 | 5.1 | 1.1 | [symbol] | [symbol] | −7.5 |
| (mg) | | | | | | |

As shown in Table 6, there was a significant increase in ear area and ear length observed in these experiments for the transgenic plants as compared to the control. There was also a noticeable decrease in the ear diameter. In this experiment, the grain yield estimate was mostly neutral between transgenic plants and the control.

Additional data was collected in the field at standard density across 8 events crossed to one tester showing a reduction in plant height, ear height, and internode length, and an increase in stem diameter and harvest index, as compared to a control (data not shown). Plant heights were measured from the ground to the uppermost ligulated leaf at R3 stage. Ear heights were measured from the ground to the ear node at R3 stage. Stalk diameters were measured at the middle of the stalk internode 2 nodes below the ear, unless otherwise indicated. These data demonstrated high penetrance of plant height and stalk traits across events, although an increase in prolificacy (or the number of secondary ears) was not significant or pronounced in these studies.

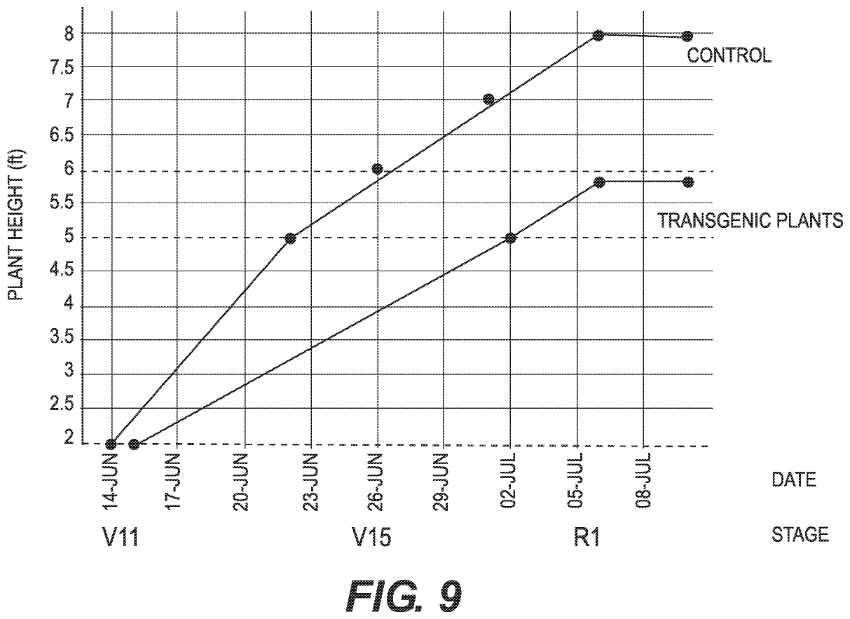

In a separate experiment, plant height growth was measured from V11 to R1 stage and beyond. FIG. 9 shows the differences in plant height between transgenic plants and the control over this time frame. Drawn on the figure are dotted lines for 5-foot and 6-foot heights for reference.

### Example 10. Transgenic Plants Exhibited Enhanced Traits Under Nitrogen and Water Stress Conditions in Controlled Environment Conditions

This example illustrates the enhanced water and nitrogen stress response of transgenic corn plants having the GA20 oxidase suppression construct described in Example 1 versus the control, in an automated greenhouse (AGH) or the field as indicated. The apparatus and the methods for automated phenotypic assaying of plants in AGH are disclosed, for example, in U.S. Patent Publication No. 2011/0135161, which is incorporated herein by reference in its entirety.

In the AGH setting, corn plants were tested under five different conditions including non-stress, mild and moderate nitrogen deficit, and mild and moderate water deficit stress conditions. The corn plants were grown under the stress-specific conditions shown in Table 7.

TABLE 7
Description of the five AGH growth conditions.

| | Volumetric Water | |
|---|---|---|
| Condition | Content (VWC) | Nitrogen Concentration |
| No stress | 50% | 8 mM |
| Water Stress: mild | 40% | 8 mM |
| Water Stress: moderate | 35% | 8 mM |
| Nitrogen Stress: mild | 50% | 6 mM |
| Nitrogen Stress: moderate | 50% | 4 mM |

Water deficit is defined as a specific Volumetric Water Content (VWC) that is lower than the VWC of a non-stressed plant. For example, a non-stressed plant might be maintained at 50% VWC, and the VWC for a water-deficit assay might be defined between 35% to 40% VWC. Data were collected using visible light and hyperspectral imaging as well as direct measurement of pot weight and amount of water and nutrient applied to individual plants on a daily basis. Nitrogen deficit is defined (in part) as a specific mM concentration of nitrogen that is lower than the nitrogen concentration of a non-stressed plant. For example, a non-stressed plant might be maintained at 8 mM nitrogen, while the nitrogen concentration applied in a nitrogen-deficit assay might be maintained at a concentration between 4 to 6 mM.

Up to ten parameters were measured for each screen. The visible light color imaging based measurements are: plant height, biomass, and canopy area. Plant Height (PlntH) refers to the distance from the top of the pot to the highest point of the plant derived from a side image (mm). Biomass (Bmass) is defined as the estimated shoot fresh weight (g) of the plant obtained from images acquired from multiple angles of view. Canopy Area (Cnop) is defined as leaf area as seen in a top-down image (mm²). Anthocyanin score and area, chlorophyll score and concentration, and water content score were measured with hyperspectral imaging. Anthocyanin Score (AntS) is an estimate of anthocyanin in the leaf canopy obtained from a top-down hyperspectral image. Anthocyanin Area (AntA) is an estimate of anthocyanin in the stem obtained from a side-view hyperspectral image. Chlorophyll Score (ClrpS) and Chlorophyll Concentration (ClrpC) are both measurements of chlorophyll in the leaf canopy obtained from a top-down hyperspectral image, where Chlorophyll Score measures in relative units, and Chlorophyll Concentration is measured in parts per million (ppm) units. Foliar Water Content (FlrWtrCt) is a measurement of water in the leaf canopy obtained from a top-down hyperspectral image. Water Use Efficiency (WUE) is derived from the grams of plant biomass per liter of water added. Water Applied (WtrAp) is a direct measurement of water added to a pot (pot with no hole) during the course of an experiment. These physiological trials were set up so that tested transgenic plants were compared to the control. Transgenic plants of two transformation events were measured in comparison with the control. All data are in percent delta difference of the transgenic plant with respect to the control. Data point with statistical p-value <0.1 were shown in bold italic font. Other data points have p-value >0.1.

Table 8 summarizes the AGH trait trial results as measured at 21 days from planting in the vegetative stage, whereas Table 9 summarizes the AGH trait trial results as measured at 55 days from planting in the reproductive stage, in transgenic plants having one of two events of the GA20 oxidase suppression construct described in Example 1 relative to control plants.

TABLE 8
Transgenic versus control plants in the greenhouse under normal and stress conditions, 21 days from planting.

| | Event-1 | | | | | Event-2 | | | | |
|---|---|---|---|---|---|---|---|---|---|---|
| | | Nitrogen stress | | Water stress | | | Nitrogen stress | | Water stress | |
| Trait Name | No stress | Mild | Moderate | Mild | Moderate | No stress | Mild | Moderate | Mild | Moderate |
| Plant height | [symbol] | [symbol] | [symbol] | [symbol] | [symbol] | [symbol] | [symbol] | [symbol] | [symbol] | [symbol] |
| Biomass | −0.06 | [symbol] | [symbol] | [symbol] | [symbol] | −0.32 | [symbol] | −1.77 | [symbol] | −1.48 |
| Canopy area | 0.79 | [symbol] | [symbol] | 1.45 | [symbol] | 0.36 | [symbol] | [symbol] | 4.38 | 2.75 |
| Foliar water | [symbol] | [symbol] | [symbol] | | 10.1 | [symbol] | [symbol] | [symbol] | [symbol] | [symbol] |
| content | | | | | | | | | | |
| Anthocyanin | [symbol] | [symbol] | [symbol] | [symbol] | [symbol] | [symbol] | [symbol] | [symbol] | [symbol] | [symbol] |
| area | | | | | | | | | | |
| Anthocyanin | −10.21 | −14.5 | −2.9 | [symbol] | 2.4 | [symbol] | 4.5 | 3.3 | [symbol] | −2.5 |
| score | | | | | | | | | | |
| Chlorophyll | 1.2 | 0.68 | 0.04 | [symbol] | 3.27 | [symbol] | −2.03 | −3.46 | −4.14 | 2.05 |
| concentration | | | | | | | | | | |

TABLE 9
Transgenic versus control plants in the greenhouse under normal and stress conditions, 55 days from planting.

| | Event-1 | | | | | Event-2 | | | | |
|---|---|---|---|---|---|---|---|---|---|---|
| | | Nitrogen stress | | Water stress | | | Nitrogen stress | | Water stress | |
| Trait Name | No stress | Mild | Moderate | Mild | Moderate | No stress | Mild | Moderate | Mild | Moderate |
| Plant height | [symbol] | N/A | N/A | [symbol] | [symbol] | [symbol] | N/A | N/A | [symbol] | [symbol] |
| Biomass | [symbol] | N/A | N/A | [symbol] | [symbol] | [symbol] | N/A | N/A | [symbol] | [symbol] |
| Ear weight | [symbol] | [symbol] | [symbol] | 10.7 | [symbol] | [symbol] | [symbol] | [symbol] | [symbol] | [symbol] |
| Stover weight | [symbol] | [symbol] | [symbol] | [symbol] | 0 | [symbol] | [symbol] | [symbol] | [symbol] | [symbol] |
| Harvest index | [symbol] | [symbol] | [symbol] | [symbol] | [symbol] | [symbol] | [symbol] | [symbol] | [symbol] | [symbol] |
| Water applied | [symbol] | N/A | N/A | [symbol] | [symbol] | [symbol] | N/A | N/A | [symbol] | −2.3 |
| WUE | [symbol] | N/A | N/A | [symbol] | [symbol] | [symbol] | N/A | N/A | [symbol] | #z,279 |

As shown in Table 8, in comparison with the control, transgenic plants exhibited some enhanced traits related to stress resistance and maintained other positive traits under stress conditions. The plant height decreased significantly across all treatments and was not affected by stress condition. Biomass and canopy area were neutral in no-stress condition but increased in more severe stress conditions. The foliar water content increased significantly in no-stress and stress conditions, indicating that the transgenic plants retained more water in leaf tissues. The anthocyanin area decreased significantly in no-stress and stress conditions, indicating there was no nitrogen deficiency in the transgenic plants.

As shown in Table 9, in comparison with the control, transgenic plants exhibited significant decrease in the trait areas of Water Applied, WUE, biomass and stover weight, indicating that the transgenic plants had improved water use efficiency, with plants of lower biomass requiring less water. Harvest index increased significantly under non-stress and stress conditions.

### Example 11. Transgenic Plants Exhibited Increased Drought Tolerance, Stomatal Conductance, and Root Front Velocity at Reproductive Stages at Both Standard and High Density in the Field

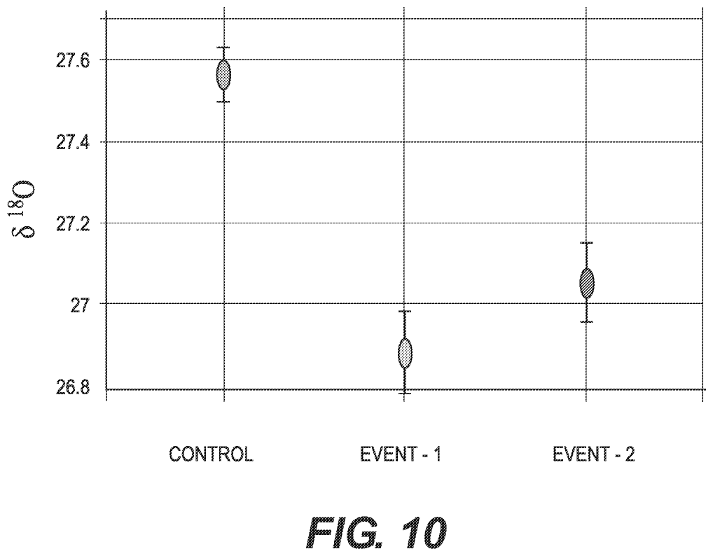

Direct observations were made of decreased leaf rolling in transgenic corn plants having the GA20 oxidase suppression construct from Example 1 under drought conditions in the field compared to control plants. Corn leaf rolling occurs when leaf water potential drops below a threshold of approximately −1.1 MPa. Stomatal conductance also decreases under water stress. Stable oxygen isotope ratios (δ¹⁸O) were used as an index of the stomatal conductance, which is inversely proportional to stomatal conductance. A significant decrease of δ¹⁸O, and thus a significant increase in the stomatal conductance, in transgenic plants over the control was observed from ear leaf samples collected at R5 stage (see FIG. 10). Data was taken from transgenic plants across two transformation events and averaged across 10 testers with 2 reps per tester. Increased δ¹⁸O in the leaf of control plants indicates that stomatal conductance was lower for the control. In conjunction with the reduced leaf rolling observed in the field, the significant increase of stomatal conductance in leaves of transgenic plants from yield trials at 15 out of 16 field locations indicates improved leaf water status during late vegetative growth for the transgenic plants.

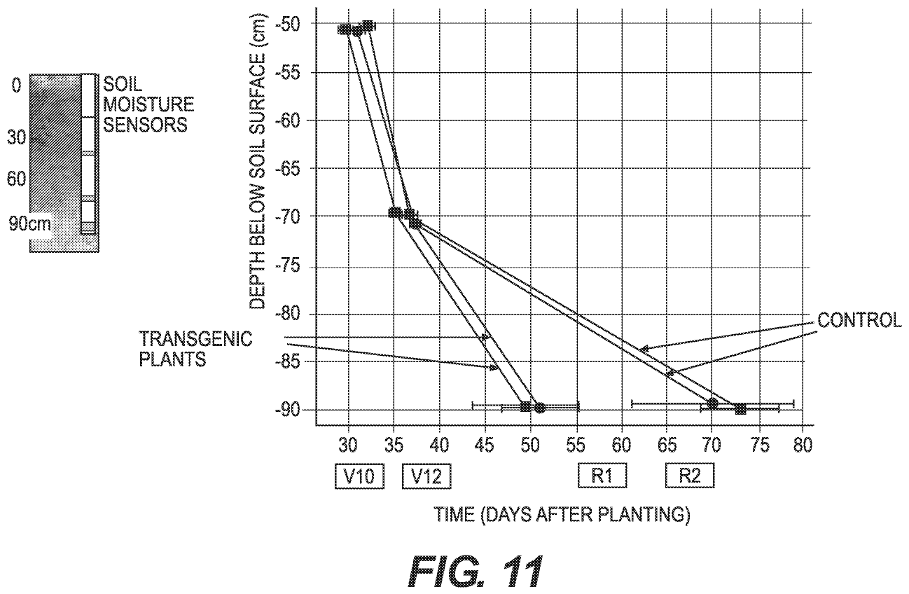

Effective water uptake by the roots is an important factor in plant growth. To measure the developmental progress of rooting depth, Sentek® SOLO soil moisture capacitance probes were installed at V4 stage within the row between plants at one field location. Soil moisture was measured on an hourly basis with capacitance sensors at depths of 10, 20, 30, 50, 70, 90, 120, and 150 cm from the ground level. The depth of the rooting front was inferred by the presence of diurnal patterns in soil moisture depletion recorded by the sensors. Root activity was already present at 10, 20, and 30 cm depth at the time of installation at V4 stage. We detected the first occurrence of soil moisture depletion at 50, 70, and 90 cm depths. The soil at 120 and 150 cam depth was saturated throughout the growing season. While root growth may have reached these depths, we were not able to detect root activity at these depths for this experiment due to the inability to detect soil moisture depletion in a saturated zone. FIG. 11 shows the time (days after planting) for the frontal root of the plant to reach various depths on the Y axis. Lines with circles are for plants at 30-inch row spacing and 34,000 plants per acre planting density, and lines with squares are for plants at 20-inch row spacing, and 55,000 plants per acre planting density. Growth stages are shown on the X axis.

As shown in FIG. 11, root growth was similar in this experiment between transgenic and control plants up to V12, with roots reaching 50 and 70 cm depth at about 30 and 36 days after planting, respectively. However, the transgenic plant roots reached 90 cm depth at or before R1 (i.e., at about day 50 after planting), or about 20 days earlier than control plant roots. The transgenic plants exhibited increased rooting front velocity after V11/V12 stage, which may lead to increased drought avoidance during the critical period of plant development around flowering. This increase in rooting front velocity may allow the transgenic plants to take advantage of deeper reserves of soil water during the critical period around R1 stage, possibly allowing drought effects on flowering and pollination to be avoided, reduced or minimized. Improved pollination under drought conditions may likely improve kernel set and yield potential.

To complement the above field experiment with moisture sensors, root front velocity for transgenic corn plants having the GA20 oxidase suppression construct from Example 1 (n=10) was measured in a root box experiment and compared to wild-type control plants (n=9). Plexiglass root boxes (5 feet tall and six-by-eight inches in cross section; ½ inch wall thickness) were filled with a mix of #10 field soil/vermiculite/perlite (1:1:1 ratio) and used for root visualization for each plant. Maximum rooting depth in each box was measured at regular intervals after planting (approximately every two days). In this experiment, median root front depth of transgenic plants was consistently greater or deeper than WT control plants starting at about 21 days after planting (i.e., at about V4 stage) and continuing until at least 34 days after planting when measurements were stopped (data not shown). This observation in controlled environment root boxes is consistent with the increased root depth observed with moisture sensors in the field and shows that deeper roots may occur at earlier developmental stages, although differences in root depth were not detected in the field experiment until after V11/V12 stage.

Although the root traits measured in the controlled environment experiments described in Example 14 below generally did not show a significant difference in root depth (or only a minimal difference), the vermiculite experiment in Example 14 was performed at V3 stage before the difference in root depth was observed in the root box experiment in this Example 11 (i.e., starting around V4 stage), and although the aeroponic apparatus experiment in Example 14 was performed at V5 stage, the aeroponic system does not have any plant-soil interaction (unlike the vermiculite experiments) that might affect normal (or more natural) root growth and development.

### Example 12. Transgenic Plants have Higher Stomatal Conductance in Normal and Drought Conditions and Maintain Higher Photosynthesis Capacity Under Drought Stress

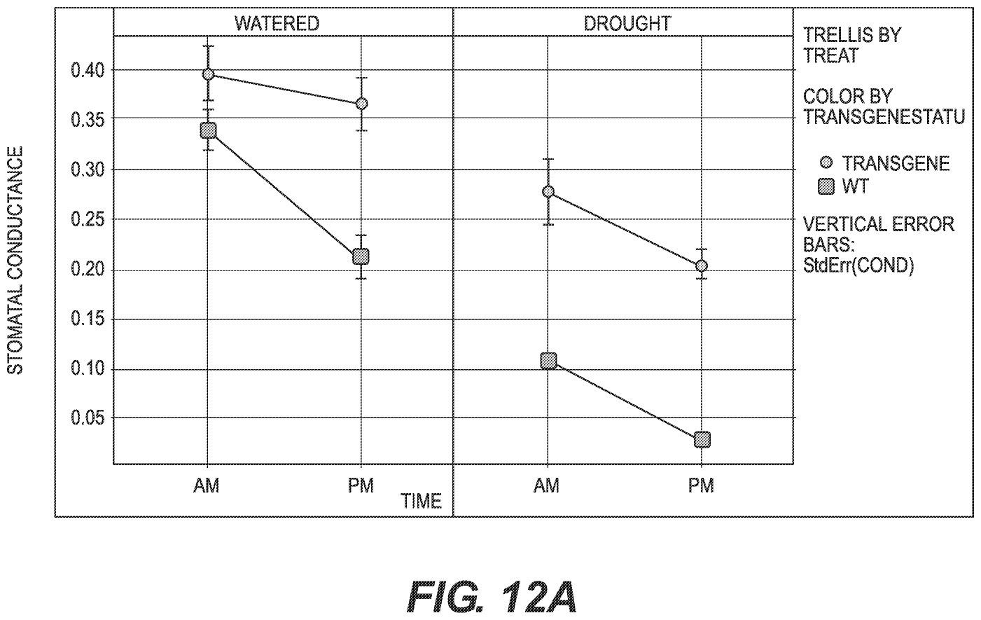

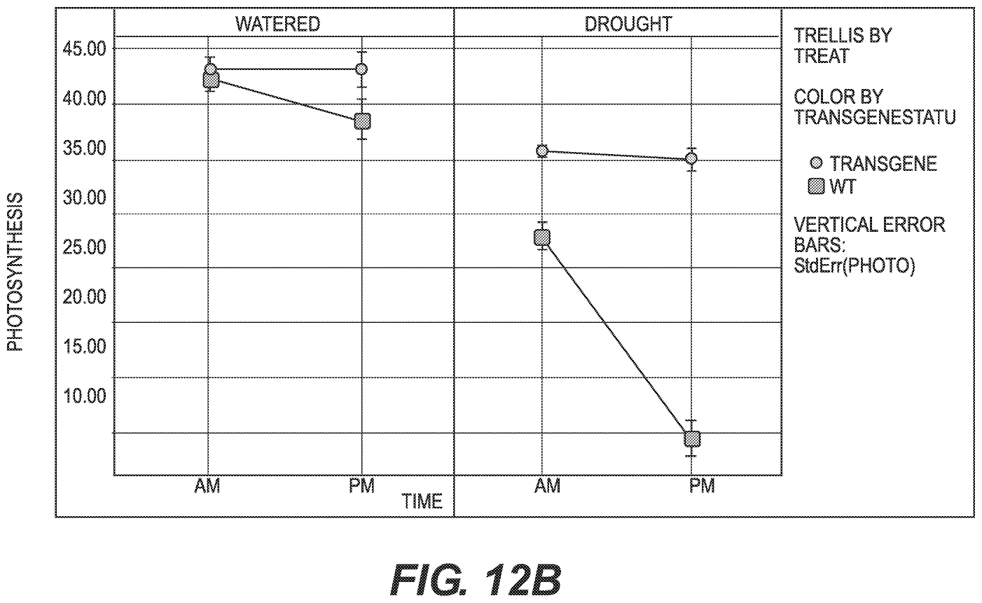

Stomatal conductance and photosynthesis levels in leaves under normal and drought conditions was also measured in the greenhouse. For this experiment, transgenic plants with the GA20 oxidase suppression construct from Example 1 and wild-type control plants were subjected to a well watered (1500 ml water per day) or limited water/chronic drought (1000 ml water per day) treatment. Twenty (20) reps of the wild-type control plants and ten (10) reps per event (two events total) for the GA20 oxidase suppression construct were subjected to the well watered treatment, and one-hundred and forty (140) reps of the wild-type control plants and seventy (70) reps per event (two events total) for the GA20 oxidase suppression construct were subjected to the limited water/chronic drought treatment. Border plants of appropriate height (hybrids for WT plants and inbreds for transgenic plants) were placed around the perimeter of the experimental plants in the greenhouse to normalize the effects of shading. Diurnal stomatal conductance and photosynthesis measurements were taken in the morning and afternoon with a LI-COR® device at V12 stage per manufacturer's instructions. As shown in FIG. 12A, stomatal conductance was found to be consistently higher for the transgenic plants under both well-watered and drought conditions at both daily time points. Transgenic plants were also observed to have less leaf rolling under the drought condition. As further shown in FIG. 12B, a higher photosynthesis rate was also observed in response to drought conditions that did not significantly respond to increased sunlight in the afternoon, unlike control plants that showed a drop in the rate of photosynthesis in the afternoon particularly under drought conditions.

These results (in combination with the separate field observations above) demonstrate that the transgenic plants with the GA20 oxidase suppression construct not only had higher gas exchange and photosynthesis in the leaf, but maintained a higher gas exchange and photosynthesis in the leaf in response to water limiting/chronic drought conditions. It was further observed that transgenic plants had a lower leaf temperature than control plants (data not shown). Thus, it is predicted that transgenic plants expressing a GA20 oxidase suppression construct may have greater drought tolerance and an ability to maintain photosynthesis under water limiting conditions as compared to controls. Without being bound by theory, it is further proposed that the deeper roots observed for transgenic plants with the GA20 oxidase suppression construct (particularly during late vegetative and early reproductive stages) may contribute to the drought tolerance of these transgenic plant.

### Example 13. Transgenic Plants Exhibited Reproductive Traits Comparable to Those of the Control in Greenhouse Conditions

Transgenic corn plants having the GA20 oxidase suppression construct described in Example 1 and control plants were grown in pots in the greenhouse to reproductive R1 stage, and reproductive traits were measured in V8 and R1 stages. Data were taken for transgenic plants of two transformation events (Table 10). The data are provided either in terms of a difference in the number of days, or as a percent difference, for the transgenic plants as compared to a wild-type control, and significant changes are in bold. Trait names are defined in Examples 9 and 10 above. Specific observations of the traits and trait classes of flowering, immature ear, mature ear and tassel are summarized in the table. Overall, reproductive development in transgenic plants was nearly equivalent to control plants with only a few slight or minor changes.

TABLE 10
Greenhouse reproductive traits of transgenic plants vs control.

| Class | Trait | Event-1 | Event-2 | Observations |
|---|---|---|---|---|
| Development | Plant Height | −17.60% | −14% | Shorter plant |
| (R1) | Leaf Tip | 2% | 1.10% | Slight increase in leaf numbers (0.3) |
| | Number | | | |
| Flowering | Days to 50% | −0.4 day | −0.5 day | Slightly delayed pollen shedding time with |
| (R1) | Silking and 50% | | | normal silking time; lower ASI |
| | Pollen Shed | | | |
| | Days to 50% | 1.10% | 1.10% | |
| | Pollen | | | |
| | Shedding | | | |
| | Days to 50% | 0.40% | −0.10% | |
| | Visible Silk | | | |
| Immature | Immature Ear | −28.50% | −22.60% | Slower initiation of ear development |
| Ear (V8) | Diameter at | | | |
| | base | | | |
| | Immature Ear | −6.10% | −4.20% | |
| | Internode | | | |
| | Length | | | |
| | Immature Ear | −42% | −31% | |
| | Length | | | |
| | Immature | −38.70% | −29.70% | |
| | Kernels/Row | | | |
| | Longitudinally | | | |
| Mature Ear | Kernels/Row | −1% | −0.40% | Properly developed mature ear |
| (R1) | Longitudinally | | | |
| | Kernel Row | 2.20% | 0% | |
| | Number | | | |
| | Total floret | 1.10% | −0.50% | |
| | number | | | |
| | Shank | −3.60% | 0.10% | |
| | internode | | | |
| | number | | | |
| Tassel (R1) | Number of | −5.40% | −3.80% | Properly developed tassel but with shorter |
| | Tassel Branches | | | first internode |
| | Primary Lateral | −10.40% | −9.10% | |
| | Tassel Branch | | | |
| | Number | | | |
| | Secondary | −17.60% | −13.70% | |
| | Lateral Tassel | | | |
| | Branch Number | | | |
| | Rachilla Floret | −8.50% | −0.40% | |
| | Density | | | |
| | First Tassel | −34.10% | −32.70% | |
| | Internode | | | |
| | Length | | | |

### Example 14. Root Traits of Transgenic and Control Plants in Greenhouse Conditions

Transgenic plants having the GA20 oxidase suppression construct described in Example 1 and control plants were grown in the greenhouse in vermiculite medium to V3 stage or in an aeroponic apparatus to V5 stage. Plants were extracted and roots washed for direct or optical imaging measurements of the root traits. Transgenic plants of 4 transformation events were tested in comparison to a control. Measurement results are summarized in Table 11 and 12 for plants from vermiculite medium growth, or in the aeroponic growth apparatus, respectively. Root Branch Point Number measures the number of root branch tip points of a plant through imaging of the plant root. The root system image was skeletonized for the root length measurement. Up to 40 images were taken at various angles around the root vertical axis and the measurement was averaged over the images. Root Total Length measures the cumulative length of roots of a plant, as if the roots were all lined up in a row, through imaging of the root system of the plant. The root system image was skeletonized for the root length measurement. Up to 40 images were taken at various angles around the root vertical axis and measurement was averaged over the images. Data in Tables 11 and 12 are the percent delta difference of the transgenic plants in comparison to the control with significant changes presented in bold.

TABLE 11
Greenhouse root traits of transgenic plants vs control at V3, in
vermiculite medium

| | Event-1 | Event-2 | Event-3 | Event-4 |
|---|---|---|---|---|
| Average Root Diameter | [symbol] | [symbol] | [symbol] | −5.9 |
| Root Branch Point Number | [symbol] | 5.8 | [symbol] | −0.4 |
| Root Dry Weight | 1 | −5.6 | −7.1 | −5 |
| Root Surface Area | 2.2 | −6.2 | −6 | 0 |
| Root Total Length | [symbol] | −1.9 | 3 | 1.4 |
| Plant Height | [symbol] | [symbol] | [symbol] | [symbol] |
| Shoot Dry Weight | −3.6 | −2 | −4.5 | −7.7 |
| Shoot to Root Ratio | −1.5 | 3.4 | 1.8 | −3.4 |

TABLE 12
Greenhouse root traits of transgenic plants vs control at V5, in
aeroponic apparatus.

| | Event-1 | Event-2 | Event-3 | Event-4 |
|---|---|---|---|---|
| Root Branch Point Number | −6.18 | 5.01 | 5.63 | 6.38 |
| Root Total Length | −1.47 | 5.43 | 2.46 | 6.92 |
| Average Root Width | −1.12 | −5.05 | −5.23 | −3.56 |
| Root Volume | −1.1 | −4.21 | −8.47 | −1.09 |
| Root Dry Weight | 5.21 | −7.51 | −2.61 | 4.52 |
| Root Surface Area | −1.51 | 0.93 | −2.71 | 3.06 |
| Plant Height | [symbol] | [symbol] | [symbol] | [symbol] |
| Shoot Dry Weight | [symbol] | [symbol] | [symbol] | [symbol] |
| Total Dry Weight | −4.41 | [symbol] | −8.54 | −3.24 |
| Shoot/Root Ratio | [symbol] | [symbol] | [symbol] | [symbol] |

As shown in Tables 11 and 12, the transgenic plants exhibited significant decrease in plant heights at V3 and V5 stages, but only minor variations in the overall root architecture were observed in these experiments between transgenic and control plants.

### Example 15. Phenotypic Observations of Transgenic Plants with Alternate Promoters

In Examples 1 through 14, transgenic plants contained a GA20 oxidase suppression element operably linked to an RTBV promoter. Corn plants were also transformed with the same suppression element operably linked to various other promoters, to test how different patterns of expression of the GA20 oxidase suppression element might affect plant height and other phenotypes.

Transgenic plants (R0 plants) regenerated from explants transformed with constructs operably linked to various promoters were observed at R5 growth stage in the greenhouse, and the ears were observed after being peeled back for dry down. The various promoters tested are identified in Table 13. Observations were made for plants of multiple transformation events for each construct containing a different promoter in comparison to control plants of the same breeding line without the GA20 oxidase suppression construct. The results of these observations are summarized in Table 13 across transformation events for each construct.

TABLE 13
Summary of R0 observations of transgenic
plants with a miRNA suppression
construct for GA20 oxidase under the control of different promoters.

| | | R0 plants |
|---|---|---|
| Promoter Name | Expression pattern | observations |
| RTBV promoter | vascular enhanced | short; no off type |
| CAMV e35S promoter | constitutive | some short (variable); |
| | | no off type |
| Coix lacryma-jobi | constitutive | some short (variable); |
| polyubiquitin promoter | | no off type |
| rice actin promoter | constitutive | some short (variable); |
| | | no off type |
| rice Gos2 promoter | constitutive | some short (variable); |
| | | no off type |
| Enhancer + RTBV | constitutive | short; no off type |
| promoter | | |
| C1 | constitutive | Short |
| corn PPDK promoter | leaf enhanced, high | mid-short; no off type |
| corn FDA promoter | leaf enhanced, medium | some short (variable); |
| | | no off type |
| rice Nadh-Gogat | leaf enhanced, low | mid-short; no off type |
| promoter | | |
| rice Cyp2 promoter | vascular enhanced | some short (variable); |
| | | no off type |
| V1 | vascular enhanced | short; no off type |
| V2 | vascular enhanced | normal height; |
| | | no off type |
| V3 | vascular enhanced | normal height; |
| | | no off-type |
| MMV.FLT promoter | stem enhanced, high | normal height; |
| | | no off-type |
| S1 | stem enhanced, medium | normal height; |
| | | no off-type |
| S2 | stem enhanced, medium | normal height; |
| | | no off-type |
| S3 | stem enhanced, medium | normal height; |
| | | no off-type |
| SETit.lfr promoter | root enhanced, high | mid-short; |
| | vascular enhanced | no off-type |
| Rice Rcc3 promoter | root enhanced, low | normal height; |
| | | no off-type |
| Rice Expb promoter | ear enhanced, high | normal height; |
| | | no off-type |
| Maize H2a promoter | ear enhanced, low | normal height; |
| | | no off-type |

As shown in Table 13, in comparison with controls, R0 transgenic plants with the GA20 oxidase suppression construct did not exhibit any significant off-types by observation for all of the promoters tested. Even expression directly in reproductive ear tissues did not cause any observable off-types. Plant heights were clearly decreased not only for the RTBV promoter construct (in the previous Examples), but also for transgenic plants having the same GA20 oxidase suppression construct operably linked to various constitutive promoters, leaf promoters at different expression levels, some vascular promoters, and a root promoter with a high expression level. An engineered promoter with constitutive expression (C1) linked to the GA20 oxidase suppression construct was tested and also found to cause a short stature phenotype. Similarly, at least one engineered promoter with vascular expression (V1) linked to the GA20 oxidase suppression construct was found to cause a short stature phenotype, in addition to the vascular rice Cyp2 promoter, although plants with two other engineered vascular promoters (V2, V3), and three engineered stem promoters (S1, S2, S3), did not have a reduced plant height. However, changing the transcriptional terminator sequence for the GA20 oxidase suppression construct under the control of the RTBV promoter did not alter the short stature phenotype (not shown in Table 11). As used herein, the term “mid-short” refers to a moderate reduction in plant height (relative to the reduction in plant height observed with the RTBV promoter), and an observation of “some short” means that there was some variation in the amount of reduction in plant height.

These results show that expression of the GA20 oxidase suppression element with constitutive promoters consistently produced a short stature phenotype, although there was some variability in the plant height phenotypes observed with these constitutive promoters. Likewise, a combination of the RTBV promoter with an enhancer element to convert the pattern of expression from vascular to constitutive still produced a short stature phenotype (indicating the sufficiency of the RTBV promoter). A few of the vascular promoters including the RTBV promoter produced a short stature phenotype, but a couple other engineered vascular promoters did not produce the short stature phenotype, which may be attributed to a lower expression level with these promoters. None of the stem promoters produced a short stature phenotype, indicating that expression of the GA20 suppression construct in the stem was not sufficient to produce this phenotype. Surprisingly, expression of the GA20 suppression construct in the leaf consistently produced short stature phenotypes with different levels of expression, although the results were somewhat variable. This data indicates that the production of active GAs in leaf tissue contributes to plant growth and ultimately plant height, even though such vertical growth occurs in the stem or stalk of the plant. Expression of the GA20 oxidase suppression construct with various root promoters generally did not produce a short stature phenotype, although one root promoter did produce a moderate phenotype, which may be due to additional expression in above-ground plant tissues.

R0 plants were then self-crossed and the resulting seeds were grown in the nursery to generate homozygous inbred progeny plants (R1 plants). Observations of R1 progeny transgenic plants with some of the promoter constructs (at least 4 transformation events per construct) were made at the R1 developmental stage, in comparison to control plants of the same breeding line without the GA20 oxidase suppression construct. Like the R0 plants, R1 progeny plants expressing the GA20 oxidase suppression construct with each of the RTBV, CAMV e35S, and Coix lacryma-jobi polyubiquitin promoters were also found to have a short stature, semi-dwarf phenotype without any significant off-types observed.

### Example 16. Phenotypic Observations of Transgenic Corn Plants with Constructs Targeting Different GA Oxidase Genes

The Examples above demonstrate that a miRNA-expressing construct targeting the GA20 oxidase_3 and GA20 oxidase_5 genes for suppression, and operably linked to a plant-expressible vascular, constitutive and/or leaf promoter, may be used to generate a short stature, semi-dwarf corn plant. To test how targeting different GA20 or GA3 oxidase genes, or different portions of the GA20 oxidase_3 and/or GA20 oxidase_5 genes, for suppression might affect plant height, several constructs were generated and transformed into corn plants. Constructs were also made with the same targeting sequence as in the above Examples, but with a different miRNA backbone sequence (two from corn miRNAs, one from a soybean miRNA, and one from a cotton miRNA—the construct in the above Examples used a rice miRNA backbone sequence). Table 14 provides a list of these additional suppression constructs, along with observations of transgenic R0 plants comprising these constructs in the greenhouse (in comparison to wild-type control plants). Constructs targeting (i) GA20 oxidase_1/GA20 oxidase_2, (ii) GA20 oxidase_3/GA20 oxidase_9, (iii) GA20 oxidase_1/GA20 oxidase_8, and (iv) GA20 oxidase_3/GA20 oxidase_5 (with different miRNA backbones), each encoded a miRNA with a single targeting sequence complementary to both gene targets, whereas the stacks of (i) the individual GA20 oxidase_3 and GA20 oxidase_5 targeting sequences, (ii) the individual GA20 oxidase_4 and GA20 oxidase_6 targeting sequences, and (iii) the individual GA20 oxidase_4 and GA20 oxidase_7/8 targeting sequences, were each expressed as a single pre-miRNA with the two targeting sequences arranged in tandem that become cleaved and separated into two mature miRNAs. Table 14 provides the miRNA targeting sequence and the cDNA sequence complementary to the miRNA targeting sequence. For the GA20 oxidase_1/GA20 oxidase_2 construct, the asterisk (*) indicates that the alignment length between the targeting sequence of the miRNA and the mRNA target or recognition site was shorter (17 vs. 20 nucleotides) for GA20 oxidase_1 than for GA20 oxidase_2. Similarly for the GA20 oxidase_3/GA20 oxidase_9 construct, the asterisk (*) indicates that the alignment length between the targeting sequence of the miRNA and the mRNA target or recognition site was shorter (17 vs. 20 nucleotides) for GA20 oxidase_9 than for GA20 oxidase_3. For each of the constructs listed in Table 14, no significant off-types were observed, apart from the observations provided in the table.

TABLE 14
Summary of R0 observations of transgenic plants with miRNA suppression
constructs targeting different GA oxidase genes.

| | | cDNA | | |
|---|---|---|---|---|
| | | Target | miRNA | |
| | mRNA | Sequence | Targeting | |
| Targeted Gene(s) | Targeted | (SEQ ID | Sequence | |
| (Construct/Promoter) | Area | NO) | (SEQ ID NO) | Observations |
| GA20 oxidase_1 and | 1: exon* | 47 | 48 | All events tall (WT) |
| GA20 oxidase_2 | 2: exon | | | |
| (RTBV promoter) | | | | |
| GA20 oxidase_3 and | 3: exon | 49 | 50 | All events tall (WT) |
| GA20 oxidase_9 | 9: exon* | | | |
| (RTBV promoter) | | | | |
| GA20 oxidase_7 and | exon | 51 | 52 | All events - tall (WT) |
| GA20 oxidase_8 | | | | |
| (RTBV promoter) | | | | |
| GA20 oxidase_3 | UTR | 53 | 54 | Events slightly shorter |
| (Individual; RTBV | | | | (~6 inches vs. WT) |
| and 35S promoter) | | | | |
| GA20 oxidase_5 | UTR | 55 | 56 | All events - tall (WT) |
| (Individual; RTBV | | | | |
| and 35S promoter) | | | | |
| GA20 oxidase_3 and | 3: UTR | 53 | 54 | All events - shorter |
| GA20 oxidase_5 | 5: UTR | 55 | 56 | |
| (Individuals; Tandem stack) | | | | |
| GA20 oxidase_3 and | 3/5: | 39 | 40 | All events/constructs - |
| GA20 oxidase_5 | exons | | | shorter |
| (Different miRNA | | | | |
| backbones) | | | | |
| (RTBV promoter) | | | | |
| GA3 oxidase_1 | UTR | 57 | 58 | All events - tall (WT) |
| (RTBV promoter) | | | | (only 3 events observed) |
| GA3 oxidase_1 | UTR | 57 | 58 | Some events - shorter |
| (CAMV e35S promoter) | | | | |
| GA3 oxidase_2 | exon | 59 | 60 | All events - shorter |
| (RTBV promoter) | | | | (darker green leaves) |
| GA3 oxidase_2 | exon | 59 | 60 | Some events - shorter |
| (CAMV e35S promoter) | | | | |
| GA20 oxidase_4 and | 4: exon | 61 | 62 | Some events - |
| GA20 oxidase_6 | 6: exon | 63 | 64 | moderately shorter (−20%) |
| (Individuals; Tandem stack) | | | | |
| GA20 oxidase_4 and | 4: exon | 61 | 62 | Some events - |
| GA20 oxidase_7/8 | 7/8: exon | 51 | 52 | moderately shorter (−20%) |
| (Individuals; Tandem stack) | | | | |

The observations summarized in Table 14 demonstrate that targeting of several other GA20 oxidase genes did not produce a short stature, semi-dwarf phenotype. None of the constructs targeting (i) the related GA20 oxidase_1 and GA20 oxidase_2 genes, (ii) the related GA20 oxidase_3 and GA20 oxidase_9 genes, (iii) the related GA20 oxidase_7 and GA20 oxidase_8 genes, or (iv) the GA20 oxidase_9 gene alone produced an observable short stature, semi-dwarf phenotype in R0 plants. In contrast, those constructs encoding a single miRNA jointly targeting the GA20 oxidase_3 and GA20 oxidase_5 genes in transgenic R0 and R1 plants did produce a short stature, semi-dwarf phenotype, even if a different transcriptional termination sequence or different miRNA backbones are used (total of 5 miRNA backbone sequences tested). In addition, targeting different sequences of the GA20 oxidase_3 and GA20 oxidase_5 genes still produced semi-dwarf plants. Interestingly, suppression constructs that were designed to target either of the GA20 oxidase_3 and GA20 oxidase_5 genes individually did not produce a short stature, semi-dwarf phenotype, unlike constructs jointly targeting the GA20 oxidase_3 and GA20 oxidase_5 genes, although the construct individually targeting the GA20 oxidase_3 gene did produce a slight reduction in plant height. However, transgenic plants having a tandem vector stack of the suppression constructs individually targeting the GA20 oxidase_3 and GA20 oxidase_5 genes did produce a short stature, semi-dwarf phenotype similar to constructs encoding a single miRNA jointly targeting the GA20 oxidase_3 and GA20 oxidase_5 genes. These data demonstrate that a short stature, semi-dwarf phenotype is observed with constructs targeting both of the GA20 oxidase_3 and GA20 oxidase_5 genes, but the full semi-dwarf phenotype is not observed with targeting of the GA20 oxidase_3 and GA20 oxidase_5 genes individually for suppression (only a slight reduction in height with targeting GA20 oxidase_3, and no plant height phenotype observed with targeting GA20 oxidase_5). Moreover, no plant height phenotype was observed with targeting the GA20 oxidase_1, GA20 oxidase_2, GA20 oxidase_6, GA20 oxidase_7, GA20 oxidase_8, and/or GA20 oxidase_9 gene(s) as described.

Apart from the GA20 oxidase_3 and GA20 oxidase_5 genes, a moderate reduction in plant height was observed in R0 transgenic plants with a suppression construct comprising two targeting sequences in tandem complementary to jointly target (i) the GA20 oxidase_4 and GA20 oxidase_6 genes, or (ii) the GA20 oxidase_4, GA20 oxidase_7 and GA20 oxidase_8 genes—one of the two targeting sequences targets both the GA20 oxidase_7 and GA20 oxidase_8 genes. Given that a separate construct that targets the GA20 oxidase_7 and GA20 oxidase_8 genes did not produce a plant height phenotype, and the suppression construct targeting the GA20 oxidase_4 and GA20 oxidase_6 genes produced a plant height phenotype that was similar to the suppression construct targeting the GA20 oxidase_4, GA20 oxidase_7 and GA20 oxidase_8 genes, it is believed that targeting of the GA20 oxidase_4 gene is largely (if not fully) responsible for the plant height phenotype observed in these transgenic plants. Furthermore, transgenic corn plants with constructs targeting the GA3 oxidase_1 or GA3 oxidase_2 genes also displayed a reduction in plant height, although there was some variability in this phenotype depending on the constitutive promoter. Thus, in addition the GA20 oxidase_3 and GA20 oxidase_5 genes, the GA20 oxidase_4, GA3 oxidase_1, and GA3 oxidase_2 genes may also be targeted for suppression to produce short stature, semi-dwarf plants.

### Example 17. Phenotypic Observations of Corn Plants Having an Edited GA20 Oxidase_3 or GA20 Oxidase_5 Gene

In addition to the above suppression constructs, several genome-edited mutations were created in the endogenous GA20 oxidase_3 and GA20 oxidase_5 genes in corn plants to test for the phenotypic effect of knocking out each of these genes. A series of ten single-chain guide RNA (sgRNAs) encoding targeting constructs were created for each of the GA20 oxidase_3 and GA20 oxidase_5 genes that target different positions along the genomic sequence for each gene. An additional series of ten sgRNAs were created that each target both of the GA20 oxidase_3 and GA20 oxidase_5 genes, at similar or different positions along the genomic sequence for each gene. Targeted genome edits were made by delivering the sgRNA along with expression of a Cas9 protein to corn explants to cause a DSB or nick to occur at or near the genomic target site for the gRNA, which may then be imperfectly repaired to introduce a mutation at or near the target site. The presence of a mutation was subsequently confirmed by RFLP analysis and/or sequencing of plants. Table 15 below provides a list of the guide RNA (gRNA) constructs that were tested, which may be used for genome editing of one or both of the GA20 oxidase_3 and GA20 oxidase_5 gene(s) with a RNA-guided endonuclease. These guide RNA constructs are generally designed to target the coding sequences of the GA20 oxidase_3 and/or GA20 oxidase_5 genes, but some of the joint targeting constructs may instead target a UTR sequence of one of the two genes. These gRNAs may be used with a suitable endonuclease to produce a double stranded break (DSB) or nick in the genome at or near the genomic target site of the respective gRNA, which may be imperfectly repaired to produce a mutation (e.g., an insertion, deletion, substitution, etc.). homozygous for an edited GA20 oxidase_3 gene or homozygous for an edited GA20 oxidase_5 gene were generated from a few of the constructs (see bold text). Events were also generated from constructs targeting both genes for editing. For the constructs jointly targeting the GA20 oxidase_3 and GA20 oxidase_5 genes, the coding sequence (CDS) coordinates are provided in reference to one of the two genes as indicated in parenthesis. Table 15 further shows which constructs produced gene editing events, whether those events were homozygous or heterozygous in the R0 plants, and the ±numbers in parenthesis indicate the likely sequence change with the mutation (e.g., +1 means an insertion of 1 nucleotide, −1 means a deletion of 1 nucleotide, etc., and larger or more complicated Indels are labeled “del.” or insert.”). For joint targeting of GA20 oxidase_3 and GA20 oxidase_5 genes, the identity of the mutated gene is also provided in parenthesis. Consistent with the results for the suppression constructs, transgenic R0 plants homozygous for an edited GA20 oxidase_3 or GA20 oxidase_5 gene did not have an observable short stature, semi-dwarf phenotype and had a normal plant height relative to control plants (See constructs GA20 oxidase_3-D and GA20 oxidase_3-G, and constructs GA20 oxidase_5-B and GA20 oxidase_5-G in Table 15), indicating that knockout of only one of these genes is not sufficient to produce the semi-dwarf phenotype.

TABLE 15
Guide RNAs (gRNAs) targeting GA20 oxidase_3 and
GA oxidase_5 genes for editing.

| | gRNA | | |
|---|---|---|---|
| | Targeting | | |
| | Sequence | | |
| | (SEQ | Gene CDS | |
| gRNA Gene Target | ID NO) | coordinates | R0 Plants Generated |
| GA20 oxidase_3-A | 138 | 552-572 | — |
| GA20 oxidase_3-B | 139 | 879-899 | — |
| GA20 oxidase_3-C | 140 | 147-167 | — |
| GA20 oxidase_3-D | 141 | 526-546 | 1. homozygous (−1) |
| | | | 2. heterozygous (−1) |
| | | | 3. bi-allelic (−2, +1) |
| GA20 oxidase_3-E | 142 | 446-466 | — |
| GA20 oxidase_3-F | 143 | 2227-2247 | — |
| GA20 oxidase_3-G | 144 | 548-568 | 1. homozygous (+1) |
| | | | 2. heterozygous (−1) |
| | | | 3. bi-allelic (+1, −1) |
| GA20 oxidase_3-H | 145 | 547-567 | — |
| GA20 oxidase_3-I | 146 | 43-63 | — |
| GA20 oxidase_3-J | 147 | 548-567 | — |
| GA20 oxidase_5-A | 148 | 356-376 (+) | 1. heterozygous (−1) |
| GA20 oxidase_5-B | 149 | 99-119 | 1. homozygous (−1) |
| | | | 2. heterozygous (+1) |
| | | | 3. heterozygous (+1, −7) |
| | | | 4. heterozygous (−3, −1) |
| GA20 oxidase_5-C | 150 | 369-389 | — |
| GA20 oxidase_5-D | 151 | 48-68 | — |
| GA20 oxidase_5-E | 152 | 356-376 (−) | — |
| GA20 oxidase_5-F | 153 | 748-768 | 1. heterozygous (−1, +1) |
| GA20 oxidase_5-G | 154 | 770-790 | 1. homozygous (−1) |
| | | | 2. homozygous (−1) |
| GA20 oxidase_5-H | 155 | 10-30 | — |
| GA20 oxidase_5-I | 156 | 262-282 | — |
| GA20 oxidase_5-J | 157 | 768-788 | — |
| GA20 oxidase_3/5-A | 158 | 290 . . . 310 | — |
| | | (GA20 Ox_3) | |
| GA20 oxidase_3/5-B | 159 | 289 . . . 309 | — |
| | | (GA20 Ox_3) | |
| GA20 oxidase_3/5-C | 160 | 270 . . . 290 | — |
| | | (GA20 Ox_5) | |
| GA20 oxidase_3/5-D | 161 | 49 . . . 69 | — |
| | | (GA20 Ox_3) | |
| GA20 oxidase_3/5-E | 162 | 265 . . . 285 | 1. heterozygous |
| | | (GA20 Ox_5) | (Ox5, +1) |
| GA20 oxidase_3/5-F | 163 | 419 . . . 439 | 1. hetero (Ox3, +1, −1) |
| | | (GA20 Ox_3) | hetero (Ox5, +1, del.) |
| | | | 2. hetero (Ox3, +1, del.) |
| | | | hetero (Ox5, +1) |
| GA20 oxidase_3/5-G | 164 | 110 . . . 130 | — |
| | | (GA20 Ox_3) | |
| GA20 oxidase_3/5-H | 165 | 634 . . . 654 | — |
| | | (GA20 Ox_5) | |
| GA20 oxidase_3/5-I | 166 | 98 . . . 118 | — |
| | | (GA20 Ox_5) | |
| GA20 oxidase_3/5-J | 167 | 517 . . . 537 | — |
| | | (GA20 Ox_5) | |

### Example 18. Identification of Corn Plants Having Various Combinations of Edited GA20 Oxidase_3 and GA20 Oxidase_5 Mutant Alleles

Corn plants were edited as described in Example 17 via a CRISPR/Cas9 based approach using guide RNAs (gRNAs) that target one of GA20 oxidase_3 and GA20 oxidase_5 genes specifically or target both of these two genes simultaneously (see Table 15). In total, 30 gRNA constructs were transformed into corn. Leaf samples from R0 plants were collected and analyzed for InDels by a Fragment Length Analysis (FLA) assay. Putative mutant alleles identified by FLA were sequenced using gene specific primers and standard deep sequencing protocols to confirmed the mutation(s). Table 16 provides a list of 12 edited mutant alleles in the GA20 oxidase_3 gene (ga20ox3-1 to ga20ox3-12) and their sequences. Table 17 provides a list of 11 edited mutant alleles in the GA20 oxidase_5 gene (ga20ox5-1 to ga20ox5-11) and their sequences. R0 plants with mutation(s) in either GA20 oxidase_3 or GA20 oxidase_5, or in both of those genes, were selfed to produce R1 plants.

R1 seeds from multiple R0 plants were planted and sampled again to confirm mutation(s) using FLA and standard sequencing protocols. Table 18 provides a list of R1 plants having mutations in GA20 oxidase_3, GA20 oxidase_5, or both genes. Table 18 also shows plant height and internode length (ear minus 2) of R1 plants measured at the R3 stage. Plant height were measured at R2/R3 growth stage from the soil line to the base of highest collared leaf. R1 plants that are homozygous or heterozygous for a mutation in the gene of interest (GA20 oxidase_3 and/or GA20 oxidase_5) were identified through sequencing and further selfed to produce R2 plants. Genotypes of the R2 plants were again determined by FLA and sequencing. Table 19 provides a list of R2 plants having mutations in GA20 oxidase_3, GA20 oxidase_5, or both genes, and their plant height at the R2/R3 stage. Table 19 also provides corresponding characterization of unedited reference control plants (wild-type inbred plants, shown as WT) and transgenic inbred corn plants having an artificial microRNA suppression construct targeting the GA20 oxidase_3 and GA20 oxidase_5 genes for suppression (SUP_GA20Ox3&Ox5 (“SUP_Ox3&Ox5”), as described in Example 1).

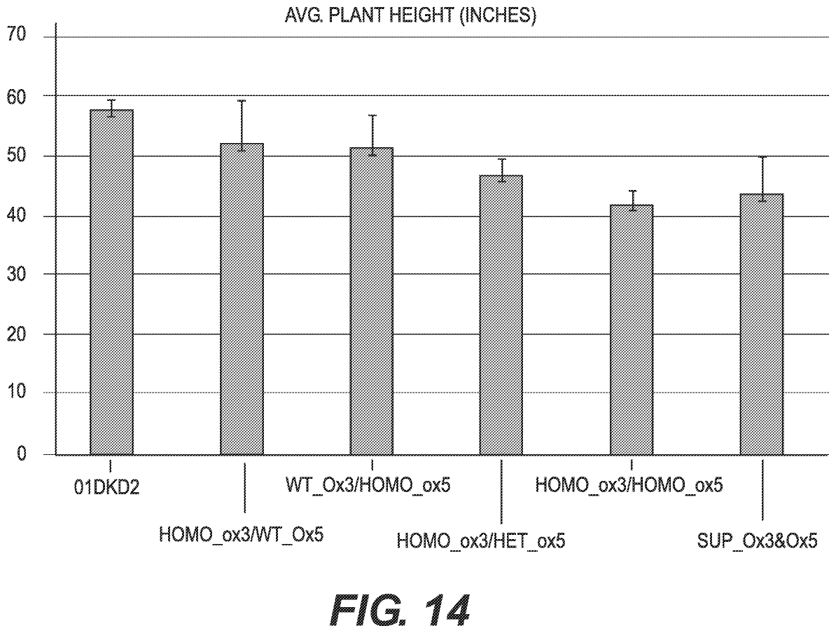

On average, R2 plants containing homozygous mutant alleles of both GA20 oxidase_3 and GA20 oxidase_5 genes (i.e., double homozygous) showed a semi-dwarf phenotype (about 27.5% reduction in plant height relative to control) with altered plant architecture similar to SUP_Ox3&Ox5 plants (comparing Homo_ox3/Homo_ox5 and SUP_Ox3&Ox5 plants in Table 20 and FIG. 14). Homozygous single ga20ox3 mutants and homozygous single ga20ox5 mutants exhibited a slight reduction (about 10-11%) in average plant height (at the R3 stage) relative to unedited reference control plants (WT inbred). In addition, corn plants with homozygous ga20ox3 mutations and heterozygous for a ga20ox5 mutation (i.e., Homo_ox3/Het_ox5 in Table 20 and FIG. 14) exhibited a moderate reduction (about 19.1%) in average plant height (at the R3 stage) relative to unedited reference control plants (WT inbred). Homo_ox3/Het_ox5 plants were slightly taller than double homozygous ga20ox3 ga20ox5 plants (Homo_ox3/Homo_ox5). Given that corn is a diploid organism, CRISPR-mediated gene editing can result in biallelic mutations in R0 plants (also known as a biallelic mutant combination or transheterozygous mutations). For simplicity, a biallelic mutant at a particular locus is treated as a homozygous mutant at that locus for genotype description and plant height calculation purposes. Detailed mutant genotypes (including biallelic mutants) are provided in Tables 18 and 19 for R1 and R2 generation plants, respectively. Both double homozygous ga20ox3/ga20ox5 mutants, and homozygous/heterozygous mutant combinations (e.g., Homo_ox3/Het_ox5 or Het_ox3/Homo ox5) also resulted in shorter, semi-dwarf plants, although plant heights in homozygous/heterozygous mutant combinations were not reduced as much as the double homozygous ga20ox3/ga20ox5 mutant plants.

TABLE 16
A list of 12 edited mutant alleles in GA20 oxidase_3 (ga20ox3-1 to ga20ox3-12)
and their sequences. The gRNA IDs shown here correspond to those in Table 15.

| | | SEQ ID | SEQ ID | SEQ ID | SEQ ID | | | |
|---|---|---|---|---|---|---|---|---|
| | | for | for | for | for | | | |
| | | Mutant | Mutant | Wild-type | Mutant | | | |
| | | Allele | Allele | Reference | Allele | | | |
| | | Sequence | Sequence | Sequence | Sequence | | | |
| | | (~30 nt | (~60 nt | (~60 nt | (genomic | Allele Description (EDIT @ | | |
| Gene | Mutant | flanking | flanking | flanking | coding | genomic coding DNA coordinate, | Edit | |
| Locus | allele | edits) | edits) | edits) | DNA) | based on SEQ ID No. 168) | Position | gRNA ID |
| GA20ox3 | ga20ox3-1 | 170 | 182 | 194 | 206 | Deletion of 13 bases starting at 536 | first exon | GA20ox3_d |
| GA20ox3 | ga20ox3-2 | 171 | 183 | 195 | 207 | Deletion of base 542 | first exon | GA20ox3_d |
| GA20ox3 | ga20ox3-3 | 172 | 184 | 196 | 208 | Insertion of CC at base 542 | first exon | GA20ox3_d |
| GA20ox3 | ga20ox3-4 | 173 | 185 | 197 | 209 | Deletion of base 541 | first exon | GA20ox3_d |
| GA20ox3 | ga20ox3-5 | 174 | 186 | 198 | 210 | Deletion of 3 nt starting at base 540 | first exon | GA20ox3_d |
| GA20ox3 | ga20ox3-6 | 175 | 187 | 199 | 211 | Deletion of 2 bases starting at base | first exon | GA20ox3_5_f |
| | | | | | | 422 | | |
| GA20ox3 | ga20ox3-7 | 176 | 188 | 200 | 212 | Insertion of an A at base 422 | first exon | GA20ox3_5_f |
| GA20ox3 | ga20ox3-8 | 177 | 189 | 201 | 213 | Insertion of a T at base 422 | first exon | GA20ox3_5_f |
| GA20ox3 | ga20ox3-9 | 178 | 190 | 202 | 214 | Deletion of base 564 | first exon | GA20ox3_g |
| GA20ox3 | ga20ox3-10 | 179 | 191 | 203 | 215 | Insertion of an A at base 564 | first exon | GA20ox3_g |
| GA20ox3 | ga20ox3-11 | 180 | 192 | 204 | 216 | Insertion of a C at base 565 | first exon | GA20ox3_g |
| GA20ox3 | ga20ox3-12 | 181 | 193 | 205 | 217 | Insertion of a C at base 63 | first exon | GA20ox3_5_e |

TABLE 17
A list of 11 edited mutant alleles in GA20 oxidase_5 (ga20ox5-1 to ga20ox5-11)
and their sequences. The gRNA IDs shown here correspond to those in Table 15.

| | | SEQ ID | SEQ ID | SEQ ID | SEQ ID | | | |
|---|---|---|---|---|---|---|---|---|
| | | for | for | for | for | | | |
| | | Mutant | Mutant | Wild-type | Mutant | | | |
| | | Allele | Allele | Reference | Allele | | | |
| | | Sequence | Sequence | Sequence | Sequence | | | |
| | | (~30 nt | (~60 nt | (~60 nt | (genomic | Allele description (EDIT @ | | |
| Gene | Mutant | flanking | flanking | flanking | coding | genomic coding DNA coordinate, | Edit | |
| Locus | Allele | edits) | edits) | edits) | DNA) | based on SEQ ID No. 169) | Position | gRNA ID |
| GA20ox5 | ga20ox5-1 | 218 | 229 | 240 | 251 | Deletion of base 644 | first exon | GA20ox3_5_f |
| GA20ox5 | ga20ox5-2 | 219 | 230 | 241 | 252 | Deletion of 2 bases starting at | first exon | GA20ox3_5_f |
| | | | | | | base 644 | | |
| GA20ox5 | ga20ox5-3 | 220 | 231 | 242 | 253 | Insertion of a T at base 644 | first exon | GA20ox3_5_f |
| GA20ox5 | ga20ox5-4 | 221 | 232 | 243 | 254 | Deletion of base 372 | first exon | GA20ox5_a |
| GA20ox5 | ga20ox5-5 | 222 | 233 | 244 | 255 | Deletion of base 786 | first exon | GA2ox5_g |
| GA20ox5 | ga20ox5-6 | 223 | 234 | 245 | 256 | Deletion of 5 bases starting at | first exon | GA2ox5_g |
| | | | | | | base 786 | | |
| GA20ox5 | ga20ox5-7 | 224 | 235 | 246 | 257 | Deletion of 2 bases starting at | first exon | GA20ox5_b |
| | | | | | | base 101 | | |
| GA20ox5 | ga20ox5-8 | 225 | 236 | 247 | 258 | Insertion of a T at base base | first exon | GA20ox5_b |
| | | | | | | 102 | | |
| GA20ox5 | ga20ox5-9 | 226 | 237 | 248 | 259 | Deletion of 3 bases starting at | first exon | GA20ox5_b |
| | | | | | | base 99 | | |
| GA20ox5 | ga20ox5-10 | 227 | 238 | 249 | 260 | Insertion of an A at base 282 | first exon | GA20ox3_5_e |
| GA20ox5 | ga20ox5-11 | 228 | 239 | 250 | 261 | Insertion of a C at base 282 | first exon | GA20ox3_5_e |

TABLE 18
A list of R1 plants having mutations in GA20 oxidase_3, GA20 oxidase_5, or both genes.

| | | Plant | Internode | | | | | | |
|---|---|---|---|---|---|---|---|---|---|
| Plant | Plant | Height | Length | GA20ox3 | GA20ox5 | ga20ox3 | ga20ox5 | Gener- | |
| No. | Genotype | (inches) | (cm) | Genotype | Genotype | Allele(s) | Allele(s) | ation | gRNA |
| 1 | single homo | 58.74 | 12 | WT | Biallelic | none | ga20ox5-6, | R1 | GA20ox5_g |
| | ga20ox5 | | | | deletion −1, | | ga20ox5-5 | | |
| | | | | | deletion −5, | | | | |
| 2 | single homo | 51.65 | 10 | WT | Biallelic | none | ga20ox5-6, | R1 | GA20ox5_g |
| | ga20ox5 | | | | deletion −1, | | ga20ox5-5 | | |
| | | | | | deletion −5, | | | | |
| 3 | single homo | 57.49 | 10.5 | WT | Biallelic | none | ga20ox5-6, | R1 | GA20ox5_g |
| | ga20ox5 | | | | deletion −1, | | ga20ox5-5 | | |
| | | | | | deletion −5, | | | | |
| 4 | single homo | 68.27 | 13.8 | WT | Biallelic | none | ga20ox5-8, | R1 | GA20ox5_b |
| | ga20ox5 | | | | deletion −2, | | ga20ox5-7 | | |
| | | | | | insertion +1, | | | | |
| 5 | single homo | 56.89 | 11.3 | WT | Biallelic | none | ga20ox5-6, | R1 | GA20ox5_g |
| | ga20ox5 | | | | deletion −5, | | ga20ox5-5 | | |
| | | | | | deletion −1, | | | | |
| 6 | hetero ga20ox3/ | 53.7 | 10 | Het insertion +1, | Biallelic | ga20ox3-12 | ga20ox5-11, | R1 | GA20ox3_5_e |
| | homo ga20ox5 | | | | insertion +1, | | ga20ox5-10 | | |
| | | | | | insertion +1, | | | | |
| 7 | homo ga20ox3/ | 53.9 | 9.5 | Biallelic | Het deletion −1, | ga20ox3-8, | ga20ox5-1 | R1 | GA20ox3_5_f |
| | hetero ga20ox5 | | | insertion +1, | | ga20ox3-7 | | | |
| | | | | insertion +1, | | | | | |
| 8 | homo ga20ox3/ | 56.69 | 10 | Biallelic | Het deletion −2, | ga20ox3-6, | ga20ox5-2 | R1 | GA20ox3_5_f |
| | hetero ga20ox5 | | | deletion −2, | | ga20ox3-8 | | | |
| | | | | insertion +1, | | | | | |
| 9 | homo ga20ox3/ | 49.09 | 9.5 | Biallelic | Het deletion −2, | ga20ox3-6, | ga20ox5-2 | R1 | GA20ox3_5_f |
| | hetero ga20ox5 | | | insertion +1, | | ga20ox3-8 | | | |
| | | | | deletion −2, | | | | | |
| 10 | homo ga20ox3/ | 54.96 | 10.3 | Biallelic | Het deletion −2, | ga20ox3-6, | ga20ox5-2 | R1 | GA20ox3_5_f |
| | hetero ga20ox5 | | | insertion +1, | | ga20ox3-8 | | | |
| | | | | deletion −2, | | | | | |
| 11 | homo ga20ox3/ | 47.13 | 9 | Homozygous | Het deletion −2, | ga20ox3-8 | ga20ox5-2 | R1 | GA20ox3_5_f |
| | hetero ga20ox5 | | | insertion +1, | | | | | |
| 12 | hetero ga20ox3/ | 53.31 | 10 | Het insertion +1, | Het deletion −5, | ga20ox3-10 | ga20ox5-6 | R1 | GA20ox3_g |
| | hetero ga20ox5 | | | | | | | | |
| 13 | homo ga20ox3/ | 56.57 | 11 | Biallelic | Het insertion +1, | ga20ox3-8, | ga20ox5-3 | R1 | GA20ox3_5_f |
| | hetero ga20ox5 | | | insertion +1, | | ga20ox3-7 | | | |
| | | | | insertion +1, | | | | | |
| 14 | homo ga20ox3/ | 49.57 | 8.1 | Biallelic | Het insertion +1, | ga20ox3-8, | ga20ox5-3 | R1 | GA20ox3_5_f |
| | hetero ga20ox5 | | | insertion +1, | | ga20ox3-7 | | | |
| | | | | insertion +1, | | | | | |
| 15 | homo ga20ox3/ | 53.35 | 9.1 | Biallelic | Het insertion +1, | ga20ox3-8, | ga20ox5-3 | R1 | GA20ox3_5_f |
| | hetero ga20ox5 | | | insertion +1, | | ga20ox3-7 | | | |
| | | | | insertion +1, | | | | | |
| 16 | homo ga20ox3/ | 59.41 | 9.6 | Biallelic | Het insertion +1, | ga20ox3-7, | ga20ox5-3 | R1 | GA20ox3_5_f |
| | hetero ga20ox5 | | | insertion +1, | | ga20ox3-8 | | | |
| | | | | insertion +1, | | | | | |
| 17 | homo ga20ox3/ | 60.75 | 11 | Biallelic | Het insertion +1, | ga20ox3-7, | ga20ox5-3 | R1 | GA20ox3_5_f |
| | hetero ga20ox5 | | | insertion +1, | | ga20ox3-8 | | | |
| | | | | insertion +1, | | | | | |
| 18 | single homo | 51.54 | 10 | WT | Homozygous | none | ga20ox5-4 | R1 | GA20ox5_a |
| | ga20ox5 | | | | deletion −1, | | | | |
| 19 | single homo | 57.4 | 12.2 | WT | Homozygous | none | ga20ox5-4 | R1 | GA20ox5_a |
| | ga20ox5 | | | | deletion −1, | | | | |
| 20 | single homo | 58.9 | 11.5 | WT | Homozygous | none | ga20ox5-4 | R1 | GA20ox5_a |
| | ga20ox5 | | | | deletion −1, | | | | |
| 21 | single homo | 50.83 | 9 | WT | Homozygous | none | ga20ox5-5 | R1 | GA20ox5_g |
| | ga20ox5 | | | | deletion −1, | | | | |
| 22 | single homo | 55.08 | 10.5 | WT | Homozygous | none | ga20ox5-5 | R1 | GA20ox5_g |
| | ga20ox5 | | | | deletion −1, | | | | |
| 23 | single homo | 54.76 | 10 | WT | Homozygous | none | ga20ox5-5 | R1 | GA20ox5_g |
| | ga20ox5 | | | | deletion −1, | | | | |
| 24 | single homo | 56.54 | 10 | WT | Homozygous | none | ga20ox5-5 | R1 | GA20ox5_g |
| | ga20ox5 | | | | deletion −1, | | | | |
| 25 | single homo | 55.12 | 10.3 | WT | Homozygous | none | ga20ox5-5 | R1 | GA20ox5_g |
| | ga20ox5 | | | | deletion −1, | | | | |
| 26 | single homo | 55.47 | 9 | WT | Homozygous | none | ga20ox5-5 | R1 | GA20ox5_g |
| | ga20ox5 | | | | deletion −1, | | | | |
| 27 | single homo | 61.02 | 10 | WT | Homozygous | none | ga20ox5-5 | R1 | GA20ox5_g |
| | ga20ox5 | | | | deletion −1, | | | | |
| 28 | single homo | 48.62 | 7 | WT | Homozygous | none | ga20ox5-5 | R1 | GA20ox5_g |
| | ga20ox5 | | | | deletion −1, | | | | |
| 29 | single homo | 63.5 | 11.5 | WT | Homozygous | none | ga20ox5-5 | R1 | GA20ox5_g |
| | ga20ox5 | | | | deletion −1, | | | | |
| 30 | single homo | 60.28 | 10.5 | WT | Homozygous | none | ga20ox5-5 | R1 | GA20ox5_g |
| | ga20ox5 | | | | deletion −1, | | | | |
| 31 | single homo | 58.12 | 11.3 | WT | Homozygous | none | ga20ox5-5 | R1 | GA20ox5_g |
| | ga20ox5 | | | | deletion −1, | | | | |
| 32 | single homo | 51.89 | 12 | WT | Homozygous | none | ga20ox5-5 | R1 | GA20ox5_g |
| | ga20ox5 | | | | deletion −1, | | | | |
| 33 | single homo | 70.08 | 14 | WT | Homozygous | none | ga20ox5-9 | R1 | GA20ox5_b |
| | ga20ox5 | | | | deletion −3, | | | | |
| 34 | single homo | 55.55 | 9.8 | WT | Homozygous | none | ga20ox5-10 | R1 | GA20ox3_5_e |
| | ga20ox5 | | | | insertion +1, | | | | |
| 35 | not available | 59.57 | 10 | not available | not available | not available | not available | R1 | GA20ox3_d |
| 36 | not available | 67.28 | 13.3 | not available | not available | not available | not available | R1 | GA20ox3_g |
| 37 | not available | 56.54 | 9 | not available | not available | not available | not available | R1 | GA20ox3_d |
| 38 | not available | 63.74 | 11.5 | not available | not available | not available | not available | R1 | GA20ox3_g |
| 39 | single homo | 65.24 | 10 | Biallelic | WT | ga20ox3-4, | none | R1 | GA20ox3_d |
| | ga20ox3 | | | deletion −1, | | ga20ox3-2 | | | |
| | | | | deletion −1, | | | | | |
| 40 | single homo | 69.49 | 9.5 | Biallelic | WT | ga20ox3-2, | none | R1 | GA20ox3_d |
| | ga20ox3 | | | deletion −1, | | ga20ox3-4 | | | |
| | | | | deletion −1, | | | | | |
| 41 | single homo | 60.12 | 9 | Biallelic | WT | ga20ox3-5, | none | R1 | GA20ox3_d |
| | ga20ox3 | | | deletion −1, | | ga20ox3-2 | | | |
| | | | | deletion −3, | | | | | |
| 42 | single homo | 53.74 | 10 | Biallelic | WT | ga20ox3-10, | none | R1 | GA20ox3_g |
| | ga20ox3 | | | deletion −1, | | ga20ox3-9 | | | |
| | | | | insertion +1, | | | | | |
| 43 | single homo | 58.43 | 9.8 | Biallelic | WT | ga20ox3-1, | none | R1 | GA20ox3_d |
| | ga20ox3 | | | deletion −13, | | ga20ox3-2 | | | |
| | | | | deletion −1, | | | | | |
| 44 | single homo | 57.28 | 11.5 | Biallelic | WT | ga20ox3-8, | none | R1 | GA20ox3_5_f |
| | ga20ox3 | | | deletion −2, | | ga20ox3-6 | | | |
| | | | | insertion +1, | | | | | |
| 45 | single homo | 56.26 | 11.5 | Biallelic | WT | ga20ox3-8, | none | R1 | GA20ox3_5_f |
| | ga20ox3 | | | insertion +1, | | ga20ox3-6 | | | |
| | | | | deletion −2, | | | | | |
| 46 | single homo | 59.8 | 10.8 | Biallelic | WT | ga20ox3-8, | none | R1 | GA20ox3_5_f |
| | ga20ox3 | | | insertion +1, | | ga20ox3-6 | | | |
| | | | | deletion −2, | | | | | |
| 47 | single hetero | 54.45 | 11 | Het insertion +1, | WT | ga20ox3-8 | none | R1 | GA20ox3_5_f |
| | ga20ox3 | | | | | | | | |
| 48 | single homo | 52.68 | 9.5 | Homozygous | WT | ga20ox3-2 | none | R1 | GA20ox3_d |
| | ga20ox3 | | | deletion −1, | | | | | |
| 49 | single homo | 64.17 | 12 | Homozygous | WT | ga20ox3-2 | none | R1 | GA20ox3_d |
| | ga20ox3 | | | deletion −1, | | | | | |
| 50 | single homo | 56.97 | 11 | Homozygous | WT | ga20ox3-9 | none | R1 | GA20ox3_g |
| | ga20ox3 | | | deletion −1, | | | | | |
| 51 | single homo | 43.19 | 12.5 | Homozygous | WT | ga20ox3-9 | none | R1 | GA20ox3_g |
| | ga20ox3 | | | deletion −1, | | | | | |
| 52 | single homo | 58.94 | 11.3 | Homozygous | WT | ga20ox3-9 | none | R1 | GA20ox3_g |
| | ga20ox3 | | | deletion −1, | | | | | |
| 53 | single homo | 61.65 | 13 | Homozygous | WT | ga20ox3-8 | none | R1 | GA20ox3_5_f |
| | ga20ox3 | | | insertion +1, | | | | | |
| 54 | single homo | 60.91 | 11.5 | Homozygous | WT | ga20ox3-10 | none | R1 | GA20ox3_g |
| | ga20ox3 | | | insertion +1, | | | | | |

TABLE 19
A list of R2 plants having edited alleles in GA20 oxidase_3, GA20 oxidase_5, or both genes. Plant No.
45 and 46 are considered outliers and not included for generating average plant height data shown in Table 20.

| | | Plant | | | | | | |
|---|---|---|---|---|---|---|---|---|
| Plant | | Height | GA20ox3 | GA20ox5 | ga20ox3 | ga20ox5 | Gener- | |
| No. | Plant Genotype | (inches) | Genotype | Genotype | Allele(s) | Allele | ation | gRNA |
| 1 | homo ga20ox3/ | 45 | Biallelic +1 | Heterozygous −1 | ga20ox3-7, | ga20ox5-1 | R2 | GA2ox3_5_f |
| | hetero ga20ox5 | | insertion | deletion | ga20ox3-8 | | | |
| 2 | homo ga20ox3/ | 45.2 | homozygous +1 | Heterozygous −1 | ga20ox3-8 | ga20ox5-1 | R2 | GA2ox3_5_f |
| | hetero ga20ox5 | | insertion | deletion | | | | |
| 3 | homo ga20ox3/ | 45 | Biallelic −2 | Heterozygous −2 | ga20ox3-6, | ga20ox5-2 | R2 | GA2ox3_5_f |
| | hetero ga20ox5 | | deletion, +1 | deletion | ga20ox3-8 | | | |
| | | | insertion | | | | | |
| 4 | homo ga20ox3/ | 42.8 | homozygous +1 | Heterozygous −2 | ga20ox3-8 | ga20ox5-2 | R2 | GA2ox3_5_f |
| | hetero ga20ox5 | | insertion | deletion | | | | |
| 5 | homo ga20ox3/hetero | 45.2 | homozygous +1 | Heterozygous −2 | ga20ox3-8 | ga20ox5-2 | R2 | GA2ox3_5_f |
| | ga20ox5 | | insertion | deletion | | | | |
| 6 | homo ga20ox3/ | 48.8 | homozygous +1 | Heterozygous −2 | ga20ox3-8 | ga20ox5-2 | R2 | GA2ox3_5_f |
| | hetero ga20ox5 | | insertion | deletion | | | | |
| 7 | homo ga20ox3/ | 51.8 | homozygous +1 | Heterozygous −2 | ga20ox3-8 | ga20ox5-2 | R2 | GA2ox3_5_f |
| | hetero ga20ox5 | | insertion | deletion | | | | |
| 8 | homo ga20ox3/ | 45.4 | homozygous +1 | Heterozygous −2 | ga20ox3-8 | ga20ox5-2 | R2 | GA2ox3_5_f |
| | hetero ga20ox5 | | insertion | deletion | | | | |
| 9 | homo ga20ox3/ | 47 | Homozygous −2 | Heterozygous −2 | ga20ox3-6 | ga20ox5-2 | R2 | GA2ox3_5_f |
| | hetero ga20ox5 | | deletion | deletion | | | | |
| 10 | homo ga20ox3/ | 49.8 | Homozygous −2 | Heterozygous −2 | ga20ox3-6 | ga20ox5-2 | R2 | GA2ox3_5_f |
| | hetero ga20ox5 | | deletion | deletion | | | | |
| 11 | single homo | 52.4 | WT | Homozygous −1 | none | ga20ox5-4 | R2 | GA2ox5_a |
| | ga20ox5 | | | deletion | | | | |
| 12 | single homo | 39.2 | WT | Homozygous −1 | none | ga20ox5-4 | R2 | GA2ox5_a |
| | ga20ox5 | | | deletion | | | | |
| 13 | single homo | 53 | WT | Homozygous −1 | none | ga20ox5-4 | R2 | GA2ox5_a |
| | ga20ox5 | | | deletion | | | | |
| 14 | single homo | 54 | WT | Homozygous −1 | none | ga20ox5-4 | R2 | GA2ox5_a |
| | ga20ox5 | | | deletion | | | | |
| 15 | single homo | 53.8 | WT | Homozygous −1 | none | ga20ox5-4 | R2 | GA2ox5_a |
| | ga20ox5 | | | deletion | | | | |
| 16 | single homo | 53.4 | WT | Homozygous −1 | none | ga20ox5-4 | R2 | GA2ox5_a |
| | ga20ox5 | | | deletion | | | | |
| 17 | Double homo | 45 | Biallelic +1 | Homozygous +1 | ga20ox3-7, | ga20ox5-3 | R2 | GA2ox3_5_f |
| | | | insertion | insertion | ga20ox3-8 | | | |
| 18 | Double homo | 37 | homozygous +1 | Homozygous −2 | ga20ox3-8 | ga20ox5-2 | R2 | GA2ox3_5_f |
| | | | insertion | deletion | | | | |
| 19 | Double homo | 39.2 | homozygous +1 | Homozygous −2 | ga20ox3-8 | ga20ox5-2 | R2 | GA2ox3_5_f |
| | | | insertion | deletion | | | | |
| 20 | Double homo | 39.8 | homozygous +1 | Homozygous −2 | ga20ox3-8 | ga20ox5-2 | R2 | GA2ox3_5_f |
| | | | insertion | deletion | | | | |
| 21 | Double homo | 40.8 | homozygous +1 | Homozygous −2 | ga20ox3-8 | ga20ox5-2 | R2 | GA2ox3_5_f |
| | | | insertion | deletion | | | | |
| 22 | Double homo | 40.8 | homozygous +1 | Homozygous −2 | ga20ox3-8 | ga20ox5-2 | R2 | GA2ox3_5_f |
| | | | insertion | deletion | | | | |
| 23 | Double homo | 41 | homozygous +1 | Homozygous −2 | ga20ox3-8 | ga20ox5-2 | R2 | GA2ox3_5_f |
| | | | insertion | deletion | | | | |
| 24 | Double homo | 41.4 | homozygous +1 | Homozygous −2 | ga20ox3-8 | ga20ox5-2 | R2 | GA2ox3_5_f |
| | | | insertion | deletion | | | | |
| 25 | Double homo | 41.8 | homozygous +1 | Homozygous −2 | ga20ox3-8 | ga20ox5-2 | R2 | GA2ox3_5_f |
| | | | insertion | deletion | | | | |
| 26 | Double homo | 42 | homozygous +1 | Homozygous −2 | ga20ox3-8 | ga20ox5-2 | R2 | GA2ox3_5_f |
| | | | insertion | deletion | | | | |
| 27 | Double homo | 42 | homozygous +1 | Homozygous −2 | ga20ox3-8 | ga20ox5-2 | R2 | GA2ox3_5_f |
| | | | insertion | deletion | | | | |
| 28 | Double homo | 42.4 | homozygous +1 | Homozygous −2 | ga20ox3-8 | ga20ox5-2 | R2 | GA2ox3_5_f |
| | | | insertion | deletion | | | | |
| 29 | Double homo | 43 | homozygous +1 | Homozygous −2 | ga20ox3-8 | ga20ox5-2 | R2 | GA2ox3_5_f |
| | | | insertion | deletion | | | | |
| 30 | Double homo | 43.8 | homozygous +1 | Homozygous −2 | ga20ox3-8 | ga20ox5-2 | R2 | GA2ox3_5_f |
| | | | insertion | deletion | | | | |
| 31 | Double homo | 46.2 | homozygous +1 | Homozygous −2 | ga20ox3-8 | ga20ox5-2 | R2 | GA2ox3_5_f |
| | | | insertion | deletion | | | | |
| 32 | single homo | 62 | Homozygous −1 | WT | ga20ox3-9 | none | R2 | GA2ox3_g |
| | ga20ox3 | | deletion | | | | | |
| 33 | single homo | 39 | Homozygous −1 | WT | ga20ox3-9 | none | R2 | GA2ox3_g |
| | ga20ox3 | | deletion | | | | | |
| 34 | single homo | 51 | Biallelic −2 | WT | ga20ox3-6, | none | R2 | GA2ox3_5_f |
| | ga20ox3 | | deletion, +1 | | ga20ox3-8 | | | |
| | | | insertion | | | | | |
| 35 | single homo | 52.8 | homozygous +1 | WT | ga20ox3-8 | none | R2 | GA2ox3_5_f |
| | ga20ox3 | | insertion | | | | | |
| 36 | single homo | 59.4 | homozygous +1 | WT | ga20ox3-8 | none | R2 | GA2ox3_5_f |
| | ga20ox3 | | insertion | | | | | |
| 37 | single homo | 46 | homozygous +1 | WT | ga20ox3-8 | none | R2 | GA2ox3_5_f |
| | ga20ox3 | | insertion | | | | | |
| 38 | single homo | 52.8 | homozygous +1 | WT | ga20ox3-8 | none | R2 | GA2ox3_5_f |
| | ga20ox3 | | insertion | | | | | |
| 39 | single homo | 51.4 | Homozygous −2 | WT | ga20ox3-6 | none | R2 | GA2ox3_5_f |
| | ga20ox3 | | deletion | | | | | |
| 40 | SUP_Ox3&Ox5 | 43.6 | WT | WT | None | none | n/a | none |
| 41 | SUP_Ox3&Ox5 | 43.8 | WT | WT | None | none | n/a | none |
| 42 | SUP_Ox3&Ox5 | 42.2 | WT | WT | None | none | n/a | none |
| 43 | WT | 56.4 | WT | WT | None | none | n/a | none |
| 44 | WT | 58.8 | WT | WT | None | none | n/a | none |
| 45 | Double homo | 61.8 | Biallelic −2 | Homozygous −2 | ga20ox3-6 | ga20ox5-2 | R2 | GA2ox3_5_f |
| | | | deletion, +1 | deletion | | | | |
| | | | insertion | | | | | |
| 46 | Double homo | 61.8 | Biallelic +1 | Homozygous −1 | ga20ox3-7, | ga20ox5-1 | R2 | GA2ox3_5_f |
| | | | insertion | deletion | ga20ox3-8 | | | |

TABLE 20
R3 stage plant height differences between greenhouse-grown inbred gene-edited plants and reference control plants.

| | Avg. Plant | | | |
|---|---|---|---|---|
| | Height | | | % |
| Plant Genotype | (inches) | Std. Dev | # of Plants | Reduction |
| WT | 57.6 | 1.7 | 2 | 0 |
| Homo_ox3/WT_Ox5 | 51.8 | 7.2 | 8 | 10.1% |
| WT_Ox3/Homo_ox5 | 51.0 | 5.8 | 6 | 11.5% |
| Homo_ox3/Het_ox5 | 46.6 | 2.7 | 10 | 19.1% |
| Homo_ox3/Homo_ox5 | 41.7 | 2.3 | 17 | 27.5% |
| SUP_Ox3&Ox5 | 43.2 | 6.6 | 3 | 25.0% |

### Example 19. Suppression Construct Targeting GA20 Oxidase_3 and GA20 Oxidase_5 Genes Reduces GA20 Oxidase Transcript and Active GA Levels in the Plant

To determine how GA20 oxidase transcript levels were affected in transgenic plants with the suppression construct targeting the GA20 oxidase_3 and GA20 oxidase_5 genes, whole tissues from various parts of transgenic plants grown in the greenhouse were taken at different vegetative stages (V3, V8, and V14), and mRNA transcript levels for each of the GA20 oxidase genes were analyzed using a TaqMan® assay. For these experiments, total RNA was extracted using a Direct-Zol RNA extraction kit from Zymo Research™ and treated with Turbo™ DNase to reduce genomic DNA contamination. RNA was then reverse transcribed to generate double-stranded cDNA. Reverse transcription quantitative PCR was performed with gene specific primers and FAM labeled TaqMan® probes on the Bio-Rad® CFX96 Real Time System. Quality control metrics were calculated using tissue specific standards to determine qPCR efficiency and total RNA that had not undergone reverse transcription to account for residual genomic DNA contamination. The difference between cycle threshold values for genes of interest versus normalizer genes determined the relative quantity of each gene transcript in each tissue. This relative quantity was calculated using either one (18S) or the geometric mean of two (18S and ELF1A) normalizer genes.

In this experiment, the level of the GA20 oxidase_3 transcript was reduced in most of the vegetative tissues at these stages, including leaf and stem tissue at V3, internode tissue at V8, and leaf and internode tissue at V14, although the level of GA20 oxidase_3 transcript in V3 root and V8 leaf appeared unchanged (data not shown). Furthermore, the level of GA20 oxidase_5 transcript for this experiment was generally unchanged in the vegetative tissues tested (data not shown), although the level of expression of the GA20 oxidase_5 transcript was relatively low in these tissues. Neither GA20 oxidase_3 nor GA20 oxidase_5 were significantly reduced in root tissue samples of transgenic plants. Each of the other GA20 oxidase genes (i. e., the 1, 2, 4 and 6-9 subtypes) were generally unchanged or increased in some tissues of the transgenic plants.

A similar experiment was conducted with reproductive tissues from transgenic plants expressing the same suppression construct. Whole tissues from various parts of transgenic plants grown in the greenhouse were taken at different reproductive stages (R1 and R3), and mRNA transcript levels for each of the GA20 oxidase genes were analyzed using a TaqMan® assay. In this experiment, the levels of GA20 oxidase_3 and GA20 oxidase_5 transcripts were mostly unchanged in R1 leaf, ear, tassel and internode and R3 leaf and internode, relative to controls (data not shown). Results for the other GA20 oxidase genes were mostly mixed or neutral (data not shown).

These data show that the level of GA20 oxidase_3 transcripts in transgenic corn plants during vegetative stages was generally reduced in this experiment but appears mostly unchanged relative to control plant tissues during later reproductive stages. Although a clear reduction in the level of GA20 oxidase_5 gene transcripts was not generally observed in these transgenic plant tissues, the expression level of this gene was relatively low. Thus, changes in its expression level may have been difficult to detect with this method. In addition, the suppression construct appears to be specific to the targeted GA20 oxidase genes since no consistent reduction in expression level was observed in this experiment for any of the other GA20 oxidase genes.

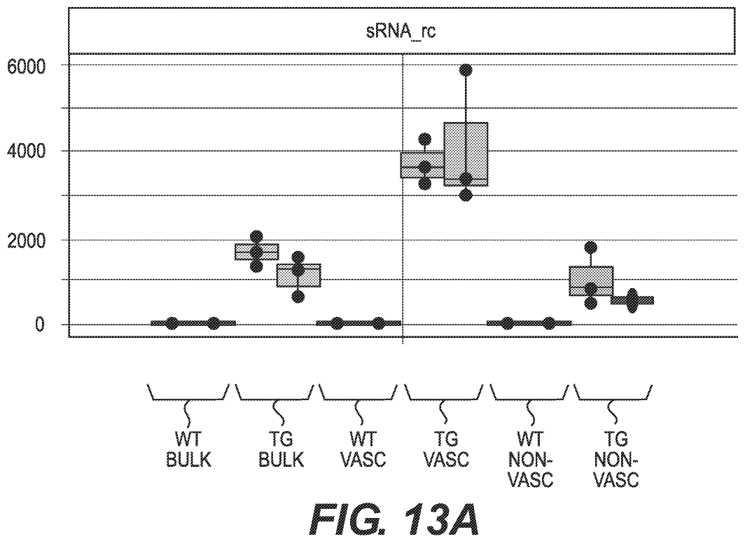

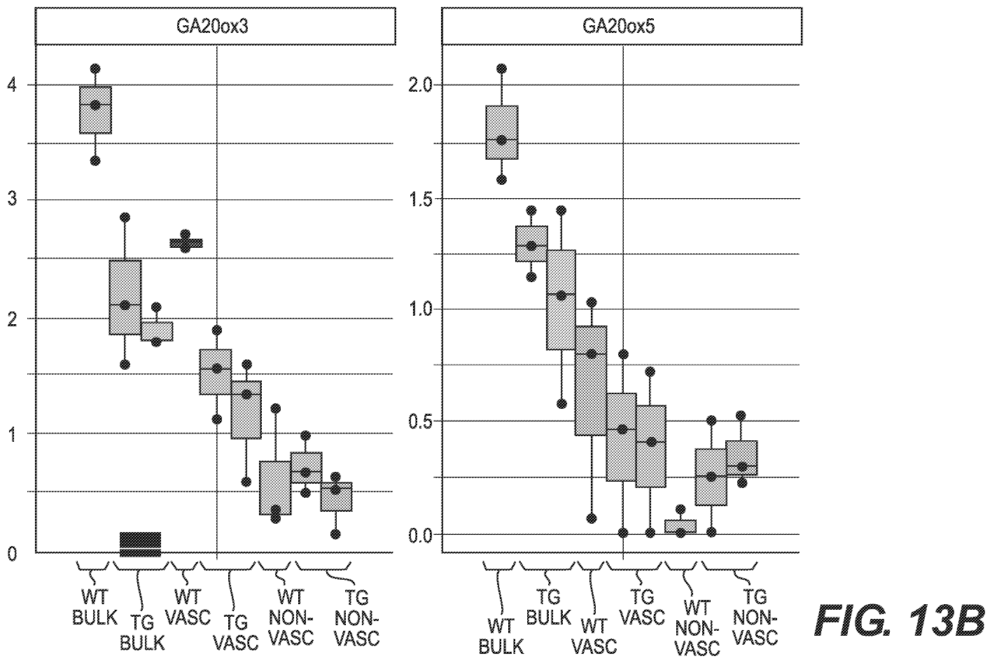

In a separate experiment, GA20 oxidase expression levels were determined in stem tissues of transgenic plants expressing the suppression construct from the prior Examples (targeting the GA20 oxidase_3 and GA20 oxidase_5 genes for suppression under the control of the RTBV promoter), in comparison to a wild-type control. Tissue samples were taken from V3-V6 stems/stalks and parts of those stems were further dissected to separate vascular and non-vascular tissues to determine differential expression levels among these tissues. Transcript expression levels were determined using a RNA sequencing (RNA-Seq) approach for quantitative comparison between transgenic and wild-type plant tissues. The data presented in FIG. 13A are generated from transgenic plants having one of two events and wild type control plants having one of two germplasms, with each bar in FIG. 13A representing one of the two transgenic events or germplasms, respectively. For these experiments, individual vascular bundles were separated from the remaining stem/stalk tissue of the samples and subjected to separate analysis. As shown in FIG. 13A, the miRNA expressed by the suppression construct was detected in bulk plant stem tissue (“bulk”; i.e., without separation of vascular and non-vascular tissues), as well as in separated vascular (“Vase”) and non-vascular (“Non-Vase”) tissues from the bulk stem/stalk sample.

However, the expression level of the miRNA was much higher in vascular tissue than in non-vascular tissue indicating the vascular expression pattern of the RTBV promoter.

The bulk stem/stalk samples and the separated vascular and non-vascular samples were also analyzed in a similar RNA-Seq experiment to measure and compare the levels of GA20 oxidase_3 and GA20 oxidase_5 gene transcripts in transgenic versus wild-type control plants (along with other GA20 oxidase genes), although only one wild-type sample is shown for each tissue type. For these experiments, stalk tissue from control or transgenic plants (two events) were sectioned to separate vascular bundles and non-vascular tissues as described above. Total sRNA and mRNA were sequenced for each sample, and data was analyzed and compared using principle component analysis.

As shown in FIG. 13B, transcript levels of the GA20 oxidase_3 gene were significantly reduced in bulk stem tissue (Bulk) and separated stem vascular tissues (Vasc) of transgenic plants (TG) relative to wild-type controls (WT), but appeared unchanged in separated non-vascular (Non-Vasc) tissue. However, transcript levels of the GA20 oxidase_5 gene were significantly reduced in bulk stem tissue (Bulk), but relatively unchanged in separated vascular (Vasc) and non-vascular (Non-Vasc) tissues of transgenic plants, although there was a downward trend line for the GA20 oxidase_5 transcript in vascular (Vasc) tissue samples from transgenic plants. The level of expression of the GA20 oxidase_5 gene was low, particularly in non-vascular tissues. All other GA20 oxidase genes did not show a significant reduction in their transcript levels in the transgenic plant tissues analyzed, although a couple GA20 oxidase genes did show a slight upward trend in their level of expression. This data further demonstrates that the expression levels of the GA20 oxidase_3 and GA20 oxidase_5 genes are decreased to varying extents in one or more tissues of transgenic plants having the suppression construct relative to controls. Indeed, the higher expression of the miRNA and greater suppression of the endogenous GA20 oxidase_3 gene in vascular tissues is consistent with the vascular pattern of expression of the RTBV promoter, and perhaps the higher levels of GA20 oxidase_3 gene expression in vascular versus non-vascular tissues of wild-type plants. A similar pattern is also observed for the GA20 oxidase_5 gene, although not as pronounced as the GA20 oxidase_3 gene between vascular and non-vascular tissues.

The short stature, semi-dwarf phenotype observed with GA20 oxidase suppression in transgenic plants is likely mediated by a reduction in the level of active GAs present in the stem or internode tissues and/or in plant tissues that produce active GAs. To determine the levels of active GAs (particularly G1, G3 and G4) relative to other inactive forms of the hormone, GA levels were measured in different tissue samples taken from transgenic and wild-type control plants at different stages of development. For these experiments, fresh frozen samples for each tissue were milled and dispensed into 96 well glass tubes along with internal standards. Samples were extracted using methanol:water:acetic acid (80:19:1 v/v/v) solvent two times for 4 hours at 4° C. Solvent was evaporated from the extract to near dryness using multi-channel SPE with nitrogen. Samples were further purified using a SPE cartridge. After purification, samples were run using standard LC-MS/MS method with Shimadzu® Nexera® UPLC and SCIEX® triple quad 5500 mass spec instrumentation. Chromatographs were analyzed and quantified using internal standards.

Two sets of experiments were performed with samples taken from various tissues of vegetative stage plants. As shown in Table 21 for one experiment in the greenhouse, reduced levels of active GAs (GA1, GA3, and GA4) were observed in various tissues of transgenic plants at different vegetative stages. The data in Table 21 is displayed as the number of transgenic plants having a significant change in the amount of each GA hormone for a given tissue (“U”=up or increased; “D”=down or decreased; “N”=neutral or no change; and “T”=total number of plants). The GAs that showed at least a partial reduction in tissue samples are presented in bold. Active GA1 was reduced in leaf and internode tissues at V8 stage and internode tissue at V14 stage, and active GA4 was reduced in V3 stem and V8 and V14 internode. However, active GA3 was not observably reduced in this experiment. Other inactive forms of GAs were altered in various tissues of transgenic plants as shown in Table 21. In general, GAs that are downstream of GA20 oxidase genes in the gibberellic acid pathway (e.g., GA9, GA20, and GA34) tended to be reduced, whereas GAs that are upstream of GA20 oxidase genes tended to be higher (e.g., GA12 and GA53), which may be due to the lower activity of GA oxidase genes causing the precursor GAs upstream to accumulate. This data is consistent with suppression of GA20 oxidase activity in these tissues and lower levels of active GA hormones in the stem and leaf of transgenic plants.

In a separate experiment, similar measurements of GA hormones were taken from various plant tissues during vegetative stages of development. As shown in Table 22 for an experiment using tissues taken from plants in the greenhouse and field, reduced levels of one or more active GAs (GA1, GA3, and GA4) were observed in the leaf and internode of transgenic plants at V3 and V8 stages. The leaf samples at V8 stage for this experiment were taken from plants in the field, unlike the other samples taken from plants in the greenhouse. The data in Table 22 is displayed in a similar manner as described for Table 21. Other inactive forms of GAs were altered in various tissues of transgenic plants as shown in Table 22. Similar to the observations above, GAs that are downstream of GA20 oxidase genes in the gibberellic acid pathway (e.g., GA9, GA20, and GA34) tended to be reduced, whereas GAs that are upstream of GA20 oxidase genes tended to be higher (e.g., GA12 and GA53). This data is again consistent with suppression of GA20 oxidase activity in these tissues and lower levels of active GA hormones in the stem and leaf of transgenic plants.

TABLE 21
Change in GA hormone levels in tissues of transgenic corn plants expressing
a GA20 oxidase suppression construct in the greenhouse.

| | Stage: | | | | | | | |
|---|---|---|---|---|---|---|---|---|
| | V3 | | | V8 | | V14 | | |
| Tissue: | Leaf | Stem | Root | Leaf | Internode | Leaf | Internode | Tassel |
| GA1 | 2N/2T | 2N/2T | 2N/2T | 2D/2T | 1D/1N/2T | 2N/2T | 2D/2T | 2N/2T |
| GA3 | 2N/2T | 2N/2T | 2N/2T | 2N/2T | 2N/2T | 2N/2T | 2N/2T | 2N/2T |
| GA4 | 2N/2T | 2D/2T | 2N/2T | 2N/2T | 2D/2T | 2U/2T | 2D/2T | 2N/2T |
| GA8 | 1U/1N/2T | 2N/2T | 1U/1N/2T | 2N/2T | 2D/2T | 1D/1N/2T | 1D/1N/2T | 1U/1N/2T |
| GA9 | 2N/2T | 2D/2T | 2N/2T | 2N/2T | 2D/2T | 2U/2T | 2D/2T | ID/IN |
| | | | | | | | | /2T |
| GA12 | 1D/1N/2T | 2U/2T | 2N/2T | 2U/2T | 2N/2T | 1U/1N/2T | 2N/2T | 2N/2T |
| GA20 | 2D/2T | 2N/2T | 2N/2T | 2D/2T | 2D/2T | 2D/2T | 1D/1N/2T | 2N/2T |
| GA34 | 2N/2T | 2D/2T | 2N/2T | 2N/2T | 2D/2T | 2N/2T | 2D/2T | 2N/2T |
| GA53 | 2U/2T | 2U/2T | 2N/2T | 2U/2T | 2N/2T | 2U/2T | 1U/1N/2T | 1U/1N/2T |

TABLE 22
Change in GA hormone levels in tissues of transgenic
corn plants expressing a
GA20 oxidase suppression construct in the greenhouse (GH) or field.

| Stage: | V3 | | V8 | |
|---|---|---|---|---|
| Tissue: | Leaf (GH) | Root (GH) | Internode (GH) | Leaf (Field) |
| GA1 | 3D/1N/4T | 2D/1U/1N/4T | 3D/1N/4T | 7D/1N/8T |
| GA3 | 3D/1N/4T | 4N/4T | 3D/1N/4T | 7D/1N/8T |
| GA4 | 4N/4T | 4N/4T | 4D/4T | 8D/8T |
| GA8 | 4N/4T | 4N/4T | 4N/4T | 4N/4T |
| GA9 | 4D/4T | 4N/4T | 4D/4T | 5U/3N/8T |
| GA12 | ND | ND | ND | 7U/1N/8T |
| GA20 | 4D/4T | 1D/3N/4T | 4D/4T | 8D/8T |
| GA34 | 1U/3N/4T | 4D/4T | 4D/4T | 4U/4N/8T |
| GA53 | 4U/4T | 2U/2N/4T | 1D/3N/4T | 8U/8T |

Suppression of the GA20 oxidase_3 and GA20 oxidase_5 genes in transgenic corn plants reduces the levels of targeted GA oxidase transcripts in various tissues including the stem, internode, vascular tissues and leaves, and suppression of these GA20 oxidase genes is further associated with reduced levels of active GAs in tissues of the transgenic plant including the stem and internode, which is the site of action for affecting plant growth during vegetative stages and ultimately plant height by later vegetative and reproductive stages. Similar to observations that GA20 oxidase transcript levels are mostly unchanged or mixed in reproductive stage tissues, the levels of GA hormones including active GAs are also mostly unchanged or mixed in reproductive stage tissues (data not shown).

### Example 20. Editing Both GA20 Oxidase_3 and GA20 Oxidase_5 Reduces Active GA Levels in the Plant

R2 plants having edited alleles in GA20 oxidase_3, GA20 oxidase_5, or both genes were tested in the field along with transgenic inbred corn plants having an artificial microRNA suppression construct targeting the GA20 oxidase_3 and GA20 oxidase_5 genes for suppression (SUP_GA20Ox3&Ox5 (“SUP_Ox3&Ox5”), as described in Example 1). Various physiological traits were measured including plant height to ear node at R3, plant height to uppermost ligule, ear height, ear length, ear diameter, kernels/ear, kernels/unit area, single kernel weight, stalk diameter, and grain yield estimate. Plants containing homozygous mutant alleles of both GA20 oxidase_3 and GA20 oxidase_5 genes (i.e., double homozygous ga20ox3/ga20ox5 mutants) showed semi-dwarf phenotypes with altered plant architecture. Homozygous single ga20ox3 mutants and homozygous single ga20ox5 mutants showed slightly taller plant height than double homozygous ga20ox3/ga20ox5 mutants. Table 23 shows key traits with percent delta relative to wild type control plants without edited allele (i.e., percent difference compared to control).

In addition, top collared leaf at V8 was collected to measure the level of a panel of Gibberellic acid hormones through standard biochemical assays. Data indicate that at V8 growth stage, top collared leaf tissues of plants with both GA20ox3 and GA20ox5 edits have significantly lower levels of GA20, GA4 and GA1, but higher levels of GA53 compared to the wild type (control). Changes in GA hormone levels observed in tissues of plants with GA20ox3 and GA20ox5 edits were similar to those observed in transgenic SUP_Ox3&Ox5 plants (Table 24).

TABLE 23
Editing GA20 oxidase_3, GA20 oxidase_5, or both genes impacts various physiological
traits (shown as average percent difference relative to a wild-type control).

| | Percent_delta relative to WT control | | |
|---|---|---|---|
| | Double homozygous | Homozygous | Homozygous |
| | ga20ox3/ga20ox5 | single ga20ox5 | single ga20ox3 |
| | (Plant #18 through | (plant #11 to 16 | (plant #32 and 33 |
| Trait | 31 in Table 19) | in Table 19) | in Table 19) |
| Plant Height to Ear Node R3 | −46.12 | −16.24 | −22.55 |
| Plant Height to Uppermost Ligulated Leaf R3 | −30.39 | −4.49 | −8.6 |
| Stalk Diameter Ear Minus Four R3 | −6.21 | −11.01 | −3.37 |
| Days to 50% Visible Silk R1 | −2.48 | −2.48 | −2.48 |
| Ear Diameter (imaging) R6 | −0.4 | −0.48 | −1.72 |
| Ear Length (imaging) R6 | −5.83 | −1.31 | −4.56 |
| Grain Yield Estimate R6 | −12.2 | −17.62 | −16.55 |
| Kernels per Ear R6 | −2.62 | −5.14 | −9.03 |
| Kernels per unit area | −10.59 | −7.08 | −11.15 |
| Single Kernel Weight R6 | −1.59 | −11.32 | −6.29 |

TABLE 24
Editing GA20 oxidase_3, GA20 oxidase_5, or both genes impacts GA hormonal levels (shown
as Average Delta, i.e., difference in pmol GA/gram of tissue and (p-value), relative to a
wild-type control). Average pmol GA/gram of tissue for wild-type hormonal levels also shown.

| | | | | Homozygous | Homozygous | Double | |
|---|---|---|---|---|---|---|---|
| | | | | single | single | homozygous | |
| | | | | ga20ox3 | ga20ox5 | ga20ox3/ga20ox5 | SUP_Ox3&Ox5 |
| Growth | | Hormone | Wild-type | (Average | (Average | (Average | (Average |
| stage | Leaf type | type | (average) | Delta) | Delta) | Delta) | Delta) |
| V8 | Leaf - top | GA12-pmole/g | 0.065 | 0.0250 | 0.0007 | 0.1382 | 0.1726 |
| | collared | | | (0.517) | (0.985) | (0.002) | (1.91E−4) |
| V8 | Leaf - top | GA1-pmole/g | 2.726 | 0.3236 | 0.0984 | −1.8405 | −1.4112 |
| | collared | | | (0.355) | (0.776) | (5.64E−5) | (7.41E−4) |
| V8 | Leaf - top | GA20-pmole/g | 2.025 | 0.6085 | 0.6311 | −1.8525 | −1.8446 |
| | collared | | | (0.017) | (0.014) | (4.85E−7) | (5.13E−7) |
| V8 | Leaf - top | GA34-pmole/g | 2.665 | −0.2339 | −0.1940 | 0.3424 | 0.2456 |
| | collared | | | (0.191) | (0.275) | (0.061) | (0.170) |
| V8 | Leaf - top | GA3-pmole/g | 0.200 | 0.1747 | 0.2062 | −0.0586 | −0.03487 |
| | collared | | | (0.006) | (0.002) | (0.312) | (0.544) |
| V8 | Leaf - top | GA4-pmole/g | 0.270 | −0.0734 | 0.0169 | −0.1473 | −0.0842 |
| | collared | | | (0.455) | (0.863) | (0.144) | (0.393) |
| V8 | Leaf - top | GA53-pmole/g | 0.355 | −0.0291 | 0.1493 | 0.9521 | 1.0875 |
| | collared | | | (0.893) | (0.492) | (3.77E−4) | (1.03E−4) |
| V8 | Leaf - top | GA8-pmole/g | 0.067 | 0.0066 | −0.0063 | −0.0065 | 0.0393 |
| | collared | | | (0.857) | (0.863) | (0.860) | (0.292) |
| V8 | Leaf - top | GA9-pmole/g | 1.894 | −1.0053 | −0.5852 | 2.5123 | 1.9821 |
| | collared | | | (0.034) | (0.201) | (1.48E−5) | (2.29E−4) |

Having described the present disclosure in detail, it will be apparent that modifications, variations, and equivalent embodiments are possible without departing from the spirit and scope of the present disclosure as described herein and in the appended claims. Furthermore, it should be appreciated that all examples in the present disclosure are provided as non-limiting examples.

What is claimed is:

1. A modified corn plant, or plant part thereof, comprising a mutant allele at GA20 oxidase_3 locus, a mutant allele at GA20 oxidase_5 locus, or both, and further comprising an expression cassette comprising a transcribable DNA sequence encoding a non-coding RNA for suppressing one or more of the GA20 oxidase_3 gene, the GA20 oxidase_4 gene, and the GA20 oxidase_5 gene.

2. The modified corn plant, or plant part thereof, claim 1, wherein said GA20 oxidase_3 locus, said GA20 oxidase_5 locus, or both comprise homozygous mutant alleles.

3. The modified corn plant, or plant part thereof, claim 1, wherein said GA20 oxidase_3 locus, said GA20 oxidase_5 locus, or both comprise heterozygous alleles.

4. The modified corn plant, or plant part thereof, claim 1, wherein said GA20 oxidase_3 locus comprises homozygous mutant alleles, and said GA20 oxidase_5 locus comprises heterozygous alleles.

5. The modified corn plant, or plant part thereof, claim 1, wherein said GA20 oxidase_5 locus comprises homozygous mutant alleles, and said GA20 oxidase_3 locus comprises heterozygous alleles

6. The modified corn plant, or plant part thereof, of any one of claims 1 to 5, wherein one or both of said GA20 oxidase_3 and GA20 oxidase_5 loci comprise a heteroallelic combination or two identical mutant alleles.

7. The modified corn plant, or plant part thereof, of any one of claims 1 to 5, wherein said mutant allele at the GA20 oxidase_3 locus comprises a mutation in a sequence region selected from the group consisting of a promoter, 5′ UTR, first exon, first intron, second exon, second intron, third exon, 3′ UTR, terminator, and any combination thereof.

8. The modified corn plant, or plant part thereof, of any one of claims 1 to 5, wherein said mutant allele at the GA20 oxidase_3 locus comprises one or more mutation types selected from the group consisting of a nonsense mutation, a missense mutation, a frameshift mutation, a splice-site mutation, and any combination thereof.

9. The modified corn plant, or plant part thereof, of any one of claims 1 to 5, wherein said mutant allele at the GA20 oxidase_3 locus results in one or more of the following: a GA20 oxidase_3 protein truncation, a non-translatable GA20 oxidase_3 gene transcript, a non-functional GA20 oxidase_3 protein, a premature stop codon in the GA20 oxidase_3 gene, and any combination thereof.

10. The modified corn plant, or plant part thereof, of any one of claims 1 to 5, wherein each mutant allele at the GA20 oxidase_3 locus comprises a mutation selected from the group consisting of a substitution, a deletion, an insertion, a duplication, and an inversion of one or more nucleotides relative to a wild-type GA20 oxidase_3 gene.

11. The modified corn plant, or plant part thereof, of any one of claims 1 to 5, wherein said mutant allele at the GA20 oxidase_3 locus comprises one or more mutations in the first exon of the GA20 oxidase_3 gene.

12. The modified corn plant, or plant part thereof, of any one of claims 1 to 5, wherein said mutant allele at the GA20 oxidase_3 locus comprises one or more mutations in the second exon of the GA20 oxidase_3 gene.

13. The modified corn plant, or plant part thereof, of any one of claims 1 to 5, wherein said mutant allele at the GA20 oxidase_5 locus comprises a mutation in a sequence region selected from the group consisting of a promoter, 5′ UTR, first exon, first intron, second exon, second intron, third exon, 3′ UTR, terminator, and any combination thereof.

14. The modified corn plant, or plant part thereof, of any one of claims 1 to 5, wherein said mutant allele at the GA20 oxidase_5 locus comprises one or more mutation types selected from the group consisting of a nonsense mutation, a missense mutation, a frameshift mutation, a splice-site mutation, and any combination thereof.

15. The modified corn plant, or plant part thereof, of any one of claims 1 to 5, wherein said mutant allele at the GA20 oxidase_5 locus results in one or more of the following: a GA20 oxidase_5 protein truncation, a non-translatable GA20 oxidase_5 gene transcript, a non-functional GA20 oxidase_5 protein, a premature stop codon in the GA20 oxidase_5 gene, and any combination thereof.

16. The modified corn plant, or plant part thereof, of any one of claims 1 to 5, wherein each mutant allele at the GA20 oxidase_5 locus comprises a mutation selected from the group consisting of a substitution, a deletion, an insertion, a duplication, and an inversion of one or more nucleotides relative to a wild-type GA20 oxidase_5 gene.

17. The modified corn plant, or plant part thereof, of any one of claims 1 to 5, wherein said mutant allele at the GA20 oxidase_5 locus comprises one or more mutations in the first exon of the GA20 oxidase_5 gene.

18. The modified corn plant, or plant part thereof, of any one of claims 1 to 5, wherein said mutant allele at the GA20 oxidase_5 locus comprises one or more mutations in the second exon of the GA20 oxidase_5 gene.

19. The modified corn plant, or plant part thereof, of any one of claims 1 to 5, wherein said expression cassette is a recombinant DNA expression cassette, and wherein the transcribable DNA sequence is operably linked to a plant-expressible promoter.

20. The modified corn plant, or plant part thereof, of claim 19, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein being at least 95%, at least 96%, at least 97%, at least 98%, at least 99%, at least 99.5%, or 100% identical to SEQ ID NO: 9, 12, or 15.

21. The modified corn plant, or plant part thereof, of claim 19, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7, 8, 10, 11, 13, or 14.

22. The modified corn plant, or plant part thereof, of claim 19, wherein the plant-expressible promoter is a vascular promoter.

23. The modified corn plant, or plant part thereof, of claim 19, wherein the plant-expressible promoter comprises one of the following: a sucrose synthase promoter, a sucrose transporter promoter, a Sh1 promoter, Commelina yellow mottle virus (CoYMV) promoter, a wheat dwarf geminivirus (WDV) large intergenic region (LIR) promoter, a maize streak geminivirus (MSV) coat protein (CP) promoter, a rice yellow stripe 1 (YS1)-like promoter, or a rice yellow stripe 2 (OsYSL2) promoter.

24. The modified corn plant, or plant part thereof, of claim 19, wherein the plant-expressible promoter is a RTBV promoter.

25. The modified corn plant, or plant part thereof, of claim 19, wherein the plant-expressible promoter is a leaf promoter.

26. The modified corn plant, or plant part thereof, of claim 19, wherein the plant-expressible promoter comprises one of the following: a RuBisCO promoter, a PPDK promoter, a FDA promoter, a Nadh-Gogat promoter, a chlorophyll a/b binding protein gene promoter, a phosphoenolpyruvate carboxylase (PEPC) promoter, or a Myb gene promoter.

27. The modified corn plant, or plant part thereof, of claim 19, wherein the plant-expressible promoter is a constitutive promoter.

28. The modified corn plant, or plant part thereof, of claim 19, wherein the plant-expressible promoter comprises one or more constitutive promoters selected from the group consisting of: an actin promoter, a CaMV 35S or 19S promoter, a plant ubiquitin promoter, a plant Gos2 promoter, a FMV promoter, a CMV promoter, a MMV promoter, a PCLSV promoter, an Emu promoter, a tubulin promoter, a nopaline synthase promoter, an octopine synthase promoter, a mannopine synthase promoter, or a maize alcohol dehydrogenase, or a functional portion thereof.

29. The modified corn plant, or plant part thereof, of claim 19, wherein the non-coding RNA molecule encoded by the transcribable DNA sequence is a precursor miRNA or siRNA that is processed or cleaved in a plant cell to form a mature miRNA or siRNA.


SEQUENCE LISTING (261 sequences)

SEQ ID NO: 1 - DNA, 1741 bp, Zea mays
gacggtagttttcatctaaagtttattcttcgtcacatgggatggccgtttgcttgtttgttgcttccgggaggcggtggtgaattgaagcagatcgacaagcatggctgcccactggtctcgatcgatcggcctgccatgccatgccatgccactagagtccgtcctgactggccgcccgttcccccgtataaaaaggcaggcaggcaggcagagcggggacgagcaagcaagcagttgcagttgcagcggcctcctcctctgcttcctccctcctcctcctcaccatggtgctggctgcgcacgatccccctccccttgtgttcgacgctgcccgcctgagcggcctctccgacatcccgcagcagttcatctggccggcggacgagagccccaccccggactccgccgaggagctggccgtgccgctcatcgacctctccggggacgccgccgaggtggtccggcaggtccggcgcgcctgcgacctgcacggcttcttccaggtggtggggcacggcatcgacgcggcgctgacggcggaggcccaccgctgcatggacgccttcttcacgctgccgctcccggacaagcagcgcgcgcagcgccgccagggggacagctgcggctacgccagcagcttcacgggccggttcgcgtccaagctgccctggaaggagacgctgtcgttccgctacaccgacgacgacgacggcgacaagtccaaggacgtcgtggcgtcctacttcgtggacaagctgggcgaggggtaccggcaccacggggaggtgtacgggcgctactgctctgagatgagccgtctgtcgctggagctcatggaggtgctaggcgagagcctgggcgtgggccggcgccacttccggcgcttcttccaggggaacgactccatcatgcgcctcaactactacccgccgtgccagcggccctacgacacgctgggcacggggccgcattgcgaccccacgtcgctcaccatcctgcaccaggacgacgtgggcggactccaggtgttcgacgccgccacgctcgcgtggcgctccatcaggccccgcccgggcgccttcgtcgtcaacatcggcgacaccttcatggcgctctccaacgggcgctacaggagctgcctccaccgcgccgtcgtcaacagccgggtggcacgccgctcgctcgccttcttcctgtgcccggagatggacaaggtggtcaggccgcccaaggagctggtggacgacgccaacccgagggcgtacccggacttcacgtggaggacgctgctggacttcaccatgaggcactacaggtcggacatgaggacgctcgaggccttctccaactggctcagcaccagtagcaatggcggacagcacctgctggagaagaagtaggcatgctatttgggtatggaagatggtggatgtaagcaaacaaagccaaattaagcagagtaggttaattaaggttggctgatgatccatttagggaaggagctgatctccctgactccctcctccaattttctcaaccaaatttatatagtataataataataataaaatagcaagtaatagttgtatcgtattattattaattaatttattagctggtaggcaagtagtattaaataccatttgtagtacgatgggcgtatttctattttggcgttttgctctgtgttttttgacgtttcctttggatttggggggacctcagatcagctcggcct

SEQ ID NO: 2 - DNA, 1116 bp, Zea mays
atggtgctggctgcgcacgatccccctccccttgtgttcgacgctgcccgcctgagcggcctctccgacatcccgcagcagttcatctggccggcggacgagagccccaccccggactccgccgaggagctggccgtgccgctcatcgacctctccggggacgccgccgaggtggtccggcaggtccggcgcgcctgcgacctgcacggcttcttccaggtggtggggcacggcatcgacgcggcgctgacggcggaggcccaccgctgcatggacgccttcttcacgctgccgctcccggacaagcagcgcgcgcagcgccgccagggggacagctgcggctacgccagcagcttcacgggccggttcgcgtccaagctgccctggaaggagacgctgtcgttccgctacaccgacgacgacgacggcgacaagtccaaggacgtcgtggcgtcctacttcgtggacaagctgggcgaggggtaccggcaccacggggaggtgtacgggcgctactgctctgagatgagccgtctgtcgctggagctcatggaggtgctaggcgagagcctgggcgtgggccggcgccacttccggcgcttcttccaggggaacgactccatcatgcgcctcaactactacccgccgtgccagcggccctacgacacgctgggcacggggccgcattgcgaccccacgtcgctcaccatcctgcaccaggacgacgtgggcggactccaggtgttcgacgccgccacgctcgcgtggcgctccatcaggccccgcccgggcgccttcgtcgtcaacatcggcgacaccttcatggcgctctccaacgggcgctacaggagctgcctccaccgcgccgtcgtcaacagccgggtggcacgccgctcgctcgccttcttcctgtgcccggagatggacaaggtggtcaggccgcccaaggagctggtggacgacgccaacccgagggcgtacccggacttcacgtggaggacgctgctggacttcaccatgaggcactacaggtcggacatgaggacgctcgaggccttctccaactggctcagcaccagtagcaatggcggacagcacctgctggagaagaagtag

SEQ ID NO: 3 - protein, 371 aa, Zea mays
MVLAAHDPPPLVFDAARLSGLSDIPQQFIWPADESPTPDSAEELAVPLIDLSGDAAEVVRQVRRACDLHGFFQVVGHGIDAALTAEAHRCMDAFFTLPLPDKQRAQRRQGDSCGYASSFTGRFASKLPWKETLSFRYTDDDDGDKSKDVVASYFVDKLGEGYRHHGEVYGRYCSEMSRLSLELMEVLGESLGVGRRHFRRFFQGNDSIMRLNYYPPCQRPYDTLGTGPHCDPTSLTILHQDDVGGLQVFDAATLAWRSIRPRPGAFVVNIGDTFMALSNGRYRSCLHRAVVNSRVARRSLAFFLCPEMDKVVRPPKELVDDANPRAYPDFTWRTLLDFTMRHYRSDMRTLEAFSNWLSTSSNGGQHLLEKK

SEQ ID NO: 4 - DNA, 1517 bp, Zea mays
caggaataaaataagcctccgcccggcttcgttgcatccacgcacgcagcaagcgatcggatttcgccagcatggcggcggcggccgtggtgttcgacgccgaggcgctgagccgggaggagcacatcccggcgcagttcgtgtggcccaccgaggagcgggcgccggcgggcggcgtggaggaggtcgccatccccgtggtcgacctcggcgagttcctccgccgcggggtgctcccgcgcggcgtggcggaggcgtgcgagcgccacggcgtcttccaggtggtgaaccacggcgtgggcgccgcgctgctcgccgaggcctaccgctgttgcgacgccttttacgcgctcccgctcgcggacaagcagcgcgcgcagcgccggcacggggagaaccacggctacgccagcagcttcacgggccgcttccactgctgcctgccgtggaaggagacgctgtccttcaactgccccgccggtgccgggactgcgcgcgccgtcgtcggctacttcgtcgacgtcctcggcgaggactaccgccacatgggggaggtgtaccaggagtactgcgacgcgatgacgcgtctggcgctggacgtgacggaggtgctggcggcagcgctggggctggaccgcggcgcactgcgcggcttcttcgagggcggcgactccgtcatgcggctgaaccactacccggcgtgccggcagccgcacctgacgctggggacgggcccgcaccgggacccgacgtcgctgacgctgctgcaccaggacgacgtgggcgggctgcaggtgcgcgccggcggcgggccgtggcgcgcggtgcggccccgcgcggacgcgttcgtggtcaacattggcgacaccttcgccgcgctcaccgacgggcgtcacaccagctgcctgcaccgcgccgtggtgaccggcggcggctcccgccggtcgctcgccttcttcctcaacccgccgctggaccgcgtcgtccgcccgccgggcgcgctcctccaggagaacaagcaggcgggccgcccgcgcgcgttcccggacttcacgtggcgcgagttcctcgagttcacgcagaagcactaccggtcggacgcgggcaccatggacgccttcgtgtcgtggatcgcgggaggccgccgccaccatggcggacaggaggagggcaactgagatcgatgcatctctagctgtaggcagcagcgcagcagctaccaagaataatggccggcgacggagatgcagctacgacgcacaaataaattgagtgtttgtggtacaataaggacgaggacgatcaatggcgacctgtaaccggtgcagttttagttaatctttcatggcgatatggcattaaccaatcgttggtgtaaaatgcgtgcatgctttgcatgccaatgttggccatgtgatggcacagcgtgagtgtagctcacccaccgtgacaacgtgctaatttcgtgtggtcctagataccaaggtcgtctaatgaacttgatggattgatgattt

SEQ ID NO: 5 - DNA, 1107 bp, Zea mays
atggcggcggcggccgtggtgttcgacgccgaggcgctgagccgggaggagcacatcccggcgcagttcgtgtggcccaccgaggagcgggcgccggcgggcggcgtggaggaggtcgccatccccgtggtcgacctcggcgagttcctccgccgcggggtgctcccgcgcggcgtggcggaggcgtgcgagcgccacggcgtcttccaggtggtgaaccacggcgtgggcgccgcgctgctcgccgaggcctaccgctgttgcgacgccttttacgcgctcccgctcgcggacaagcagcgcgcgcagcgccggcacggggagaaccacggctacgccagcagcttcacgggccgcttccactgctgcctgccgtggaaggagacgctgtccttcaactgccccgccggtgccgggactgcgcgcgccgtcgtcggctacttcgtcgacgtcctcggcgaggactaccgccacatgggggaggtgtaccaggagtactgcgacgcgatgacgcgtctggcgctggacgtgacggaggtgctggcggcagcgctggggctggaccgcggcgcactgcgcggcttcttcgagggcggcgactccgtcatgcggctgaaccactacccggcgtgccggcagccgcacctgacgctggggacgggcccgcaccgggacccgacgtcgctgacgctgctgcaccaggacgacgtgggcgggctgcaggtgcgcgccggcggcgggccgtggcgcgcggtgcggccccgcgcggacgcgttcgtggtcaacattggcgacaccttcgccgcgctcaccgacgggcgtcacaccagctgcctgcaccgcgccgtggtgaccggcggcggctcccgccggtcgctcgccttcttcctcaacccgccgctggaccgcgtcgtccgcccgccgggcgcgctcctccaggagaacaagcaggcgggccgcccgcgcgcgttcccggacttcacgtggcgcgagttcctcgagttcacgcagaagcactaccggtcggacgcgggcaccatggacgccttcgtgtcgtggatcgcgggaggccgccgccaccatggcggacaggaggagggcaactga

SEQ ID NO: 6 - protein, 368 aa, Zea mays
MAAAAVVFDAEALSREEHIPAQFVWPTEERAPAGGVEEVAIPVVDLGEFLRRGVLPRGVAEACERHGVFQVVNHGVGAALLAEAYRCCDAFYALPLADKQRAQRRHGENHGYASSFTGRFHCCLPWKETLSFNCPAGAGTARAVVGYFVDVLGEDYRHMGEVYQEYCDAMTRLALDVTEVLAAALGLDRGALRGFFEGGDSVMRLNHYPACRQPHLTLGTGPHRDPTSLTLLHQDDVGGLQVRAGGGPWRAVRPRADAFVVNIGDTFAALTDGRHTSCLHRAVVTGGGSRRSLAFFLNPPLDRVVRPPGALLQENKQAGRPRAFPDFTWREFLEFTQKHYRSDAGTMDAFVSWIAGGRRHHGGQEEGN

SEQ ID NO: 7 - DNA, 1522 bp, Zea mays
gcacactcgcagctcgcacatctcatggtgtcctaagaacggcaagagccagctctgcctagcagcagcgcacagccacatccatggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaacgagccggccgtctctttcgccggggcccgcgcggggttcgcccacgtggtgatcaggtggcagacatgtggcccacgggccccgcgccgccttccccatttttggacgaccctactgctactactactagtgtacatatgcaaaaaaatacatatatatataggtactttctctaatatttttatatataagcaaggcggcctggtgttcttttctttgttttgtcgacaactgtttgatcccatcctatggacgatggatagttcaatgtttgtac

SEQ ID NO: 8 - DNA, 1161 bp, Zea mays
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 9 - protein, 386 aa, Zea mays
MDASPTPPLPLRAPTPSIDLPAGKDRADAAANKAAAVFDLRREPKIPEPFLWPHEEARPTSAAELEVPVVDVGVLRNGDGAGLRRAAAQVAAACATHGFFQVCGHGVDAALGRAALDGASDFFRLPLAEKQRARRVPGTVSGYTSAHADRFASKLPWKETLSFGFHDGAAAPVVVDYFTGTLGQDFEPVGRVYQRYCEEMKELSLTIMELLELSLGVERGYYREFFEDSRSIMRCNYYPPCPVPERTLGTGPHCDPTALTILLQDDVGGLEVLVDGEWRPVRPVPGAMVINIGDTFMALSNGRYKSCLHRAVVNRRQERQSLAFFLCPREDRVVRPPASAAPRQYPDFTWADLMRFTQRHYRADTRTLDAFTRWLSHGPAAAAPCT

SEQ ID NO: 10 - DNA, 1457 bp, Zea mays
taatcacctcatcacaggtccccccagcctcactctcgcgccggctcaaggtacattgcgtgtcctagccaagacacgcagctcatctcagcctcacacgcacagcaagagcgaggcgtgattcgccatgggcggcctcactatggaccaggccttcgtgcaggcccccgagcaccgccccaagcccatcgtcaccgaggccaccggcatccctctcatcgacctctcgcctctggccgccagcggcggcgccgtggacgcgctggccgccgaggtgggcgcggcgagccgggactggggcttcttcgtggtcgtgggccacggcgtgcccgcagagaccgtggcgcgcgcgacggaggcgcagcgagcgttcttcgcgctgccggcagagcggaaggccgccgtgcggaggaacgaggcggagccgctcgggtactacgagtcggagcacaccaagaacgtgagggactggaaggaggtgtacgacctcgtgccgcgcgagccgccgccgccggcagccgtggccgacggcgagcttgtgttcgataacaagtggccccaggatctaccgggcttcagagaggcgctggaggagtacgcgaaagcgatggaagagctggcgttcaagctgctggagctgatcgcccggagcctgaagctgaggcccgaccggctgcacggcttcttcaaggaccagacgaccttcatccggctgaaccactaccctccttgcccgagccccgacctggccctcggcgtggggcggcacaaggacgccggcgccctgaccatcctgtaccaggacgacgtcggggggctcgacgtccggcggcgctccgacggcgagtgggtccgcgtcaggcccgtgcccgactcgttcatcatcaacgtcggcgacctcatccaggtacgagagcgcggagcaccgggtgtcggtgaactcggcgagggagaggttctccatgccctacttcttcaacccggcgacctacaccatggtggagccggtggaggagctggtgagcaaggacgatccgcccaggtacgacgcctacaactggggcgacttcttcagcaccaggaagaacagcaacttcaagaagctcaacgtggagaacattcagatcgcgcatttcaagaagagcctcgtcctcgcctaactactgctactgctaggatccatgccattgccatgtcgtcttcagattcagagcacgccatgtcgtcgctagcttcgtggtagaacaaataatgatgtgcgtgctgtgtgtaagcatggatatggatgtgaatatgtaatatgatgagcactcctactttggtatgtttgggaataacagacttgtgttggtctggttcattatttgtaagaaaatcaaaaagagttagtagggcaggaggctaaccacagtcatgctgcaccacatccctggtggaaagctggccgggttacgcta

SEQ ID NO: 11 - DNA, 1116 bp, Zea mays
atgggcggcctcactatggaccaggccttcgtgcaggcccccgagcaccgccccaagcccatcgtcaccgaggccaccggcatccctctcatcgacctctcgcctctggccgccagcggcggcgccgtggacgcgctggccgccgaggtgggcgcggcgagccgggactggggcttcttcgtggtcgtgggccacggcgtgcccgcagagaccgtggcgcgcgcgacggaggcgcagcgagcgttcttcgcgctgccggcagagcggaaggccgccgtgcggaggaacgaggcggagccgctcgggtactacgagtcggagcacaccaagaacgtgagggactggaaggaggtgtacgacctcgtgccgcgcgagccgccgccgccggcagccgtggccgacggcgagcttgtgttcgataacaagtggccccaggatctaccgggcttcagagaggcgctggaggagtacgcgaaagcgatggaagagctggcgttcaagctgctggagctgatcgcccggagcctgaagctgaggcccgaccggctgcacggcttcttcaaggaccagacgaccttcatccggctgaaccactaccctccttgcccgagccccgacctggccctcggcgtggggcggcacaaggacgccggcgccctgaccatcctgtaccaggacgacgtcggggggctcgacgtccggcggcgctccgacggcgagtgggtccgcgtcaggcccgtgcccgactcgttcatcatcaacgtcggcgacctcatccaggtacgagagcgcggagcaccgggtgtcggtgaactcggcgagggagaggttctccatgccctacttcttcaacccggcgacctacaccatggtggagccggtggaggagctggtgagcaaggacgatccgcccaggtacgacgcctacaactggggcgacttcttcagcaccaggaagaacagcaacttcaagaagctcaacgtggagaacattcagatcgcgcatttcaagaagagcctcgtcctcgcctaactactgctactgctaggatccatgccattgccatgtcgtcttcagattcagagcacgccatgtcgtcgctagcttcgtggtag

SEQ ID NO: 12 - protein, 371 aa, Zea mays
MGGLTMDQAFVQAPEHRPKPIVTEATGIPLIDLSPLAASGGAVDALAAEVGAASRDWGFFVVVGHGVPAETVARATEAQRAFFALPAERKAAVRRNEAEPLGYYESEHTKNVRDWKEVYDLVPREPPPPAAVADGELVFDNKWPQDLPGFREALEEYAKAMEELAFKLLELIARSLKLRPDRLHGFFKDQTTFIRLNHYPPCPSPDLALGVGRHKDAGALTILYQDDVGGLDVRRRSDGEWVRVRPVPDSFIINVGDLIQVRERGAPGVGELGEGEVLHALLLQPGDLHHGGAGGGAGEQGRSAQVRRLQLGRLLQHQEEQQLQEAQRGEHSDRAFQEEPRPRLTTATARIHAIAMSSSDSEHAMSSLASW

SEQ ID NO: 13 - DNA, 1733 bp, Zea mays
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaa

SEQ ID NO: 14 - DNA, 1392 bp, Zea mays
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 15 - protein, 463 aa, Zea mays
MRPRLPPNVPSLPSSLSLLANSLSSPVTNTPTRPDSFPAYLQLAHLMVSQERQEPAVPSSSSSSAKRAATSMDASPAPPLLLRAPTPSPSIDLPAGKDKADAAASKAGAAVFDLRREPKIPAPFLWPQEEARPSSAAELEVPMVDVGVLRNGDRAGLRRAAAQVAAACATHGFFQVCGHGVDAALGRAALDGASDFFRLPLAEKQRARRVPGTVSGYTSAHADRFAAKLPWKETLSFGYHDGAASPVVVDYFVGTLGQDFEPMGWVYQRYCEEMKELSLTIMELLELSLGVELRGYYREFFEDSRSIMRCNYYPPCPEPERTLGTGPHCDPTALTILLQDDVGGLEVLVDGEWRPVRPVPGAMVINIGDTFMALSNGRYKSCLHRAVVNQRRARRSLAFFLCPREDRVVRPPASAAPRRYPDFTWADLMRFTQRHYRADTRTLDAFTRWLSHGPAQAAAPPCT

SEQ ID NO: 16 - DNA, 1510 bp, Zea mays
aaagagcgcgcgacggcggcccctgggagagccatgcgagactggaggcggaaccgcgcacgacaccaagctgccgcgccggactgctgcacgcaagcgcagcgcaggaccgaccgacctccgtaggcacgcacggcgccggcggcatggcggagcacctcctgtcgacggccgtgcacgacacgctgccggggagctacgtgcggccggagccggagcgcccgcgcctcgcggaggtcgtgaccggcgcgcgcatccccgtcgtggacctgggcagccccgaccgcggcgcggtcgtggccgccgtcggcgacgcctgccgctcgcacggcttcttccaggtcgtcaaccacgggatacacgccgccctggtcgcggcggtgatggccgcggggcgcggcttcttccggctgccccccgaggagaaggccaagctctactccgacgaccccgccaggaagatccggctgtccaccagcttcaacgtgcgcaaggagacggtgcacaactggcgcgactacctccgcctgcactgccatcccctcgacgagttcctgcccgattggccgtccaacccgcccgatttcaaggagaccatgggcacctactgcaaggaggtccgggagctcgggttcaggctgtacgccgcgatctcggagagcctgggcctagaggcgagctacatgaaggaagcgctgggggagcaggagcagcacatggcggtcaacttctacccgccgtgcccggagccggagctcacctacggcctcccggcgcacaccgaccccaacgcgctcaccatcctgctcatggacccggacgtcgccggcctgcaggtgctccacgccggccagtgggtcgccgtcaacccgcagcccggcgcgctcatcatcaacatcggcgaccagctgcaggcgctgagcaacgggcagtaccggagcgtgtggcaccgcgcggtggtgaactcggaccgggagcgcatgtccgtggcgtcgttcctgtgcccgtgcaaccacgtcgtgctcggccccgcgcggaagctcgtcaccgaggacaccccggccgtgtacaggaactacacgtacgacaagtactacgccaagttctggagcaggaacctggaccaggagcactgcctcgagctcttcagaacctagcgaatcggatacggatggatggatacattacatacgcgccctctgtttttctccatgacgttagaagaacacgttctgcaatgtttgtccattcaaggtggtatcaatcaaggctgtggtcgttgcaattcttccgctccatatacatgattaaatgctttgaaagaaaaagaaaaaaaagaaacacaagtattatggcactactagtgtttttaggaacaaggaaagaggggttgcccctgctggctatatatattaaatataaataaaggtaaggctgtagacattggtgaataagagaaagtatttgagtttctctattgtcactccagaacagactcctttgcctcgat

SEQ ID NO: 17 - DNA, 1011 bp, Zea mays
atggcggagcacctcctgtcgacggccgtgcacgacacgctgccggggagctacgtgcggccggagccggagcgcccgcgcctcgcggaggtcgtgaccggcgcgcgcatccccgtcgtggacctgggcagccccgaccgcggcgcggtcgtggccgccgtcggcgacgcctgccgctcgcacggcttcttccaggtcgtcaaccacgggatacacgccgccctggtcgcggcggtgatggccgcggggcgcggcttcttccggctgccccccgaggagaaggccaagctctactccgacgaccccgccaggaagatccggctgtccaccagcttcaacgtgcgcaaggagacggtgcacaactggcgcgactacctccgcctgcactgccatcccctcgacgagttcctgcccgattggccgtccaacccgcccgatttcaaggagaccatgggcacctactgcaaggaggtccgggagctcgggttcaggctgtacgccgcgatctcggagagcctgggcctagaggcgagctacatgaaggaagcgctgggggagcaggagcagcacatggcggtcaacttctacccgccgtgcccggagccggagctcacctacggcctcccggcgcacaccgaccccaacgcgctcaccatcctgctcatggacccggacgtcgccggcctgcaggtgctccacgccggccagtgggtcgccgtcaacccgcagcccggcgcgctcatcatcaacatcggcgaccagctgcaggcgctgagcaacgggcagtaccggagcgtgtggcaccgcgcggtggtgaactcggaccgggagcgcatgtccgtggcgtcgttcctgtgcccgtgcaaccacgtcgtgctcggccccgcgcggaagctcgtcaccgaggacaccccggccgtgtacaggaactacacgtacgacaagtactacgccaagttctggagcaggaacctggaccaggagcactgcctcgagctcttcagaacctag

SEQ ID NO: 18 - protein, 336 aa, Zea mays
MAEHLLSTAVHDTLPGSYVRPEPERPRLAEVVTGARIPVVDLGSPDRGAVVAAVGDACRSHGFFQVVNHGIHAALVAAVMAAGRGFFRLPPEEKAKLYSDDPARKIRLSTSFNVRKETVHNWRDYLRLHCHPLDEFLPDWPSNPPDFKETMGTYCKEVRELGFRLYAAISESLGLEASYMKEALGEQEQHMAVNFYPPCPEPELTYGLPAHTDPNALTILLMDPDVAGLQVLHAGQWVAVNPQPGALIINIGDQLQALSNGQYRSVWHRAVVNSDRERMSVASFLCPCNHVVLGPARKLVTEDTPAVYRNYTYDKYYAKFWSRNLDQEHCLELFRT

SEQ ID NO: 19 - DNA, 1387 bp, Zea mays
gttttctttttgaacgtaactgacagaagctatctgcctagctacggcgtgtcggttgcttgtctcaccaaagcagcgacatggaagcctgacagctcgtcgcgtcgcgccatttccacccaacaaagcggcggcgccagcacgcactgcttctgcttgtgcgtgctcctccgttccgggcacgcctctaaagtctatacagcctcgaatccatcccggccgccgctcctgggggatactacagcgagccgaagcggggatggcggagatccctgtgatcgacctgcgcgtcgccggctcggcggccgaggagtccgcgcggctgcgggccgcgtgcgagcgcctgggctgcttccgggtgaccggccacggcgtgccctcggtgctcctggcagagatgaaggccgccgtgcgcgcgctcttcgacctccccgacgacgccaagcgccgcaacgccgacgtcatcaccggcagcggctacgtcgcccccagcccgaccaacccgctctacgaggccttcgggctcctcgacgccgccgtgcccaccgacgtcgacgccttttgcgcgctcctcgacgcgccgcccaacatcagggagaccgtcaaggcctacgcggagaagatgcacgatgtgatcgttggcgtcgcccgcgagctggcgtctagcctggggctagtcgaggagcactcgttccaggactggccgtgccagttccgcatcaacaggtacaactacacgcgggagacggtgggctcctccggcgtgcagacccacacggactcgggcttcctcaccgtgctccatgaggacgagtgtgtcggcggcctcgaggtcctggacccgggcaccggcgagttcgtgcccgtggaccccgtcgcgggctcctttctcgtaaacatcggcgacgtcggcacggcgtggagcaacgggaggctgcacaacgtgaagcaccgggtgcggtgcgtcgcacccgtgccgcgcatctccatcgccatgttcctgctcgcacccaaggacgacagcgtgagcgcaccggcggcgttcgtggacgcggaccacccgcgcaggtacaaggtgttcaactacaacgactatcggaggctcagactgtccaccggcgagcacgcaggcgaggcgctcgcacggatggcggcgtgacgtggctggagctgcaaattggattggaagccgagacaagccgttagttatttaccatgcccgtgcgttcaccgcacacaatcatattcaaaagccataaaataaaaaataattttaatatcagtcaacatatggtttaaatatcatatggagtacaatattccgaatttttttttgtaatttagtctgtcttttgaaaaaaatgcacatctagacctccggatgact

SEQ ID NO: 20 - DNA, 900 bp, Zea mays
atggcggagatccctgtgatcgacctgcgcgtcgccggctcggcggccgaggagtccgcgcggctgcgggccgcgtgcgagcgcctgggctgcttccgggtgaccggccacggcgtgccctcggtgctcctggcagagatgaaggccgccgtgcgcgcgctcttcgacctccccgacgacgccaagcgccgcaacgccgacgtcatcaccggcagcggctacgtcgcccccagcccgaccaacccgctctacgaggccttcgggctcctcgacgccgccgtgcccaccgacgtcgacgccttttgcgcgctcctcgacgcgccgcccaacatcagggagaccgtcaaggcctacgcggagaagatgcacgatgtgatcgttggcgtcgcccgcgagctggcgtctagcctggggctagtcgaggagcactcgttccaggactggccgtgccagttccgcatcaacaggtacaactacacgcgggagacggtgggctcctccggcgtgcagacccacacggactcgggcttcctcaccgtgctccatgaggacgagtgtgtcggcggcctcgaggtcctggacccgggcaccggcgagttcgtgcccgtggaccccgtcgcgggctcctttctcgtaaacatcggcgacgtcggcacggcgtggagcaacgggaggctgcacaacgtgaagcaccgggtgcggtgcgtcgcacccgtgccgcgcatctccatcgccatgttcctgctcgcacccaaggacgacagcgtgagcgcaccggcggcgttcgtggacgcggaccacccgcgcaggtacaaggtgttcaactacaacgactatcggaggctcagactgtccaccggcgagcacgcaggcgaggcgctcgcacggatggcggcgtga

SEQ ID NO: 21 - protein, 299 aa, Zea mays
MAEIPVIDLRVAGSAAEESARLRAACERLGCFRVTGHGVPSVLLAEMKAAVRALFDLPDDAKRRNADVITGSGYVAPSPTNPLYEAFGLLDAAVPTDVDAFCALLDAPPNIRETVKAYAEKMHDVIVGVARELASSLGLVEEHSFQDWPCQFRINRYNYTRETVGSSGVQTHTDSGFLTVLHEDECVGGLEVLDPGTGEFVPVDPVAGSFLVNIGDVGTAWSNGRLHNVKHRVRCVAPVPRISIAMFLLAPKDDSVSAPAAFVDADHPRRYKVFNYNDYRRLRLSTGEHAGEALARMAA

SEQ ID NO: 22 - DNA, 1496 bp, Zea mays
gtcggtctcttgtctcaccaaaccggcgacatggtacatggaggccagcccgtcgcttggcgccacaagtctcggtgccgtccgtccgacaagcggcgccagcgcacgctggctgctcgtgcacgcctctaaatacggccccggacccgccaccaagcgaaggccaatcccgtccgccgccccccaccaaccacgaaccacgcaagcgaacccggccggcgcggggcagcggcgatggcggagatcccggtgatcgacctgcgcctcgccggctcgtcgcccgacgagtcggcgcggctgcgcgacgcgtgcgagcgcctgggctgctttcgggtgaccggccacggcgcgcccgcggggctcctggccgacatgaaggccgccgtgcgcgcgctcttcgacctccccgacgacgccaagcgccgcaacgccgacgtcatccccggcagcggctacgtcgcgccctgccccgccaacccgctctacgaggccttcgggctcctcgacgccgccgcgcccgccgacgtcgacgccttctgcgcgcgcctcgacgcgccgcccaaagtcagggagaccgtcaagacctacgcggagaagatgcacgacgtgatcgtcggcgtcgccggcgagctggccaccagcctggggctgggcctggaggagcactcgttccaggactggccgtgccagttccgcatcaacaggtacaactacacgcaggagacggtgggctcctccggcgtgcagacccacacggactcgggcttcctcaccgtgctccaggaggacgagtgcgtcggcggcctcgaggtgctggaccccgccgccggtgagttcgtgcccgtggaccccgtcgccggctccttcctcgtcaacatcggcgacgtcggcacggcgtggagcaacgggaggctccacaacgtgaagcaccgggtgcggtgcgtcgcgcccgtgccgcgcatctccatcgccatgttcctgctggcgcccaaggacgaccgcgtgagcgccccggaggcgttggtcgacgcgggccacccgcgtcggtacaagccgttcaactacgacgactaccggaggctccggctgtccaccggcgagcgcgcaggcgaggcgctcgcgcggatggcggcgtgatgtcgtcacgcacgtgcaagccgttaattataggctcgcgcatgcatacgcctacacgagaggttgtctcgttaagccgttctattaaaatgtgtgggggagaaagatgactaccgtggtgccatgtggattgctatcgggtctgatcaataaaatcttgcaacacttgcacgtgcgattccatatcctagcacgggtgggcgccacgctagtaggtagagaccggagcggccaaaaaatggctacagcaccagtaggtgaactctcaagcaacactggctatcccacttctgacgttgtctctctcatcactatgtatgaccagcgaatgaagtgtttaaaaatctgacgccgtgaaa

SEQ ID NO: 23 - DNA, 903 bp, Zea mays
atggcggagatcccggtgatcgacctgcgcctcgccggctcgtcgcccgacgagtcggcgcggctgcgcgacgcgtgcgagcgcctgggctgctttcgggtgaccggccacggcgcgcccgcggggctcctggccgacatgaaggccgccgtgcgcgcgctcttcgacctccccgacgacgccaagcgccgcaacgccgacgtcatccccggcagcggctacgtcgcgccctgccccgccaacccgctctacgaggccttcgggctcctcgacgccgccgcgcccgccgacgtcgacgccttctgcgcgcgcctcgacgcgccgcccaaagtcagggagaccgtcaagacctacgcggagaagatgcacgacgtgatcgtcggcgtcgccggcgagctggccaccagcctggggctgggcctggaggagcactcgttccaggactggccgtgccagttccgcatcaacaggtacaactacacgcaggagacggtgggctcctccggcgtgcagacccacacggactcgggcttcctcaccgtgctccaggaggacgagtgcgtcggcggcctcgaggtgctggaccccgccgccggtgagttcgtgcccgtggaccccgtcgccggctccttcctcgtcaacatcggcgacgtcggcacggcgtggagcaacgggaggctccacaacgtgaagcaccgggtgcggtgcgtcgcgcccgtgccgcgcatctccatcgccatgttcctgctggcgcccaaggacgaccgcgtgagcgccccggaggcgttggtcgacgcgggccacccgcgtcggtacaagccgttcaactacgacgactaccggaggctccggctgtccaccggcgagcgcgcaggcgaggcgctcgcgcggatggcggcgtga

SEQ ID NO: 24 - protein, 300 aa, Zea mays
MAEIPVIDLRLAGSSPDESARLRDACERLGCFRVTGHGAPAGLLADMKAAVRALFDLPDDAKRRNADVIPGSGYVAPCPANPLYEAFGLLDAAAPADVDAFCARLDAPPKVRETVKTYAEKMHDVIVGVAGELATSLGLGLEEHSFQDWPCQFRINRYNYTQETVGSSGVQTHTDSGFLTVLQEDECVGGLEVLDPAAGEFVPVDPVAGSFLVNIGDVGTAWSNGRLHNVKHRVRCVAPVPRISIAMFLLAPKDDRVSAPEALVDAGHPRRYKPFNYDDYRRLRLSTGERAGEALARMAA

SEQ ID NO: 25 - DNA, 1614 bp, Zea mays
accacacgaattgcacatctccacagctcacgattccaacactagctacatatatatgtagctttctaggctactatatacactcaccaccaagtgtgaagtgtgtatatatagtgacagctactgcaatatatacatacgcgtcacctatatattagccaagctagctatatgagcttggttgcggcgccaatggcgatcgtcgacgtggccaacgcccagctgcagcaagcagcagcagcagctgccaagaaagacgaggacggccatgagcagcaggagtcgtcctacgactacggcgcgctgatgaaaggcgtgaggcacctgtcggacagcggcattaccaggctgcccgacaggtacgtcctgcccgcgtccgaccgccccggcgtccttgccgtctcgtcgtccgtggcgggcagcggcagggtcaagctccctgtcgtcaacctcgccggcctccgcgacccctgccagcgcgccgccgtgctggccacgctcgacgccgcgtgccgggagtacggcttctttcaggtggtaaaccacgggttcgggagcgacgtgagcggcgggatgctggacgtggcgcagcgcttcttcgagctgccgctggccgagcgagcgcggcacatgtcggcggacgtgcgggcgccggtgcgctacggcaccagcttcaaccaggccaaggacgacgtgctctgctggcgcgacttcctcaagctcgtctgccagccgctgcaggcggtgctcccgtactggccccagcagccggcggacctcagggacgtggccaccaggtacgccacggcgagccaccggctgttcatggaggtcatggaggcggcgctggaggccctgggcatccccacggccggcggcgtgctcggggagctggcagcgtcgtcgtcgcacatgatgacggtgaactgctacccggcgtgcccgcagcctgagctcacgctggggatgccctcgcactcggactacggcctcttcacgttcgtcctgcaggaccacgtcgagggcctccaggtcatgcacgacggccgctggctcaccatcgaccccatcccgggatcgttcgtcgtcaacgtcggcgaccacctagagatctacagcaacgggcggtacaagagcgcgctgcaccgggtgcacgtgaactccacgcggccgcgcatctcggtggcgtcgttccacagcctgccggcggagcgagtgatcgggccggcgccggagctggtggacgacgaggccggcaacccgcggcggtacatggacaccgacttcgctaccttcctcgcctacctcgcatccgcggacggcaagaacaagaccttcctccagtcaaggaagctgcctgctgctgctcctccatgcctctagctaactagatagctgcttattaatctgacagaataaaattaatcagttcagcgcacaattccacaagcgaaaacaaacctggatttgttttaattagctctgcccttcattattacattcaagctagctcttggtcaacgcatgcacacaagcttgagcattgactggtcccttttcaatcggttgcattgtactccctccgtaccaaaattggttgtcgctatagtattt

SEQ ID NO: 26 - DNA, 1212 bp, Zea mays
atgagcttggttgcggcgccaatggcgatcgtcgacgtggccaacgcccagctgcagcaagcagcagcagcagctgccaagaaagacgaggacggccatgagcagcaggagtcgtcctacgactacggcgcgctgatgaaaggcgtgaggcacctgtcggacagcggcattaccaggctgcccgacaggtacgtcctgcccgcgtccgaccgccccggcgtccttgccgtctcgtcgtccgtggcgggcagcggcagggtcaagctccctgtcgtcaacctcgccggcctccgcgacccctgccagcgcgccgccgtgctggccacgctcgacgccgcgtgccgggagtacggcttctttcaggtggtaaaccacgggttcgggagcgacgtgagcggcgggatgctggacgtggcgcagcgcttcttcgagctgccgctggccgagcgagcgcggcacatgtcggcggacgtgcgggcgccggtgcgctacggcaccagcttcaaccaggccaaggacgacgtgctctgctggcgcgacttcctcaagctcgtctgccagccgctgcaggcggtgctcccgtactggccccagcagccggcggacctcagggacgtggccaccaggtacgccacggcgagccaccggctgttcatggaggtcatggaggcggcgctggaggccctgggcatccccacggccggcggcgtgctcggggagctggcagcgtcgtcgtcgcacatgatgacggtgaactgctacccggcgtgcccgcagcctgagctcacgctggggatgccctcgcactcggactacggcctcttcacgttcgtcctgcaggaccacgtcgagggcctccaggtcatgcacgacggccgctggctcaccatcgaccccatcccgggatcgttcgtcgtcaacgtcggcgaccacctagagatctacagcaacgggcggtacaagagcgcgctgcaccgggtgcacgtgaactccacgcggccgcgcatctcggtggcgtcgttccacagcctgccggcggagcgagtgatcgggccggcgccggagctggtggacgacgaggccggcaacccgcggcggtacatggacaccgacttcgctaccttcctcgcctacctcgcatccgcggacggcaagaacaagaccttcctccagtcaaggaagctgcctgctgctgctcctccatgcctctag

SEQ ID NO: 27 - protein, 403 aa, Zea mays
MSLVAAPMAIVDVANAQLQQAAAAAAKKDEDGHEQQESSYDYGALMKGVRHLSDSGITRLPDRYVLPASDRPGVLAVSSSVAGSGRVKLPVVNLAGLRDPCQRAAVLATLDAACREYGFFQVVNHGFGSDVSGGMLDVAQRFFELPLAERARHMSADVRAPVRYGTSFNQAKDDVLCWRDFLKLVCQPLQAVLPYWPQQPADLRDVATRYATASHRLFMEVMEAALEALGIPTAGGVLGELAASSSHMMTVNCYPACPQPELTLGMPSHSDYGLFTFVLQDHVEGLQVMHDGRWLTIDPIPGSFVVNVGDHLEIYSNGRYKSALHRVHVNSTRPRISVASFHSLPAERVIGPAPELVDDEAGNPRRYMDTDFATFLAYLASADGKNKTFLQSRKLPAAAPPCL

SEQ ID NO: 28 - DNA, 1863 bp, Zea mays
tgccaccataccactagtgcaaggtcctagatttacacttggtgctacaccttgcttcgcccccttccttccttccttccttccttccctccttccttggtctctaggcagctagcagtgtggtgctgctgccggccgcctattggccgcctgggactgggatccattaattactgcgcgcgcgcggctaaccaaccaatcccagcgtgcgtaatctattgcccacatgccgacgccgtcgcacctcaacaagaacccgcgctacctggacttccgggcggcgcggcgggtgccggagtcgcacgcctggccgggcctgcacgaccaccccgtcgtggacggcggcgcgccgggccccgacgccgtgccggtggtggacctgggcgccgcggacccggcgccggcgccggcggcggcggtggcccgcgccgccgagcaatggggcgcgttcctgctcacgggccacggcgtccccgcggacctgctggcgcgcgtggaggaccggatcgccaccatgttcgcgctgccggccgacgacaagatgcgcgccgtgcgcgggcccggcgacgcctgcggctacggctccccgcccatctcctccttcttctccaagtgcatgtggtccgagggctacaccttctcgccggcctccctccgcgccgacctccgcaagctctggcccaaggccggcgacgactacaccagcttctgtgatgtgatggaggagttccacaagcacatgcgcgccctcgcggacaagctgctggagctgttcctcatggcgctggggctcaccgacgagcaggccagcgccgtcgaggccgagcggaggatcgccgagacgatgaccgccaccatgcatctcaactggtacccgaggtgcccggacccgcggcgcgcgctggggctgatcgcgcacaccgactcgggcttcttcaccttcgtgatgcagagcctcgtgcccgggctgcagctcttccgccacgccccggaccggtgggtggcggtgccggccgtgccgggcgccttcgtcgtcaacgtgggcgacctcttccacatcctcaccaacggccggttccacagcgtgtaccaccgcgccgtcgtgaaccgggacctcgacaggatctcgctcggctacttcctcggcccgccgccgcacgccaaggtggcgccgctgcgcgaggccgtgccgcccggccgggcccccgcgtaccgcgccgtcacgtggcccgagtacatgggcgtccgcaagaaggccttcaccaccggcgcctccgcgctcaagatggtcgccctcgccgccgccgccgacctcgacgacgacggcgacgccgccgtcgtccatcagcagcagcagctagtcgtctcgtcgtagccgagaccgatcgccggagactgatgctgatgatgatgcatatatacatgagagaaatcgtcgagtagactagccgattgcaaaagcaaccccagctgccgaaacctggcatatcgatcccattctctgctgcgcacatgtatgcatgcatgcgcttcgtccgttcgactcgtgtgtgcttgcttgcttgcgcgtgcagcagaactaattccgttccgcagctagctgctctgctctgctctgctggaatgtaattaagtagtagtatatggtagtagagaaaagattagctaggcgatcgatatagatgacgggccggggaagaagacgaattaattaagatcgatcgacgacgacgagctgtgcgtggctggctgtgttcttctctagcctagttacagaggccggctgctgctgcttccaatcgggctgcttgtcgctactgacgatcgttagtggatccattaactaatctggaattctggatt

SEQ ID NO: 29 - DNA, 1149 bp, Zea mays
atgccgacgccgtcgcacctcaacaagaacccgcgctacctggacttccgggcggcgcggcgggtgccggagtcgcacgcctggccgggcctgcacgaccaccccgtcgtggacggcggcgcgccgggccccgacgccgtgccggtggtggacctgggcgccgcggacccggcgccggcgccggcggcggcggtggcccgcgccgccgagcaatggggcgcgttcctgctcacgggccacggcgtccccgcggacctgctggcgcgcgtggaggaccggatcgccaccatgttcgcgctgccggccgacgacaagatgcgcgccgtgcgcgggcccggcgacgcctgcggctacggctccccgcccatctcctccttcttctccaagtgcatgtggtccgagggctacaccttctcgccggcctccctccgcgccgacctccgcaagctctggcccaaggccggcgacgactacaccagcttctgtgatgtgatggaggagttccacaagcacatgcgcgccctcgcggacaagctgctggagctgttcctcatggcgctggggctcaccgacgagcaggccagcgccgtcgaggccgagcggaggatcgccgagacgatgaccgccaccatgcatctcaactggtacccgaggtgcccggacccgcggcgcgcgctggggctgatcgcgcacaccgactcgggcttcttcaccttcgtgatgcagagcctcgtgcccgggctgcagctcttccgccacgccccggaccggtgggtggcggtgccggccgtgccgggcgccttcgtcgtcaacgtgggcgacctcttccacatcctcaccaacggccggttccacagcgtgtaccaccgcgccgtcgtgaaccgggacctcgacaggatctcgctcggctacttcctcggcccgccgccgcacgccaaggtggcgccgctgcgcgaggccgtgccgcccggccgggcccccgcgtaccgcgccgtcacgtggcccgagtacatgggcgtccgcaagaaggccttcaccaccggcgcctccgcgctcaagatggtcgccctcgccgccgccgccgacctcgacgacgacggcgacgccgccgtcgtccatcagcagcagcagctagtcgtctcgtcgtag

SEQ ID NO: 30 - protein, 382 aa, Zea mays
MPTPSHLNKNPRYLDFRAARRVPESHAWPGLHDHPVVDGGAPGPDAVPVVDLGAADPAPAPAAAVARAAEQWGAFLLTGHGVPADLLARVEDRIATMFALPADDKMRAVRGPGDACGYGSPPISSFFSKCMWSEGYTFSPASLRADLRKLWPKAGDDYTSFCDVMEEFHKHMRALADKLLELFLMALGLTDEQASAVEAERRIAETMTATMHLNWYPRCPDPRRALGLIAHTDSGFFTFVMQSLVPGLQLFRHAPDRWVAVPAVPGAFVVNVGDLFHILTNGRFHSVYHRAVVNRDLDRISLGYFLGPPPHAKVAPLREAVPPGRAPAYRAVTWPEYMGVRKKAFTTGASALKMVALAAAADLDDDGDAAVVHQQQQLVVSS

SEQ ID NO: 31 - DNA, 1439 bp, Zea mays
gacctccattttgattatctctatcctgtacgtgccgagagtccttcaaagccgacgacgagacgacgatgcagtcgtcgtcgtcatcagcctcgacgccggctgccgcttccggcctcgtcttcgatctcgggtctgcggcgggcgtgccggagacacacgcgtggccgggggtgaacgagtacccgtcggtggagtccgctggccgcgacgtggtcccggtggtggacatgggggtggcctgcccggacgcgacgcgggcgttggcgcgcgccgcagacgagtggggcgtgtttctgctcgtcggccacggcgtgccccgggaagtggcggcgcgtgccgaggagcaggtcgcgcgcctgttcgtgctcccggctcctgacaaggcccgcgcggggcgccgccccggggagcccacggccaccggctacggcaggccgcccctggcactccgcttctccaagctcatgtggtccgaggggtacacgttccgcgccgccaccgtccgcgaagagttccgccgcgtctggcccgacggcggcgacgactacctccgcttctgcgacgtgatggaggagtacgacagagagatgagggctctcggtggcaggctgctcgacctcttcttcatggcgctcggcctcaccgacgtccagttcgccaccggcgagacggagcggaggatccgcgagacctggacggcgacgatgcacccaatcctgtgtccggaaccggagcgcgccatcgggctgacggcgcacacggactcgggcttcatcacgctcatcatgcagagccccgtgcccgggctgcagctgctccgccgcgggccggaccggtgggtgacggtgccggcgccgccgggcgcgctcatcgtcatgctcggcgacctgttccaggtgctcacgaacggccgcttccggagccctatccaccgcgccgtcgtaagccgagagcgcgagcggatctccgtgccctacttcctctgcccgccggaggacatgacggtggcgccgctcgcgtccgctctgctgccggggaggaaggccgtgttccgggccgtgacgtggccagagtacatggaggtcaagcacaaggtgttcggcacggatgcgccggccctggagatgctgcagctgcaggtggatgaggaagaacaaggtgaaagggccgccaccacctaagccctaaggaactactagctgaatccataaactaataaagaattcgtgaataagggcgttggaagactggacacaacacaagagagttgctatatatcgtatttctgaaatttaaggcaaatatcttagttaaaaaactggtatatttaaatagacaatatatatctaaaataaagatagttcaccatttttacggtcgaacaatgataaagttatatattgtctgaatagtaacaaattaaagatttccaggag

SEQ ID NO: 32 - DNA, 1116 bp, Zea mays
atgcagtcgtcgtcgtcatcagcctcgacgccggctgccgcttccggcctcgtcttcgatctcgggtctgcggcgggcgtgccggagacacacgcgtggccgggggtgaacgagtacccgtcggtggagtccgctggccgcgacgtggtcccggtggtggacatgggggtggcctgcccggacgcgacgcgggcgttggcgcgcgccgcagacgagtggggcgtgtttctgctcgtcggccacggcgtgccccgggaagtggcggcgcgtgccgaggagcaggtcgcgcgcctgttcgtgctcccggctcctgacaaggcccgcgcggggcgccgccccggggagcccacggccaccggctacggcaggccgcccctggcactccgcttctccaagctcatgtggtccgaggggtacacgttccgcgccgccaccgtccgcgaagagttccgccgcgtctggcccgacggcggcgacgactacctccgcttctgcgacgtgatggaggagtacgacagagagatgagggctctcggtggcaggctgctcgacctcttcttcatggcgctcggcctcaccgacgtccagttcgccaccggcgagacggagcggaggatccgcgagacctggacggcgacgatgcacccaatcctgtgtccggaaccggagcgcgccatcgggctgacggcgcacacggactcgggcttcatcacgctcatcatgcagagccccgtgcccgggctgcagctgctccgccgcgggccggaccggtgggtgacggtgccggcgccgccgggcgcgctcatcgtcatgctcggcgacctgttccaggtgctcacgaacggccgcttccggagccctatccaccgcgccgtcgtaagccgagagcgcgagcggatctccgtgccctacttcctctgcccgccggaggacatgacggtggcgccgctcgcgtccgctctgctgccggggaggaaggccgtgttccgggccgtgacgtggccagagtacatggaggtcaagcacaaggtgttcggcacggatgcgccggccctggagatgctgcagctgcaggtggatgaggaagaacaaggtgaaagggccgccaccacctaa

SEQ ID NO: 33 - protein, 371 aa, Zea mays
MQSSSSSASTPAAASGLVFDLGSAAGVPETHAWPGVNEYPSVESAGRDVVPVVDMGVACPDATRALARAADEWGVFLLVGHGVPREVAARAEEQVARLFVLPAPDKARAGRRPGEPTATGYGRPPLALRFSKLMWSEGYTFRAATVREEFRRVWPDGGDDYLRFCDVMEEYDREMRALGGRLLDLFFMALGLTDVQFATGETERRIRETWTATMHPILCPEPERAIGLTAHTDSGFITLIMQSPVPGLQLLRRGPDRWVTVPAPPGALIVMLGDLFQVLTNGRFRSPIHRAVVSRERERISVPYFLCPPEDMTVAPLASALLPGRKAVFRAVTWPEYMEVKHKVFGTDAPALEMLQLQVDEEEQGERAATT

SEQ ID NO: 34 - DNA, 4095 bp, Zea mays
taaatttgtgatccttgtgaagttgttatatcatgaattgtgaacttgttgcatttgtgatcttttgtcaactttgttgtattgtgaagtttgatatgtttaccgatcgtattttagatttcgatcgttaccggtgtattttccgcaccaaacttttgtttccgatgttttcgaaataccgatatcgtttccgtttctatagttacccttttcaattttatttccgattaaaaatatgaaaacggtaatggttttagtgtttatcgaccgttttcatctctaatcatccctgccggtgaagtttaatttttcccttggctaaagagatgcaagctgctgtaaaatacgttaaaacaggcaaggcagccccagcagccagcatcgcgtgcccgtctatgtacatcagtggatacgtagcatctctagtgagtaatataacgattgcatttggctggaggacgtatgttatataagtatgtcatttaccagttgcattagtatcttccctaactcctataataactctcttcgtggaatggacgtagacgtatgctatataagtattaaaaaatagttttttaagctggtgtcctcaattttgctattgttctcgtttttatctttagttgtgtcacaaatttaatccgtacaacaaatcaaaaataccatacccttcttatattaattttctaacataacatttgtttagatattttcagtcgtgaaaatacaattctaattctaacgtcgtagtatcaaatcaaaccatccagaatttgaccaagcttaattataaaaaatataaaatttatgatactgaatagatagcattagatttgttatataatatatttttataaaataccatttttatggtataaatattggtactcctttactttaaactatagatagttttgactaaggatgcaactagaattgcatcctcttttcactgcaccttcattagttttaatatttatttagatgggcccttgcaaactgtagatatcatctcttgcaacattctttctatagcaccacgaaaatgtattgcggctttgaaattataattgaattagttgtatcatttctttcaccgatgcgttaaattcaaaattaagtgttatatttcttcataatttgttaaatatatagaccctataatccaccattatttactataatagcatacattaacattggttttagcctacactacgacactcgaggcattgaattttcctctatcaaagaattatatgtgtagtagtattgttcttgacaaaaagggggattaaaattaaactaccaatattgatacttatcttatcacatccatgaatacaatcaacactcttacaaaagataagatacaagattaaaaagtaccatgataatacattaagattattagcaatgcattaaattaaataaatgtgcaagtgaatcatgattttagttttatctattttacttttaaaatatgatattctctgactacttctaagcataaatgtgattctaagtcatgaccgatcgtgcttattcagaaaaatgaaggagacacagatttctataaaaaaaggttgtcatgggactattgggtcaaccatcttattcatttgggaaaataagtttagaacacatcaacccattttagatgttgagtttggccctaatggtccattgaccttacttttgtgggttgacatagaccatctatcccaagttattgttgtgtcacattccctgatatcatgaatctatattttagctttccgttttcatatttttagtcgttacatattttttatccgcgtactagattaaaactctagttgttgcaatacattttgttcatttttttctatttcttctttactaacaacatattctagttcctagctacattcttaagtaccatagtgctataaacattttttatcctacattattccacttaagaaattgaattttctgcataaaaaaattatatgtccagtagtgttgtcttataaaagcataaagtgattaaaattaaaaccattattgatatcttatttttcaaaaaaaaatataagcttatagaaagtgaattaatttcatggtaaattaatatagtttaaattgaattattagtgttattactatgtttattatcaatgaaacatttttcatggttgatataacttagtgttacttattttagtattttttatataattctagttaacttttagtttttgatttaaaaaaacgagaattgtgtccttttgtggagtgagtataaagaaagtaatatctgttcatcataatttggttttttaaggtacgtgaaacttgctttatatttggactcaagctatgtctaaatacatagtaaaaaagcaatatttctagaaaagacaaaacatcttataatttagaatcaaggaaatatatagattttatgtgcagtgagaagccatttacaatggaacgttcaacgttgggccaatagatattttgcgatatgatgatgggcatatttttgcatggttgtccctccactagctatagtttgatgatacgatacgctgcacacaccattgggttgtaccatgttagtgtagcaacagtagaaacccaattgtggccgtgaaccatgataatactaggtagagtgctagctagaggtttcaggctattgatgcgtgaattaaactttctgttgtgttgcgaggaaacgagtattgtgaaatatttgaaacggttttttttgtgaaagatttgaaacggtatttttgttgtgaaataaagatcaaggctaaataaattcaaactaataaaacatattaattgacggcctgaagcccccgcccccatggccccatgccatagcatcaggtcccacatgacatgaggccgcgcctccctctatgttggctccctgccttcgccgttgtcgtcgctcccgaactccctctcctcccctgttacaaatacccccacccgcccggacagcttccctgcacactcgcagctcgcacatctcatggtgtcctaagaacggcaagagccagctctgcctagcagcagcgcacagccacatccatggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttca

SEQ ID NO: 35 - DNA, 7404 bp, Zea mays
cctattttgtgtctaatactcttcttatattaattgtttggtcaaactttagataaatttgactaatgatgcaattaaaactgcatcacctttactaaggtactgctttatatgtttcgacaaaattttcaattattctctatgtgttttaatctttgcgctacacctccattgatttaaatactcatttattttaaaccataacttaaattatatcggatctttgcatcctttctatggcaccatacatgaatcgatattttggctgcaaatttttaatcatgttagttttagcattttttcatatccatgtgttaagtttgaatcatgtgttgtttttatataatttattgaaaatatagatcctaaacttcactaatacttacaacaatagcatcatcatgtgttttaatccacgccacaacactcaaggcattgaattttcttctaccaaagagttgtatgtgtgtattgttctttaaaaaatagagtgattataattaaactaccagtattcatatgtaaaatgtatagacatctaaaataaaatttgcaaaaaacattgttgcagactttcaatataattaagaatgggttttagggtcatgatatatggtttgttaaagaaacttgtttttttttgcaattgataaactataaaatacattttcactattgtgtgcatatgtacttggtatacatagtggcatatatcatttttgtttactttgaggtttgaattatctatgttaaaattggataacatagatacattggtgtgcgtcctttggcccatttacttgactgaggagcaatactataaagtaaaacatatttggatattttatcttaaactcctagcataatattgatttaattatgaacaaatatatgtttaggtgatagtttcatgggtggtaaactatataagaaggcttaccatgatctttgcaaactctaggctatgaaagagttccatgatttgtcttagaagcatagacaaaacagtgataatgatctaaatcacacttatggcactgatgaccatatatgcaaagctaaatgcatgttaagttgtattatatcatatgtttacaatgactatcgcatataacgaggaatacattgtctatatagatagctattactgtagtagtgccaaatgttggacaacatgaatcataatcttcaaacctagagaaattgtagtcagtcgtacacatatcgtctagtaagttgtctatactttttatttattgtatcaaattttattgttatcttgcttgcttgtttgtttgtaccatagacacaatatggtcaaaaagtggtcaatcgattcgaagaagattgcaattgacgagtgctaacagttgatccttttgttgtgcacgctagcggagtagcatgaaaagagtaaaatatgaaattagcgttctaaactgtttgtgctataggtacttcgtatttaatggagtgactaactataggaaggtgagagctcagaagtcagcaccctcacacagagttctagagttagtggtcatcgaaccacgacaaactacatgatgagcagaagaggcaacatcaagactatgatcaatagtttcgggtcaatgaatgacatcgtgatgagtatttatctaactatatagaacaacaacacatgatgttttaagtaagttcaactgatcttctattgctatctttaagtatttaacgtagcgaataatgttttatctatttcattcataaataatgttgtgacaaaaggggataaccatcacttttaccatgttctagataccacaaccatctccaccatcataatgggttcttcattggtgcttggacctcaaataatcatatctatagccaacttagctcaattctaataaaattaggcaacttggcttcattgtagcaaaaatagccaacttagctcaattttatctaaacttagctaatctagcacaacttagatcaatattaggaaaaactaatcaatctaatctagctcaactatagcgaaagatagatattgtagcataacttagtagatctatctcaaattttagcaaaaactaatcaatttagataaactctataaaattttaatcattatgacttatttccaactaattgtaacttgcatgatttttatgttccttctttataattagcaacacctaaagacacgaatgatgaggggtctaacgcattcattaaccagttgttaaataatactctaggtagatgataagaactctaattattctatgaatctaagctaaaagatgtttaatatttaagtattggtgtttattatgttatttagaacgattcatgttacttaaagatttgttatgatttttaaatatgattatgataatttatgtggtgtggattaacttgtgaacatatgtgatgtagatgaatatgtatgttgtggatggaaccatatgaatatatatacacactcatatactattcgttggtgtaggtaaagcttcatccatcggtaattactaaatggtcttcagtcattaccactaggtgaagcttcacacgaccgataattattgaagaacgctcattaatttccggtaatggcttattggccttcactagtcggtgaaaattagctatttttataccaataaaaattagctaatatatgtaaaccaggtctaatttttatgggcctcttaccgaccaaaattgattagattattgttacaatagttttagtcaaaagctagctatgctataaaaattttgaattaaagtgagtttcgtaataaaaattgcatacttttaaaataaaataattaaaaaacagtttttagaaatacaatcaaacaccttatgctataaaaaaattgtaatgtacctacaaatatataatactttactttaaaataggcctgtgccttctcggctctatatgggctgcctccaacgaagcgccatggccatgggctccactgtgtcgggtcccacatgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcaggtttccgaattccaaacgtttttgtggcgtgcgtccatggggcgcccccaaacttcggacgtttccggcgctccaacaaatcttctcgcttcacacgtcaccgtcgtcccggattcatttgcctcgtcgctccaccattcgctgctctcctctccacgtactcttaccctgacctttgggaaagaactgaacattcgagatgcacaacagttcaaatataacatatgcagcacaagatcgttcgactgctatccgacaagccaacaacgtgcccagtagaactgaatgtacctgtgatttccagcactaacttacagcaacgttgtgaaaaaacaaaaacgaaaacaaacggcagaaaaaacagatgtattgttctacagttacaccaaatattttctggtcctttcagcaccaacaagagccatacgcatatctagaagacaaaattcctctaatttcacccctacgtggtagcagttcctcctcaacacagttcacgtgctagcgtcgagttctttgggccgccacatcgacttctcgacgcagagcaggccctcgctgcccttggtgtaggtcatccgcacctcccactgcacggacttggccatgctctccagctcatttatcgtgtccgcggtgtccctcacgatcagcttgccctgtggcctcagtacacggtcgacctcggcgaaaactgcagccagtttgcatctgtaaacaggcaacacagatttttagtatctaaaacactgcaggcaaacgccacaggttttagtcgcaagaagcaataaaagcatgcaaacaatgctacgtgtacgtatcaaaggaacatgtcaaaactcgttgcatgaacgatcattgatgtttccttgctgaactagtcacatcagtctgcttcaacttctgggtttcactagtagatataccagaagggtagaataatgtgaagagcaagaaatacagacctctttctgagctttgagaacagatggtccgcgtgcagaaggtcatacgttcttgggtaagtgctgaaagactcgcaccagtcatggtacatgccaaacaaaccgcgctcgtagatgatgggcagcgtgtctggtgaatcgatcggcacgatattcatgacccagaccttttggtccctcagagctgcagcaaaactgccatgcaacaatgtaaagcattagtcaagaagaaggtgtacagtgcatttctccttgtcaacagtcttcagtaacaaaaaaaaagtgttatgcttgactgaatctttcaaagaaatatgcttgatgacttatggtggacaagttgcctgttatagtgttatgttttaattaactatgtgccagcttgggtaactagtagttatgtagtgtgatctgaattaccaaaatataaataaataaataaacatgcccaagaaactacgaaaaccatttacttaccctccatagacagctctcatgtccatgacatttctcactttggaccagtcaattcccatgccattcacatacgatttacttacaacccgtttccagtgggcattatctgcctcaaaatcttcatttgcaggctttccatagacaccaaccttggaaccatcaatccagaaaggggtcttctcaagcctttgcggccataactctggccattttgatcctcggacttttgagccaccaggcagtttgtgcatgcatgcttccaacggtacattcctgcaaatcaaaaggctgtgtaagcaaagcagagaagcacttttctccattgaaaatatactcttctcaaagaaccgaaaccataccaagcagcatctgcatcatcagattccttgcacaatggcgggctgttttcagatcttttctcatagcaaatattgtccattggtttctgatatatgaccataccaacttggtttaacttatccttagtcttgttgaccatcttccagcacatggactttgtcaaagtagacatggctgaaaagggtatgtggccacatgttatgttagaaataaaattcaattttgaacagttggtccatagcatgtattttgaacaaatgcaatccttctccatccatgaaagaagttgacccttcatacttaggattattcagtactttcactcatgtctgctgaatttgttctcttggtagttgctatacaagaaagggggaagtacagagtagctaaacttatacaagctatagtctgatatttgtatgaaacataaattttggtatggatgtcttattaaaatgggaggttgtataatatttttctagcctacctcaacttgcttgagactaaaaggctttgttgttgttgttgaggctgtatggtgctttgactttacaaatcaagttatcagctaccctacttatggatatacacctctcataaaatgatggtaagaagtttcgatatgtcacattaacataagaacttcattcagttagggtacaacgaagttaagtagttacggaaataccattccaaatctcaacatcctctgggagcttttggtaaacaggagtggcagaccagacaaagtaaccaccagggcgtaacaagcggttcaattccagcaaaagcatgccacctaaaagtagcgagccagcaataagattcagttctatagcaaatcaataaatgaaaggaggacatgtcaatatgtaaccagcaggacaaaccttcgatgtgccaaggga

SEQ ID NO: 36 - DNA, 1788 bp, Zea mays
aatcccagcgtgcgtaatctattgcccacatgccgacgccgtcgcacctcaacaagaacccgcgctacctggacttccgggcggcgcggcgggtgccggagtcgcacgcctggccgggcctgcacgaccaccccgtcgtggacggcggcgcgccgggccccgacgccgtgccggtggtggacctgggcgccgcggacccggcgccggcgccggcggcggcggtggcccgcgccgccgagcaatggggcgcgttcctgctcacgggccacggcgtccccgcggacctgctggcgcgcgtggaggaccggatcgccaccatgttcgcgctgccggccgacgacaagatgcgcgccgtgcgcgggcccggcgacgcctgcggctacggctccccgcccatctcctccttcttctccaagtgcatgtggtccgagggctacaccttctcgccggcctccctccgcgccgacctccgcaagctctggcccaaggccggcgacgactacaccagcttctggtacgttgcgttgcgtgcttgtgtgcgcgcacacctgccgaccgcggccacaccgtacgcaacccacgcgtacgtacgtgcgctagctacctgcttcgctcgcttcgctcctctcgcctcgccatgcatatgcacgtacggccgtacaggtacagcagcaggtcacacgcacgaacgcacgcacgcaccagcaccgatatgatacatcatcgacgtgtcgtccccccgtctaaggccatgcatgcatgcaagcacgcctagctagcccttttggcttgctagctgacgaggggagctaggacgagcatacttactgtgcgcgtcatgctcaattgctcacactatactactacttgttactacagtgatgtgatggaggagttccacaagcacatgcgcgccctcgcggacaagctgctggagctgttcctcatggcgctggggctcaccgacgagcaggccagcgccgtcgaggccgagcggaggatcgccgagacgatgaccgccaccatgcatctcaactggtgggtatatattattgtctgtcatgttgtcgtcgtcgtacgcgttgcggttgggtgtacatgtatataacacaaacaacaaaaaactaacgccgtgccgacgacgacgacgatcatcaggtacccgaggtgcccggacccgcggcgcgcgctggggctgatcgcgcacaccgactcgggcttcttcaccttcgtgatgcagagcctcgtgcccgggctgcagctcttccgccacgccccggaccggtgggtggcggtgccggccgtgccgggcgccttcgtcgtcaacgtgggcgacctcttccacatcctcaccaacggccggttccacagcgtgtaccaccgcgccgtcgtgaaccgggacctcgacaggatctcgctcggctacttcctcggcccgccgccgcacgccaaggtggcgccgctgcgcgaggccgtgccgcccggccgggcccccgcgtaccgcgccgtcacgtggcccgagtacatgggcgtccgcaagaaggccttcaccaccggcgcctccgcgctcaagatggtcgccctcgccgccgccgccgacctcgacgacgacggcgacgccgccgtcgtccatcagcagcagcagctagtcgtctcgtcgtagccgagaccgatcgccggagactgatgctgatgatgatgcatatatacatgagagaaatcgtcgagtagactagccgattgcaaaagcaaccccagctgccgaaacctggcatatcgatcccattc

SEQ ID NO: 37 - DNA, 1698 bp, Zea mays
cgtgccgagagtccttcaaagccgacgacgagacgacgatgcagtcgtcgtcgtcatcagcctcgacgccggctgccgcttccggcctcgtcttcgatctcgggtctgcggcgggcgtgccggagacacacgcgtggccgggggtgaacgagtacccgtcggtggagtccgctggccgcgacgtggtcccggtggtggacatgggggtggcctgcccggacgcgacgcgggcgttggcgcgcgccgcagacgagtggggcgtgtttctgctcgtcggccacggcgtgccccgggaagtggcggcgcgtgccgaggagcaggtcgcgcgcctgttcgtgctcccggctcctgacaaggcccgcgcggggcgccgccccggggagcccacggccaccggctacggcaggccgcccctggcactccgcttctccaagctcatgtggtccgaggggtacacgttccgcgccgccaccgtccgcgaagagttccgccgcgtctggcccgacggcggcgacgactacctccgcttctggtacgtacgagcgccatgtcacgtgcttgtgctttcatgcctcgtaccgtcgtcgtgctgtacgtgttatgtttatcggccggtacgtcacgcgtgctacactggttaacgacgtgagcgtgcccacgttgactgcatgcatgtgcatgcgcgcgcccagcgacgtgatggaggagtacgacagagagatgagggctctcggtggcaggctgctcgacctcttcttcatggcgctcggcctcaccgacgtccagttcgccaccggcgagacggagcggaggatccgcgagacctggacggcgacgatgcacccaatcctgtacgtacgtcaaaaacgaatatctgaccaatgcaaacgtttttctgcaatgccagtcatccactcatcctgtacgtacctctggactctgcttgtccatctactgatgacacgtatggtaggtaccccaggtgtccggaaccggagcgcgccatcgggctgacggcgcacacggactcgggcttcatcacgctcatcatgcagagccccgtgcccgggctgcagctgctccgccgcgggccggaccggtgggtgacggtgccggcgccgccgggcgcgctcatcgtcatgctcggcgacctgttccaggtgctcacgaacggccgcttccggagccctatccaccgcgccgtcgtaagccgagagcgcgagcggatctccgtgccctacttcctctgcccgccggaggacatgacggtggcgccgctcgcgtccgctctgctgccggggaggaaggccgtgttccgggccgtgacgtggccagagtacatggaggtcaagcacaaggtgttcggcacggatgcgccggccctggagatgctgcagctgcaggtggatgaggaagaacaaggtgaaagggccgccaccacctaagccctaaggaactactagctgaatccataaactaataaagaattcgtgaataagggcgttggaagactggacacaacacaagagagttgctatatatcgtatttctgaaatttaaggcaaatatcttagttaaaaaactggtatatttaaatagacaatatatatctaaaataaagatagttcaccatttttacggtcgaacaatgataaagttatatattgtctgaatagtaacaaattaaagatttccagg

SEQ ID NO: 38 - DNA, 4095 bp, Zea mays
cggtctaagtgaccgtttgagagaggaaaagggttgaaagagacccggtctttgtgaccacctcaacggggagtaggtttataagaaccgaacctcggtaaaacgaatcaccgtgtcatccgccttatttgcttgtgatttgttttcgccctctctttcggactcgtttatatttctaacgctaaccccgacttgtagttgtgcttaaagtttgtaaatttcagattcgccctattcaccccctctaggcgactttcatataaatattgggagaaatatgaaaaacaaatgaaggtcgaacgagtcagagacaccataaaaaagaggtcgtcttaactagggtgctaaacctcaacattgtagtagatcttagtactgagtttgacatctttgacaccaacaagatggtgatacgttactttctacgttaacttgggtaggtatatcgactatagtggcctataacactaggctatgtaatatgatattgtgttgagtctttataaacatgattttttttaaaaaaaagagctaaaataaaaaatagaaatcgacggtacgatgcaagttcttctcaagacaaccaaacgcacccttgcccctttattgaaattgaagtatgtgctttatcaaatgtttaaatactaattataagtattaaatataatttaattataatactaattatatagataaagactaaataacaagacaaatttattaaatataattaattcattattaacaaatacttaatgtagcacgatcgaatcatggactaattagtcttgatagactcgtcttaccatttaatcataattagttttgtatactgtttataatatttctaactagctagtattaaacttttgatgtaacctaactaaagtttagtcacgccaatacataaggactcggatcgttcgatcacccatgacatcacgtatactaagagcatctccaaaagctctccagaagtctcccctaaatctatttttttgggaaaaacacaaaaacatgtctccaacagttcccttaaagcgcccccaactttttcatagcccttaaaactccctcatttgtagctacaaatgaggggttttttgggctccccagaaacaaactgttgatttaagggatctgttggagaaaggattaaaatttaccctcacttattatttagatgtcccttaaaactgattttgaggagtcgttttatgtagagctcttggagatgctctaacacaccgagcacaaccgcatcatcaatcaaaacaacccaaagtttgttcggtacaagtcatcagcctgtgtacacacatcagcctcggccccgggagaagcgctagcaaacaaggttcacctaaaaatccatccagattcattgaatccaaccagcacaaacgtcccatttattaatcacctcatcacaggtccccccagcctcactctcgcgccggctcaaggtacattgcgtgtcctagccaagacacgcagctcatctcagcctcacacgcacagcaagagcgaggcgtgattcgccatgggcggcctcactatggaccaggccttcgtgcaggcccccgagcaccgccccaagcccatcgtcaccgaggccaccggcatccctctcatcgacctctcgcctctggccgccagcggcggcgccgtggacgcgctggccgccgaggtgggcgcggcgagccgggactggggcttcttcgtggtcgtgggccacggcgtgcccgcagagaccgtggcgcgcgcgacggaggcgcagcgagcgttcttcgcgctgccggcagagcggaaggccgccgtgcggaggaacgaggcggagccgctcgggtactacgagtcggagcacaccaagaacgtgagggactggaaggaggtgtacgacctcgtgccgcgcgagccgccgccgccggcagccgtggccgacggcgagcttgtgttcgataacaagtggccccaggatctaccgggcttcaggtgacgaaattaactatatatccctttcgatcatagttgcgttaataaattaagggaatcgtgagcgtacgtacgtaagtttccgcagagaggcgctggaggagtacgcgaaagcgatggaagagctggcgttcaagctgctggagctgatcgcccggagcctgaagctgaggcccgaccggctgcacggcttcttcaaggaccagacgaccttcatccggctgaaccactaccctccttgcccgagccccgacctggccctcggcgtggggcggcacaaggacgccggcgccctgaccatcctgtaccaggacgacgtcggggggctcgacgtccggcggcgctccgacggcgagtgggtccgcgtcaggcccgtgcccgactcgttcatcatcaacgtcggcgacctcatccaggtacgtgcccacctgatgaactgagctgaacgtaggttgcatgcactgcatgtgtataggcttctcagatcgcttcgtgtggcgtaaggtgtggagcaacgacaggtacgagagcgcggagcaccgggtgtcggtgaactcggcgagggagaggttctccatgccctacttcttcaacccggcgacctacaccatggtggagccggtggaggagctggtgagcaaggacgatccgcccaggtacgacgcctacaactggggcgacttcttcagcaccaggaagaacagcaacttcaagaagctcaacgtggagaacattcagatcgcgcatttcaagaagagcctcgtcctcgcctaactactgctactgctaggatccatgccattgccatgtcgtcttcagattcagagcacgccatgtcgtcgctagcttcgtggtagaacaaataatgatgtgcgtgctgtgtgtaagcatggatatggatgtgaatatgtaatatgatgagcactcctactttggtatgtttgggaataacagacttgtgttggtctggttcattatttgtaagaaaatcaaaaagagttagtagggcaggaggctaaccacagtcatgctgcaccacatccctggtggaaagctggccgggttacgctacgctcgtgcagccagattactgcagggccgggatatgcttccggtggaaggaaggggacggtggctgaggaccatggggctggagcctgggagagaggtcgagctagaagaaagggggagagagaagacgcacaacgaagatgggtcagccagggatttcgacccaagggggagctagtggattttgggagaaaacagaaaagagaaaagagaaaagaagaaaaatttgttggtgtgaacacaaggttgatttgtcttttcttatttggattgatgatgagtcgtggactaaccgacccgtgagctattgtgtcgtataatcatgtctctcggtttctggtgtgcaggtttgaagcacagagacggtggtcgacgcaaaggtgaacgtcatgcaggttcgtgccgatggaccgggagcagtgaaagacgagcgttgggacttgaacaagggaccagagtcgccggatgactagccgcagtggctgacgcctggaacacgcatagacgtgaggacgtggtagagcaggtgaaaatcgcctagagggggggggggtgaatagacaaaacctaaaaattataaactttgaacacaaactttacctgaggttaccgttagaacgagtattaatgaaatcggagtgcggaaggcaagttcttcttgctacgagttgcttaatcaatattgataactttgggagtcaactcaaaatgatcacaagcaaaagaactagagagagaggagaggaagaatcaactcgcaaagtaatgatcaacacaaatgaacacaatgatttatttctcgaggtttggttccgaagaacctactccccgttcaggagtccacataggacatgtctctttcaaccctttctctctctcaaatggtcacatagactggttcagttgagagcacctagaggggggtgaataggtgatcttgtaaaatcaaacactaatagccacaaaacttagtttaaagtgttagtacggctaagtagctttgaagcgagttattgtgaacacaacaat

SEQ ID NO: 39 - DNA, 21 bp, Artificial Sequence
  suppression oligo
ctccatcatgcggtgcaacta

SEQ ID NO: 40 - RNA, 21 nt, Artificial Sequence
  suppression oligo
uaguugcaccgcaugauggag

SEQ ID NO: 41 - DNA, 19 bp, Artificial Sequence
  suppression oligo
ggtactgcgaggagatgaa

SEQ ID NO: 42 - RNA, 21 nt, Artificial Sequence
  suppression oligo
uucaucuccucgcaguaccua

SEQ ID NO: 43 - DNA, 19 bp, Artificial Sequence
  suppression oligo
caggcgccatggtcatcaa

SEQ ID NO: 44 - RNA, 21 nt, Artificial Sequence
  suppression oligo
uugaugaccauggcgccugga

SEQ ID NO: 45 - DNA, 19 bp, Artificial Sequence
  suppression oligo
tcatgcggtgcaactacta

SEQ ID NO: 46 - RNA, 21 nt, Artificial Sequence
  suppression oligo
uaguaguugcaccgcaugaua

SEQ ID NO: 47 - DNA, 19 bp, Artificial Sequence
  suppression oligo
tcgctcgccttcttcctca

SEQ ID NO: 48 - RNA, 21 nt, Artificial Sequence
  suppression oligo
ugaggaagaaggcgagcgaca

SEQ ID NO: 49 - DNA, 19 bp, Artificial Sequence
  suppression oligo
tccaacgggcggtacaaga

SEQ ID NO: 50 - RNA, 21 nt, Artificial Sequence
  suppression oligo
ucuuguaccgcccguuggacc

SEQ ID NO: 51 - DNA, 19 bp, Artificial Sequence
  suppression oligo
gcatcaacaggtacaacta

SEQ ID NO: 52 - RNA, 21 nt, Artificial Sequence
  suppression oligo
uaguuguaccuguugaugcga

SEQ ID NO: 53 - DNA, 19 bp, Artificial Sequence
  suppression oligo
tggacgatggatagttcaa

SEQ ID NO: 54 - RNA, 21 nt, Artificial Sequence
  suppression oligo
uugaacuauccaucguccauc

SEQ ID NO: 55 - DNA, 19 bp, Artificial Sequence
  suppression oligo
tggaccatggatacttcaa

SEQ ID NO: 56 - RNA, 21 nt, Artificial Sequence
  suppression oligo
uugaaguauccaugguccauc

SEQ ID NO: 57 - DNA, 19 bp, Artificial Sequence
  suppression oligo
gcaaggtcctagatttaca

SEQ ID NO: 58 - RNA, 21 nt, Artificial Sequence
  suppression oligo
uguaaaucuaggaccuugcaa

SEQ ID NO: 59 - DNA, 19 bp, Artificial Sequence
  suppression oligo
cagagtacatggaggtcaa

SEQ ID NO: 60 - RNA, 21 nt, Artificial Sequence
  suppression oligo
uugaccuccauguacucugga

SEQ ID NO: 61 - DNA, 19 bp, Artificial Sequence
  suppression oligo
ccatgccctacttcttcaa

SEQ ID NO: 62 - RNA, 21 nt, Artificial Sequence
  suppression oligo
uugaagaaguagggcauggaa

SEQ ID NO: 63 - DNA, 19 bp, Artificial Sequence
  suppression oligo
acatggcggtcaacttcta

SEQ ID NO: 64 - RNA, 21 nt, Artificial Sequence
  suppression oligo
uagaaguugaccgccauguga

SEQ ID NO: 65 - DNA, 726 bp, Rice tungro bacilliform virus
tcctacaaaagggagtagtaatatttaatgagcttgaaggaggatatcaactctctccaaggtttattggagacctttatgctcatggttttattaaacaaataaacttcacaaccaaggttcctgaagggctaccgccaatcatagcggaaaaacttcaagactataagttccctggatcaaataccgtcttaatagaacgagagattcctcgctggaacttcaatgaaatgaaaagagaaacacagatgaggaccaacttatatatcttcaagaattatcgctgtttctatggctattcaccattaaggccatacgaacctataactcctgaagaatttgggtttgattactacagttgggaaaatatggttgatgaagacgaaggagaagttgtatacatctccaagtatactaagattatcaaagtcactaaagagcatgcatgggcttggccagaacatgatggagacacaatgtcctgcaccacatcaatagaagatgaatggatccatcgtatggacaatgcttaaagaagctttatcaaaagcaactttaagtacgaatcaataaagaaggaccagaagatataaagcgggaacatcttcacatgctaccacatggctagcatctttactttagcatctctattattgtaagagtgtataatgaccagtgtgcccctggactccagtatataaggagcaccagagtagtgtaatagat

SEQ ID NO: 66 - DNA, 165 bp, Rice tungro bacilliform virus
acgaatcaataaagaaggaccagaagatataaagctggaacatcttcacatgctaccacatggctagcatctttactttagcatctctattattgtaagagtgtataatgaccagtgtgcccctggactccagtatataaggagcaccagagtagtgtaatagat

SEQ ID NO: 67 - DNA, 2000 bp, Zea mays
ctgcaatatatacaccaaaagtattataaactgtcatatatatgaccaaaacctttttattttagaaaagtatattaatcatggtatattaatcaaagttgttgttggggctgcaaaaatcatacccttcttccacaagctgttccttgaactgcaggtactcaggaactctcagctcctcaacagcgagctcactgacgttgaccctcacatactcccagacaccaggcctagggcggatggcaagtgcaacccatggggggatgacaatcgcctcctgtaagataatagagctagaatgattaaagaaggtgcacactacaaaaggaacagtgctgtccagcgagatctgaatctgatgcaaacctgagctgccctcaggacatcctcaaaagcaccatccttgagcttctcgcgctcagcctcagggatcgcattgttgtactcggcaatgatctggtggggctgcagcataccctttccaaggtttttcagcctgcgcaaaacgatgtgccaaataacatcagactatgccagatctataaactcatcaaacatatacaatttcaagaaatagtttagacgtatgatcagcagtcagtagcgtgggaacatatgcaacatagcgaagaggcacaacagcaaattcattcgaaaaaatgaaaacaaagattcctctcttttaactgaacttctcgaaacccctttcatgcctacacatccgatctagtcagatgcctatgcgttcatgctgaacagaacgtgtcagaactaagcataaactggttagcaagcattatcgtattcgatagaccctttagtaacaagctatacattgggtaagttcagactccaatcattctgttcagaaacatcgtattgaatataaaactaaagaacacacatgcaggtgcagccagatctaacagcagtttacagtcggtactaaaaaaagcatggtgtatgtatgtatcatcagtatccagtactaggtttcgacaaaatcctggatgctaattaaatactcatcttattagggaacacaggaacattatgtctacagcattgaatgatggccacatcatgctagatctaacaatacataatatgatggaactggtcttaaaaagtcgcattcgctcaaataatacccgtagcaaaataaatgtaaacttgcagacgaagcgggggaaatgagggcagacctggtgaagacggcgacaagctcattggggtgggcagagagtgagtcgccaatgcgctccctgacgctgtggaggcggctcaggacacggtcacctgcaccttcccccattgctgtcctcttcctggatcctcaggcctgcacagcgaaaccgaaacggaagcggaagcttcagtcagcagagaaaactgaaaccgaaaaacggttcagatccgttgacataaaagctgcgatgacatcctaaaactaaaacccctccagcaagacataaacccaactgccaacaaccagtcttttaagtctcgacacacccttgacgctgcgccacgaaactatattgcaggcaagaaaccaacagaacctaactctggaaggggggaaagaaacggcagacaggagcaagacccaaaaaaaaacgactcagatcctggtactatagtcctagtacctagaccagaaagaagaaacaaccaatacaacaagaggcatacaagaactgaatcgatgaactgaaacgcttcagaggaccgaggaatggcggagaagggaggcgcctatttatacagatctgacgagagaaccgaacaaaaacacatcgatgggaaccatggagaagaaaagggctggccgcatggcaccaatggcctcggcctccaaaaagccgttgaatccaaagcaggcgaggacgaagcgtgacgcggcagggtacttctctagaaaagcacggcatcagcaaggtgggggggctggggttccttattgcaggcaatcacgaggtgattagcacaaacggaag

SEQ ID NO: 68 - DNA, 2000 bp, Oryza sativa
ctgaaatatacatcagagatattacaatgacatatatatcgataaaagaaaaataataaaattaagttttaaattttaagaatatatgtttttagtatcccaattatgcagatttcatacccttcttccacaagctgttccttgaactgcaagtactcggggactgtcagcaactcaacagcgagctcgctcacattgaccctcacatactcccagacaccgggcctcgggcggatggcaagggcaacccatggggagataacaatcccctcctgcatgataaaaaacaattacaagttaagttagagcaagcggtagagtaaagatggatctctgtgatgcaatgaaatctgaatctgattcaaacctgtgcactcctcaggacatcctcaaaagcaccgtccttcagcttctcacgatcagcctcagagattgcgttgttgtactcagcaatgatctggtgggcctgaagcattccctttccgaggttaaccagcctgcgcaaataacagtgtcaacaaaaatatcaggccagatctatcaactcagcctataaatatctcaataagataattttagcacttgagcatttgcgcataataagaaaatttgctattagccacttaaaaagaccatatatgatctgtttgcattgagatgaattaaaaatttcattgtagatatgaaatgattagttttgaccatttaattggacttaatgaaatatgcgcgataatcagatctacgcgctcgcgccaatagatctagtaagatgtaggttttttattttttttgtgaaactttgctaccacaacaagcatctgtaccagtgcagaattcattacttgtattcagtttgtaaaccgtatatataatataaataacatgcacatgcagtcagatctagcactaccagtccacagtaatccaaaactacatttgtatatttcatcattattcagtagtactaggtttgtacaaaatcttggctgcagaaggccgcacttaaatattcattctaatcagaaacttaaaaaaaaagtgactacaaaatgattgcatccaattcagtaaatatgagccattcctggccagatctaacaatctcaacaacaaagatcctatatgaacatctccttctaaaagaaaatacagtaacatctgaaggcagtagactagaaaccaacaaaatctaatgctgggaaatcactaaatcagcacgaacctggtgaagacggcgacgagctcattggggtgggcggagagggagtcgccgatgcgctccctgacgctgtggaggcggctcaggacgcggtcgccggcagcttcccccattgctctcctcttcctcttggctcctcaagcctgcgtgcacaaccaaccaccatcatcagatacatccagacccagtcaacacaatcactccaggaaaaaaaaaagtcaagccataaaccccaaccaaaaaccacgcctttgacaaacactggaagaaaaagaaaatcgcagctttttcacaagcaatctagaagaaaagaaaaagaaaagactacatagcagctataattgactgagaagcatacaggaatcaaacaatggagaaggggagggaggaagaacaatgatgctccaggctgaggaccgaggaactgggtgaagcggggtaggcgcgtatttatgcagatctgaggagagaaaccaccaaaacaatccgatggtttcaacgaaaaagatcgtcgcttcttgctgcaccagctcacccatagccgttgagatcgaagctaagctagcagcagcaaagctggaacgaagagtgacgcattcaagctcctctcctctcctctcctctcctccggagcacgaggccagcatgggatggattggggtttcttgttggccatggcaaaggaggaggtcattaacgttgacacggcgtaatttaattaaatcttatcttaaaatatgatttaagtggtagtaacaaggaagattaatactatgaa

SEQ ID NO: 69 - DNA, 2000 bp, Oryza sativa
gctgtttaagaaaaacagaagtaaaattcagtcactgttattttgcttcagttataatctgcaaatcgtcgttctggtacttactgtccatcaacaagctgttccttgaatgccaagtactcagaaacactcagctcttccactgccaactcacttacattcacccgaatgtagtcccagacaccaggccttggcctgatggccagtgcaacccagggcggcagcacaatggcttcctacatacagtcaaggaagtaagttagaaagactggtatttgactttgagttgactatcataaccatcctggctcattgccaaatttacctgagcagcccggagaatgtcttcaaagggagcatatttctctttgtcagcttcgatcaaggcatcgaactccgcaagcagctggtgacgctggagcattccctttccctggttaacatacctgcatagagtgatatttaagaaatagaaccaatgcttagatctcacatcctttctgcggctgaactatgttaatggcactaccacataaacctgatttttacttcttatttttaagaccacatgatctgtacttaatctagctatgaacaaacaatatttcaacatcatctaagattcatgactcaagacaaaaatgttagagctcatcacagattattatagataccatcattaaaactaaagagatgcataaccttgtcagctaagaatttgtaacatactaacatgttatcgtttcacatctgggttgactaagaactaaccaactgtatggataaaatcattgaaaactcaaaacaattagtagcaggttccaagaagacacaagatattatattgagatcttcacctagagaagagtgcaatcaactcattgggatgagacgagaaggtggcaccgaggcgttcgcggagactgtggaggcgagctagcttggcagccatgactcaatttcaggaactgcaaagaaaggttacacttagcaacacgtaccaaaaccactcacttgcacaagaataattagtcaacagccatcactaagcattgcaagactatctctgaacaggaaagccatgctaaatcaacactaataacatcacacaaaagcattggaagatcaaaacataactaaaaacagctgtttcatctacacaactgaaagcatctatggtttacgaagcagagtgcgagtactgattcaaaataaatcaacctgaaccaatatactctgacaatgttttcaaagggataaaagaaccagctttatcaaatggatttgttgggttttagtaagtatcattgagataccgatggcatatctcaaactttgcaaaattataatggcatggttccaaattaccctttagtattagcaccagttagatcctaattcctaaatccgataggacagagcgaaagatccctggagatatgaagatttggctacagattaagcagagccaacatgaagttccgaatattatgaatccgcaagcggggagatcaaagagaagaatacggaaggtcgcgactccatgaaagaatccaaccaaaaacccaaagatttttctcagttcaaaaaaaaaaaacccttcatttttggttcgccatccaccgacaggcaccaagacattcctcaggaagcaaaaaagattaagcagaacaagtgataagcaagacacagtatcaacggactacgagtcgagaaaatcactgaggcgcgattcttactgcaccaagtaaaaaaaaatttgggggcaaaaagaactctgcaatggggcggagcaacgtggcagcaaaactaaaggtcgaggatttgaggttttttgccggttttcctcgaaaccccgaatccgctcatagtaaacccactaaactgcagcagaaacccccctcttggttcagatttaccgaaagcagtaaacccaagaacatgtcagcaaaaactcctgcaagattcagctgacgacccaccaaagaatcgcaagaaat

SEQ ID NO: 70 - DNA, 2000 bp, Oryza sativa
tggcggacgcgccacgcacaaacacaaacctgcacacccctgtgtcagaggaggagaggccaagaaaggaaatcgagtggaggaagtgaggagcggcggagacgtaggaggaggagggggagatggaaatggaaagccgcgcgagagaggaggcgcgtgctggatgggaggaggaggaggaggtggtgggtttgtgtttggagagacgagcgagagaggcgaagcatttaaagggaggaagagggggagagagagagagagagagagagagagagagagagagagagaaaggaggaatataataaagggtggtgcacctgccaactgctatgctcaccaacactttgtacacacccagttacacccccctgcctttattatttccagtgcagtaataacttcaacaattattgaaatgaaaatggaattaatggagttagtatcggattagcgacacgcttgccgagcttctagacggtgcgattatttcagcgggaacgactttctgtaggtgaatttaatagaggagtgttttaaatccactcgacgttgtaatagctggtttaattcgtttgtactgtcgagtagttatccaaaatcaattttggatatttaaaagaaaaaaaaacagatccgaagtattggacctactggcaaataggaattttgctatatataggtgtgcgttcatttataatggagtagcatggagttttattaatccagtaaatgttttcattgatttaattaatataacgaatttcgcttgaggccatatttgttaaacgcttttatctctatcatcattcatcctaccagtaaagagcaccggagatcgcacttcatttaaatatatgtccatgttggataaaccatagtttattatagtgttcttttatatgttttgtggggaatttagattgtttaatatggcatacatatccatccatcattattatattctaacacaactggataagtgttctaaactattgtagaataactttgtagtatgatcgatcttgtggaataaaaaaagtctgacaataacctttcataaaggaatatgaatacccgtaatcaacgcatcaaatcattcacggtgtacgcctagcgaattcgttggcgagtgctcgtgcggccgtgggctcgctgtgatgcatgcatggctctctggctacgtcgagatagcgattagtagcaaaattaagcaagccacttattaattaatctttggagatatcatatgattaaggcattaattcgtacgtactcgtcgtcagcgttttctgcaaagtccactacagttttttctttctttgctgaaaatgctgatgtgttggagatggagtgacgtgcacaacctgccgccacgtggatggttgctggagcctacgtgtcatcttaatttgaacaaaaaaaaaagaggaataatacatcaatacattttcgaatttcagttctgccattgaccagtaatacacatgtcggcctcacattttaccctgatcttagtaacgggtggtcgcctggtcggtcactgaaaaaagttcaggaaattatagtcaaactgaaacgaacatattcactccttaaaaaaactaaatctttttatatatttgtgatattgtaaaatagctacgggataatgatatagatatatatagtgataagggatagatggatcgagatatggagttgtgctttctttaatttccactacttgggctaccatattatggtagttggtatgaaaagatacacagcagtatagtgatgtgatcaatgacatgtatatctcacatgctcccatgttggagtcaaattttgctagactaaaatccaattccaagcagtccctagccaagaacaaacaaaattcagtgaggtcactgctgcaccaaggactgcatgcatgcaggagaagggcattttctcttttttcttttggagactcgattcaattcggtcggtcggtcgcaatggtcagcttaattaaa

SEQ ID NO: 71 - DNA, 2000 bp, Oryza sativa
tgtgaaaggtggcggcaccagcttagccgcagcttctctcgtcgtctccctgaaacgagagggaggaagttggtagcgtgatatatttaggcatgtcatctcttgtataagaagtcttatctgtgctaattcacacggttctctaatctctctccattctgtttttgtaaattggttcagtagatagcgtagggttatgcttatatatactccgtgaagtatatatttaaaaattagtcacacgtaaagtactatacatgttttatcgtctaataacaataaaaacactaatcataaaatttttttaaataatacgaatggttaaacgttgaatatgaaccgtgcaaaactatatttattttgtaacagagaaaatatttcacattaattagattgttgttttatggaaggttggagagctgcgccgccgttgcgcagacctaggaggctgcttataagttataatcaatcaattcacggatgccggctgggacgcggcccatcgtccgggaagacgacaactcaacgcaaaaagccgatatgcctccaaattgccattgccacctctacggctgtttatactgctccaaatcaaaagcgtccatggaagaatctagtatttcccgcaaagacgatgatgatatgcaggattggatatatagggggttgttgcatgattgctagaactcccgtttccgaagttgttcgtccatttttaaagctgccaaataggaatttattttgttttcaagtgtaatagagttctgtccagatgagtgaattataatttggttcacattttatttgctaagtttcagtttgaacattctcaaataacttttttcttcactttttaaccgagtaacttagttattttttccgtttggaccacccaacaatttgttgctaagtgcatctcacccgtcaaataattcctttgaatccaaattcaattatatcccaaaaataaaaaacttctgaattccacatcaattcaaaccccaaccattttaatttctctccatattttccatttctctatttttacctttctcttttttccatctatttatttttttccttttctatttctttctttctccttcctttctctgtttccttcttcttctcctcggctaggcccgagccagcccgtgccgcctcgcgccaaccctgtgccgccttacgccgcgcttgcgtgcgctcgcgcccacctcgtgcccaacccgcgcacgccacacgcacacacgaggacgatcgacggacgaatgcaatcatatccccttccttactcagctagaaggctcaagaaccgcaactttgatctcttccaccctctcaaatccgccccaacccctgctgactcaatcgccattaccggaggaaaaatccccgaaaccctattaccggcgccactaacagagctccaaaattcgtcgcataattcgaaaatattctgaaattgaaggtaaaaatggaatctacatgcgaagtactccctttcccctccaatccgtcactggaacgccgccggcgccgcctcccgctgccactgccctgtttggccgccgacagccgcacggcgcgccgctgctccaggccgccctagcttcaaccaccgccacctttggctccgcctccctcctcttatgctcaccaagcccgcctccctcgccggagatcgccggaaccaccgccgccatggccgccaccgcctcctgcttctggccgccgccgccagcctcgccaccggcgcctatgccaccgccgaccacggaaacggagtccctacaccttggggaccacaaaaccggcggcatccctcccaaaaccggcctcctccaccgccggcgttcgtgggattccggccagttctgtgcagagcgagagaagaagaggaaaaatagattttcctattgaaagataaatcagaaaattcctttttcttttcctatcaagttgaccatccgtttgacctcaaaatcaaaatctgagaccta

SEQ ID NO: 72 - DNA, 786 bp, Zea mays
gacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagagaccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggatt

SEQ ID NO: 73 - DNA, 1160 bp, Zea mays
atgtgctggtgccccataaggtaggcacctaggtctgtgtttgaagcatcgacagatttgtaaacatgttcctatgaacctatttctgattgataatttgtcaaaactcatcatttgtcttcatccttgcctgcttgcgttcacgtgacaaagtacgtgtatgtcttcggcctttgctgtgtatgtttcgcattgcttagatgtggtgaaagaacatcagaagatgcattgatggcgtgcttaaaccagtgatgtgctccaggtgttcctgcagtctgcagagatatttactcttgtagtcttgttgacagcacagttgtatgtgatttcttggatgtaatgtaaaccaaatgaaagataggaacagttcgtcctcttccgtatacgaaggtcactgtatcatttgtcgtggcacaagatgatctgcaggcaggactgcaacatggtttcttggactgtcctgaatgcccgttcttgttctttagttgagccagagcagcagcctggtgtcggtgcctgagacctgacgaagcacacggcaaacaaacaagtcgcagcagctagcaggggcgttgccatcgccacaagcccccaagagacccgccgaggaaaagaaaaaaaaactacggccgccgttgccaagccgagcgtgcgaaccgatccacggatgggagatcagagatcacccaccgcaggcgggcggcagtggctggcgaggtgcgtccacagaacctgctgcaggtccctgtccgtcccggcgaccccttttctaggcgagcaactccccatggcagagctgcacgcagcagggcccgtcgttggttgcagctttaaccctttttgttttaaccatacaatgcagagtcgcagaggtgaaacaggacggaaattacagaaaagatggtggtgtgccagcagccccagcatgaagaagatcaggacaaaagaaaagcttgtgattggtgacagcaacaggattggattggagccaagctaggcagtgagaggcaggcagcaagacgcgtcagccactgaaatccagagggcaacctcggcctcacaactcatatccccttgtgctgttgcgcgccgtggttagccaggtgtgctgcaggcctcctccttgtttatatatgggagatgctctcaccctctaaggt

SEQ ID NO: 74 - DNA, 1532 bp, Oryza sativa
tagtcctctaatatatgaaattttgatataggtaaagaagggtattgcaaggataagaatgtaaaaagaaataagagtaatccttaccgataatagtattccttctctaccgttaaaagttaaacctgtgcgtgtagcattttaatccaggatctatcgaatccgtccctcgttggcgtgggcgacgaacacgtgcagaagaagctttccccagaaagcacctcaccgcctcgccgtctggcagactggcacgcggggccctaccctcgctgcgcctgggcccgtccgccttctgcacactgtcacgcccccacccgctcgccgcctcgcgcctctctctccgcctccgccgcggccgcccgacgtgatagcgacacgtaggactcgccaaacacaaaaaatccatcgcgatttttggaattttgttacaaaccaaatcccgcattagagatttaatttgatttaatttaattacgtaggagtaccagataaggagatcgagttaaaaaagctaacggcgcggcgtggttatctccgaatcggctgtggctccccgcgtcggcgtcggcgcggcggcggcgcgccggccgaaccctggccgtcggatcgggcgtcgtcctgggccccacgcgccacgggcggctgtcgtttgctcctcggagcggggtgggcccaccatggccaccaccacaggtcgcggtcgcggctgacctggcggtggtcccgtgctcgcggtgttttttttttttcactctctttctctcggtggacagtagcgggggccgcggcccgcgggggcagagattgcaaaaacagcggaaacggaagattgcaaaattgcaactgctttcctgtttttaattcgggatcaaaaagattctttcgtcggggtccccgtgccattgttgtattgcgcgtaggtccttgcttgtaaaagataatctccttaattttttctttgtactactagtgtatatgcagtaagaatataccatgagtaaaatgaaccacaaaactaattacgatataccattctcatgtagacgttctcttttcttttgctagtcatacgtgcatatataaccaaacaaaaaaatgtttgaagtactcctatccaatttattactccagtagacaacaaaagaaaatgtttgaagtaataactgatccatggtacagtagggttgtcgtcaatcttgtgtttctttcattccattgtacttacaatcgtactccagctagcacagcacaatgggcttaagctttggaccccaaattctgatcttgtcggggacccgtacgaaaatactcccgtagagatgcagataccgtcacaacctacaaccaacgaatgttaagaaaacaaagggaaaaaaaaagaggcgaattcggaggagaaaaaacggtggctaaaatatagtgcgggtgtggggacgcgacgcgagcgacgaaagaggagagaggatgggttggcctgcccccccctcccctgtctataaatgcagaggcgccgagtgccctagtcgccgctc

SEQ ID NO: 75 - DNA, 841 bp, Oryza sativa
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccct

SEQ ID NO: 76 - DNA, 1392 bp, Oryza sativa
ctcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgattttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacaccccggcactatcaccgtgagttgtccgcaccaccgcacgtctcgcagccaaaaaaaaaaaaagaaagaaaaaaaagaaaaagaaaaaacagcaggtgggtccgggtcgtgggggccggaaaagcgaggaggatcgcgagcagcgacgaggccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcgtccctctcctctttctttctccgttttttttttccgtctcgtctcgatctttggccttggtagtttgggggcgagaggcggcttcgtcgcccagatcggtgcgcgggaggggcgggatctcgcggctgggtctcggcgtgcggccggatcctcgcggggaatggggctctcggatgtagatctgatccgccgttgttgggggagatgatggggcgtttaaaatttcgccatgctaaacaagatcaggaagaggggaaaagggcactatggtttatatttttatatatttctgctgctgctcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagatggctgacgccgaggata

SEQ ID NO: 77 - DNA, 743 bp, Oryza sativa
gaattcccggacctccatgcctacatcaactaatttgattccttgagtttacgtttagtgatatgtctatttttagagcttgttggggcttcggcctcagctctagccagccaaacatgttctaccaagtaccctatgttggcatgatatagtgatgcattataacaataaatgagcgagggattgctggctgaaaaagctatactagctgcatttggttatagttaaccgaactattaattgcgtgtacaacaaaataaaaaaaatgcatgttgcacattctttcattaacattatgttttggtagtgtgaattagaaatttgattgacagtagatcgacaaacatagtttcaatatgcttaagttagttatgactttaacatatcagtctccttgatattttcgttttagattcgtctctctactagtgtgtatgtccaccttccatagcagtgaagggttccattccatccctggtaaaaaaaaatcaaccactactatttatttcctaaaaagcaaaatgataaaatatcatttttttaataaaaataaaaaaattttggggtacataattgatgttgccccttgggattaaccttaaaaaagggcgaattttctagggtttggccaagttttgcaatgcaccaaattattccccttgggccggccgccaccccaaaaaaaaccccaacccccaactttccattgaaggccgggcccccttaaatcctcatccccccaa

SEQ ID NO: 78 - DNA, 144 bp, Oryza sativa
taaaaaagggcgaattttctagggtttggccaagttttgcaatgcaccaaattattccccttgggccggccgccaccccaaaaaaaaccccaacccccaactttccattgaaggccgggcccccttaaatcctcatccccccaa

SEQ ID NO: 79 - DNA, 612 bp, Cauliflower mosaic virus
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 80 - DNA, 837 bp, Coix lacryma-jobi
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacat

SEQ ID NO: 81 - DNA, 947 bp, Oryza sativa
aatccgaaaagtttctgcaccgttttcacgtcctaactaacaatatagggaacgtgtgctaaatataaaatgagaccttatatatgtagcgctgataactagaactatgtaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagatatttttttttaaaaaaaaatagaatgaagatattctgaacgtatcggcaaagatttaaacatataattatataattttatagtttgtgcattcgttatatcgcacgtcattaaggacatgtcttactccatctcaatttttatttagtaattaaagacaattgacttatttttattatttatcttttttcgattagatgcaaggtacttacgcacacactttgtgctcatgtgcatgtgtgagtgcacctcctcaatacacgttcaactagcgacacatctccaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagag

SEQ ID NO: 82 - DNA, 721 bp, Mirabilis mosaic caulimovirus
tggagattcagaaaaatctccatcaacaaataatccaagtaaggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatctctctctaccttctctctaaaaaacacttagatgtgtgagtaatcacccacttggggttgtaatatgtagtagtaaataagggaaccttagggtataccattgttgtaatattattttcagtatcaataaaataatctttcagtttatcttatattcatttgtgtgacaccgtattcccataaaaccgatcctaatctctcc

SEQ ID NO: 83 - DNA, 352 bp, Peanut chlorotic streak caulimovirus
acagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacgatcgagaagacacggccatttggacgatcatttgagagtctaaaagaacgagtcttgtaatatgtttt

SEQ ID NO: 84 - DNA, 1648 bp, Sorghum bicolor
cactcgcacatctcatggtgtcccaagaacggcaagagccagcactgcctctgcctagcaacagcagcagcgccaagcgagcagccgcgtccatggacgccagcagcccggccccgccgctcctcctccgcgcccccactcccagtcccagcattgacctccccgctgccgctggcaaggccgcggccgtgttcgacctgcggcgggagcccaagatcccggcgccattcctgtggccgcacgaggaggcgcgcccgacctcggccgcggagctggaggttccggtggtggacgtgggcgtgctgcgcaatggcgaccgcgcggggctgcggcgcgccgcggcgcaggtggcctcggcgtgcgcgacgcacgggttcttccaggtgtgcgggcacggcgtggacgcggccctggggcgcgccgcgctggacggcgccagcgacttcttccggctgccgctggccgacaagcagcgcgcccggcgcgtccccggcaccgtgtccgggtacacgagcgcgcacgccgaccggttcgcgtccaagctcccctggaaggagaccctgtccttcggcttccacgacggcgccgcgtcgcccgtcgtcgtggactacttcaccggcaccctcggccaagatttcgagccaatggggcgggtgtaccagaggtactgcgagaagatgaaggagctgtcgctgacgatcatggagctgctggagctgagcctgggcgtggagcgcggctactaccgggagttcttcgaggacagccgctccatcatgcggtgcaactactacccgccgtgcccggagccggagcgcacgctgggcacgggcccgcactgcgaccctacggcgctgaccatcctcctgcaggacgacgtcggcgggctggaggtgctggtggacggcgagtggcgccccgtccggcccgtcccaggcgccatggtcatcaacatcggcgacaccttcatggcgctgtcgaacgggcggtacaagagctgcctgcaccgcgcggtggtgaaccagcggcaggagcggcggtcgctggccttcttcctgtgcccgcgcgaggaccgggtggtgcggccgccggccagcagcgccacgccgcggcagtacccggacttcacctgggccgacctcatgcgcttcacgcagcgccactaccgcgccgacacccgcacgctggacgccttcacccgctggctctcccacggcccagtcccagcccaggaggcggcggctccctgcacctagcgagcgagcgagccgggccaaacaaacaaggggcaaaggccatctctttcgccggggcccgcgcgcggggttcgcccacgtgcgcgcccaggtgggcgctggccgcgggcaggtggcggacatgtggcctgcgggccccgcgccgccttcccatttttggacgctgccgcgcatgccgcatgcgtgcgtcgacggccctactacttctactactgctactgcgactactagtgtacatacgcaaaaatacatatatacgtattttctatatatatatatataagcaaggcggccccccggtgaccttttctttgtttttgtcgacaactgtgttttgatcccattctagctgttctatggaccatggatggttcgttcaatgtttgtacgta

SEQ ID NO: 85 - DNA, 1242 bp, Sorghum bicolor
atggtgtcccaagaacggcaagagccagcactgcctctgcctagcaacagcagcagcgccaagcgagcagccgcgtccatggacgccagcagcccggccccgccgctcctcctccgcgcccccactcccagtcccagcattgacctccccgctgccgctggcaaggccgcggccgtgttcgacctgcggcgggagcccaagatcccggcgccattcctgtggccgcacgaggaggcgcgcccgacctcggccgcggagctggaggttccggtggtggacgtgggcgtgctgcgcaatggcgaccgcgcggggctgcggcgcgccgcggcgcaggtggcctcggcgtgcgcgacgcacgggttcttccaggtgtgcgggcacggcgtggacgcggccctggggcgcgccgcgctggacggcgccagcgacttcttccggctgccgctggccgacaagcagcgcgcccggcgcgtccccggcaccgtgtccgggtacacgagcgcgcacgccgaccggttcgcgtccaagctcccctggaaggagaccctgtccttcggcttccacgacggcgccgcgtcgcccgtcgtcgtggactacttcaccggcaccctcggccaagatttcgagccaatggggcgggtgtaccagaggtactgcgagaagatgaaggagctgtcgctgacgatcatggagctgctggagctgagcctgggcgtggagcgcggctactaccgggagttcttcgaggacagccgctccatcatgcggtgcaactactacccgccgtgcccggagccggagcgcacgctgggcacgggcccgcactgcgaccctacggcgctgaccatcctcctgcaggacgacgtcggcgggctggaggtgctggtggacggcgagtggcgccccgtccggcccgtcccaggcgccatggtcatcaacatcggcgacaccttcatggcgctgtcgaacgggcggtacaagagctgcctgcaccgcgcggtggtgaaccagcggcaggagcggcggtcgctggccttcttcctgtgcccgcgcgaggaccgggtggtgcggccgccggccagcagcgccacgccgcggcagtacccggacttcacctgggccgacctcatgcgcttcacgcagcgccactaccgcgccgacacccgcacgctggacgccttcacccgctggctctcccacggcccagtcccagcccaggaggcggcggctccctgcacctag

SEQ ID NO: 86 - protein, 413 aa, Sorghum bicolor
MVSQERQEPALPLPSNSSSAKRAAASMDASSPAPPLLLRAPTPSPSIDLPAAAGKAAAVFDLRREPKIPAPFLWPHEEARPTSAAELEVPVVDVGVLRNGDRAGLRRAAAQVASACATHGFFQVCGHGVDAALGRAALDGASDFFRLPLADKQRARRVPGTVSGYTSAHADRFASKLPWKETLSFGFHDGAASPVVVDYFTGTLGQDFEPMGRVYQRYCEKMKELSLTIMELLELSLGVERGYYREFFEDSRSIMRCNYYPPCPEPERTLGTGPHCDPTALTILLQDDVGGLEVLVDGEWRPVRPVPGAMVINIGDTFMALSNGRYKSCLHRAVVNQRQERRSLAFFLCPREDRVVRPPASSATPRQYPDFTWADLMRFTQRHYRADTRTLDAFTRWLSHGPVPAQEAAAPCT

SEQ ID NO: 87 - DNA, 12906 bp, Sorghum bicolor
cactcgcacatctcatggtgtcccaagaacggcaagagccagcactgcctctgcctagcaacagcagcagcgccaagcgagcagccgcgtccatggacgccagcagcccggccccgccgctcctcctccgcgcccccactcccagtcccagcattgacctccccgctgccgctggcaaggccgcggccgtgttcgacctgcggcgggagcccaagatcccggcgccattcctgtggccgcacgaggaggcgcgcccgacctcggccgcggagctggaggttccggtggtggacgtgggcgtgctgcgcaatggcgaccgcgcggggctgcggcgcgccgcggcgcaggtggcctcggcgtgcgcgacgcacgggttcttccaggtgtgcgggcacggcgtggacgcggccctggggcgcgccgcgctggacggcgccagcgacttcttccggctgccgctggccgacaagcagcgcgcccggcgcgtccccggcaccgtgtccgggtacacgagcgcgcacgccgaccggttcgcgtccaagctcccctggaaggagaccctgtccttcggcttccacgacggcgccgcgtcgcccgtcgtcgtggactacttcaccggcaccctcggccaagatttcgagccaatggggtaagcgaagcaccgatttacatttaccgcgcgtcggcccctgaggcctgggtcttagtcttagcactgcatatacggtcggtagctctggatatgatacgtatatatgaaaccccgttccaatcccatgcacggtgtacacaggcgggtgtaccagaggtactgcgagaagatgaaggagctgtcgctgacgatcatggagctgctggagctgagcctgggcgtggagcgcggctactaccgggagttcttcgaggacagccgctccatcatgcggtgcaactactacccgccgtgcccggagccggagcgcacgctgggcacgggcccgcactgcgaccctacggcgctgaccatcctcctgcaggacgacgtcggcgggctggaggtgctggtggacggcgagtggcgccccgtccggcccgtcccaggcgccatggtcatcaacatcggcgacaccttcatggtaacccctgctctgttttttcttgtcctcctcttgtcctgtgtgtgtgtatattcacttctctctgtttttttgccccgaatcctagtggacctaactggacggattacagcacgcacacgtaggcatgtcatgtagcagcagtctgcagcactgtagtacttagcgatgcaatagagacatgcgttccagtcggttccatctcggtgggctacagctacagtcctacacggacgcggctcgtagtcgtagggacgggcgcgttctctgtatccacacacggctgcgcccaggccgaggcttccgccgcgggaaagttgcgacaacagaacggggtttgtgccgttggagcgttgcggagaggcagaggcttggggggacgggggcgcgatacgctgcgatgggtgggtgaccgaggcgacgctttcggcgggggcccgggcctgcccaggtgcgcgcggcctcgtcgccttcccctgtttttttgatgccgccgctcggtcctcggtgttctggctccgcccgcccgctcgctgggtgcccatcccatctgatccgatccgctccgctccgcggtggcggtcctatgcgatgccgccgcacgagcgcggggggccgcccgtggaggagtagaaagtggtacaaggttggttggaacttggaattgtggggggttactgctgctggtggctgctgctttgcaacttgccaggctgctgcctgttgccccccgcgttttctagccgtttccgctcgcgatccggcacgcggcgcccacaccggggctccagctcggccccttggccgtgtaggtagcaggcacttgcatctgtccgttcgacacgatgattcttgtgcactgtgtacgtatgtactaaccctttctggtatgatgtacgcatggcatgcaggcgctgtcgaacgggcggtacaagagctgcctgcaccgcgcggtggtgaaccagcggcaggagcggcggtcgctggccttcttcctgtgcccgcgcgaggaccgggtggtgcggccgccggccagcagcgccacgccgcggcagtacccggacttcacctgggccgacctcatgcgcttcacgcagcgccactaccgcgccgacacccgcacgctggacgccttcacccgctggctctcccacggcccagtcccagcccaggaggcggcggctccctgcacctagcgagcgagcgagccgggccaaacaaacaaggggcaaaggccatctctttcgccggggcccgcgcgcggggttcgcccacgtgcgcgcccaggtgggcgctggccgcgggcaggtggcggacatgtggcctgcgggccccgcgccgccttcccatttttggacgctgccgcgcatgccgcatgcgtgcgtcgacggccctactacttctactactgctactgcgactactagtgtacatacgcaaaaatacatatatacgtattttctatatatatatatataagcaaggcggccccccggtgaccttttctttgtttttgtcgacaactgtgttttgatcccattctagctgttctatggaccatggatggttcgttcaatgtttgtacgtactccacgtaaccaaactactctagtggactagtagatcgggctcatgtgatgaaactggaccgacgcggacgtcacgtgcgtcacccgcgtctggtagcggtagcgcacgagcgccgaatgtttcctgggcccgcaagagaatcgcttctcatctcctctcaccatgaatggggaaaaatgctgcgtcgaaagttccagacgtttccaaattccaaacggttttgtggcgtccgatccatggggcgccccaaacttccaagacgttttcaggttccaaatcttcgtgctccacatcaccttcttcccagattcatttgcctcgtcgcttgctctcctgtgttattcacgggtcccactgttgccccgtctgcgagaaagaaatttattagagttgaagcattcgacatttcgactgactgattgttagtatcactaaattttgtgcacatgtttctttggtcattcatctctggatattttttttagataatggatataaatatcgggcctctacatctgaggaagtacacagccaattattttcatctctggacatgggacgatggaagaggcagatagatttaggagacccttcaattcagaatttcaggtgcacaaggcctgcctggcttgcccggattcttgtttcggacatgaccaactaggccgcactacttgcactgatagctggagaaaaaacaaaactttgcaaacagcaggattatctacaagggaaactccatccacgtgaaccagcatttcagggagagatgcgacaaaaaaaaagaggcggcaacaaaaaaatcttactgcaattttatctctgcattgaacctcttccaaccatgccgcatcctgtactgttttgtatctttcccggtggtccgttgcgttctcacgcagttgataacatgcagtcacgcaccaccgaatccagtgtactaggggtagtgacttgtcacgcggaacaacaggtcggtagcaccaagcaagtcgctgtagacttgggcgtttaacaacgacttgcacaacagttcaaatatagcatatgcaattatgcacaagattgttcgactgctatccgacaaactgaagaagctgcccaattgaacagaatgtaccagtgatttccagcacactatcttacagcagcgttgagaatgaaacaacaaatgggggaaaacagatgtgtattattctacagttacaccaaagagtttgtcctttcagcatcaacaagaatcatatgcatatctagtgacaaaaattcctctaattttaccctacttggtaacagttctcttcaacacatatatttcacgtgcttgcatcgagttccttgggccgccacatcgacttctcgacgcaaagcaagccctcgttgcccttggtgtaggtcattcgcacctcccactgcagggacttggccatgctttccagttcgtttattgtgtccgcagtgtccctcacaatcagtttgccttggggcctcagtacacgatcaacctcggcaaaaactgccatcaatttgcatctgtaaacaagcaacacagatttagcatctgtaaacaccacaggtttcattgcaagaagcataaagcatgcaaacatgctacttgtacatgtcaaagaaacatgtcaaactcaaacacatgaaaatcattattattgttttcttgctgaactgatcacattagttggtttcaatttctgagttccactagtaatctataccagaaggatagaataatgtcaagaacaagagatacaaacctctttgtgagctttgagaatagatggtccgcgtgcagaaggtcataagttcttgggtaagtgctcaaagactcgcaccagtcatggtacataccaaacaaaccacgctcgtaaatgatgggcagtgtgtctggtgaatcaatcggcacaatattcatgacccagaccttttggtccctcagagctgcagcaaaactgccatgcaacgatgtaaagcattagtaaaaatattgggttttttaaaccaaaaccaagaaagataattcctccagcttaactgaaagaaagaaagaaaaaaactgcttaatgacttatggtggacaagttgcctgttatgttttatgatagctatgtgccagcttggctaactggtagttatgtagtgtgatctgaattaccaaaaaagagaagaaaaaaaaatcatgcccaagaaactgagaaagacacccatttacttaccctccatacacagctctcatgtccattacatttctcactttggaccagtcaattcccatgccattcacatacgatttacttacaacccttttccagtgagcattatctgcctcgaaatcttcatttgcaggctttccatagaccccaaccttggaaccatcaatccagaaaggagtcttctcaagcctttgtggccaaaactctggccattttgaccctcgaactttcgagccaacaggcagtttgtgcatgcatgcttccaaaggtacattcctgcaaatcaaaagattgtgtaagcaaagcagaggaagcacttcgccgcattgaaaatacgttcttctcaaagaaacaaaaccataccaagctgcatctgcatcatcagattccttgcacaatggtgggttgttttcggatcttttctcatagcaaatgttgtccattggtttctgaaatatgaccataccaacttgatttaacttatccttagtcttgttgaccatcttccagcacatggactttgtcaaagtggacatcgctgaaaagattaaggggtcatatgttatgatagaaataaaattcaattttgcactgttggtacatagcatctgttttgaacaaatgcaatccttccttatccatgaaagaagttaacccctgatacttaggattattcagtactttcactcatgaactgctgaatttgttctgccagtagttgctatactagaaatgttcagtgtaccaaacataaatttggtacgggttccttattaaagatgggaggctgtatggtatttcgacgtaacaaatcaagttagcagctaccctacttatggatatacacttctcaaaatgaatatacatagttttgataggtgacattaattaatataagaacttcatgcagttagggtgaaactaaactaagcagttacggaaataccattccaaatctcaacatcctctgggagcttttggtaaacaggagtggcagaccagacaaagtaaccaccagggcgtaacaagcggttcaattccagcaaaagcatgccacctaaaaggagtcagtaataagattcagttctatagcaaatcaataaatgaaaggaagacatgtcaccaacaagacaaaccttcaatgtgccaagggaccctgcagcgagcgcaatgaatgacatcaaagactctgctggggtatggaagtctcttggtgcccatcacagctgatattgctggaattcccctttctaatgcaaattgtacttgagcttcatgctcatctttcggagcaaaagacatggtaagcacatctctatcaaacatgtagcctccaaagctggcaactccacaaccgacatctagaatgacgcggcttcgtttgccccatgcaatatcaggcagtgcctgtgaatgacagtttaatcagcatatgatgaaagcaagtgtgataatatcaagttcaaagatgcaacatgaaactttcataatcatggacagtactaagcttgcttgatagattaatgtatggatgagactaaaaaaaaggaaagttgtatccatcagaacgagaggctgaaaacacatggctggctgtgaaagcctgatgtcgtttagtctagcataaacaaactgtcctcagcatgtagatttccatagggtggcatttgacaaattatgattgtggactagcgaatcaatcactgattctcaaaagtgtgagacagatgagttcaagtctaaggggtgactaatatgggatgctgggatgatgatgatgatatatacctgctgaatagtatcaatatagtggagggcaccattcttgaactgagtcccacccccagggaacaggaggtagtcacctgatactttaacccaattttgatgtcccttgtactctgcgagcctagtgtgaggaacattgctgtaccatacctgcaaaaagcagcacaagatggtaataagtaaacagagatcttggtcagctaaagatgattcagtgtcgtacaatttagaatagacagaatcaccttgtccctgctccttggccactcaattgggcgtttatatccttctgggagtggaacaaggcaggtaggaggctcctcagggcaatgcctctcacgatgttcataatgtttagtagttcgaagcttcttgatagccttctcgttgtcaaggcaaggtatgtaatctgtcgaggcactgctattacatagtttccaggaatagctagtcgcatcacctgaagactttgatgacgcttggacttccttttcattcttggactctgcagcctgtgtggggaatgaaccattctgggtatttgactccttcagaagctctgattgggccccatcaggaaatacctcgttggagtttgagctctgatccttctctccattttcttccaccttctcttctatctgaggttgctcctcctgagtggcatcaccttcaggcttctcttcttgatcatccttgctctctccatcaggtttttcatcaccactctcatttgtgatttcatcatctttcttctccccactcttctccccgtcaccatcgttcttcatgtcatctgaccgcccttctgattttccatttgcatcatcaaacatatccttggtctcagctttctctgtcggcacttccggctccttctcttcaggcttctcctctggcttctcagtgaacttctcttccatcgtggcatccttgttattcggctcctccggcatcgtggcatcattgttgtcggtgtcctcaaatttctcagaaccttcaccagcattgtcctgtgaggccccaaaattgacaggcgcaggctgctgcttcaccaccggcttcttattcgaggagatctccagcgggaagacagtggacgaggtcatcatccacgcgccgaccaggcagagcgccacaaagacgacgaccgtggtggtcgtgcagaacgacgacgacgtcgaggacggccggcggccgtccatcttcccacctcggccaaatgccattagtgcctggcgaacatgtaccagagcaccgaccttcacgcgatttatctccaccaactactgctggaccaagaacccccaaaaaaatcgcacctttgtctgctttgtgctgctacagccgcgcggcacctgaagcaaaccacaaaaaaaacttaaatcgccgcggacataaatcaaggtgctggatctaaagaacaaacgctggatctactcaagcaacaacggaaggaagatccgctattggtgctagtattagcttcttgtttcctagtactacagcggctctttcccagtataagaacacgggaaaacgcggagaaatcccccttcgtggccaaacatggaaagaaaattagtaaagcgtgtgctttaaaaccccctcgttctgttccttccgcggagagctaccgcatcttccaattgagctggttctcagctgggcgcaaaacgcgcactaatcaatgtccgattccatccacaaagaaaaaaaagacgggaacagctaatccagcagctcgctcgctagctagctagctcatcggcggaaggacggaaccagctttgctggatccaggacagcaagagtgtgcaaggagaaagaacggagcagcaatgcggattgcggaggcggtggattggtacctcgccggaaccgaccggagtggtcgcggtggccctccgcgcggatctcgaagaggagcgaggaaggggaaggcggatgcgcgtccttgggttctctgccaccgcactgggcctcgccgcgttataaaggcgggcgggcgggcgggcagcgcagtgtgagtggagtgcaatctgttgtgtagtgtgtgaagaggcggaagcggaagcggaggagatgggttcgcattagacgaccgtacgtaattatacgctatactagtacttgggttagattactcgggagatcttggccaaaatgtccggtctgagtgtttggtagttttatggatttgcccttttaagatgttggtatttctccgggagcttagaaagaagaaatggcgatgctttaggccttgtttagatgcgaaaaaaatttggatttcgctactgtggcatttttatttgtttgtagcaaatattgtccaaacacggactaactaagattcatctcgcgatttacagttaaactgtacaattagtttttattttcatttatatttaatgtttcatggatgtgtcgaaagatacgatatgatagaaaattttgaaaactttttagttattgaggttaactaaacaatgccttaattgagaatttactcgagcaaaaagagttaggtcagtctcagtggagagtttcatggtgttgtttccaagactgccatatcatgtgaaatgaaatgaaacttggttgaaacactcactctcaatggagagtttcattttatagtttcatgggcatttaatttcaatactcatagagagttgatatcgtgccaactcatttcttctctctcttcttaaatacacagtcatatcatcaaaaaaaatcctatgtagcaacatatttaatgcaaataaaactcatatggtggactgtaggagtagcattaggccaagggcacacacacggtcacggtgtgagtgcgacggtgcgagtgggcccgcggcggtagtaagtgcgtgcgcgcccggcgcccccctccgcggcgacgacgcagcggcagcgcgtcgtccagtgcaccgtctgctgttcggcgctgcgggtcctccgcgccacggcgcagtgaaccgggcgcgtgcatcccgggagcggcggcttggcactcccctgcttgtcggtggcggccgtcggcatcgctcggccccggagcgtcacgaggctgctgattgggagcgagagcgagtagtggggctggttggggacaatcccattcccacccggcccaccaggctgggactggcccactagtcactagtgggtggctcatgggtgtgggtgggctggctaatgccgcctgcccaacaaccaacccaacccctgtggacgctggtaccggtagttgccgcgccatggtggactgctgccgcctgatgcctttgcctgccacgctccacgagttgaggcgcaccaaactgtgctgtgctcctgatttgtgctaatcggccgacgcgtaccattctttctttctttcgtctacgcgcagagaggccggttgactgtttcttcgttggagggccatgttgactcgtactaataataaaaataataatactaggttgactttttcaattccaacgcagcagtgcaaagctgcccacctatgagcacaggtccttttttaactccgtttttgtacgtacacacgtactgtccagcctgtgtctaataatcttaccaaaaacctgtcatctcactatcaaccaatcaggctctccgcctgttcgtcgaggaacagcagttgttttccctactccaacatagagtacactatggacgcacattaccatgccagcttgagcttagcattgcccaccgttggataactgccatgccattctcaggccctgtttagttcccatctaaaaatttttcatccattccatcgaatctttggacacatgcatggaacattaaatgtagataaaaaaataaactaattacacagtttagttgagaatcgcgagacgaatcttttaagtctagttactccataattagccttaagtgctacagtaatccacatatactaatgacagattaattatgcttaataaatttgtcttacagtttcctgacgagctatgtaatttgtttttttattagtttctaaaaacccctcccgacatccttccgacatatccgatgtgacaaccaaaaaattttcatcttcaatctaaacaggccctcactctcatcatctcatgccggggcagcaggtccgtcgtcaggtctgtcgtcccgtcccgtgccgtctgaagcaacaggcgagagaaacgccgttccatcggtttgccgagcgtgcagaggatagagctatactcgatccggagaggattgtgaaacgaagcacggttaagcagtgccgcgcacgtgctgctctgctcctggatccgatccagatcgactcggggcgtctcggcctcagcggcgatggcaatcatcgcgcgcgctgctggagctggacgttttcgtcttgcattgcaggaggcggaacagaacggagaaagccacggcgcgctttgccgacgccacgcgctgacacgagggacccgttcagcggccagcacgcagcctaatcatgcctgtcggggggagctcatccgttcctgaatttgggtcatgctccagtatcaggtattcaggtactagtactcctgagccatgtgctgcgacaaaaaagcgaggctcctgtagtagagccttgtttacttacaaaattttttacattctcagttatattaaatcttgtgacacatgcataaagcattaaatatacataaaagaaataattatttacacagttacttataatttgcgaaacgaatcttttaagactggttagtttatgattagataatatttattaaatacaaatgaaagtaatattatttatattttgcaaaaagtaaataagacctaggtagctaggccaacgtgagcatgtcggacccggaccggttcgttctacggcgcgtcccgcaaacctgcagccaggtagtagtagtacaccgtgcacgggagaggtgcgccatgcatgctcgggcaaaagatcatagagaaaggtgcagcgtttcagttgcacacctgaccgagtgacgcctcgccttgtttggctttgttcccaaaattttttaaaattcctcatcacattaaatctttgaacgaatatatggagcattaaatataaataaaagaaataattaatcatacaatttgtctgtaatttgcgagacgaatcttttgagcctagttagtttataattaaataatatttgttaaatacaaacgaaaatgctacgttagccaaaactaaaatttttctccaaacgtgacccagcaccttccgatcaatcatcactcagcgggtcacgtcagaagatcagatggaccttgccgtccgggcctgtctctcggcctcctccccatctggaacgaacagaggtccagtcctgtttcgagtcgagctgagtcgatcagatgggcctaaataggccgaagacgtaggcaaagggcccgctgatttatctgattcttctaggaccgtgcatgcgcggatgggcctaggtggaaacccaacagatgtgaggcttcaaagaggaagaagtccgttacacatggagagttagtctataatgggataatatttaccacaaacaaataaaaatactacagtagcgaaatccaaaatttttcacatctaaacaaggccctagatgttttgtcagtgccagaccagagaaaatctcgtcttctgctgtcaatagctttgatgattcctggcggcagaggtaaagcttgcctgggccttgtttagttccgaaaagtgaaaagttttcggtactgtagcacttttgtttgttcgtgacaaatatcatccaattatggactaactagaattaaaagattcgtctcgtgatctacagctaaactgtgtaattagtttttgttttcgtctatatttaatgtttcatgcatgtgccacaagattcgatgtgacggagaattttgaaaattttttggttttcagagtgaactaaacaaggcccagatgtaattgaccatgccatcgagcgcgagttgactagagtgagtcggccctgatggttaagtagtgcagactgccaagtggacaaccgtctatcaactttgcagagtggggcgaatgcactgaggatgttggagaggggcaagccaaggtaaacttgaggaaagatgcttgttgacactgtagtatgtgaacaatcctgtttaattttgtgtcctcgacg

SEQ ID NO: 88 - DNA, 1790 bp, Setaria italica
tctcatggtgtcccaagcacagcaagagccagctctgcctcacagcagcagcaccgccaagcgcgcagccgcgtcactcatggacgcccgcccggcccagcctctcctcctccgcgccccgactcccagcattgacctccccgcgtccaagccggacagggccgccgcggcggccggcaaggccgccgccgcctccgtgttcgacctgcggcgggagcccaagatcccggcgccattcgtgtggccgcacgacgacgcgcggccggcgtcggcggcggagctggacgtgccgttggtggacgtgggcgtgctgcgcaatggcgaccgcgcggggctgcggcgcgctgcggcgcaggtggccgcggcgtgcgcgacgcacgggttcttccaggtgtgcgggcacggcgtgggcgcggacctggcgcgcgcggcgctggacggcgccagtgacttcttccggctgccgctggcggagaagcagcgcgcccggcgcgtcccggggaccgtgtccgggtacacgagcgcgcacgccgaccggttcgcgtccaagctcccctggaaggagaccctctccttcgggttccacgacggcgccgcgtcgcccgtcgtcgtcgactacttcgccggcaccctcgggcaggacttcgaggcagtggggcgggtgtaccagaggtactgcgaggagatgaaggctctgtcgctgacgatcatggagctcctggagctgagcctgggcgtggagcgcggctactaccgcgacttcttcgaggacagccgctccatcatgcggtgcaactactacccgccgtgcccggagccggagcgcacgctgggcacgggcccgcactgcgaccccaccgcgctgaccatcctcctccaggacgacgtcggcgggctcgaggtcctcgtcgacggcgactggcgccccgtccgccccgtccccggcgccatggtcatcaacatcggcgacaccttcatggctctgtccaacgggcggtacaagagctgcctgcaccgggcggtggtgaaccagcggcaggagcggcggtcgctggccttcttcctgtgcccgcgcgaggaccgggtggtgcgcccgccggccagcggcgccgtcggcgaggcgccccgccgctacccggacttcacctgggccgacctcatgcgcttcacgcagcgccactaccgcgccgacacccgcacgctggacgccttcacacgctggctctcccacggcccggcccaggacgcgccagtggcggcggcggcttccacctagctagcggcgcggatccgaccgagcccattgacgacgccgtccctttccgccgccgccggggcccgcgcgggggttcaccccacgtgcgcgcccaggtgggcgaggtggcggcctcgtggcccgcgggccccgcgccgccttcccatttttgggcgctgccgccccgcgcgcatgccggatgcgtgcgtccacggcctactgctgctactagtgtacatatacaaacatacatatatacgtagtataaatatataagcaagcggcccggtgccccttttcgttttcttgttttgtcgatcacaatctctggattcgatggatggataaatgtttgtacgcatgcatgtagatgggctcatgaaatttcagaatctggaacggacgaggagctcacgtgcctcttccgtgtctggtagcggtagctgcgtgccaaatgtctggtgggcccaaagaaattctagtgccacccgtccggatccggcatccgaaagttcccgacggttcgacacccgaa

SEQ ID NO: 89 - DNA, 1272 bp, Setaria italica
atggtgtcccaagcacagcaagagccagctctgcctcacagcagcagcaccgccaagcgcgcagccgcgtcactcatggacgcccgcccggcccagcctctcctcctccgcgccccgactcccagcattgacctccccgcgtccaagccggacagggccgccgcggcggccggcaaggccgccgccgcctccgtgttcgacctgcggcgggagcccaagatcccggcgccattcgtgtggccgcacgacgacgcgcggccggcgtcggcggcggagctggacgtgccgttggtggacgtgggcgtgctgcgcaatggcgaccgcgcggggctgcggcgcgctgcggcgcaggtggccgcggcgtgcgcgacgcacgggttcttccaggtgtgcgggcacggcgtgggcgcggacctggcgcgcgcggcgctggacggcgccagtgacttcttccggctgccgctggcggagaagcagcgcgcccggcgcgtcccggggaccgtgtccgggtacacgagcgcgcacgccgaccggttcgcgtccaagctcccctggaaggagaccctctccttcgggttccacgacggcgccgcgtcgcccgtcgtcgtcgactacttcgccggcaccctcgggcaggacttcgaggcagtggggcgggtgtaccagaggtactgcgaggagatgaaggctctgtcgctgacgatcatggagctcctggagctgagcctgggcgtggagcgcggctactaccgcgacttcttcgaggacagccgctccatcatgcggtgcaactactacccgccgtgcccggagccggagcgcacgctgggcacgggcccgcactgcgaccccaccgcgctgaccatcctcctccaggacgacgtcggcgggctcgaggtcctcgtcgacggcgactggcgccccgtccgccccgtccccggcgccatggtcatcaacatcggcgacaccttcatggctctgtccaacgggcggtacaagagctgcctgcaccgggcggtggtgaaccagcggcaggagcggcggtcgctggccttcttcctgtgcccgcgcgaggaccgggtggtgcgcccgccggccagcggcgccgtcggcgaggcgccccgccgctacccggacttcacctgggccgacctcatgcgcttcacgcagcgccactaccgcgccgacacccgcacgctggacgccttcacacgctggctctcccacggcccggcccaggacgcgccagtggcggcggcggcttccacctag

SEQ ID NO: 90 - protein, 423 aa, Setaria italica
MVSQAQQEPALPHSSSTAKRAAASLMDARPAQPLLLRAPTPSIDLPASKPDRAAAAAGKAAAASVFDLRREPKIPAPFVWPHDDARPASAAELDVPLVDVGVLRNGDRAGLRRAAAQVAAACATHGFFQVCGHGVGADLARAALDGASDFFRLPLAEKQRARRVPGTVSGYTSAHADRFASKLPWKETLSFGFHDGAASPVVVDYFAGTLGQDFEAVGRVYQRYCEEMKALSLTIMELLELSLGVERGYYRDFFEDSRSIMRCNYYPPCPEPERTLGTGPHCDPTALTILLQDDVGGLEVLVDGDWRPVRPVPGAMVINIGDTFMALSNGRYKSCLHRAVVNQRQERRSLAFFLCPREDRVVRPPASGAVGEAPRRYPDFTWADLMRFTQRHYRADTRTLDAFTRWLSHGPAQDAPVAAAAST

SEQ ID NO: 91 - DNA, 2888 bp, Setaria italica
tctcatggtgtcccaagcacagcaagagccagctctgcctcacagcagcagcaccgccaagcgcgcagccgcgtcactcatggacgcccgcccggcccagcctctcctcctccgcgccccgactcccagcattgacctccccgcgtccaagccggacagggccgccgcggcggccggcaaggccgccgccgcctccgtgttcgacctgcggcgggagcccaagatcccggcgccattcgtgtggccgcacgacgacgcgcggccggcgtcggcggcggagctggacgtgccgttggtggacgtgggcgtgctgcgcaatggcgaccgcgcggggctgcggcgcgctgcggcgcaggtggccgcggcgtgcgcgacgcacgggttcttccaggtgtgcgggcacggcgtgggcgcggacctggcgcgcgcggcgctggacggcgccagtgacttcttccggctgccgctggcggagaagcagcgcgcccggcgcgtcccggggaccgtgtccgggtacacgagcgcgcacgccgaccggttcgcgtccaagctcccctggaaggagaccctctccttcgggttccacgacggcgccgcgtcgcccgtcgtcgtcgactacttcgccggcaccctcgggcaggacttcgaggcagtggggtaagtatgtaggaatgaacttggcacgcattgcatccacatggcgtgctgatcgaacgagctgagccaaccggcatgcacacatggcgtggcaggcgggtgtaccagaggtactgcgaggagatgaaggctctgtcgctgacgatcatggagctcctggagctgagcctgggcgtggagcgcggctactaccgcgacttcttcgaggacagccgctccatcatgcggtgcaactactacccgccgtgcccggagccggagcgcacgctgggcacgggcccgcactgcgaccccaccgcgctgaccatcctcctccaggacgacgtcggcgggctcgaggtcctcgtcgacggcgactggcgccccgtccgccccgtccccggcgccatggtcatcaacatcggcgacaccttcatggtacggccgccgctaatccatccttttgttgctcttatctcctctggcgagtgcgagtaacgaaagcgctagctcccctgctccttgtcctgctctgtttcccaagtcctaatggagctaaccgggcagactgcaacacgcacgcgtaggcatgtcacgtagccaccacttgcactgtgctgcgcagcgacgacgcaacgcggacgtgcgttcgagtcggttccatctcggcgccgctacacgcggccgcggctcctagcctcctagggctccctgatccctatccccgagcccttccgcgggaaaagttcgttggcgacggcagaggagagccgacgggtccgtgccgttggagcgtggcggcaggagaggccgggagggtgttttgttgcgttgcgcggcggcgcggaggatgcgatggcgcgggcgggcggcgctttcggcggtggcccccgcgacccacgtgcgcgcgcggtctcgtcgccttccctgttttggtgccacctctctgtgtccgggaatgggttggcttagcggcgaccgagaccgggcggtggtctggcctgctcccggcgcccatcccgcctggtctctcatcctgctcctcctatgcgcgagggggcctgtagcggctggagtacaagcagattggttgggttgggttgctgctgcttggctgttgcccgcccgctttctagccgtttccgctcgccatccggcacgcggcgcccacgccggggctccagctcggcccctttggccgtgtgggtggcaggcacccctgcatcgtctcgtgcgtccggtttccgcgcctggccccccgccttgaggtttccctgtgcttttgacaagactttcgtagatatatgtgtgtgtatgtgtgtgtgtgcgtgcgcgcgtgtgtgtatatatatatataaataaataacatctgtgaatgatggattacacgtgtagctgaccggctgattgtgttcgcgtgtgtgtcttcgatgcattgcaggctctgtccaacgggcggtacaagagctgcctgcaccgggcggtggtgaaccagcggcaggagcggcggtcgctggccttcttcctgtgcccgcgcgaggaccgggtggtgcgcccgccggccagcggcgccgtcggcgaggcgccccgccgctacccggacttcacctgggccgacctcatgcgcttcacgcagcgccactaccgcgccgacacccgcacgctggacgccttcacacgctggctctcccacggcccggcccaggacgcgccagtggcggcggcggcttccacctagctagcggcgcggatccgaccgagcccattgacgacgccgtccctttccgccgccgccggggcccgcgcgggggttcaccccacgtgcgcgcccaggtgggcgaggtggcggcctcgtggcccgcgggccccgcgccgccttcccatttttgggcgctgccgccccgcgcgcatgccggatgcgtgcgtccacggcctactgctgctactagtgtacatatacaaacatacatatatacgtagtataaatatataagcaagcggcccggtgccccttttcgttttcttgttttgtcgatcacaatctctggattcgatggatggataaatgtttgtacgcatgcatgtagatgggctcatgaaatttcagaatctggaacggacgaggagctcacgtgcctcttccgtgtctggtagcggtagctgcgtgccaaatgtctggtgggcccaaagaaattctagtgccacccgtccggatccggcatccgaaagttcccgacggttcgacacccgaa

SEQ ID NO: 92 - DNA, 1567 bp, Oryza sativa
tgcccagacagctcgccctgcacacacacacacactcacactcacacacgctctcaactcactcccgctcaacacagcgctcacttctcatctccaatctcatggtggccgagcaccccacgccaccacagccgcaccaaccaccgcccatggactccaccgccggctctggcattgccgccccggcggcggcggcggtgtgcgacctgaggatggagcccaagatcccggagccattcgtgtggccgaacggcgacgcgaggccggcgtcggcggcggagctggacatgcccgtggtcgacgtgggcgtgctccgcgacggcgacgccgaggggctgcgccgcgccgcggcgcaggtggccgccgcgtgcgccacgcacgggttcttccaggtgtccgagcacggcgtcgacgccgctctggcgcgcgccgcgctcgacggcgccagcgacttcttccgcctcccgctcgccgagaagcgccgcgcgcgccgcgtcccgggcaccgtgtccggctacaccagcgcccacgccgaccgcttcgcctccaagctcccatggaaggagaccctctccttcggcttccacgaccgcgccgccgcccccgtcgtcgccgactacttctccagcaccctcggccccgacttcgcgccaatggggagggtgtaccagaagtactgcgaggagatgaaggagctgtcgctgacgatcatggaactcctggagctgagcctgggcgtggagcgaggctactacagggagttcttcgcggacagcagctcaatcatgcggtgcaactactacccgccatgcccggagccggagcggacgctcggcacgggcccgcactgcgaccccaccgccctcaccatcctcctccaggacgacgtcggcggcctcgaggtcctcgtcgacggcgaatggcgccccgtcagccccgtccccggcgccatggtcatcaacatcggcgacaccttcatggcgctgtcgaacgggaggtataagagctgcctgcacagggcggtggtgaaccagcggcgggagcggcggtcgctggcgttcttcctgtgcccgcgggaggacagggtggtgcggccgccgccgagcgccgccacgccgcagcactacccggacttcacctgggccgacctcatgcgcttcacgcagcgccactaccgcgccgacacccgcacgctcgacgccttcacgcgctggctcgcgccgccggccgccgacgccgccgcgacggcgcaggtcgaggcggccagctgatcgccgaacggaacgaaacggaacgaacagaagccgatttttggcggggcccacgcccacgtgaggccccacgtggacagtgggcccgggcggaggtggcacccacgtggaccgcgggccccgcgccgccttccaattttggaccctaccgctgtacatattcatatattgcaagaagaagcaaaacgtacgtgtgggttgggttgggcttctctctattactaaaaaaaatataatggaacgacggatgaatggatgcttatttatttatctaaattgaattcgaattcggctca

SEQ ID NO: 93 - DNA, 1170 bp, Oryza sativa
atggtggccgagcaccccacgccaccacagccgcaccaaccaccgcccatggactccaccgccggctctggcattgccgccccggcggcggcggcggtgtgcgacctgaggatggagcccaagatcccggagccattcgtgtggccgaacggcgacgcgaggccggcgtcggcggcggagctggacatgcccgtggtcgacgtgggcgtgctccgcgacggcgacgccgaggggctgcgccgcgccgcggcgcaggtggccgccgcgtgcgccacgcacgggttcttccaggtgtccgagcacggcgtcgacgccgctctggcgcgcgccgcgctcgacggcgccagcgacttcttccgcctcccgctcgccgagaagcgccgcgcgcgccgcgtcccgggcaccgtgtccggctacaccagcgcccacgccgaccgcttcgcctccaagctcccatggaaggagaccctctccttcggcttccacgaccgcgccgccgcccccgtcgtcgccgactacttctccagcaccctcggccccgacttcgcgccaatggggagggtgtaccagaagtactgcgaggagatgaaggagctgtcgctgacgatcatggaactcctggagctgagcctgggcgtggagcgaggctactacagggagttcttcgcggacagcagctcaatcatgcggtgcaactactacccgccatgcccggagccggagcggacgctcggcacgggcccgcactgcgaccccaccgccctcaccatcctcctccaggacgacgtcggcggcctcgaggtcctcgtcgacggcgaatggcgccccgtcagccccgtccccggcgccatggtcatcaacatcggcgacaccttcatggcgctgtcgaacgggaggtataagagctgcctgcacagggcggtggtgaaccagcggcgggagcggcggtcgctggcgttcttcctgtgcccgcgggaggacagggtggtgcggccgccgccgagcgccgccacgccgcagcactacccggacttcacctgggccgacctcatgcgcttcacgcagcgccactaccgcgccgacacccgcacgctcgacgccttcacgcgctggctcgcgccgccggccgccgacgccgccgcgacggcgcaggtcgaggcggccagctga

SEQ ID NO: 94 - protein, 389 aa, Oryza sativa
MVAEHPTPPQPHQPPPMDSTAGSGIAAPAAAAVCDLRMEPKIPEPFVWPNGDARPASAAELDMPVVDVGVLRDGDAEGLRRAAAQVAAACATHGFFQVSEHGVDAALARAALDGASDFFRLPLAEKRRARRVPGTVSGYTSAHADRFASKLPWKETLSFGFHDRAAAPVVADYFSSTLGPDFAPMGRVYQKYCEEMKELSLTIMELLELSLGVERGYYREFFADSSSIMRCNYYPPCPEPERTLGTGPHCDPTALTILLQDDVGGLEVLVDGEWRPVSPVPGAMVINIGDTFMALSNGRYKSCLHRAVVNQRRERRSLAFFLCPREDRVVRPPPSAATPQHYPDFTWADLMRFTQRHYRADTRTLDAFTRWLAPPAADAAATAQVEAAS

SEQ ID NO: 95 - DNA, 3140 bp, Oryza sativa
tgcccagacagctcgccctgcacacacacacacactcacactcacacacgctctcaactcactcccgctcaacacagcgctcacttctcatctccaatctcatggtggccgagcaccccacgccaccacagccgcaccaaccaccgcccatggactccaccgccggctctggcattgccgccccggcggcggcggcggtgtgcgacctgaggatggagcccaagatcccggagccattcgtgtggccgaacggcgacgcgaggccggcgtcggcggcggagctggacatgcccgtggtcgacgtgggcgtgctccgcgacggcgacgccgaggggctgcgccgcgccgcggcgcaggtggccgccgcgtgcgccacgcacgggttcttccaggtgtccgagcacggcgtcgacgccgctctggcgcgcgccgcgctcgacggcgccagcgacttcttccgcctcccgctcgccgagaagcgccgcgcgcgccgcgtcccgggcaccgtgtccggctacaccagcgcccacgccgaccgcttcgcctccaagctcccatggaaggagaccctctccttcggcttccacgaccgcgccgccgcccccgtcgtcgccgactacttctccagcaccctcggccccgacttcgcgccaatggggtaattaaaacgatggtggacgacattgcatttcaaattcaaaacaaattcaaaacacaccgaccgagattatgctgaattcaaacgcgtttgtgcgcgcaggagggtgtaccagaagtactgcgaggagatgaaggagctgtcgctgacgatcatggaactcctggagctgagcctgggcgtggagcgaggctactacagggagttcttcgcggacagcagctcaatcatgcggtgcaactactacccgccatgcccggagccggagcggacgctcggcacgggcccgcactgcgaccccaccgccctcaccatcctcctccaggacgacgtcggcggcctcgaggtcctcgtcgacggcgaatggcgccccgtcagccccgtccccggcgccatggtcatcaacatcggcgacaccttcatggtaaaccatctcctattctcctctcctctgttctcctctgcttcgaagcaacagaacaagtaattcaagcttttttttctctctcgcgcgaaattgacgagaaaaataagatcgtggtaggggcggggctttcagctgaaagcgggaagaaaccgacctgacgtgatttctctgttccaatcacaaacaatggaatgccccactcctccatgtgttatgatttatctcacatcttatagttaataggagtaagtaacaagctacttttttcatattatagttcgtttgattttttttttttaaagtttttttagttttatccaaatttattgaaaaacttagcaacgtttataataccaaattagtctcatttagtttaatattgtatatattttgataatatatttatgttatattaaaaatattactatatttttctataaacattattaaaagccatttataatataaaatggaaggagtaattaatatggatctcccccgacatgagaatattttccgatggtgtgacgacgccatgtaagcttcggtgggcctggacggccagaggtgccaacagccacgtccaacaacccctgggtccccccctaacactccaaacagtagtgagtagtgtctcgtcgcgttttagtatttgatgacaaacaaagtgtgagttgagttagccaccaccaacttgcacacgagcacatacatttgtgtccattctcgccagtcacttccatctctagtcctaactcctatctagcgatgtaagcggataatttcatcatccgtatataaacctgtttgttatagttaatttcctatataatactataacagtatacattttaaaagaaaacaaaattaggataaacaggccctgctcctatccatccatggcacttggaaggaccagactcggtcatgccatgccaagccaagatatgggttatggaagagtagagaagaggagagatgagagataagcatgcgttctcctcctcgttggatgtgtattttggagggatttgtgtagtagtagcagcggcgccgcggggacggatgcggatggtggcgctttcggtggcgttttcccgggggggttttggtttggcgcttgggggggatggcatggcgcggcgtgcggctgcacgccacacacacgcgcgcgcacgcacgtacgtcgtcgtcgccgcgggcggacggtagcttagggtggtgtgttccgcgcgcgggcgcggattgttccatgccgatcgatttggcgccaccctcgccgcggctcttgtcgcgtcgtgcgcctctctcgcgcggtttgtccttgtcgcgttgctcagccggcgacgggggcacggacattggcgatgtagccctgcacgtgtcggcctctccgttgatgaatgatgatgtatgtatgtatttttttttgtctgaaggaatttgtggggaattgttgtgtgtgcaggcgctgtcgaacgggaggtataagagctgcctgcacagggcggtggtgaaccagcggcgggagcggcggtcgctggcgttcttcctgtgcccgcgggaggacagggtggtgcggccgccgccgagcgccgccacgccgcagcactacccggacttcacctgggccgacctcatgcgcttcacgcagcgccactaccgcgccgacacccgcacgctcgacgccttcacgcgctggctcgcgccgccggccgccgacgccgccgcgacggcgcaggtcgaggcggccagctgatcgccgaacggaacgaaacggaacgaacagaagccgatttttggcggggcccacgcccacgtgaggccccacgtggacagtgggcccgggcggaggtggcacccacgtggaccgcgggccccgcgccgccttccaattttggaccctaccgctgtacatattcatatattgcaagaagaagcaaaacgtacgtgtgggttgggttgggcttctctctattactaaaaaaaatataatggaacgacggatgaatggatgcttatttatttatctaaattgaattcgaattcggctca

SEQ ID NO: 96 - DNA, 1170 bp, Triticum aestivum
atggacaccagccctgcaactcccctgctcctccagcctcctgctcccagcattgacccgttcgccgccaaggcggccgtcaacaagggcggcggcgcggcaaccgcggtgtacgacctccggagggagccgaagatccccgccccgttcgtgtggccgcacgccgaggtgcgccccaccacggcccaggagctggccgtgccggtggtggacgtgggcgtgctgcgcaatggcgacgccgcggggctccgccgcgccgtggcgcaggtggccgcggcgtgcgccacgcacgggttcttccaggtgtccgggcacggcgtggacgaggccctggcgcgcgcggcgctggacggcgcgagcggcttcttccggctgccgctggccgagaagcagcgcgcgcggcgcgtcccggggaccgtgtccgggtacacgagcgcgcacgccgaccggttcgcctccaagctcccctggaaggagaccctctccttcggcttccacgaccgcgccggcgccgcgcccgtcgtggtggactacttcaccagcaccctcgggccggactacgagccaatggggagggtgtaccaggagtactgcgggaagatgaaggagctgtcgctgaggatcatggagctgctggagctgagccagggcgtggagaagcgcgggtactaccgggagttcttcgcggacagcagctccatcatgcggtgcaactactacccgccgtgcccggagccggagcgcacgctgggcacgggcccgcactgcgaccccacggcgctcaccatcctactgcaggacgacgtgggcgggctggaggtcctcgtcgacggcgactggcgccccgtccgccccgtccccggcgccatggtcatcaacatcggcgacaccttcatggcgctgtcgaacgggcggtacaagagctgcctgcaccgcgcggtggtgaaccggcggcaggagcggcggtcgctggccttcttcctgtgcccgcgcgaggaccgcgtggtgcggccgccgccgggcctgaggagcccgcggcggtacccggacttcacctgggctgacctcatgcgcttcacgcagcgccactaccgcgccgacacgcgcaccctcgacgccttcacccagtggttctcctcctcctcctcctcggcccaggaggcggcctga

SEQ ID NO: 97 - protein, 389 aa, Triticum aestivum
MDTSPATPLLLQPPAPSIDPFAAKAAVNKGGGAATAVYDLRREPKIPAPFVWPHAEVRPTTAQELAVPVVDVGVLRNGDAAGLRRAVAQVAAACATHGFFQVSGHGVDEALARAALDGASGFFRLPLAEKQRARRVPGTVSGYTSAHADRFASKLPWKETLSFGFHDRAGAAPVVVDYFTSTLGPDYEPMGRVYQEYCGKMKELSLRIMELLELSQGVEKRGYYREFFADSSSIMRCNYYPPCPEPERTLGTGPHCDPTALTILLQDDVGGLEVLVDGDWRPVRPVPGAMVINIGDTFMALSNGRYKSCLHRAVVNRRQERRSLAFFLCPREDRVVRPPPGLRSPRRYPDFTWADLMRFTQRHYRADTRTLDAFTQWFSSSSSSAQEAA

SEQ ID NO: 98 - DNA, 3050 bp, Triticum aestivum
ctcatggtgctccagaccgctcagcaagaaccatccctgacgcgtccgcctcactgcagcgtcgccagcgcgcgctcgccggcggccatggacaccagccctgcaactcccctgctcctccagcctcctgctcccagcattgacccgttcgccgccaaggcggccgtcaacaagggcggcggcgcggcaaccgcggtgtacgacctccggagggagccgaagatccccgccccgttcgtgtggccgcacgccgaggtgcgccccaccacggcccaggagctggccgtgccggtggtggacgtgggcgtgctgcgcaatggcgacgccgcggggctccgccgcgccgtggcgcaggtggccgcggcgtgcgccacgcacgggttcttccaggtgtccgggcacggcgtggacgaggccctggcgcgcgcggcgctggacggcgcgagcggcttcttccggctgccgctggccgagaagcagcgcgcgcggcgcgtcccggggaccgtgtccgggtacacgagcgcgcacgccgaccggttcgcctccaagctcccctggaaggagaccctctccttcggcttccacgaccgcgccggcgccgcgcccgtcgtggtggactacttcaccagcaccctcgggccggactacgagccaatggggtaatatatccacccgcccacacccctatccggccagcacgaatccatccccgccactgcatttttttccttttgtttccgcgcgaccgtacgttcgatcggcgcccacgtacgtacgtgcgtacgcagtagcagtacttgaagccgccgtactacgtgctgagtagtgacaactgaacacgtgcaggagggtgtaccaggagtactgcgggaagatgaaggagctgtcgctgaggatcatggagctgctggagctgagccagggcgtggagaagcgcgggtactaccgggagttcttcgcggacagcagctccatcatgcggtgcaactactacccgccgtgcccggagccggagcgcacgctgggcacgggcccgcactgcgaccccacggcgctcaccatcctactgcaggacgacgtgggcgggctggaggtcctcgtcgacggcgactggcgccccgtccgccccgtccccggcgccatggtcatcaacatcggcgacaccttcatggtaattactcctctctcagcgttgctttcgctgattaattgcagaaacagtagtcaactacccatgctctgttccgctgtgctctgcttcccaacgagcgaaccggcccataaaaactgccttgctgtcttggaaccaagaggaaagggaccgtgggagcctaccgacacgacgtgattgcactctgcttcctaacaagcgagccgccggtagggctatcaccgtaagggctcctttgattcaaaggaatttcttaggatttctgaaggattgaaatccttaggattttttcctatgttggtacttcgattcataggattgaatcccataggatttttttcctatgaaatcttctgtactacatttcataggaaatctaacatccactccaacctttttttatatttcctttgtttttcatgtgccatcaaacactccttgttaatcctataggattcaagtgggcatgccactccaatcctatacttttcccattcctacgttttcaaaatcctacgaatcaaagaggccctaaagctgctgacatgacgtgattttttttttcttttctttctttctttctcagctccaatcaacgctggttattagatcattagagtggacaggttgaattaacatgcagtagttagtagttagcagccacaaacgggtcccgttctctgaagtctgaactgacataagtcctgatcatcgaccattctttgcttcctaggacgatgcctgttggaacttgcgtccaatgcccgttagggagtggtaattgtcatcacttttagactcgtcgattccactgatgaagacgtagcacatggatgagccaacgtatccgtttctagtggtctcgaaaagtagggtttcattcattctatctatctatccgtccgtccaaaagggctgcgatgcgagcacttgagtcggagccaatcagagcgcgagaaaagatagggggggtagcaagccatgtcggaggggcgtttgcttccggcaggtttggattcttgtggtaggcgggcggctctgtacagtagcggcggtgacggtgaggtggcggcgctttcggtggcgggccaacccaggtgcatgcacgcgcgctcgtcgttttcccgcctgaatctgccgctgcgcccatggcaagggggtgggtgctgccgccgggcgatggagtagatcacggtcgccgtcgggctcggccagttgatcacggttcgttcgtgcggtactaggttcccccacggcactgtgactgcatcgttccggccctcgccattggcgatcgggcaatctcctgttcatccgtcgctgttgattcctcggccacgatagaccatgcgcgtgccggtcgtcgccccgtcgcgctcgcttcacgtgctcgtcgcgtggctcccgtcccacacgaggccgccgctttctgacccagtggagcgcgtgatttacagtttatatatgtcgctgcatttttctttttgtgtgctgctcattttgcttggacggagaccgggaacgattagccacggatctaacgcgttgttgcttgttttcaatgcatgcatgcaggcgctgtcgaacgggcggtacaagagctgcctgcaccgcgcggtggtgaaccggcggcaggagcggcggtcgctggccttcttcctgtgcccgcgcgaggaccgcgtggtgcggccgccgccgggcctgaggagcccgcggcggtacccggacttcacctgggctgacctcatgcgcttcacgcagcgccactaccgcgccgacacgcgcaccctcgacgccttcacccagtggttctcctcctcctcctcctcggcccaggaggcggcctgattctgctctgccacgaaacgatcggtccaca

SEQ ID NO: 99 - DNA, 1486 bp, Hordeum vulgare
gaccagtagcatatagtttttcttgtgtttgccatggtggacgtgtcgaactttgtagaagccaatggcaatgcagcagtatcgattcctgccatggaagttgctgggagtcctcacgtcccgttcgttcctcgggacgcgaacgcgacagacagcaagaatgccaaggacgtcctcgacctctggcggcagcagaaacaaatcccggctcccttcatctggccccacgccgacgcgcggccgtcgtcgatcttggagctggacgtgcccgtggtcgacatcggcgcggccctgcacagcgccgccgggatggcccgcgccgcggcgcaggtggccgaggcatgcgcgagccacggcttcttccaggtgaccgggcacggcgtcgaccccgcgctggcccaagcagcgctcgacggcgcagcggacttcttccgcctgccgctcgccaccaagcagcgcgcccgccgatccccggggaccgtcaaagggtacgcctccgcccacgccgaccgcttcgccgccaagcttccctggaaggagactctctccttcatccacaaccacgtccacgaggacgtcggcgcccgcgcaagcagtcacgtcgtcgactacttcacctccgcccttggcgacgacttcatgcacctaggggaggtgtaccaggagtactgtgaggcgatggaggacgcgtcgctggcgataatggaggtgctgggggtgagcctggggctggggagagggtactacagggacttcttcgccgacggcagctccatcatgaggtgcaactactacccgcggtgcccggagccggaccggacgctggggacggggccgcactgcgacccgtcggcgctgaccatcctgctgcaggacggcgaggtggacgggctccaggtgctcgtcgacggcgcatggcgctccgtgcggcccaagcccggcgagctcgtcgtaaacatcggcgacaccttcatggcgctgtcgaacggccggtacaagagctgcctccaccgcgcggtggtgcaccgggagaaggagcgccggtcgctggcctacttcctcgccccgcgggaggaccgggtggttcgcccgccgccttcgccggcgccggcgccgcggctctacccggacttcacctgggcggagctcatgcgattcacgcagcgccactaccgcgccgacgcccgcacgctcgacgccttcgcgtgctggctcgacctgcccagctgccccaccacgccccaggcccaagggactgtctagtgtctgtgatgtatcatctgtctcagctgttgtatacgaccacttgtgtctgctagctctgcgcttgtgtttcttatgtgagctaactaactaaatagtgtgtatatttcttgccgcgccttatgcaagccctagtctagaacatgtaataattaacttaagcatatacgttgatctttggtgtatttttcatatttccttcataatgaataatctattatgc

SEQ ID NO: 100 - DNA, 1230 bp, Hordeum vulgare
atggtggacgtgtcgaactttgtagaagccaatggcaatgcagcagtatcgattcctgccatggaagttgctgggagtcctcacgtcccgttcgttcctcgggacgcgaacgcgacagacagcaagaatgccaaggacgtcctcgacctctggcggcagcagaaacaaatcccggctcccttcatctggccccacgccgacgcgcggccgtcgtcgatcttggagctggacgtgcccgtggtcgacatcggcgcggccctgcacagcgccgccgggatggcccgcgccgcggcgcaggtggccgaggcatgcgcgagccacggcttcttccaggtgaccgggcacggcgtcgaccccgcgctggcccaagcagcgctcgacggcgcagcggacttcttccgcctgccgctcgccaccaagcagcgcgcccgccgatccccggggaccgtcaaagggtacgcctccgcccacgccgaccgcttcgccgccaagcttccctggaaggagactctctccttcatccacaaccacgtccacgaggacgtcggcgcccgcgcaagcagtcacgtcgtcgactacttcacctccgcccttggcgacgacttcatgcacctaggggaggtgtaccaggagtactgtgaggcgatggaggacgcgtcgctggcgataatggaggtgctgggggtgagcctggggctggggagagggtactacagggacttcttcgccgacggcagctccatcatgaggtgcaactactacccgcggtgcccggagccggaccggacgctggggacggggccgcactgcgacccgtcggcgctgaccatcctgctgcaggacggcgaggtggacgggctccaggtgctcgtcgacggcgcatggcgctccgtgcggcccaagcccggcgagctcgtcgtaaacatcggcgacaccttcatggcgctgtcgaacggccggtacaagagctgcctccaccgcgcggtggtgcaccgggagaaggagcgccggtcgctggcctacttcctcgccccgcgggaggaccgggtggttcgcccgccgccttcgccggcgccggcgccgcggctctacccggacttcacctgggcggagctcatgcgattcacgcagcgccactaccgcgccgacgcccgcacgctcgacgccttcgcgtgctggctcgacctgcccagctgccccaccacgccccaggcccaagggactgtctag

SEQ ID NO: 101 - protein, 409 aa, Hordeum vulgare
MVDVSNFVEANGNAAVSIPAMEVAGSPHVPFVPRDANATDSKNAKDVLDLWRQQKQIPAPFIWPHADARPSSILELDVPVVDIGAALHSAAGMARAAAQVAEACASHGFFQVTGHGVDPALAQAALDGAADFFRLPLATKQRARRSPGTVKGYASAHADRFAAKLPWKETLSFIHNHVHEDVGARASSHVVDYFTSALGDDFMHLGEVYQEYCEAMEDASLAIMEVLGVSLGLGRGYYRDFFADGSSIMRCNYYPRCPEPDRTLGTGPHCDPSALTILLQDGEVDGLQVLVDGAWRSVRPKPGELVVNIGDTFMALSNGRYKSCLHRAVVHREKERRSLAYFLAPREDRVVRPPPSPAPAPRLYPDFTWAELMRFTQRHYRADARTLDAFACWLDLPSCPTTPQAQGTV

SEQ ID NO: 102 - DNA, 1423 bp, Sorghum bicolor
cctctcatcacaggccccagcctcactcttctcacagcaagacatcgcagcctcacaaccacacagcaacgtgatcgccatgggcgggctcaccatggagcaggccttcgtgcaggcccccgagcaccgccccaagcccaccgtcaccgaggccaccggcatcctggtcatcgacctctcgcctctcaccgccagcgacaccgacgcggccgcggtggacgcgctggccgccgaggtgggcgcggcgagccgggactggggcttcttcgtggtggttggccacggcgtgcccgcggagaccgtggcgcgcgcgacggcggcgcagcgcgcgttcttcgcgctgccggcggagcggaaggccgccgtgcggaggagcgaggcggagccgctcgggtactacgagtcggagcacaccaagaacgtcagggactggaaggaggtgttcgacctcgtcccgcgcgatccgccgccgccagcagccgtggccgacggcgagctcgtcttcaagaacaagtggccccaggatctgccgggcttcagagaggcgctggaggagtacgcggcagcgatggaggagctgtcgttcaagctgctggagctgatcgcccggagcttgaagctgaggcccgaccggctgcacggcttcttcaaggaccagacgacgttcatccggctgaaccactaccctccatgcccgagcccggacctggcgctgggagtggggcggcacaaggacgcgggggcgctgaccatcctgtaccaggacgaagtgggcgggctggacgtccggcggcgctcctccgacggcggcggcggcgagtgggtgcgggtgaggcccgtgccggagtcgttcgtcatcaacgtcggcgacctcgtccaggtgtggagcaacgacaggtacgagagcgcggagcaccgggtgtcggtgaactcggcgagggagaggttctccatgccctacttcttcaacccggcgagctacaccatggtggagccggtggaggagctggtgagcgacgacgacccgcccaggtacgacgcctacagctggggcgagttcttcagcaccaggaagaacagcaacttcaagaagctcagcgtggagaacattcagatcgcgcatttcaagaagaccctcgtcctcgcctagataagcagcaggatactacaggtctacaggactaggacaagccgatcgaggtgaccggccgtcgtcttcagattcagtatatgcgtgtcgccgttcgtgttagaacaaattaataatgtgcgcgctgtgtgctgtgtgtgtggagtaaaaaaaaactaaacatggatgtgcatgttcaaaaaaaaaaacatggatgcgagtatgtttgggaataataacaggcttgtgacggtctggtttatttgcaaattcaaaccgaattggttgatcttc

SEQ ID NO: 103 - DNA, 1071 bp, Sorghum bicolor
atgggcgggctcaccatggagcaggccttcgtgcaggcccccgagcaccgccccaagcccaccgtcaccgaggccaccggcatcctggtcatcgacctctcgcctctcaccgccagcgacaccgacgcggccgcggtggacgcgctggccgccgaggtgggcgcggcgagccgggactggggcttcttcgtggtggttggccacggcgtgcccgcggagaccgtggcgcgcgcgacggcggcgcagcgcgcgttcttcgcgctgccggcggagcggaaggccgccgtgcggaggagcgaggcggagccgctcgggtactacgagtcggagcacaccaagaacgtcagggactggaaggaggtgttcgacctcgtcccgcgcgatccgccgccgccagcagccgtggccgacggcgagctcgtcttcaagaacaagtggccccaggatctgccgggcttcagagaggcgctggaggagtacgcggcagcgatggaggagctgtcgttcaagctgctggagctgatcgcccggagcttgaagctgaggcccgaccggctgcacggcttcttcaaggaccagacgacgttcatccggctgaaccactaccctccatgcccgagcccggacctggcgctgggagtggggcggcacaaggacgcgggggcgctgaccatcctgtaccaggacgaagtgggcgggctggacgtccggcggcgctcctccgacggcggcggcggcgagtgggtgcgggtgaggcccgtgccggagtcgttcgtcatcaacgtcggcgacctcgtccaggtgtggagcaacgacaggtacgagagcgcggagcaccgggtgtcggtgaactcggcgagggagaggttctccatgccctacttcttcaacccggcgagctacaccatggtggagccggtggaggagctggtgagcgacgacgacccgcccaggtacgacgcctacagctggggcgagttcttcagcaccaggaagaacagcaacttcaagaagctcagcgtggagaacattcagatcgcgcatttcaagaagaccctcgtcctcgcctag

SEQ ID NO: 104 - protein, 356 aa, Sorghum bicolor
MGGLTMEQAFVQAPEHRPKPTVTEATGILVIDLSPLTASDTDAAAVDALAAEVGAASRDWGFFVVVGHGVPAETVARATAAQRAFFALPAERKAAVRRSEAEPLGYYESEHTKNVRDWKEVFDLVPRDPPPPAAVADGELVFKNKWPQDLPGFREALEEYAAAMEELSFKLLELIARSLKLRPDRLHGFFKDQTTFIRLNHYPPCPSPDLALGVGRHKDAGALTILYQDEVGGLDVRRRSSDGGGGEWVRVRPVPESFVINVGDLVQVWSNDRYESAEHRVSVNSARERFSMPYFFNPASYTMVEPVEELVSDDDPPRYDAYSWGEFFSTRKNSNFKKLSVENIQIAHFKKTLVLA

SEQ ID NO: 105 - DNA, 1499 bp, Sorghum bicolor
cctctcatcacaggccccagcctcactcttctcacagcaagacatcgcagcctcacaaccacacagcaacgtgatcgccatgggcgggctcaccatggagcaggccttcgtgcaggcccccgagcaccgccccaagcccaccgtcaccgaggccaccggcatcctggtcatcgacctctcgcctctcaccgccagcgacaccgacgcggccgcggtggacgcgctggccgccgaggtgggcgcggcgagccgggactggggcttcttcgtggtggttggccacggcgtgcccgcggagaccgtggcgcgcgcgacggcggcgcagcgcgcgttcttcgcgctgccggcggagcggaaggccgccgtgcggaggagcgaggcggagccgctcgggtactacgagtcggagcacaccaagaacgtcagggactggaaggaggtgttcgacctcgtcccgcgcgatccgccgccgccagcagccgtggccgacggcgagctcgtcttcaagaacaagtggccccaggatctgccgggcttcaggtgacgaaatcaacttatcttttcgatcatattttaccatttaatagtttaacaataattgaacttttttttgcagagaggcgctggaggagtacgcggcagcgatggaggagctgtcgttcaagctgctggagctgatcgcccggagcttgaagctgaggcccgaccggctgcacggcttcttcaaggaccagacgacgttcatccggctgaaccactaccctccatgcccgagcccggacctggcgctgggagtggggcggcacaaggacgcgggggcgctgaccatcctgtaccaggacgaagtgggcgggctggacgtccggcggcgctcctccgacggcggcggcggcgagtgggtgcgggtgaggcccgtgccggagtcgttcgtcatcaacgtcggcgacctcgtccaggtgtggagcaacgacaggtacgagagcgcggagcaccgggtgtcggtgaactcggcgagggagaggttctccatgccctacttcttcaacccggcgagctacaccatggtggagccggtggaggagctggtgagcgacgacgacccgcccaggtacgacgcctacagctggggcgagttcttcagcaccaggaagaacagcaacttcaagaagctcagcgtggagaacattcagatcgcgcatttcaagaagaccctcgtcctcgcctagataagcagcaggatactacaggtctacaggactaggacaagccgatcgaggtgaccggccgtcgtcttcagattcagtatatgcgtgtcgccgttcgtgttagaacaaattaataatgtgcgcgctgtgtgctgtgtgtgtggagtaaaaaaaaactaaacatggatgtgcatgttcaaaaaaaaaaacatggatgcgagtatgtttgggaataataacaggcttgtgacggtctggtttatttgcaaattcaaaccgaattggttgatcttc

SEQ ID NO: 106 - DNA, 1490 bp, Setaria italica
accccacacacacacccgcactgcatgcggcgtcctagctaatcagtcgctgctggcagcctcacaagtcacacaactccgacgcaggaaagctcgatccatcgccatgggcggcttctccatggatcagtccttcgtgcaggcccccgagcaccgccccaagcccaccgtcaccgaggccacgggcatcccgctcatcgacctctcgccactcaccggcggtggcggcggcgacgcggccgccgtggacgcgctggccgccgaggtgggcgcggcgagccgggactggggcttcttcgtggtggtggggcacggtgtgccggcggagaccgtggcgcgcgccacggaggcgcagcgcgcgttcttcgccctgccggcggagcggaaagccgccgtgcggaggagcgaggcggagccgctcgggtactacgagtcggagcacaccaagaacgtcagggactggaaggaggtgtacgacctcgtcccgggcgggcttcagccgccgatagccgtggccgacggcgaggtcgtgttcgaaaacaagtggcccgaagacctgccgggattcagagaggcgttggaggagtacatgcaagcgatggaagagctggcattcaagatactggagctgatcgcccggagcctgaacctgaggcctgacagactgcacggcttcttcaaggaccagaccaccttcatccggctcaaccactaccctccctgcccgagccccgacctcgccctcggcgtcggccggcacaaggacgccggagcactgaccatcctctaccaggacgacgtcggcgggctcgacgtccggcgccgttccgacggcgattgggtccgcgtcaagcctgtccccgactccttcatcatcaacgtcggcgacctcatccaggtttggagcaacgacaggtacgagagcgcggagcaccgggttacggtgaactcggccaaggagaggttctccaggccctacttcttcaacccggcgggctacaccatggtggagccggtggaggagctggtgagcgaggaggacccgccccggtacgacgcctacaactggggcaacttcttcagcaccaggaagaacagcaacttcaagaagctgagcgtggagaacatccagatcgcgcatttcaagaggagcgtcgccgcctaggatacgcacagaaagatcccatatgctgacttgctgatgaggcgacaggcggccgtgtcgtcttcagattcagagactgggagtaaacatttgtgcggtgttctgtaatcgtgatgtgacgagaactttagatatatgtttggaaataacagccttgtgttggtctggcttatccgcaaagtcaagattttcttctacattttgggattattgttggtaagcattaagcaacgtccagttcttacttcttagctcgatcagtggacgtaggaccggcctctgatgacaagggtgatttatgagaaatgtcatgtatatatgttcc

SEQ ID NO: 107 - DNA, 1059 bp, Setaria italica
atgggcggcttctccatggatcagtccttcgtgcaggcccccgagcaccgccccaagcccaccgtcaccgaggccacgggcatcccgctcatcgacctctcgccactcaccggcggtggcggcggcgacgcggccgccgtggacgcgctggccgccgaggtgggcgcggcgagccgggactggggcttcttcgtggtggtggggcacggtgtgccggcggagaccgtggcgcgcgccacggaggcgcagcgcgcgttcttcgccctgccggcggagcggaaagccgccgtgcggaggagcgaggcggagccgctcgggtactacgagtcggagcacaccaagaacgtcagggactggaaggaggtgtacgacctcgtcccgggcgggcttcagccgccgatagccgtggccgacggcgaggtcgtgttcgaaaacaagtggcccgaagacctgccgggattcagagaggcgttggaggagtacatgcaagcgatggaagagctggcattcaagatactggagctgatcgcccggagcctgaacctgaggcctgacagactgcacggcttcttcaaggaccagaccaccttcatccggctcaaccactaccctccctgcccgagccccgacctcgccctcggcgtcggccggcacaaggacgccggagcactgaccatcctctaccaggacgacgtcggcgggctcgacgtccggcgccgttccgacggcgattgggtccgcgtcaagcctgtccccgactccttcatcatcaacgtcggcgacctcatccaggtttggagcaacgacaggtacgagagcgcggagcaccgggttacggtgaactcggccaaggagaggttctccaggccctacttcttcaacccggcgggctacaccatggtggagccggtggaggagctggtgagcgaggaggacccgccccggtacgacgcctacaactggggcaacttcttcagcaccaggaagaacagcaacttcaagaagctgagcgtggagaacatccagatcgcgcatttcaagaggagcgtcgccgcctag

SEQ ID NO: 108 - protein, 352 aa, Setaria italica
MGGFSMDQSFVQAPEHRPKPTVTEATGIPLIDLSPLTGGGGGDAAAVDALAAEVGAASRDWGFFVVVGHGVPAETVARATEAQRAFFALPAERKAAVRRSEAEPLGYYESEHTKNVRDWKEVYDLVPGGLQPPIAVADGEVVFENKWPEDLPGFREALEEYMQAMEELAFKILELIARSLNLRPDRLHGFFKDQTTFIRLNHYPPCPSPDLALGVGRHKDAGALTILYQDDVGGLDVRRRSDGDWVRVKPVPDSFIINVGDLIQVWSNDRYESAEHRVTVNSAKERFSRPYFFNPAGYTMVEPVEELVSEEDPPRYDAYNWGNFFSTRKNSNFKKLSVENIQIAHFKRSVAA

SEQ ID NO: 109 - DNA, 1886 bp, Setaria italica
accccacacacacacccgcactgcatgcggcgtcctagctaatcagtcgctgctggcagcctcacaagtcacacaactccgacgcaggaaagctcgatccatcgccatgggcggcttctccatggatcagtccttcgtgcaggcccccgagcaccgccccaagcccaccgtcaccgaggccacgggcatcccgctcatcgacctctcgccactcaccggcggtggcggcggcgacgcggccgccgtggacgcgctggccgccgaggtgggcgcggcgagccgggactggggcttcttcgtggtggtggggcacggtgtgccggcggagaccgtggcgcgcgccacggaggcgcagcgcgcgttcttcgccctgccggcggagcggaaagccgccgtgcggaggagcgaggcggagccgctcgggtactacgagtcggagcacaccaagaacgtcagggactggaaggaggtgtacgacctcgtcccgggcgggcttcagccgccgatagccgtggccgacggcgaggtcgtgttcgaaaacaagtggcccgaagacctgccgggattcaggtgaatcaacttgcgcatattgttgtttctggcattgcatatgatcgtcgtgccagtatgttttgacaatatttttgttttcatatttttggtgaagatgggaaaatctttgttgaaataatcagggaattttcacatctttttttaatcaaagatagaataggttcggttactgaattttgatgatggacagaaaaagctgtgttttcactttccatctcagcgatgtttttttgtggatgaattctcctaaatttttgtcttttcatgttaaaacttgaacgggaattctcgcagagaggcgttggaggagtacatgcaagcgatggaagagctggcattcaagatactggagctgatcgcccggagcctgaacctgaggcctgacagactgcacggcttcttcaaggaccagaccaccttcatccggctcaaccactaccctccctgcccgagccccgacctcgccctcggcgtcggccggcacaaggacgccggagcactgaccatcctctaccaggacgacgtcggcgggctcgacgtccggcgccgttccgacggcgattgggtccgcgtcaagcctgtccccgactccttcatcatcaacgtcggcgacctcatccaggtacaacaaacaaaaacacacgtcattctcaaatcttttcgtgctgttaatgctcattcacgaattgatatcttacatgaacgactgagactttttcaggtttggagcaacgacaggtacgagagcgcggagcaccgggttacggtgaactcggccaaggagaggttctccaggccctacttcttcaacccggcgggctacaccatggtggagccggtggaggagctggtgagcgaggaggacccgccccggtacgacgcctacaactggggcaacttcttcagcaccaggaagaacagcaacttcaagaagctgagcgtggagaacatccagatcgcgcatttcaagaggagcgtcgccgcctaggatacgcacagaaagatcccatatgctgacttgctgatgaggcgacaggcggccgtgtcgtcttcagattcagagactgggagtaaacatttgtgcggtgttctgtaatcgtgatgtgacgagaactttagatatatgtttggaaataacagccttgtgttggtctggcttatccgcaaagtcaagattttcttctacattttgggattattgttggtaagcattaagcaacgtccagttcttacttcttagctcgatcagtggacgtaggaccggcctctgatgacaagggtgatttatgagaaatgtcatgtatatatgttcc

SEQ ID NO: 110 - DNA, 1379 bp, Oryza sativa
aagccacacgcacacacacacacacgctgacacacgagacgaacacttgtgctacagcttctcgccaccagctactgatcgaccatgggcggcctctccatggaccaggcgttcgtgcaggcccccgagcaccgccccaaggcgtccgtcgccgaggccgacggcatcccggtcatcgacctctcccctctcctcgccgccggcgatggcgacgccgacggggtggacgcgctcgcggcggaggtcgggagggcgagccgggactggggcttcttcgtggtggtgcgccacggtgtgcccgcggaggcggtggcgcgcgcggcggaggcgcagaggacgttcttcgcgctgccgccggagcggagggcggccgtggcgcggagcgaggcggcgccgatggggtactacgcgtccgagcacaccaagaacgtcagggactggaaggaggtgttcgacctcgtcccgcgccagacgccgccgccgccgacgaccgccgtggccgacggcgacctggtgttcgacaacaagtggcccgacgacctgccgggattcagggaggcaatggaggagtacggcgaagcggtggaggagctggcgttcaagctgctggagctgatcgccaggagcctcggcctgagacccgaccgcctccatggcttcttcaaggacgaccagaccaccttcatccggctcaaccactaccctccctgcccgagccccgacctcgccctcggcgtcggccgccacaaggacgccggcgcgctcaccgtgctctaccaggacgatgtcggcggcctcgacgtccgccgccgatccgacggcgagtgggtgcgcgtcaggcccgtccctcactccttcatcatcaacgtcggcgacatcatccaggtgtggagcaatgacaggtacgagagcgcggagcaccgggtggcggtgaacgtggagaaggagaggttctccatccctttcttcttcaacccggcgggccacaccatggtggagccactggaggaggtcgtgagcgacgagagcccggccaggtacaacccctacaactggggcgaattcttcagcaccaggaagaacagcaacttcaagaagctggacgtggagaacgtccagatcacgcatttcaggaagaattaacgcgccggctagatcatgttcagtaaattttcagatgatgatgcgtggacaaccatatagcctttgcgtcataagttaataatgtctgtgacagtatatcatgtaaacaatcgtatgatgtggcttctctatctgccggtgatggtaatgtgacattgtagaagagggtttgtgagatacttccttcacttaacttttacgaatgaatatagacaaccacaacatccttgtcgtga

SEQ ID NO: 111 - DNA, 1059 bp, Oryza sativa
atgggcggcctctccatggaccaggcgttcgtgcaggcccccgagcaccgccccaaggcgtccgtcgccgaggccgacggcatcccggtcatcgacctctcccctctcctcgccgccggcgatggcgacgccgacggggtggacgcgctcgcggcggaggtcgggagggcgagccgggactggggcttcttcgtggtggtgcgccacggtgtgcccgcggaggcggtggcgcgcgcggcggaggcgcagaggacgttcttcgcgctgccgccggagcggagggcggccgtggcgcggagcgaggcggcgccgatggggtactacgcgtccgagcacaccaagaacgtcagggactggaaggaggtgttcgacctcgtcccgcgccagacgccgccgccgccgacgaccgccgtggccgacggcgacctggtgttcgacaacaagtggcccgacgacctgccgggattcagggaggcaatggaggagtacggcgaagcggtggaggagctggcgttcaagctgctggagctgatcgccaggagcctcggcctgagacccgaccgcctccatggcttcttcaaggacgaccagaccaccttcatccggctcaaccactaccctccctgcccgagccccgacctcgccctcggcgtcggccgccacaaggacgccggcgcgctcaccgtgctctaccaggacgatgtcggcggcctcgacgtccgccgccgatccgacggcgagtgggtgcgcgtcaggcccgtccctcactccttcatcatcaacgtcggcgacatcatccaggtgtggagcaatgacaggtacgagagcgcggagcaccgggtggcggtgaacgtggagaaggagaggttctccatccctttcttcttcaacccggcgggccacaccatggtggagccactggaggaggtcgtgagcgacgagagcccggccaggtacaacccctacaactggggcgaattcttcagcaccaggaagaacagcaacttcaagaagctggacgtggagaacgtccagatcacgcatttcaggaagaattaa

SEQ ID NO: 112 - protein, 352 aa, Oryza sativa
MGGLSMDQAFVQAPEHRPKASVAEADGIPVIDLSPLLAAGDGDADGVDALAAEVGRASRDWGFFVVVRHGVPAEAVARAAEAQRTFFALPPERRAAVARSEAAPMGYYASEHTKNVRDWKEVFDLVPRQTPPPPTTAVADGDLVFDNKWPDDLPGFREAMEEYGEAVEELAFKLLELIARSLGLRPDRLHGFFKDDQTTFIRLNHYPPCPSPDLALGVGRHKDAGALTVLYQDDVGGLDVRRRSDGEWVRVRPVPHSFIINVGDIIQVWSNDRYESAEHRVAVNVEKERFSIPFFFNPAGHTMVEPLEEVVSDESPARYNPYNWGEFFSTRKNSNFKKLDVENVQITHFRKN

SEQ ID NO: 113 - DNA, 2027 bp, Oryza sativa
aagccacacgcacacacacacacacgctgacacacgagacgaacacttgtgctacagcttctcgccaccagctactgatcgaccatgggcggcctctccatggaccaggcgttcgtgcaggcccccgagcaccgccccaaggcgtccgtcgccgaggccgacggcatcccggtcatcgacctctcccctctcctcgccgccggcgatggcgacgccgacggggtggacgcgctcgcggcggaggtcgggagggcgagccgggactggggcttcttcgtggtggtgcgccacggtgtgcccgcggaggcggtggcgcgcgcggcggaggcgcagaggacgttcttcgcgctgccgccggagcggagggcggccgtggcgcggagcgaggcggcgccgatggggtactacgcgtccgagcacaccaagaacgtcagggactggaaggaggtgttcgacctcgtcccgcgccagacgccgccgccgccgacgaccgccgtggccgacggcgacctggtgttcgacaacaagtggcccgacgacctgccgggattcaggtcaggtcaccacatcgatcgatcgtcttcttcatcctcgcatcaattcagttcaacctcatcgaattcttgagcagggaggcaatggaggagtacggcgaagcggtggaggagctggcgttcaagctgctggagctgatcgccaggagcctcggcctgagacccgaccgcctccatggcttcttcaaggacgaccagaccaccttcatccggctcaaccactaccctccctgcccgagccccgacctcgccctcggcgtcggccgccacaaggacgccggcgcgctcaccgtgctctaccaggacgatgtcggcggcctcgacgtccgccgccgatccgacggcgagtgggtgcgcgtcaggcccgtccctcactccttcatcatcaacgtcggcgacatcatccaggtacttttttttttgagcagctacatatttatcaacaaattttcttctaacaatttatcggacataaatatattacaatgaaagaataattgtatcataacttgtgtgtccttatatgtaagttttagaaatcctatagtaacatggtattttcgcgaaagcggagattgtgagaccgtatcttttcacccatgcgcgtcatatgatttttttttcttgccaacttaaataaatttcaaagtaaatctaatagattaaaattatgtgaaacttacatataagttttctacggtaacacgctattttcacgaaacggaggtcgttccaagttgaatgaatcttgaagtaaatctaacgatttaaaattatgtgcatacacgttatattacagttatatacaagttataatataattacactacaattataacggtattcatagttgacaaacttttaaaagagaattagttaataaatatataacaacattgtagtttaattgttactatttgacatcatttttatttgcattttgaatttgactgaaaaaattgagagtgcgcttgtccaggtgtggagcaatgacaggtacgagagcgcggagcaccgggtggcggtgaacgtggagaaggagaggttctccatccctttcttcttcaacccggcgggccacaccatggtggagccactggaggaggtcgtgagcgacgagagcccggccaggtacaacccctacaactggggcgaattcttcagcaccaggaagaacagcaacttcaagaagctggacgtggagaacgtccagatcacgcatttcaggaagaattaacgcgccggctagatcatgttcagtaaattttcagatgatgatgcgtggacaaccatatagcctttgcgtcataagttaataatgtctgtgacagtatatcatgtaaacaatcgtatgatgtggcttctctatctgccggtgatggtaatgtgacattgtagaagagggtttgtgagatacttccttcacttaacttttacgaatgaatatagacaaccacaacatccttgtcgtga

SEQ ID NO: 114 - DNA, 1747 bp, Triticum aestivum
tcactcaaggccacaacacactcgccagtccatcgccaccatacgtgacaacttgagttacttgatctgttgctcatcgatctcgacatcgccatgggcggcctctccatggaccaggccttcgtgcaggcccccgagcatcgcaccaaggcgaacctcgccgacgcggccggcatcccggtcatcgacctctcccctctcgccgccggcgacaaggccggcctggacgccctcgcggccgaggtgggcagggcgagccgtgactgggggttcttcgtggtggtgcgccacggcgtgccggcggagacggtggcgcgggcgctggaggcgcagagggccttcttcgcgctgcccgcggaccggaaggcggccgtgcggagggacgaggcggcgccgctggggtactacgagtcggagcacaccaagaacgtcagggactggaaggaggtgttcgacctcgtcccccgcgagccgccgccgcctgccgcggttgccgacggcgagctcatgttcgagaacaagtggcccgaggacctgccggggttcagagaggctctcgaagagtacgagaaagcgatggaggagctggcgttcaagctgctggagctgatcgcccggagcctgggactgagaccggaccggctgcacggcttcttcaaggaccagaccaccttcatccggctgaaccactacccgccctgccccagccccgacctcgccctcggcgtcggtcgccacaaggacgccggcgcgctcaccatcctctaccaggacgacgtcggcgggctcgacgtccggcgccgctccgacggcgagtgggtgcgcgtcaggcctgtcccggactcctacgtcatcaacgtcggcgacatcatccaggtgtggagcaacgacaggtacgagagcgcggagcacagggtgtcggtgaactcgcacaaggagaggttctccatgccctacttcttcgaccccgggagcgacgccatgatcgagccgttggaggagatggtgagcgacgaaaggccggccaggtacgacgcctacaactggggcaacttcttcagcaccaggaagaacagcaacttcaggaagctcgccgtcgaaaacgtccagatcgcacacttcagaaaggaccgaccttaaatgaaggatccctcatgaattcatgatccttccgctctcctcagtgatcctagtgctacaactacaagcatctccccgtttgtagtaatcatatataaataagtattccctccgtaaactaatataagagcatttaaaacactactctagtgatctaaatgctcttatattagtttacagagagagtattgtgtattaataatgactttctctgtttcaaaataagtgatgacgtggttttagttcaattttttttagagaggaggcatctgacgggccttaaactgaggaccttagagtacaaacaaggttcgacgaaagtaagtttaagggatacaaggccgtagccaacaaaacgcgacgcagcgcgcaatctaaaatcagcgtgctgtcaaggtagctggagacgtccatgccgttaatctctctcaagaagctcgccgaagctcagtgcaccttgcgtgcactcttgtgaagagcaccttcacgtgtcctttgtcctgagattttgtcaacagtttccatgactgcaagaaaaacactagtttgtataatagctcagcgggatgtcgaatgaattgcccctcaatcaaagctttatttctag

SEQ ID NO: 115 - DNA, 1047 bp, Triticum aestivum
atgggcggcctctccatggaccaggccttcgtgcaggcccccgagcatcgcaccaaggcgaacctcgccgacgcggccggcatcccggtcatcgacctctcccctctcgccgccggcgacaaggccggcctggacgccctcgcggccgaggtgggcagggcgagccgtgactgggggttcttcgtggtggtgcgccacggcgtgccggcggagacggtggcgcgggcgctggaggcgcagagggccttcttcgcgctgcccgcggaccggaaggcggccgtgcggagggacgaggcggcgccgctggggtactacgagtcggagcacaccaagaacgtcagggactggaaggaggtgttcgacctcgtcccccgcgagccgccgccgcctgccgcggttgccgacggcgagctcatgttcgagaacaagtggcccgaggacctgccggggttcagagaggctctcgaagagtacgagaaagcgatggaggagctggcgttcaagctgctggagctgatcgcccggagcctgggactgagaccggaccggctgcacggcttcttcaaggaccagaccaccttcatccggctgaaccactacccgccctgccccagccccgacctcgccctcggcgtcggtcgccacaaggacgccggcgcgctcaccatcctctaccaggacgacgtcggcgggctcgacgtccggcgccgctccgacggcgagtgggtgcgcgtcaggcctgtcccggactcctacgtcatcaacgtcggcgacatcatccaggtgtggagcaacgacaggtacgagagcgcggagcacagggtgtcggtgaactcgcacaaggagaggttctccatgccctacttcttcgaccccgggagcgacgccatgatcgagccgttggaggagatggtgagcgacgaaaggccggccaggtacgacgcctacaactggggcaacttcttcagcaccaggaagaacagcaacttcaggaagctcgccgtcgaaaacgtccagatcgcacacttcagaaaggaccgaccttaa

SEQ ID NO: 116 - protein, 348 aa, Triticum aestivum
MGGLSMDQAFVQAPEHRTKANLADAAGIPVIDLSPLAAGDKAGLDALAAEVGRASRDWGFFVVVRHGVPAETVARALEAQRAFFALPADRKAAVRRDEAAPLGYYESEHTKNVRDWKEVFDLVPREPPPPAAVADGELMFENKWPEDLPGFREALEEYEKAMEELAFKLLELIARSLGLRPDRLHGFFKDQTTFIRLNHYPPCPSPDLALGVGRHKDAGALTILYQDDVGGLDVRRRSDGEWVRVRPVPDSYVINVGDIIQVWSNDRYESAEHRVSVNSHKERFSMPYFFDPGSDAMIEPLEEMVSDERPARYDAYNWGNFFSTRKNSNFRKLAVENVQIAHFRKDRP

SEQ ID NO: 117 - DNA, 1863 bp, Triticum aestivum
tcactcaaggccacaacacactcgccagtccatcgccaccatacgtgacaacttgagttacttgatctgttgctcatcgatctcgacatcgccatgggcggcctctccatggaccaggccttcgtgcaggcccccgagcatcgcaccaaggcgaacctcgccgacgcggccggcatcccggtcatcgacctctcccctctcgccgccggcgacaaggccggcctggacgccctcgcggccgaggtgggcagggcgagccgtgactgggggttcttcgtggtggtgcgccacggcgtgccggcggagacggtggcgcgggcgctggaggcgcagagggccttcttcgcgctgcccgcggaccggaaggcggccgtgcggagggacgaggcggcgccgctggggtactacgagtcggagcacaccaagaacgtcagggactggaaggaggtgttcgacctcgtcccccgcgagccgccgccgcctgccgcggttgccgacggcgagctcatgttcgagaacaagtggcccgaggacctgccggggttcaggtacggtcatcaactcaatcaattctgcgaccccgagagaaatggttcactattattcgtggttcatacgtatgattcagacgttaatctcgatgcaaattgatttgtgcatgcagagaggctctcgaagagtacgagaaagcgatggaggagctggcgttcaagctgctggagctgatcgcccggagcctgggactgagaccggaccggctgcacggcttcttcaaggaccagaccaccttcatccggctgaaccactacccgccctgccccagccccgacctcgccctcggcgtcggtcgccacaaggacgccggcgcgctcaccatcctctaccaggacgacgtcggcgggctcgacgtccggcgccgctccgacggcgagtgggtgcgcgtcaggcctgtcccggactcctacgtcatcaacgtcggcgacatcatccaggtgtggagcaacgacaggtacgagagcgcggagcacagggtgtcggtgaactcgcacaaggagaggttctccatgccctacttcttcgaccccgggagcgacgccatgatcgagccgttggaggagatggtgagcgacgaaaggccggccaggtacgacgcctacaactggggcaacttcttcagcaccaggaagaacagcaacttcaggaagctcgccgtcgaaaacgtccagatcgcacacttcagaaaggaccgaccttaaatgaaggatccctcatgaattcatgatccttccgctctcctcagtgatcctagtgctacaactacaagcatctccccgtttgtagtaatcatatataaataagtattccctccgtaaactaatataagagcatttaaaacactactctagtgatctaaatgctcttatattagtttacagagagagtattgtgtattaataatgactttctctgtttcaaaataagtgatgacgtggttttagttcaattttttttagagaggaggcatctgacgggccttaaactgaggaccttagagtacaaacaaggttcgacgaaagtaagtttaagggatacaaggccgtagccaacaaaacgcgacgcagcgcgcaatctaaaatcagcgtgctgtcaaggtagctggagacgtccatgccgttaatctctctcaagaagctcgccgaagctcagtgcaccttgcgtgcactcttgtgaagagcaccttcacgtgtcctttgtcctgagattttgtcaacagtttccatgactgcaagaaaaacactagtttgtataatagctcagcgggatgtcgaatgaattgcccctcaatcaaagctttatttctag

SEQ ID NO: 118 - protein, 349 aa, Hordeum vulgare
MGGLSMGQAFVQAPEHRTKPTLADADGIPVIDLSPLAAGDEAGVDALAAEVGRASRDWGFFVVVRHGVPAETVARALEAQRAFFALPAERKAAVRRDEAAPLGYYESEHTKNVRDWKEVFDFVPREPPPPAAVADGELVFENKWPEDLPGFRVAFEEYAKAMEELAFKLLELIARSLGLTPDRLNGFFKDHQTTFIRLNHYPPCPSPDLALGVGRHKDAGALTVLYQDDVGGLDVRHRSDGEWVRVRPVPDSYVINVGDIIQVWSNDRYESAEHRVSVNSDKERFSMPYFFNPGSDAMVEPLEEMVSDERPARYDAYNWGHFFSTRKNSNFKKLDVENVQIAHFRKLHL

SEQ ID NO: 119 - DNA, 1963 bp, Sorghum bicolor
tataaataccacgccatgtacttctctgcttctacacttctccagcttctctcatgccataccactagtgcaaggtcctagatttacacttggtgctacagcttcttcctccctccctcccctctctaggcagctagcacgcagcgcagcacacgaaacatctattgaccggccgcctccgccggggatccataattactatactaccaatcggccagcgtcatgccgacgccgtcgcacctcgcgaacccgcgctacttcgacttccgtgcggcgcggcgggtgccggagacgcacgcctggccggggctgcacgaccaccccgtcgtggacggcggcgcgccggggccagacgccgtccccgtggtggacctcgcgggggcggcggacgagccgagagccgcggtggtggcccaagtggcgcgcgccgccgagcaatggggcgcgttcctgctcacggggcacggcgtccccgcggagctgctggcgcgcgtcgaggaccggatcgccaccatgttcgcgctgccagcggacgacaagatgcgcgccgtgcgcgggcctggcgacgcctgcggctacggctccccgcccatctcctccttcttctccaagtgcatgtggtcggagggatacaccttctcgccggccaacctccgcgccgacctccgcaagctctggcctaaggccggcgacgactacaccagcttctgtgatgtgatggaggagttccacaagcacatgcgtgccctcgcggacaagctgctggagctgttcctcatggcgctggggctcaccgacgagcaggtcggcggcgtggaggcggagcggaggatcgccgagacgatgaccgccaccatgcacctcaactggtaccctcggtgcccggacccgcgccgcgcgctggggctgatcgcgcacaccgactcgggcttcttcaccttcgtgctgcagagcctcgtcccggggctgcagctcttccgccacgccccggaccggtgggtggcggtgccggcggtaccgggcgccttcgtcgtcaacgtgggcgacctcttccacatcctcaccaacggccggttccacagcgtgtaccaccgcgccgtcgtgaaccgggacctcgacaggatatctctcggctacttcctcggcccgccgccgcacgccaaggtggcgccgctaagggaggccgtgccgcccggccgcacccccgcgtaccgcgccgtcacgtggcccgagtacatgggcgtccgcaagaaggccttcaccaccggcgcatccgcgctcaagatggtcgccctcgccgccgccgccgccgccgccgacctcgacgatgacgccggtgctggcgccgccgccgaacctgtcgtccatcagcagctactcgtctcgtcgtagccgatcgatcgccggatcggtcgagactgatgatgatgatgcatatatactcgtcgatggagtagacagactaatcaagcaaccctgaaactatgaatgcatgcgtgcgcttcgtgcttgcttgcgcatgcagctagcaggcttcattccgttccgcagctgctctgctccaacctgctctgctggattgatgtatatggtagaagaattaagagatcgatggatgacggaggaagaagaagacgaagacgacgatgaggaaaaggacacgctgtacgtagctggttcttctagtctagtttacagcaggccgggcggccggctgctgcttccaatcgagtttgtcgttactgacgattgttagtggatcgattaactaatctggaattctggattattaatataatgcatgtggtttggcatctggcgtaaagcaggtaatggtacctagccagtagccagtagccaggctggtcaatgataggtctataccctgatcctgtactgttgtttctttcggtctttctgagagagaaaaaaaacgaatatatggcgtactcaattcatcaaa

SEQ ID NO: 120 - DNA, 1170 bp, Sorghum bicolor
atgccgacgccgtcgcacctcgcgaacccgcgctacttcgacttccgtgcggcgcggcgggtgccggagacgcacgcctggccggggctgcacgaccaccccgtcgtggacggcggcgcgccggggccagacgccgtccccgtggtggacctcgcgggggcggcggacgagccgagagccgcggtggtggcccaagtggcgcgcgccgccgagcaatggggcgcgttcctgctcacggggcacggcgtccccgcggagctgctggcgcgcgtcgaggaccggatcgccaccatgttcgcgctgccagcggacgacaagatgcgcgccgtgcgcgggcctggcgacgcctgcggctacggctccccgcccatctcctccttcttctccaagtgcatgtggtcggagggatacaccttctcgccggccaacctccgcgccgacctccgcaagctctggcctaaggccggcgacgactacaccagcttctgtgatgtgatggaggagttccacaagcacatgcgtgccctcgcggacaagctgctggagctgttcctcatggcgctggggctcaccgacgagcaggtcggcggcgtggaggcggagcggaggatcgccgagacgatgaccgccaccatgcacctcaactggtaccctcggtgcccggacccgcgccgcgcgctggggctgatcgcgcacaccgactcgggcttcttcaccttcgtgctgcagagcctcgtcccggggctgcagctcttccgccacgccccggaccggtgggtggcggtgccggcggtaccgggcgccttcgtcgtcaacgtgggcgacctcttccacatcctcaccaacggccggttccacagcgtgtaccaccgcgccgtcgtgaaccgggacctcgacaggatatctctcggctacttcctcggcccgccgccgcacgccaaggtggcgccgctaagggaggccgtgccgcccggccgcacccccgcgtaccgcgccgtcacgtggcccgagtacatgggcgtccgcaagaaggccttcaccaccggcgcatccgcgctcaagatggtcgccctcgccgccgccgccgccgccgccgacctcgacgatgacgccggtgctggcgccgccgccgaacctgtcgtccatcagcagctactcgtctcgtcgtag

SEQ ID NO: 121 - protein, 389 aa, Sorghum bicolor
MPTPSHLANPRYFDFRAARRVPETHAWPGLHDHPVVDGGAPGPDAVPVVDLAGAADEPRAAVVAQVARAAEQWGAFLLTGHGVPAELLARVEDRIATMFALPADDKMRAVRGPGDACGYGSPPISSFFSKCMWSEGYTFSPANLRADLRKLWPKAGDDYTSFCDVMEEFHKHMRALADKLLELFLMALGLTDEQVGGVEAERRIAETMTATMHLNWYPRCPDPRRALGLIAHTDSGFFTFVLQSLVPGLQLFRHAPDRWVAVPAVPGAFVVNVGDLFHILTNGRFHSVYHRAVVNRDLDRISLGYFLGPPPHAKVAPLREAVPPGRTPAYRAVTWPEYMGVRKKAFTTGASALKMVALAAAAAAADLDDDAGAGAAAEPVVHQQLLVSS

SEQ ID NO: 122 - DNA, 2321 bp, Sorghum bicolor
tataaataccacgccatgtacttctctgcttctacacttctccagcttctctcatgccataccactagtgcaaggtcctagatttacacttggtgctacagcttcttcctccctccctcccctctctaggcagctagcacgcagcgcagcacacgaaacatctattgaccggccgcctccgccggggatccataattactatactaccaatcggccagcgtcatgccgacgccgtcgcacctcgcgaacccgcgctacttcgacttccgtgcggcgcggcgggtgccggagacgcacgcctggccggggctgcacgaccaccccgtcgtggacggcggcgcgccggggccagacgccgtccccgtggtggacctcgcgggggcggcggacgagccgagagccgcggtggtggcccaagtggcgcgcgccgccgagcaatggggcgcgttcctgctcacggggcacggcgtccccgcggagctgctggcgcgcgtcgaggaccggatcgccaccatgttcgcgctgccagcggacgacaagatgcgcgccgtgcgcgggcctggcgacgcctgcggctacggctccccgcccatctcctccttcttctccaagtgcatgtggtcggagggatacaccttctcgccggccaacctccgcgccgacctccgcaagctctggcctaaggccggcgacgactacaccagcttctggtacgtgcacccgccggccgcgcgccgccacacaccgtacccacacacgtgcgcgctcgcgcctagctactagtagctgctttgctttgcttacctttgattctcgcctttgccatgcatatgcatgatgcacgtacaggtactgcaggtacaacatgtcacacgcacgcacgcacgcacaacccatagtccgatacgatacatcatcgatcgacgtgtcgtcaccgtctaaggccatgcatgcatgcaagcacacgcctagacctttttagcatgctggctgacgaggagtatactagctaataagctacttgtcactgcgcgtcttgcttaattacactagtgcatatttctacagtgatgtgatggaggagttccacaagcacatgcgtgccctcgcggacaagctgctggagctgttcctcatggcgctggggctcaccgacgagcaggtcggcggcgtggaggcggagcggaggatcgccgagacgatgaccgccaccatgcacctcaactggtaccctcggtgcccggacccgcgccgcgcgctggggctgatcgcgcacaccgactcgggcttcttcaccttcgtgctgcagagcctcgtcccggggctgcagctcttccgccacgccccggaccggtgggtggcggtgccggcggtaccgggcgccttcgtcgtcaacgtgggcgacctcttccacatcctcaccaacggccggttccacagcgtgtaccaccgcgccgtcgtgaaccgggacctcgacaggatatctctcggctacttcctcggcccgccgccgcacgccaaggtggcgccgctaagggaggccgtgccgcccggccgcacccccgcgtaccgcgccgtcacgtggcccgagtacatgggcgtccgcaagaaggccttcaccaccggcgcatccgcgctcaagatggtcgccctcgccgccgccgccgccgccgccgacctcgacgatgacgccggtgctggcgccgccgccgaacctgtcgtccatcagcagctactcgtctcgtcgtagccgatcgatcgccggatcggtcgagactgatgatgatgatgcatatatactcgtcgatggagtagacagactaatcaagcaaccctgaaactatgaatgcatgcgtgcgcttcgtgcttgcttgcgcatgcagctagcaggcttcattccgttccgcagctgctctgctccaacctgctctgctggattgatgtatatggtagaagaattaagagatcgatggatgacggaggaagaagaagacgaagacgacgatgaggaaaaggacacgctgtacgtagctggttcttctagtctagtttacagcaggccgggcggccggctgctgcttccaatcgagtttgtcgttactgacgattgttagtggatcgattaactaatctggaattctggattattaatataatgcatgtggtttggcatctggcgtaaagcaggtaatggtacctagccagtagccagtagccaggctggtcaatgataggtctataccctgatcctgtactgttgtttctttcggtctttctgagagagaaaaaaaacgaatatatggcgtactcaattcatcaaa

SEQ ID NO: 123 - DNA, 1796 bp, Setaria italica
actagtgcaaggtcctagatttacacttggtgcttgcttgtttcttcctagttgctactggtagcacgcagtggctggctggccgtaatctattgtctgggctcgatcggtgattaggaagtagccaaagcaagctaaggccgccgccgccgccgccatgccgacgccgtcgcacctcaagaacccgctctacttcgacttccgcgccgcgcggcgggtgccggagtcccacgcctggccggggctcgacgaccaccccgtggtggacggcggcggcgcgccggggtccccggacgccgtgccggtggtggacctgcgcgagccgggcgccgcggcggtggcccgcgtggcgcgcgccgccgagcagtggggcgcgttcctgctcaccggccacggcgtccccgcggagctcctggcgcgcgtcgaggaccgcgtcgcgtgcatgttcgcgctgccggccgccgacaagatgcgcgccgtgcgcgggccgggggacgcctgcggctacggctcgccgcccatctcctccttcttctccaagtgcatgtggtccgagggctacaccttctcgccggcctccctccgccgcgacctccgcaagctctggcccaaggccggcgacgactacgacagcttctgtgacgtgatggaggagttccacaaggagatgcgcgccctcgccgacaggctcctggagctgttcctcagggcgctcgggctcaccggcgagcaggtcggcgccgtcgaggcggagcggaggatcggcgagacgatgaccgccaccatgcacctcaactggtatccgaggtgcccggacccgcggcgcgcgctggggctgatcgcgcacacggactcgggcttcttcaccttcgtgctgcagagcctcgtgccggggctgcagctgttccggcacggccccaaccggtgggtggcggtgccggccgtgccgggcgccttcgtcgtcaacgtcggcgacctcttccacatcctcacgaacggccgcttccacagcgtgtaccaccgcgccgtcgtcaaccgggacctcgaccggatatcgctcggctacttcctcggcccgccgccccacgccaaggtggcgccgctccgggaggtcgtgccgccgggccgggcccccgcctaccgcgccgtcacgtggcccgagtacatgggcgtccgcaagaaggccttcaccaccggcgcctccgcgctcaagatggtcgccgccgccgccgccgccaccgaatccgacgacaccgacgcagccgccgccgccgttcaccagccgccggtcgtcgtctcatcgtagccgatcgatcgccggaaacacagacgatgcataccgtaccccgagcaatctaatcaaaacaaggcatccattctcgcgcgcatgcagcggccagccgggcttccgcagctgctcggcctcctctgctggctgtggaaatggaaaattttaatctgagatgaagacgaagacgaagacgaaacggagaggaaaaggacatgctgtagctgtttcttctagttgcgcaggccgctcccagtcgagtttgtcgttactgacgattattactctgatgaaaactaatctgaattaatgcatgtagtttggcaatttggtactaaaggtaggcacctagccaggctggtcaatgataggtctataacctgatcctgttctctgttgttttcctttgtctgagaaaaaatggaaataattgatccggccggacgggtgtactgataggtgatgctgaattgctgatgcaagaggttgcgagctgcagtgagcagca

SEQ ID NO: 124 - DNA, 1149 bp, Setaria italica
atgccgacgccgtcgcacctcaagaacccgctctacttcgacttccgcgccgcgcggcgggtgccggagtcccacgcctggccggggctcgacgaccaccccgtggtggacggcggcggcgcgccggggtccccggacgccgtgccggtggtggacctgcgcgagccgggcgccgcggcggtggcccgcgtggcgcgcgccgccgagcagtggggcgcgttcctgctcaccggccacggcgtccccgcggagctcctggcgcgcgtcgaggaccgcgtcgcgtgcatgttcgcgctgccggccgccgacaagatgcgcgccgtgcgcgggccgggggacgcctgcggctacggctcgccgcccatctcctccttcttctccaagtgcatgtggtccgagggctacaccttctcgccggcctccctccgccgcgacctccgcaagctctggcccaaggccggcgacgactacgacagcttctgtgacgtgatggaggagttccacaaggagatgcgcgccctcgccgacaggctcctggagctgttcctcagggcgctcgggctcaccggcgagcaggtcggcgccgtcgaggcggagcggaggatcggcgagacgatgaccgccaccatgcacctcaactggtatccgaggtgcccggacccgcggcgcgcgctggggctgatcgcgcacacggactcgggcttcttcaccttcgtgctgcagagcctcgtgccggggctgcagctgttccggcacggccccaaccggtgggtggcggtgccggccgtgccgggcgccttcgtcgtcaacgtcggcgacctcttccacatcctcacgaacggccgcttccacagcgtgtaccaccgcgccgtcgtcaaccgggacctcgaccggatatcgctcggctacttcctcggcccgccgccccacgccaaggtggcgccgctccgggaggtcgtgccgccgggccgggcccccgcctaccgcgccgtcacgtggcccgagtacatgggcgtccgcaagaaggccttcaccaccggcgcctccgcgctcaagatggtcgccgccgccgccgccgccaccgaatccgacgacaccgacgcagccgccgccgccgttcaccagccgccggtcgtcgtctcatcgtag

SEQ ID NO: 125 - protein, 382 aa, Setaria italica
MPTPSHLKNPLYFDFRAARRVPESHAWPGLDDHPVVDGGGAPGSPDAVPVVDLREPGAAAVARVARAAEQWGAFLLTGHGVPAELLARVEDRVACMFALPAADKMRAVRGPGDACGYGSPPISSFFSKCMWSEGYTFSPASLRRDLRKLWPKAGDDYDSFCDVMEEFHKEMRALADRLLELFLRALGLTGEQVGAVEAERRIGETMTATMHLNWYPRCPDPRRALGLIAHTDSGFFTFVLQSLVPGLQLFRHGPNRWVAVPAVPGAFVVNVGDLFHILTNGRFHSVYHRAVVNRDLDRISLGYFLGPPPHAKVAPLREVVPPGRAPAYRAVTWPEYMGVRKKAFTTGASALKMVAAAAAATESDDTDAAAAAVHQPPVVVSS

SEQ ID NO: 126 - DNA, 2146 bp, Setaria italica
actagtgcaaggtcctagatttacacttggtgcttgcttgtttcttcctagttgctactggtagcacgcagtggctggctggccgtaatctattgtctgggctcgatcggtgattaggaagtagccaaagcaagctaaggccgccgccgccgccgccatgccgacgccgtcgcacctcaagaacccgctctacttcgacttccgcgccgcgcggcgggtgccggagtcccacgcctggccggggctcgacgaccaccccgtggtggacggcggcggcgcgccggggtccccggacgccgtgccggtggtggacctgcgcgagccgggcgccgcggcggtggcccgcgtggcgcgcgccgccgagcagtggggcgcgttcctgctcaccggccacggcgtccccgcggagctcctggcgcgcgtcgaggaccgcgtcgcgtgcatgttcgcgctgccggccgccgacaagatgcgcgccgtgcgcgggccgggggacgcctgcggctacggctcgccgcccatctcctccttcttctccaagtgcatgtggtccgagggctacaccttctcgccggcctccctccgccgcgacctccgcaagctctggcccaaggccggcgacgactacgacagcttctggtacgtcgtcgtctatagctagtagctagccgccggcacacgtgcgcctgacctgctccgccatgcatggtgcacgtatgcagatcgatcacacgcaccgatcgatcgacgtgtcccggtcaaggccatgcatgcatgcaagcaaccaacagcacgcctcctgatactgcttgttgcttacaccgttggtatgtgcctgttgcctacagtgacgtgatggaggagttccacaaggagatgcgcgccctcgccgacaggctcctggagctgttcctcagggcgctcgggctcaccggcgagcaggtcggcgccgtcgaggcggagcggaggatcggcgagacgatgaccgccaccatgcacctcaactggtatgtgccatgccatgaccacctgcgtctatgaactaacggaagcttccatcgcgtgtccatgacgatttagaagctgtagtccagagcttgagacaaacgaaacgaagcttacatggtggcgtgacgtgtcgcgtgcaggtatccgaggtgcccggacccgcggcgcgcgctggggctgatcgcgcacacggactcgggcttcttcaccttcgtgctgcagagcctcgtgccggggctgcagctgttccggcacggccccaaccggtgggtggcggtgccggccgtgccgggcgccttcgtcgtcaacgtcggcgacctcttccacatcctcacgaacggccgcttccacagcgtgtaccaccgcgccgtcgtcaaccgggacctcgaccggatatcgctcggctacttcctcggcccgccgccccacgccaaggtggcgccgctccgggaggtcgtgccgccgggccgggcccccgcctaccgcgccgtcacgtggcccgagtacatgggcgtccgcaagaaggccttcaccaccggcgcctccgcgctcaagatggtcgccgccgccgccgccgccaccgaatccgacgacaccgacgcagccgccgccgccgttcaccagccgccggtcgtcgtctcatcgtagccgatcgatcgccggaaacacagacgatgcataccgtaccccgagcaatctaatcaaaacaaggcatccattctcgcgcgcatgcagcggccagccgggcttccgcagctgctcggcctcctctgctggctgtggaaatggaaaattttaatctgagatgaagacgaagacgaagacgaaacggagaggaaaaggacatgctgtagctgtttcttctagttgcgcaggccgctcccagtcgagtttgtcgttactgacgattattactctgatgaaaactaatctgaattaatgcatgtagtttggcaatttggtactaaaggtaggcacctagccaggctggtcaatgataggtctataacctgatcctgttctctgttgttttcctttgtctgagaaaaaatggaaataattgatccggccggacgggtgtactgataggtgatgctgaattgctgatgcaagaggttgcgagctgcagtgagcagca

SEQ ID NO: 127 - DNA, 1933 bp, Oryza sativa
actactcattccactattgtaaagtcatagaaaaaatttatatagagagaaaaaattagtgttgttattgttactggctttctgccagacgagacgagcgagcgcgcgagtgtgttgctctctggtcatcgtcgtcgtcgtcgcgatgccgacgccgtcgcacttgaagaacccgctctgcttcgacttccgggcggcgaggcgggtgccggagacgcacgcgtggccggggctggacgaccacccggtggtggacggcggcggcggcggcggcgaggacgcggtgccggtggtggacgtcggggcgggcgacgcggcggcgcgggtggcgcgggcggcggagcagtggggcgcgttccttctggtcgggcacggcgtgccggcggcgctgctgtcgcgcgtcgaggagcgcgtcgcccgcgtgttctccctgccggcgtcggagaagatgcgcgccgtccgcggccccggcgagccctgcggctacggctcgccgcccatctcctccttcttctccaagctcatgtggtccgagggctacaccttctccccttcctccctccgctccgagctccgccgcctctggcccaagtccggcgacgactacctcctcttctgtgacgtgatggaggagtttcacaaggagatgcggcggctagccgacgagttgctgaggttgttcttgagggcgctggggctcaccggcgaggaggtcgccggagtcgaggcggagaggaggatcggcgagaggatgacggcgacggtgcacctcaactggtacccgaggtgcccggagccgcggcgagcgctggggctcatcgcgcacacggactcgggcttcttcaccttcgtgctccagagcctcgtcccggggctgcagctgttccgtcgagggcccgaccggtgggtggcggtgccggcggtggcgggggccttcgtcgtcaacgtcggcgacctcttccacatcctcaccaacggccgcttccacagcgtctaccaccgcgccgtcgtgaaccgcgaccgcgaccgggtctcgctcggctacttcctcggcccgccgccggacgccgaggtggcgccgctgccggaggccgtgccggccggccggagccccgcctaccgcgctgtcacgtggccggagtacatggccgtccgcaagaaggccttcgccaccggcggctccgccctcaagatggtctccaccgacgccgccgccgccgccgacgaacacgacgacgtcgccgccgccgccgacgtccacgcataagctatagctactagctacctcgatctcacgcaaaaaaaaaaagaaacaattaatagagcaaaaaaaaaaagaagagaaaatggtggtacttgtgtttaaggtttcctccatgcaaaatggtttgcatgcatgcatgcaaagctagcatctgcagctgcaagaattacaagagcagagaagcagacagctagatggagataattaattaattaattaatctaattaagcatgcaataattaagattattattctgatttcagaactgaaaaaaaaagtgtggttaattaattattggttaggcttaattttatctagatgtagaaaaagaatcaagatcttcaagcaagagagaagaggatcgaagaagaaggaaaagaaaacgaaaaggacatgctgtgttgtctcttctagttgtaccctggctgctgattaagtgctttgttttgttgctgcaagcttgtcgttactgattattagttagttatgcatctaattgattaaactaatctgtttggcattttggctcgagctaagctatagccaggctggtcaatgataggaacttgtacaatttaagcaattgaacctgatcctgtactggcatgtatgtatatatgcaagtgatgagaaccactagctagtatagctagacatgtatttgtata

SEQ ID NO: 128 - DNA, 1122 bp, Oryza sativa
atgccgacgccgtcgcacttgaagaacccgctctgcttcgacttccgggcggcgaggcgggtgccggagacgcacgcgtggccggggctggacgaccacccggtggtggacggcggcggcggcggcggcgaggacgcggtgccggtggtggacgtcggggcgggcgacgcggcggcgcgggtggcgcgggcggcggagcagtggggcgcgttccttctggtcgggcacggcgtgccggcggcgctgctgtcgcgcgtcgaggagcgcgtcgcccgcgtgttctccctgccggcgtcggagaagatgcgcgccgtccgcggccccggcgagccctgcggctacggctcgccgcccatctcctccttcttctccaagctcatgtggtccgagggctacaccttctccccttcctccctccgctccgagctccgccgcctctggcccaagtccggcgacgactacctcctcttctgtgacgtgatggaggagtttcacaaggagatgcggcggctagccgacgagttgctgaggttgttcttgagggcgctggggctcaccggcgaggaggtcgccggagtcgaggcggagaggaggatcggcgagaggatgacggcgacggtgcacctcaactggtacccgaggtgcccggagccgcggcgagcgctggggctcatcgcgcacacggactcgggcttcttcaccttcgtgctccagagcctcgtcccggggctgcagctgttccgtcgagggcccgaccggtgggtggcggtgccggcggtggcgggggccttcgtcgtcaacgtcggcgacctcttccacatcctcaccaacggccgcttccacagcgtctaccaccgcgccgtcgtgaaccgcgaccgcgaccgggtctcgctcggctacttcctcggcccgccgccggacgccgaggtggcgccgctgccggaggccgtgccggccggccggagccccgcctaccgcgctgtcacgtggccggagtacatggccgtccgcaagaaggccttcgccaccggcggctccgccctcaagatggtctccaccgacgccgccgccgccgccgacgaacacgacgacgtcgccgccgccgccgacgtccacgcataa

SEQ ID NO: 129 - protein, 373 aa, Oryza sativa
MPTPSHLKNPLCFDFRAARRVPETHAWPGLDDHPVVDGGGGGGEDAVPVVDVGAGDAAARVARAAEQWGAFLLVGHGVPAALLSRVEERVARVFSLPASEKMRAVRGPGEPCGYGSPPISSFFSKLMWSEGYTFSPSSLRSELRRLWPKSGDDYLLFCDVMEEFHKEMRRLADELLRLFLRALGLTGEEVAGVEAERRIGERMTATVHLNWYPRCPEPRRALGLIAHTDSGFFTFVLQSLVPGLQLFRRGPDRWVAVPAVAGAFVVNVGDLFHILTNGRFHSVYHRAVVNRDRDRVSLGYFLGPPPDAEVAPLPEAVPAGRSPAYRAVTWPEYMAVRKKAFATGGSALKMVSTDAAAAADEHDDVAAAADVHA

SEQ ID NO: 130 - DNA, 2040 bp, Oryza sativa
actactcattccactattgtaaagtcatagaaaaaatttatatagagagaaaaaattagtgttgttattgttactggctttctgccagacgagacgagcgagcgcgcgagtgtgttgctctctggtcatcgtcgtcgtcgtcgcgatgccgacgccgtcgcacttgaagaacccgctctgcttcgacttccgggcggcgaggcgggtgccggagacgcacgcgtggccggggctggacgaccacccggtggtggacggcggcggcggcggcggcgaggacgcggtgccggtggtggacgtcggggcgggcgacgcggcggcgcgggtggcgcgggcggcggagcagtggggcgcgttccttctggtcgggcacggcgtgccggcggcgctgctgtcgcgcgtcgaggagcgcgtcgcccgcgtgttctccctgccggcgtcggagaagatgcgcgccgtccgcggccccggcgagccctgcggctacggctcgccgcccatctcctccttcttctccaagctcatgtggtccgagggctacaccttctccccttcctccctccgctccgagctccgccgcctctggcccaagtccggcgacgactacctcctcttctggtatatatacatatatactctcccatgcattccatgcacatacactctacgtatatatctacctctacgtatatatctacgtattgatctacgtataatatacgcagtgacgtgatggaggagtttcacaaggagatgcggcggctagccgacgagttgctgaggttgttcttgagggcgctggggctcaccggcgaggaggtcgccggagtcgaggcggagaggaggatcggcgagaggatgacggcgacggtgcacctcaactggtacccgaggtgcccggagccgcggcgagcgctggggctcatcgcgcacacggactcgggcttcttcaccttcgtgctccagagcctcgtcccggggctgcagctgttccgtcgagggcccgaccggtgggtggcggtgccggcggtggcgggggccttcgtcgtcaacgtcggcgacctcttccacatcctcaccaacggccgcttccacagcgtctaccaccgcgccgtcgtgaaccgcgaccgcgaccgggtctcgctcggctacttcctcggcccgccgccggacgccgaggtggcgccgctgccggaggccgtgccggccggccggagccccgcctaccgcgctgtcacgtggccggagtacatggccgtccgcaagaaggccttcgccaccggcggctccgccctcaagatggtctccaccgacgccgccgccgccgccgacgaacacgacgacgtcgccgccgccgccgacgtccacgcataagctatagctactagctacctcgatctcacgcaaaaaaaaaaagaaacaattaatagagcaaaaaaaaaaagaagagaaaatggtggtacttgtgtttaaggtttcctccatgcaaaatggtttgcatgcatgcatgcaaagctagcatctgcagctgcaagaattacaagagcagagaagcagacagctagatggagataattaattaattaattaatctaattaagcatgcaataattaagattattattctgatttcagaactgaaaaaaaaagtgtggttaattaattattggttaggcttaattttatctagatgtagaaaaagaatcaagatcttcaagcaagagagaagaggatcgaagaagaaggaaaagaaaacgaaaaggacatgctgtgttgtctcttctagttgtaccctggctgctgattaagtgctttgttttgttgctgcaagcttgtcgttactgattattagttagttatgcatctaattgattaaactaatctgtttggcattttggctcgagctaagctatagccaggctggtcaatgataggaacttgtacaatttaagcaattgaacctgatcctgtactggcatgtatgtatatatgcaagtgatgagaaccactagctagtatagctagacatgtatttgtata

SEQ ID NO: 131 - DNA, 1332 bp, Hordeum vulgare
acactcactcctcaatccatccgtctccaccattgctcgctagctcgagctctactagctagcactgcaaagtcagccgggccggagttgatttggtccttgttagcttgaccgatcgtatacgtatcgccaggatgccgacgccgtcgcacctgagcaaggacccgcactacttcgacttccgggcggcgcggcgggtgccggagacacacgcgtggccggggctgcacgaccacccggtggtggacggcggcggcgcgggcggagggccggacgcggtgccggtggtggacatgcgcgacccgtgcgccgcggaggcggtggcgctggccgcgcaggactggggcgccttcctcttgcagggccacggcgtcccgttggagctgctggcccgcgtggaggccgcgatagcgggcatgttcgcgctgccggcgtcggagaagatgcgcgccgtgcggcggcccggcgactcgtgcggctacgggtcgccgcccatctcctccttcttctccaagtgcatgtggtccgagggctacaccttctccccggccaacctccgctccgacctccgcaagctctggcccaaggccggccacgactaccgccacttctgtgccgtgatggaggagttccacagggagatgcgcgttctggccgacaagctgctggagctgttcctggtggccctcgggctcaccggcgagcaggtcgccgccgtcgagtcggagcacaagatcgccgagaccatgaccgccacaatgcacctcaactggtaccccaagtgcccggacccgaagcgagcgctgggcctgatcgcgcacacggactcgggcttcttcaccttcgtgctccagagcctggtgcccgggctgcagctgttccggcacggccccgaccgttgggtgacggtgcccgccgtgccgggcgccatggtcgtcaacgtcggcgacctcttccacatcctcaccaatggccgcttccacagcgtctaccaccgcgccgtcgtcaaccgcgacagcgaccggatatcgctggggtacttcctcggcccgcccgcccacgttaaggtggcgccgctcagggaggccctcgccggcacgcccgctgcctaccgcgccgtcacgtggcccgagtacatgggcgtgcgcaagaaggccttcaccaccggcgcctccgcgctcaagatggtcgccatctccaccgacgacgccgccgacgtcctccccgacgtcctctcgtcgtagatcggcgccggccatcacccggccggccaagagaccgatctatacaaacaattagtgaacaaaaaaaaaaaaaaaaaaaaaaaaaaaa

SEQ ID NO: 132 - DNA, 1110 bp, Hordeum vulgare
atgccgacgccgtcgcacctgagcaaggacccgcactacttcgacttccgggcggcgcggcgggtgccggagacacacgcgtggccggggctgcacgaccacccggtggtggacggcggcggcgcgggcggagggccggacgcggtgccggtggtggacatgcgcgacccgtgcgccgcggaggcggtggcgctggccgcgcaggactggggcgccttcctcttgcagggccacggcgtcccgttggagctgctggcccgcgtggaggccgcgatagcgggcatgttcgcgctgccggcgtcggagaagatgcgcgccgtgcggcggcccggcgactcgtgcggctacgggtcgccgcccatctcctccttcttctccaagtgcatgtggtccgagggctacaccttctccccggccaacctccgctccgacctccgcaagctctggcccaaggccggccacgactaccgccacttctgtgccgtgatggaggagttccacagggagatgcgcgttctggccgacaagctgctggagctgttcctggtggccctcgggctcaccggcgagcaggtcgccgccgtcgagtcggagcacaagatcgccgagaccatgaccgccacaatgcacctcaactggtaccccaagtgcccggacccgaagcgagcgctgggcctgatcgcgcacacggactcgggcttcttcaccttcgtgctccagagcctggtgcccgggctgcagctgttccggcacggccccgaccgttgggtgacggtgcccgccgtgccgggcgccatggtcgtcaacgtcggcgacctcttccacatcctcaccaatggccgcttccacagcgtctaccaccgcgccgtcgtcaaccgcgacagcgaccggatatcgctggggtacttcctcggcccgcccgcccacgttaaggtggcgccgctcagggaggccctcgccggcacgcccgctgcctaccgcgccgtcacgtggcccgagtacatgggcgtgcgcaagaaggccttcaccaccggcgcctccgcgctcaagatggtcgccatctccaccgacgacgccgccgacgtcctccccgacgtcctctcgtcgtag

SEQ ID NO: 133 - protein, 369 aa, Hordeum vulgare
MPTPSHLSKDPHYFDFRAARRVPETHAWPGLHDHPVVDGGGAGGGPDAVPVVDMRDPCAAEAVALAAQDWGAFLLQGHGVPLELLARVEAAIAGMFALPASEKMRAVRRPGDSCGYGSPPISSFFSKCMWSEGYTFSPANLRSDLRKLWPKAGHDYRHFCAVMEEFHREMRVLADKLLELFLVALGLTGEQVAAVESEHKIAETMTATMHLNWYPKCPDPKRALGLIAHTDSGFFTFVLQSLVPGLQLFRHGPDRWVTVPAVPGAMVVNVGDLFHILTNGRFHSVYHRAVVNRDSDRISLGYFLGPPAHVKVAPLREALAGTPAAYRAVTWPEYMGVRKKAFTTGASALKMVAISTDDAADVLPDVLSS

SEQ ID NO: 134 - DNA, 1653 bp, Triticum aestivum
  misc_feature (1594)..(1600): n is a, c, g, or t
  misc_feature (1641)..(1641): n is a, c, g, or t
cacgagatccatccgtctccaccattgctcgctagctcgagctcctagctagtactgcaaagtcagccggggagttgatttggtccttcttggcttgaccgatcgtacgtgccgccaggatgccgacgccggcgcacctgagcaaggacccgcgctacttcgacttccgggcggcgcggcgggtgccggagacgcacgcgtggcccgggctgcacgaccaccccgtggtggacggcagcggcgcgggcggagggccggacgcggtgccggtggtggacatgcgcgacccgtgcgcggcggaggcggtggcgctggcggcgcaggactggggcgccttcctcctggagggccacggcgtcccgttggagctgctggcgcgcgtggaggccgcgatcgcgggcatgttcgcgctgccggcgtcggagaagatgcgcgccgtgcggcggcccggcgactcgtgcggctacgggtcgccgcccatctcctccttcttctccaagtgcatgtggtccgagggctacaccttctccccggccaacctccgctccgacctccgcaagctctggcccaaggccggccacgactaccgccacttctgcgccgtgatggaggagttccacagggagatgcgcgcgctggccgacaagctgctggagctgttcctggtggccctcgggctcaccggcgagcaggtcgccgccgtcgagtccgagcagaagatcgccgagaccatgaccgccacaatgcacctcaactggtaccccaagtgcccggacccgaagcgggcgctgggcctgatcgcgcacacggactcgggcttcttcaccttcgtgctgcagagccttgtgcccgggctgcagctgttccggcacggccccgaccggtgggtgacggtgcccgccgtgccgggggccatggtcgtcaacgtcggcgacctcttccagatcctcaccaacggccgcttccacagcgtctaccaccgcgccgtcgtcaaccgcgacagcgaccggatatcgctcggctacttcctcggcccgcccgcccacgtcaaggtggcgccgctcagggaggccctggccggcacgcccgccgcctaccgcgccgtcacgtggcccgagtacatgggcgtgcgcaagaaggccttcaccaccggcgcctccgcgctcaagatggtcgccatctccactgacaacgacgccgccaaccacacggacgacctgatctcgtcgtagatcggcgccggccatcaccggccggccaagggatcgatctacacacacaattagtgaacaaaaaaatgccagagatggtgcatggtgggctggtagcttagctgaggtagctaggaggaagagcgcgcgtgcggctgtcgttcgtgcggctgttcccgcaaaaaaaaaaaaggtttcctccatatatgtctccatgcagaactgcagatgctggtggtggatgcgtccatgcagcagggaacgaactaattgtaagaaaatcaagcaaacttagttctacatctgtaattaagtatgcatgccacttggtttaattcaattcaagtgcagaaaaaattatgatgggaaaaaaaaagacatgnnnnnnnaaaaaaaaaaaaaaaaaaaaaaaaaaaaaaaaaaaaaaaanaaaaaaaaaaaa

SEQ ID NO: 135 - DNA, 1113 bp, Triticum aestivum
atgccgacgccggcgcacctgagcaaggacccgcgctacttcgacttccgggcggcgcggcgggtgccggagacgcacgcgtggcccgggctgcacgaccaccccgtggtggacggcagcggcgcgggcggagggccggacgcggtgccggtggtggacatgcgcgacccgtgcgcggcggaggcggtggcgctggcggcgcaggactggggcgccttcctcctggagggccacggcgtcccgttggagctgctggcgcgcgtggaggccgcgatcgcgggcatgttcgcgctgccggcgtcggagaagatgcgcgccgtgcggcggcccggcgactcgtgcggctacgggtcgccgcccatctcctccttcttctccaagtgcatgtggtccgagggctacaccttctccccggccaacctccgctccgacctccgcaagctctggcccaaggccggccacgactaccgccacttctgcgccgtgatggaggagttccacagggagatgcgcgcgctggccgacaagctgctggagctgttcctggtggccctcgggctcaccggcgagcaggtcgccgccgtcgagtccgagcagaagatcgccgagaccatgaccgccacaatgcacctcaactggtaccccaagtgcccggacccgaagcgggcgctgggcctgatcgcgcacacggactcgggcttcttcaccttcgtgctgcagagccttgtgcccgggctgcagctgttccggcacggccccgaccggtgggtgacggtgcccgccgtgccgggggccatggtcgtcaacgtcggcgacctcttccagatcctcaccaacggccgcttccacagcgtctaccaccgcgccgtcgtcaaccgcgacagcgaccggatatcgctcggctacttcctcggcccgcccgcccacgtcaaggtggcgccgctcagggaggccctggccggcacgcccgccgcctaccgcgccgtcacgtggcccgagtacatgggcgtgcgcaagaaggccttcaccaccggcgcctccgcgctcaagatggtcgccatctccactgacaacgacgccgccaaccacacggacgacctgatctcgtcgtag

SEQ ID NO: 136 - protein, 370 aa, Triticum aestivum
MPTPAHLSKDPRYFDFRAARRVPETHAWPGLHDHPVVDGSGAGGGPDAVPVVDMRDPCAAEAVALAAQDWGAFLLEGHGVPLELLARVEAAIAGMFALPASEKMRAVRRPGDSCGYGSPPISSFFSKCMWSEGYTFSPANLRSDLRKLWPKAGHDYRHFCAVMEEFHREMRALADKLLELFLVALGLTGEQVAAVESEQKIAETMTATMHLNWYPKCPDPKRALGLIAHTDSGFFTFVLQSLVPGLQLFRHGPDRWVTVPAVPGAMVVNVGDLFQILTNGRFHSVYHRAVVNRDSDRISLGYFLGPPAHVKVAPLREALAGTPAAYRAVTWPEYMGVRKKAFTTGASALKMVAISTDNDAANHTDDLISS

SEQ ID NO: 137 - DNA, 1884 bp, Triticum aestivum
tatatatacagctccttgtacttctctcgttcttacactcactcctcaatccatccgtctccaccattgctcgctagctcgagctcctagctagtactgcaaagtcagccggggagttgatttggtccttcttggcttgaccgatcgtacgtgccgccaggatgccgacgccggcgcacctgagcaaggacccgcgctacttcgacttccgggcggcgcggcgggtgccggagacgcacgcgtggcccgggctgcacgaccaccccgtggtggacggcagcggcgcgggcggagggccggacgcggtgccggtggtggacatgcgcgacccgtgcgcggcggaggcggtggcgctggcggcgcaggactggggcgccttcctcctggagggccacggcgtcccgttggagctgctggcgcgcgtggaggccgcgatcgcgggcatgttcgcgctgccggcgtcggagaagatgcgcgccgtgcggcggcccggcgactcgtgcggctacgggtcgccgcccatctcctccttcttctccaagtgcatgtggtccgagggctacaccttctccccggccaacctccgctccgacctccgcaagctctggcccaaggccggccacgactaccgccacttctggtacgtacgccggccgccgatgcgcatatacacgtcatagtacggcacctacctaactggctctggccaaccgtccgtacacacgtgaaggggcgacgtgtccgactccgaccatgcatgcatgcacgcgcgcgaaacttgttactcctgttctgctatggcagcagctagccgcgtgtgtccgttcgtaggagtagttacttacacagttacacttacgccgtccgtcgtgttcctcgacgtgcagcgccgtgatggaggagttccacagggagatgcgcgcgctggccgacaagctgctggagctgttcctggtggccctcgggctcaccggcgagcaggtcgccgccgtcgagtccgagcagaagatcgccgagaccatgaccgccacaatgcacctcaactggtacgttccactactactccagtagtacaagtacaatatatagaatacaaatggcagcagccacgacgacacgtactccaccatgcagcaaagcatatattgtcggtgcggcggttgacacggagttgtgtcgtgtcgttgattcacaggtaccccaagtgcccggacccgaagcgggcgctgggcctgatcgcgcacacggactcgggcttcttcaccttcgtgctgcagagccttgtgcccgggctgcagctgttccggcacggccccgaccggtgggtgacggtgcccgccgtgccgggggccatggtcgtcaacgtcggcgacctcttccagatcctcaccaacggccgcttccacagcgtctaccaccgcgccgtcgtcaaccgcgacagcgaccggatatcgctcggctacttcctcggcccgcccgcccacgtcaaggtggcgccgctcagggaggccctggccggcacgcccgccgcctaccgcgccgtcacgtggcccgagtacatgggcgtgcgcaagaaggccttcaccaccggcgcctccgcgctcaagatggtcgccatctccactgacaacgacgccgccaaccacacggacgacctgatctcgtcgtagatcggcgccggccatcaccggccggccaagggatcgatctacacacacaattagtgaacaaaaaaatgccagagatggtgcatggtgggctggtagcttagctgaggtagctaggaggaagagcgcgcgtgcggctgtcgttcgtgcggctgttcccgcaaaaaaaaaaaggtttcctccatatakgtccccakscaaaatsgmaawgctgggg

SEQ ID NO: 138 - RNA, 20 nt, Artificial Sequence
  suppression oligo
acggguucuuccaggugugc

SEQ ID NO: 139 - RNA, 20 nt, Artificial Sequence
  suppression oligo
cacggguucuuccaggugug

SEQ ID NO: 140 - RNA, 20 nt, Artificial Sequence
  suppression oligo
cauugaccuccccgcuggca

SEQ ID NO: 141 - RNA, 20 nt, Artificial Sequence
  suppression oligo
ccagcggggaggucaaugcu

SEQ ID NO: 142 - RNA, 20 nt, Artificial Sequence
  suppression oligo
cccagcauugaccuccccgc

SEQ ID NO: 143 - RNA, 20 nt, Artificial Sequence
  suppression oligo
cgcgcucguguacccggaca

SEQ ID NO: 144 - RNA, 20 nt, Artificial Sequence
  suppression oligo
cucccggcgcaggucgaaca

SEQ ID NO: 145 - RNA, 20 nt, Artificial Sequence
  suppression oligo
guguacccggacacggugcc

SEQ ID NO: 146 - RNA, 20 nt, Artificial Sequence
  suppression oligo
ugcagggaagcuguccgggc

SEQ ID NO: 147 - RNA, 20 nt, Artificial Sequence
  suppression oligo
uucuuccaggugugcgggca

SEQ ID NO: 148 - RNA, 20 nt, Artificial Sequence
  suppression oligo
agauccccgcgccauuccug

SEQ ID NO: 149 - RNA, 20 nt, Artificial Sequence
  suppression oligo
augcagggaagcuguccggg

SEQ ID NO: 150 - RNA, 20 nt, Artificial Sequence
  suppression oligo
auuccuguggccgcaggaag

SEQ ID NO: 151 - RNA, 20 nt, Artificial Sequence
  suppression oligo
cagcggggaggucaaugcug

SEQ ID NO: 152 - RNA, 20 nt, Artificial Sequence
  suppression oligo
caggaauggcgcggggaucu

SEQ ID NO: 153 - RNA, 20 nt, Artificial Sequence
  suppression oligo
gacuacuucgucggcacccu

SEQ ID NO: 154 - RNA, 20 nt, Artificial Sequence
  suppression oligo
gccaggauuucgagccaaug

SEQ ID NO: 155 - RNA, 20 nt, Artificial Sequence
  suppression oligo
ggaacauuuggagggaggcg

SEQ ID NO: 156 - RNA, 20 nt, Artificial Sequence
  suppression oligo
gggaggucaaugcuggggcu

SEQ ID NO: 157 - RNA, 20 nt, Artificial Sequence
  suppression oligo
uuggcucgaaauccuggccg

SEQ ID NO: 158 - RNA, 20 nt, Artificial Sequence
  suppression oligo
acggguucuuccaggugugc

SEQ ID NO: 159 - RNA, 20 nt, Artificial Sequence
  suppression oligo
cacggguucuuccaggugug

SEQ ID NO: 160 - RNA, 20 nt, Artificial Sequence
  suppression oligo
cauugaccuccccgcuggca

SEQ ID NO: 161 - RNA, 20 nt, Artificial Sequence
  suppression oligo
ccagcggggaggucaaugcu

SEQ ID NO: 162 - RNA, 20 nt, Artificial Sequence
  suppression oligo
cccagcauugaccuccccgc

SEQ ID NO: 163 - RNA, 20 nt, Artificial Sequence
  suppression oligo
cgcgcucguguacccggaca

SEQ ID NO: 164 - RNA, 20 nt, Artificial Sequence
  suppression oligo
cucccggcgcaggucgaaca

SEQ ID NO: 165 - RNA, 20 nt, Artificial Sequence
  suppression oligo
guguacccggacacggugcc

SEQ ID NO: 166 - RNA, 20 nt, Artificial Sequence
  suppression oligo
ugcagggaagcuguccgggc

SEQ ID NO: 167 - RNA, 20 nt, Artificial Sequence
  suppression oligo
uucuuccaggugugcgggca

SEQ ID NO: 168 - DNA, 2488 bp, Zea Mays
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 169 - DNA, 2473 bp, Zea Mays
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 170 - DNA, 30 bp, Artificial Sequence
  mutant sequence by gene editing
tcgtggactacttcaccaagatttcgagcc

SEQ ID NO: 171 - DNA, 30 bp, Artificial Sequence
  mutant sequence by gene editing
actacttcaccggcacctcggccaagattt

SEQ ID NO: 172 - DNA, 32 bp, Artificial Sequence
  mutant sequence by gene editing
actacttcaccggcaccccctcggccaagatt

SEQ ID NO: 173 - DNA, 30 bp, Artificial Sequence
  mutant sequence by gene editing
gactacttcaccggcccctcggccaagatt

SEQ ID NO: 174 - DNA, 30 bp, Artificial Sequence
  mutant sequence by gene editing
ggactacttcaccggcctcggccaagattt

SEQ ID NO: 175 - DNA, 30 bp, Artificial Sequence
  mutant sequence by gene editing
tccccggcaccgtgtgggtacacgagcgcg

SEQ ID NO: 176 - DNA, 31 bp, Artificial Sequence
  mutant sequence by gene editing
tccccggcaccgtgtaccgggtacacgagcg

SEQ ID NO: 177 - DNA, 31 bp, Artificial Sequence
  mutant sequence by gene editing
tccccggcaccgtgttccgggtacacgagcg

SEQ ID NO: 178 - DNA, 30 bp, Artificial Sequence
  mutant sequence by gene editing
ccaagatttcgagccgtggggtgagtaaag

SEQ ID NO: 179 - DNA, 32 bp, Artificial Sequence
  mutant sequence by gene editing
ccaagatttcgagccaagtggggtgagtaaag

SEQ ID NO: 180 - DNA, 32 bp, Artificial Sequence
  mutant sequence by gene editing
caagatttcgagccacgtggggtgagtaaaga

SEQ ID NO: 181 - DNA, 32 bp, Artificial Sequence
  mutant sequence by gene editing
cagcattgacctcccccgctggcaaggacagg

SEQ ID NO: 182 - DNA, 47 bp, Artificial Sequence
  mutant sequence by gene editing
ccgcggcgcccgtcgtcgtggactacttcaccaagatttcgagccag

SEQ ID NO: 183 - DNA, 59 bp, Artificial Sequence
  mutant sequence by gene editing
ccgcggcgcccgtcgtcgtggactacttcaccggcacctcggccaagatttcgagccag

SEQ ID NO: 184 - DNA, 62 bp, Artificial Sequence
  mutant sequence by gene editing
ccgcggcgcccgtcgtcgtggactacttcaccggcaccccctcggccaagatttcgagccag

SEQ ID NO: 185 - DNA, 59 bp, Artificial Sequence
  mutant sequence by gene editing
ccgcggcgcccgtcgtcgtggactacttcaccggcccctcggccaagatttcgagccag

SEQ ID NO: 186 - DNA, 57 bp, Artificial Sequence
  mutant sequence by gene editing
ccgcggcgcccgtcgtcgtggactacttcaccggcctcggccaagatttcgagccag

SEQ ID NO: 187 - DNA, 58 bp, Artificial Sequence
  mutant sequence by gene editing
ccggcgcgtccccggcaccgtgtgggtacacgagcgcgcacgccgaccggttcgcgtc

SEQ ID NO: 188 - DNA, 61 bp, Artificial Sequence
  mutant sequence by gene editing
ccggcgcgtccccggcaccgtgtaccgggtacacgagcgcgcacgccgaccggttcgcgtc

SEQ ID NO: 189 - DNA, 61 bp, Artificial Sequence
  mutant sequence by gene editing
ccggcgcgtccccggcaccgtgttccgggtacacgagcgcgcacgccgaccggttcgcgtc

SEQ ID NO: 190 - DNA, 59 bp, Artificial Sequence
  mutant sequence by gene editing
ctacttcaccggcaccctcggccaagatttcgagccgtggggtgagtaaagaagaagat

SEQ ID NO: 191 - DNA, 61 bp, Artificial Sequence
  mutant sequence by gene editing
ctacttcaccggcaccctcggccaagatttcgagccaagtggggtgagtaaagaagaagat

SEQ ID NO: 192 - DNA, 61 bp, Artificial Sequence
  mutant sequence by gene editing
ctacttcaccggcaccctcggccaagatttcgagccacgtggggtgagtaaagaagaagat

SEQ ID NO: 193 - DNA, 61 bp, Artificial Sequence
  mutant sequence by gene editing
tccccctccgcgccccaactcccagcattgacctcccccgctggcaaggacagggccgacg

SEQ ID NO: 194 - DNA, 60 bp, Zea mays
ccgcggcgcccgtcgtcgtggactacttcaccggcaccctcggccaagatttcgagccag

SEQ ID NO: 195 - DNA, 60 bp, Zea mays; identical to SEQ ID NO: 194

SEQ ID NO: 196 - DNA, 60 bp, Zea mays; identical to SEQ ID NO: 194

SEQ ID NO: 197 - DNA, 60 bp, Zea mays; identical to SEQ ID NO: 194

SEQ ID NO: 198 - DNA, 60 bp, Zea mays; identical to SEQ ID NO: 194

SEQ ID NO: 199 - DNA, 60 bp, Zea mays
ccggcgcgtccccggcaccgtgtccgggtacacgagcgcgcacgccgaccggttcgcgtc

SEQ ID NO: 200 - DNA, 60 bp, Zea mays; identical to SEQ ID NO: 199

SEQ ID NO: 201 - DNA, 60 bp, Zea mays; identical to SEQ ID NO: 199

SEQ ID NO: 202 - DNA, 60 bp, Zea mays
ctacttcaccggcaccctcggccaagatttcgagccagtggggtgagtaaagaagaagat

SEQ ID NO: 203 - DNA, 60 bp, Zea mays; identical to SEQ ID NO: 202

SEQ ID NO: 204 - DNA, 60 bp, Zea mays; identical to SEQ ID NO: 202

SEQ ID NO: 205 - DNA, 60 bp, Zea mays
tccccctccgcgccccaactcccagcattgacctccccgctggcaaggacagggccgacg

SEQ ID NO: 206 - DNA, 2475 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 207 - DNA, 2487 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcacctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 208 - DNA, 2490 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 209 - DNA, 2487 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 210 - DNA, 2485 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 211 - DNA, 2486 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 212 - DNA, 2489 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtaccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 213 - DNA, 2489 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgttccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 214 - DNA, 2487 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccgtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 215 - DNA, 2489 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccaagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 216 - DNA, 2489 bp, Artificial Sequence
  mutant sequence by gene editing
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccacgtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 217 - DNA, 2489 bp, Artificial Sequence
  mutant sequence by gene editing
atggacgccagcccgaccccaccgctccccctccgcgccccaactcccagcattgacctcccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 218 - DNA, 30 bp, Artificial Sequence
  mutant sequence by gene editing
tccccggcaccgtgtcgggtacacgagcgc

SEQ ID NO: 219 - DNA, 30 bp, Artificial Sequence; identical to SEQ ID NO: 175
  mutant sequence by gene editing

SEQ ID NO: 220 - DNA, 30 bp, Artificial Sequence
  mutant sequence by gene editing
tccccggcaccgtgttccgggtacacgagc

SEQ ID NO: 221 - DNA, 30 bp, Artificial Sequence
  mutant sequence by gene editing
gatccccgcgccattctgtggccgcaggaa

SEQ ID NO: 222 - DNA, 30 bp, Artificial Sequence
  mutant sequence by gene editing
ccaggatttcgagccatggggtaagtaagg

SEQ ID NO: 223 - DNA, 31 bp, Artificial Sequence
  mutant sequence by gene editing
tcggccaggatttcgatggggtaagtaaggt

SEQ ID NO: 224 - DNA, 32 bp, Artificial Sequence
  mutant sequence by gene editing
atacccccacccgccgacagcttccctgcata

SEQ ID NO: 225 - DNA, 33 bp, Artificial Sequence
  mutant sequence by gene editing
atacccccacccgccctggacagcttccctgca

SEQ ID NO: 226 - DNA, 32 bp, Artificial Sequence
  mutant sequence by gene editing
aaatacccccacccgggacagcttccctgcat

SEQ ID NO: 227 - DNA, 31 bp, Artificial Sequence
  mutant sequence by gene editing
cagcattgacctcccacgctggcaaggacaa

SEQ ID NO: 228 - DNA, 31 bp, Artificial Sequence
  mutant sequence by gene editing
cagcattgacctcccccgctggcaaggacaa

SEQ ID NO: 229 - DNA, 59 bp, Artificial Sequence
  mutant sequence by gene editing
ccggcgcgtccccggcaccgtgtcgggtacacgagcgcgcacgccgaccggttcgcggc

SEQ ID NO: 230 - DNA, 58 bp, Artificial Sequence
  mutant sequence by gene editing
ccggcgcgtccccggcaccgtgtgggtacacgagcgcgcacgccgaccggttcgcggc

SEQ ID NO: 231 - DNA, 61 bp, Artificial Sequence
  mutant sequence by gene editing
ccggcgcgtccccggcaccgtgttccgggtacacgagcgcgcacgccgaccggttcgcggc

SEQ ID NO: 232 - DNA, 59 bp, Artificial Sequence
  mutant sequence by gene editing
cgacctgcgccgggagcccaagatccccgcgccattctgtggccgcaggaagaggcgcg

SEQ ID NO: 233 - DNA, 59 bp, Artificial Sequence
  mutant sequence by gene editing
ctacttcgtcggcaccctcggccaggatttcgagccatggggtaagtaaggtagtaaga

SEQ ID NO: 234 - DNA, 55 bp, Artificial Sequence
  mutant sequence by gene editing
ctacttcgtcggcaccctcggccaggatttcgatggggtaagtaaggtagtaaga

SEQ ID NO: 235 - DNA, 58 bp, Artificial Sequence
  mutant sequence by gene editing
gttacaaatacccccacccgccgacagcttccctgcatacttgcagctcgcacatctc

SEQ ID NO: 236 - DNA, 61 bp, Artificial Sequence
  mutant sequence by gene editing
gttacaaatacccccacccgccctggacagcttccctgcatacttgcagctcgcacatctc

SEQ ID NO: 237 - DNA, 57 bp, Artificial Sequence
  mutant sequence by gene editing
gttacaaatacccccacccgggacagcttccctgcatacttgcagctcgcacatctc

SEQ ID NO: 238 - DNA, 61 bp, Artificial Sequence
  mutant sequence by gene editing
tccgcgcccccactcccagccccagcattgacctcccacgctggcaaggacaaggccgacg

SEQ ID NO: 239 - DNA, 61 bp, Artificial Sequence
  mutant sequence by gene editing
tccgcgcccccactcccagccccagcattgacctcccccgctggcaaggacaaggccgacg

SEQ ID NO: 240 - DNA, 60 bp, Zea mays
ccggcgcgtccccggcaccgtgtccgggtacacgagcgcgcacgccgaccggttcgcggc

SEQ ID NO: 241 - DNA, 60 bp, Zea mays; identical to SEQ ID NO: 240

SEQ ID NO: 242 - DNA, 60 bp, Zea mays; identical to SEQ ID NO: 240

SEQ ID NO: 243 - DNA, 60 bp, Zea mays
cgacctgcgccgggagcccaagatccccgcgccattcctgtggccgcaggaagaggcgcg

SEQ ID NO: 244 - DNA, 60 bp, Zea mays
ctacttcgtcggcaccctcggccaggatttcgagccaatggggtaagtaaggtagtaaga

SEQ ID NO: 245 - DNA, 60 bp, Zea mays; identical to SEQ ID NO: 244

SEQ ID NO: 246 - DNA, 60 bp, Zea mays
gttacaaatacccccacccgcccggacagcttccctgcatacttgcagctcgcacatctc

SEQ ID NO: 247 - DNA, 60 bp, Zea mays; identical to SEQ ID NO: 246

SEQ ID NO: 248 - DNA, 60 bp, Zea mays; identical to SEQ ID NO: 246

SEQ ID NO: 249 - DNA, 60 bp, Zea mays
tccgcgcccccactcccagccccagcattgacctccccgctggcaaggacaaggccgacg

SEQ ID NO: 250 - DNA, 60 bp, Zea mays; identical to SEQ ID NO: 249

SEQ ID NO: 251 - DNA, 2472 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtcgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 252 - DNA, 2471 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 253 - DNA, 2474 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgttccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 254 - DNA, 2472 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 255 - DNA, 2472 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 256 - DNA, 2468 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 257 - DNA, 2471 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgccgacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 258 - DNA, 2474 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgccctggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 259 - DNA, 2470 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 260 - DNA, 2474 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctcccacgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 261 - DNA, 2474 bp, Artificial Sequence
  mutant sequence by gene editing
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctcccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag